SEQUENCE LISTING (50 sequences)

SEQ ID NO: 1 - DNA, 231 bp, synthetic construct
  misc_feature 1..231: note = Description of sequence: Based on Lactobacillus reuteri sequence
  source 1..231: mol_type = other DNA
atggacaaagaagaattagaaaaaattgtaggtaataactttgaggaaatgagtttacaaaaaatgacagaaattcaaggtatgggtgaataccaagtggattcaacaccagcagcttctgcgatttcacgggcaacaattcaagtatcacgtgcatcttctggaaaatgtctaagttggggtagtggtgcagcatttagtgcttattttactcataaaagatggtgctag

SEQ ID NO: 2 - protein, 76 aa, synthetic construct
  REGION 1..76: note = Description of sequence: Based on Lactobacillus reuteri sequence
MDKEELEKIVGNNFEEMSLQKMTEIQGMGEYQVDSTPAASAISRATIQVSRASSGKCLSWGSGAAFSAYFTHKRWC

SEQ ID NO: 3 - DNA, 186 bp, synthetic construct
  misc_feature 1..186: note = Description of sequence: Based on Lactobacillus reuteri sequence
  source 1..186: mol_type = other DNA
atggaagaaaaagaattagaaggtgtaatagggaattcgtttgaaagtatgactgtagaggaaatgacaaaaattcaaggtatgggtgaatatcaagtagattcgacgcctggatattttatggaaagtgctgccttttcagctcttacagccaatataacaagacatgctatgcatcatcattaa

SEQ ID NO: 4 - protein, 61 aa, synthetic construct
  REGION 1..61: note = Description of sequence: Based on Lactobacillus reuteri sequence
MEEKELEGVIGNSFESMTVEEMTKIQGMGEYQVDSTPGYFMESAAFSALTANITRHAMHHH

SEQ ID NO: 5 - protein, 373 aa, synthetic construct
  REGION 1..373: note = Description of sequence: Based on Lactobacillus reuteri sequence
MVEIAHFGVEAWLNKWEKSATYDISQSTIASLSMHDLLNLDGNNGEEFYEMLDKQQMNYGWIEGSPEFKEEVAKLYHHVDPENILQTNGATGANILALYALINPGDHVIAEYPSYQQLYDIPKSLGADVDYWHIHEEDNWYPRIDDLKAMVKPNTKMICLNNANNPTGTVLDKEFLEQVVEIAKSVDAYVLVDEVYLPLDHPEKFAQIIDLYDKGISTNSLSKTYSVPGVRIGWTATNAEVADIFRKFRDYTMICGGVFNDQLATYVLRHRDQVLARNRKLVLGNLAIYKDWIDHEDRASVIMPQAVSTSFPKLDVPVDIHTFCENLLHDEGVLLVPGDAFDTPGHVRLGYCAPEATLKEGLKRLSKYMHQYD

SEQ ID NO: 6 - protein, 178 aa, synthetic construct
  REGION 1..178: note = Description of sequence: Based on Lactobacillus reuteri sequence
MILTTFIILILMGCFINGHRRGLLTMTLMLGTYIVAWIVARQGAQLIGGWLKSLLPSIGTPATFSESLLANVNSNLFFYNGIAFMIIFTIVSILCHWGIRQLNWIKRIPVVGTVDKIAGGLISFLIGYLIIYVVLLIMQLFPAGWWQMQIANSELARFMINQTPGIAHLVIDTLVQGG

SEQ ID NO: 7 - DNA, 4653 bp, synthetic construct
  misc_feature 1..4653: note = Description of sequence: Based on Lactobacillus reuteri sequence
  source 1..4653: mol_type = other DNA
ttggcaatctttcacctatcagcaaaaatcattagtcgaggaaaagggcaatcagcaattgcctcggctgcttatcgttcagggaacaaacttcacgatgaacgatacgacgaaacacaagattacacaaacaaacgtttcatcgaacactcagaaatccaacttccagagaatgccccagctaagtatcaagatagggcaaccctttggaacagcgtagaaaaggcagaaaaagctaagaattcgcagttagctagagaaattgaaatagccttaccacgagagctaacaccagaacaacgagttaaattagtccacgattacgttcagaaaacctttgtcgataagggaatggtagccgattggtctattcacaacccacaaccagataaggataatccagaaaagccagcaaacccgcacgctcatatcatgctcacactgcgtagcttgcgttctaacggctcttgggcaccaaagaagacaagtcactatgaactagacgaaaacggccagaaggtgcctgtaatcgatcctgagacaggcaaacagaagttaggggcgaaaaaccaaaaaatctggaaacgagtaattaccccaactaatgactggaataaccccaaaaacgttgaaaaatggcgggctgaatgggcgaagacttgtaataagtacttggctcctgaccaccagattgaccaccgcagttataaacggcagggcaaaaaacaaattccaacaatccatgagggctatgttgctcgtaaaatggaacgagaagccacaggaagctcagagagggcttcttttaaccaaatcgtaaaatatatcaacaacgagttaaaatcgcttagaaagcaaattaggggcattatacgcgaaatacggaatctagaaaaaggacgtgaccaagatgagaaagttcgacttcagcaagacacccgagcaaatcaaacaagaacaagcagagatcaagcgctgggaggaggagttcaacgcacgattacagacaatcgggagcaatccggaaacggtagagccaacggaactgccgaagaagcccaacgaacagatcctaatcaacttcatcaacagtctttcggagaaacagcaagaagaattttccgagatttccggctatttgcggagcgtcaacgagaagctcgacagcgacaacgccgacttagccaaggcttacaacatcatcaagaaacagaaaaacgacttgaagcagggcaacaacaacttactcgaacaaaacatacagctgacgaacgaaaacaacagcttacagaacgacaaattcgagttagaaaagcttttagcgacgcttctaagtccaaagtcttggaacgatactacactcacttccgtcaaacaaaaactcaagcaaatcatgcaaaaggattagactaccaaggaccaattctgggacattcccgaggacgctcaaggtagttatgctagaatgattaagccgtccgtggcaactcctacgtaaaggaggtggcaacatggcacatatactcctctcagcagttttgagccttctgggttccttgataactgcactgttcgctgattggcttcgtcggcgaaaatagtttttgacggcatgaagcgcaaattaaaagccactcaactattcgcactagttgggtggcttttttgcaacgtggcaatatacttctctcggttaaattataacatggttgttccagaagtgcattaggtaggcggtttgtccccccccatgggttttggcttcgaaaatcaatcgttgaagtgaccagcgaagctcagctgatggagccctcatattcaagaagcaggtcaacgaatgccaattccaacatgagggccatgacttaggccactgatactcgcaaatgactgtcacaccaacggtattagccattctgataagcgagtctaacgacttttgacgcataatcgaccacctaccttattttcaaagcgcctcagagctaattagagtattcttcaaggagcttagcgaggattatagacgcttggcggacttcactttcggcttgattgacccgtttagcgatcttgtcatcatggcgagccaccttttccaagtcatggagattatcagcgatttggtcagtcatgacggagtagttcttaatacccgacagcaggctttcaacgttagtattctcaaggttttcgttgaatatcatggttattcctccttatcctcaaacttagtcatcttctgtctcctgttttagagaattttcaatcatgtctaataatgttgacatgtcaatgttaaatgaatcaaggctaccataccgcttgatttgagttttaacactatccaatccatattcttcaagtaatgcttttaatttaccagtaactttttcacctaaatgcccaaaatactgaagccagctcttaagtaattctcggtactggtcaaatttttctttttcagtaagctgtttctcaacttcttcttggtcctcagaaccgttaactactttccagaggtcagggtcagtagtacccaacttataaccaagtacccgatcatatgctctggccttttcctttggagtcaaatcaggattaaggtcaatattatctagcttcatacgttggtcagcataacgacctttttcaaagtcatcggcattcttggcttcaggttgccaagtgaactcatagccaacaacaggacgtcctctaccagctcctcgaatagtttttacggttaagccacgaataacaggggttagctcttctttaattggacgtaacactctttttcttacgttaccctggtctttcttgtagctcttgggtagatctagttgcttaaataattcgtcactggttaacttcaaccgccctactgttcgatattgtttaattaacactctttttcttacgttaccctggtctttcttgtagctcttgggtagatctagttgcttaaataattcgtcactggttaacttcaaccgccctactgttcgatattgtttaatcaagcgaaacatgttcttagcataagcagattgaagactattaaactgagctagttgaaatcttgtccaatgacttaaatcattgaatagcttctgaaatagagggttaacttgaacagtcagaatctgtttactcctctgaatctgaaatacattccaacaagcaaattgagtaatggtgtctccatcatcggtgtaggcattaatctctaatagctttttattagttttcatcaaatcttttacaaaattagatgtactccgttcatacttactaagactttgtagttgcgtaaatgaaaactttaatttctgagtacccttttgatacgactgctgaactagtgtaataaatagatttagctcattagagttcagtcggccaagtggaatagtattaagccgattactatacttcacaatttcattactcaattttctcacctcaaatatattataacattttatcgtacacaatcaagagtacgtacttatagcacacgtacagtattagcgcgtatattaagacaaaaatttagacaaaatgtaccttataacggacaaaatgtaccttataaaccggacaaaatgtaccttataacggacaaaatgtaccttataaaaaacgaaactcttactctcccaaggagtttcaaacccctaaagagtatttaaagaatatatataaagatatttaaagaggcaccatacggaaaatctcctttctgatttcaatttcagagaggagattatcactaaatttcaaaattcaatttttcgccaaaaacttttttagtagttttcggtaactagcaaaatcaacttcgttgattattttgactatttcgaaattcatcaaaaactgaatgcccgtctttatagtgaatttgtttagtgaagttcactaactgcttcagcatattcagttgaccacgtaaccattgttcactatcagcaggactaatttgttctgataattgctctttttggtggttataagccttataagcctgatatttagcataaccttgtgggtcatctgatttagccactctagccactttaggtacttttctaagccccttagcttgagcttgaatatcttctaaaaccgcccctttttcgatgagacgcttatttcgttgctttctcttttcagtattgatgcgagccatttgacgctctcgacgagccttaaacactgcttccttttcttgaaggcgttcaagttctgctttcattttttcaatatcagccataacaaatcctcctaatgtgaataatgttttctgaaattcaaatgttatctagattatatgatgttacaattgatttaaccactacgtttggtctccctgttaagggcgcacttatacacattcaaagaatgtaattacgcctgacggcgacgtgcgctctccgaggggacttacccagtcggggcgacttcatcgagaccatgtcatggagattcagtttcaccgaaacggaccactaaccccgtggaggggagaagtttcacttctagggaccattccatggagggtcgctttttacaaaagcaacgcaaaacaaaagactagctcccaagcttacgcaaggtcgttagtccctaaggaggcagcacaga

SEQ ID NO: 8 - DNA, 1026 bp, synthetic construct
  misc_feature 1..1026: note = Description of sequence: Based on Lactobacillus reuteri sequence
  source 1..1026: mol_type = other DNA
ttagtcatcttctgtctcctgttttagagaattttcaatcatgtctaataatgttgacatgtcaatgttaaatgaatcaaggctaccataccgcttgatttgagttttaacactatccaatccatattcttcaagtaatgcttttaatttaccagtaactttttcacctaaatgcccaaaatactgaagccagctcttaagtaattctcggtactggtcaaatttttctttttcagtaagctgtttctcaacttcttcttggtcctcagaaccgttaactactttccagaggtcagggtcagtagtacccaacttataaccaagtacccgatcatatgctctggccttttcctttggagtcaaatcaggattaaggtcaatattatctagcttcatacgttggtcagcataacgacctttttcaaagtcatcggcattcttggcttcaggttgccaagtgaactcatagccaacaacaggacgtcctctaccagctcctcgaatagtttttacggttaagccacgaataacaggggttagctcttctttaattggacgtaacactctttttcttacgttaccctggtctttcttgtagctcttgggtagatctagttgcttaaataattcgtcactggttaacttcaaccgccctactgttcgatattgtttaattaacactctttttcttacgttaccctggtctttcttgtagctcttgggtagatctagttgcttaaataattcgtcactggttaacttcaaccgccctactgttcgatattgtttaatcaagcgaaacatgttcttagcataagcagattgaagactattaaactgagctagttgaaatcttgtccaatgacttaaatcattgaatagcttctgaaatagagggttaacttgaacagtcagaatctgtttactcctctgaatctgaaatacattccaacaagcaaattgagtaatggtgtctccatcatcggtgtaggcattaatctctaatagctttttattagttttcat

SEQ ID NO: 9 - protein, 332 aa, synthetic construct
  REGION 1..332: note = Description of sequence: Based on Lactobacillus reuteri sequence
MNEYNAEMAKLNQGANAPVITTNSVNQALSLKPENNATVDIEALNPRITFKRVEEGTKYAGYQIFDKNNAYVNNIDGEFLRVTYTNLKNSTYKGSKISKIVVTYSDSTPTGNRITQSGLNAVTEGANDNFLVVFEDPVRGDMHSTTVTATYQYYDANGNLIDFSGTNNAWLSVGSLNFDQGNDYQGGKNEGNPTSGISEGVKLISGAQIKQLAGSSISVHDDGWAYAGFNNYSGTGMNNGINTDNGGSGWDMDGSPNAYYGAIVFQLTGSSVSLRQGLVSWGGADIASQYNNQFLNNAWFTAGTTLPETQIKQPIRKTSETHYHYNPSVIRL

SEQ ID NO: 10 - protein, 2059 aa, synthetic construct
  REGION 1..2059: note = Description of sequence: Based on Lactobacillus reuteri sequence
MAQKLMSANSTDKNFKMYKSKKSWVFAYSTTLALAAVAGITLSTTNVHADTTNGGDNQVNATAVTQNTTSNTVDQIAANTAQTDNTSTSINIRSLMDDLASGDDTSSSQNGQEQSQNYASSNQNSQTQQENGTTGQSTASQNGTTSDQTNSDQSDKNYYVISTRDLDKNGNVNYLTQKNYTSIKGQEVADGTVVTWPLSVSALPANRAQDLKSHVISETLDPHLEYLHYRAYLTNTDGTVTDVTNHVNLNRSGQTLIFTDDNYLLSIYNNNRYRVQNLPVIKLVTKANGNGYIIPNAFKSSYVFNDGSHDVSFTTTSNNVQIKTFNPGNSKDVEIGGNVQGDPSGTINGQVVADGSVVTWPMSVGDLPANRAQDVLSHIETDTLYNGLNYEGYHAYLPQADGSFQDVSSHINVQQNGQDLTFIADDYLIGLYNQDKSTAFKMPIIDLITSVHGTSIIAPNKFNSQLAFKDGNGQTVINNTSNQVQISTYHPTNTKDVELGGNVQGDTPNSINDKVVANGAIVTWPMASSELPANRVQDLQSRVISETLDSHLQYQGYKAWLQNADGKYTDVTSHVKLTQDGQNLTFADDEYLLNLYNSNKGTAYKLPIIDLVTKVNGAGITAPNSYTTKYVYSDGDGNTTINVTSNTVKISTFNPTTNKDVELGDNIHGDTESSIAGKLVSEGTIVTWPLSTSDLPANRAQDVVSHTAVDALEPTLQYISYTAWLPDSNGQLQDVTSHVKMTRDGQKLTFTDDDYLIGLYNQNKDIALKMPIIDLVTKATGNTKLLPNSFDSQFVYNDVDGNTIINVSSNKPTVETFDPTVHKDVELGGNNVQGDTPNSIDGKIVAQGTVVTWPMSTSDLPANRTQDVVSHSTSETLNQNLQYVGYHAYMPDANGKLQDVTSHVQLQQNGQNLVFTDDSYLINLYNQDKSIAFKMPIIDLMTKAISDSATIPNTFESQYVFNDGNGNTTFKSTSNTVQIITYKPKTTKDVELGDNIHGDTNASIAGQMITDGTVVTWPMSTSDLPANRTQDLQQHVVTDNLNDNLIFQGYTAWLPTANGLVDVTNHIELTRDGQNLTFTDDAYLLNLYNQNKDTAYKLPIIDLVTKANGNTKLIPNNFDSMFVYNDGDQQTTVNVTSNTVNISTYDPTATKDVELGDDIEGDTADTINNLMVQIGTKMTYPLTVSDLPANRADEITAHQSVDTLSDYLEYQGYKAYLPDADGKLQDITEHVNLKREGQKLSFNDDDYLINLYNNSKATKQALPVIDLVAKVTGSNDGKKVHIIPNHFDSTITTKDGKINTTSNTVVINSNDPEAVKDVELGDNVVGDTPNSVTGTTVADGTIVTWPMSVGSLGANRAQNVIKHTETENLDSGLTYLSFKAYLPDADGKMQDITEHINIQQDGQKLVFTDDDYLISLYNKDKSQRFALPVIDLVTRVNGDNKIIPNTFVSQFTFNDGKGNTITSVTSNQVNVSTFKSNPEKHVTLGTDIEGDDAENADGTVVAQGSEVTWPLSDKSPLPANRSQDVKSHTLVDKLDDNLQYNSYKAYLKGTDGKLQDVTDHIKLTRDGQNLTFIDDDYLLDLYNKDKSTAFNLPIIDLVTTVVGNDKLIPNKFDSNFVFSDGNKDTSMKTTSNEVSISTYTPVTNKDAELGDNVVGDTSDSIANETVPDGTIVTWPLSVSSLPANRSQDVFKHVIEDILDGNLTYNSFKAYLKDAAGNLQEVTDHVKLAQEGQHLTFTDDDYLINLYNSSKNKEQSLPIIDLVTTVHGDSKLIPNEFDNVFVFKDGKGQTTVKTTSNKVTIKTASLPTPTKEETDDQGNNINGNEVKAGEHVNYTLNWDLSNDKDVKATPEMIKKGFFFIDPIDSRALSVDDLSKAKVVDQNGNKVDGISFHLYNSLSEVPEFIQEQVKANNLQDKITGPFVVAQADDLQAFFDKYVKTGAKLKVTIPTIVKSGFTGEFSNTAYQFGFGKATPTNTVTNYVKPMHKPASPETPAAIAPQVISATAQPMTSDAPVTPSEKTAKLPQTGNADEGALLGLAAVSLVGSLGLAALGLKQNRNDD

SEQ ID NO: 11 - DNA, 44932 bp, synthetic construct
  misc_feature 1..44932: note = Description of sequence: Based on Lactobacillus reuteri sequence
  source 1..44932: mol_type = other DNA
ctattcaaatttgaaattttgaacaactttatcaaggtcttcttgttcaatccctagatagtggcgcgtatttcgttcagaggagtgattaaataactgcgaaatgatttcaacgttaacacctttcttatagagttgtcgaccaaaagtttttctaaaagagtgtgttccaattttagcaacgattttattattttccgtttttctagccattcgttgcaacatttcgtaaaagccatgtactgaaaaatgaccttcttgtttcccagggaaaagataatcattctcgtctttataattcaagtcattaagataatcaataatttcggcaagacaattattccaaaagagcgttttagccttacctgttttttgctcaataacacgggttttagttttatttagaacatgaccaacccttaacgttacaacgtctgatgctcgcaatccgttattaagggcaattgcaattagtaataggtttcggttaaccaattcaggacgcgaatgcagcttaattgtctcacgtaactgatcatattcttcgagagtcaagaatttaacattttctacccatattttccgtttacccttgatttgaccattagttttatttttgggaatataaactcgttttttagttgacataatatttcttcctttgttaatttgcgaggtaatcaactaaagcagggacgatttcctctccttctttccgcccagtttcataaagtgctcgaattttcttaatatttccttcaatccgcttaattttaattggtttagagggagcaatattaaagatctttccttcacaagacaaacgattaatttcagcagtctgttggttgtagagaaggggacgatcaatccccgctttagcaaattcgggataatctttatataccatgtcgtacaacttctttacagcagaactagatggcttctttcggtagttaatatccctggtccgtacaacaacaatcttgtcatagccttgtcgttgtgcaatatcaaatggaattgaatctgtgatcccaccatcaaggcaaggcccttgagaagtttcctgtggatctgaaagaaatggcattgatgaggaagctttgagggcattggttagctcctcaccaacaggatccgtaaataaaactgtttttcctgttttcacagaagtagcaactgctgtgaaatgagatgctgaccgtcgatatgcagcttcattaaaattctgccaagaatatccgtgatcttcaaaaagataatcaagattaattatttgctttttaaatacgcgagccatcgagatatattggcgatcgtgccgatgatcaatattaatattggcagcgcgtccgtattgcttagagacgaaattaactccacaaagtgaaccagcagaaacgccaattacacttcgaaattcaatatggtgagcgagaaatgtatcaacaattccggcagtatattgtccacggaatgcgccaccttctaaaactaatgctgcattatataacataaaatgaaataactcctttttgtccgccaaaaatagcaaaaatgacctaatttttagtgagacactttttcaataaaattatacaacagtctcacacgaaaatataggattttaaaagcttgttatatcaacgtttattctcggttaccgtaagactcttttatcaaaagagtcttactgattttgaaataacgcaaaagtaggggcaaaaacggattttgcctctcagacgaactgaaattcattttgatgagtagtaggtcgttacaattcgtctcagaagaaaaacgcatattgatatcaaaaaatgagtaaatagacagtaaatatttaaatatgaactttttatgaagaaactattgataatttaagataatcataataaaatagtgcacagttaattgttaaacgtttagcaaaaaaggtaaagaaacggggttatttctatgctagaacgcaaggaacataagaaaatgtataaaagtggtaaaaattgggcagttgttacactttctacggctgcactggtgtttggtgtaacaactgtaaacgcatccgcagacacgaatactgaaaacaatgattcttctactgtacaggttacaacaggggataatgatattgctgttaaaagtgtgacacttggtagtggtcaggttagtgcagctagtgatgcgactaaaaattctgctaatgcaaatagtgcttcttctgccgctaatacacaaaattctaacagtcaagtagcaagttctgctgcaacagcttcatctacaagttcagcatcttcatcaactaacacagatagtaaagcagctacagaaaatactaatgtagccaaagaggatgatacacaaaaagctgcacccgctaacgaatcctctgaagccaaaaaagaaccagctgtaaacactaatgattcttcagctgccaaaaacgatgatcaacaatccagtaaaaagaatactgccgctaagttaaacaaggatgctgaaaacgttgtaaaaaaggcgtggattgatcctaatagtttaactgatgaccagattaaagcattacattaaataagatgaacttctcgaaagctgcaaagtctggtacacaaatgacttataatgaccttaaaaagattggtgaggctttagttgatcaagatcctaaatacgctattccttattttaatgcaagtcaaataaagaatatgcccgctgcatatactagggatgctgaaacaggtgaatatgctgatttagatatttgggattcatggccagttcaagatccagtgactggttatgtttctaattggaatggctatcaacttgtgatcgctatgatgggacggcctcatcacgaagataatcatatttatttactttataataagtatggtgataatgatttttcacactggcgaaatgcaggatctatatttggatataatgaatctccactaacacaagaatggtccgggtctgctattgttaattctgacaactctattcaactcttttatactatcaatgatacaaataatgctattaaccatcaaaaattagcaagtgctactatgtacttaacagccgacaatgatggtgtccatattaataatgtagagaataatcatgtggtatttgcaggtgatggttatcattaccaaacttatgatcaatggaaagctgcaaatagttttgctgacaactacactttgcgggatggacatgttgtacaaatgccaaatggtgatcggtatttagtatttgaaggaaatactggaactgaaaattatcaaggtgaagatcaattatataattggtcaaattatggtggtaacgatcgctttaatattgaaagcttgtttcatcttttaagtagtgatgttgattataaaaaagctatctttgctaacggggcacttggaattattaaactaacgaatgatgaaaagaacccgcaagtagaagaagtatatactccattagttacatcaaatatggttagtgatgaacttgaacgtcctaatgttgttaaacttggagataagtattatctattctcggctacacgtttaagcagaggaactaatatcgatactcttaataaagctaataaagttgtcggagataatgttgcaatgattggttacgtagccgatagtcttacaggtccatataaaccattaaatggttcaggggttgtagtgacagcttctgttcctgccaattggcgtaccgctacttattcttactatgctgttccagtagaaggaaaagaaaatcaattactcattacttcgtatatgacaaatagaggcgaagttgctggaaaaggtatgaattcaacatgggcaccaagttttattgtgcaaattaatcctgatgatacaactatggtattagctaaagtaactaaccaaggggattggatttgggacgaatcaagtaacaataataatatgttaggaaacattcaaacagctgctttacctggggaatttggtaaaccaattgattgggatttaattggtggttatggattaaaaccgcatgatcctgctacaccaaatgatcctgaaacgccaactacaccagaaacccctgagacacctaatactcccgaaacaccaaagactcctgaaaatcctgggacacctcaaactcccgatacacctaatactccggaagttcctttaactccagaaacgcctaagcaacctgaaacccaaactaataatcgtttaccacaaactggaaataacgccaataaagccatgattggcctaggtatgggaacattgcttagtatgtttggtcttgcaggaattaacaaacgtcgagttaactaaatactttaaaataaaaccgctaagccttaaattcagcttaacggttttttattttgaaagtttttattatcgaaaaaaacaaatcctcgttaatcctttaatgcaattgttgtaaaaccttgcgacagtaataacagtggatttgcccatctttgtcagctaacttccgtgcatgcattgcagaaaaagtatagtgctcatgacaaaatggacaaacatattgctttttcccaaatagtgaggtaataaagcccaaaattttctcctccataaaaaaattatacaccccttaacattatagcgaagattatctttatagaaaaggcttttattcatttgtttttttatatgaagtttcactaatttctttaagagcatcacgctgctgtttctggtcaacgtgagtatataaatcggtagcagatgttcctttttgtcccaactgttgggccactaatacttgatctttcgttacctcatacatttcagaagcaagggtgtgccgtaatttatggggagtaagaggatggccaaatgcggttgaatactttttgaccattttttcaattgcattagctgtcatccgtcgagtttgcttgtgataaacggttaggaagaaagcagtatccttttttaaagcgtggtatcgttgtgcacgaatcgcttggtaagtttggatataagtaatagcccaaggagcaattggtaccgaatccctctggccaccttttcttgttacatctagcagcgattgttttaagtttaaatcaccaaggtttacatttgccgcctcggatacccggacgcctgttcctaaaataagagcaataattgcaatatctcgttccttattgaccttataagaggggagagctcttttatcacatttattagggtattctttttcaatgaaagtaataaaatcaaacttcatttgtccccggtacatgtgtgaagccagcgtatgagcccgataatttaatgtttttgtatcattaagggagtcaattttcagcattacattacggtcaaaataagattcaccattattgttatctgcggtaactgttaaaaatttatacaatgaccttaatgcattaatagatcgattgattgaagtaggagaatttaaccgtccttgtgcattggtagtatgctgcaaataatcgatatacaacattacatcgctacgccgtagattcgccagtgtatcaattggtaagtccttattagaattaacagagacgagtccacttgaccgtaaccaatcaaagaatcgacggatttcagttaagtactgataggttgttgtgactgcatggttggtccctaaataatactcgttaacataatcaggaaggttctgaagctcctcttgaattagctttaaatatttatctgcttccatttttatatcctccttgatactaaaaagagtcgcttgaaagaaaactttcaacgactctctaaacctatcgggaaaacaggattcgaacctgcgaccccctggtcccaaaccaggtgctctaccaagctgagctatttcccgttaataacgaacaaatattattctaccaattctcaattaattagtaaaggaatattttaatttaaagtgattaatagttaatgattaaatgacataatagtaaaattcctattaattgcaaaaaaatcatgattaaccgaaatccttttctagcatctaatgaagatgataaataattttattttcgactagttataaaagatacctttccgaaaataatttgacattcaaaatacttttgaatataatttgattatcgaatattttgatactcgaaatattttccgaaggcaggtgaatctttttggcaataatgaatgcgcaggagattatggaattaattcctaatcgatatccgatttgctatatcgattatgtagataaattagttcctgaagaaaagattaccgcaacaaaaaatgtcacaattaatgaatcattttttcgcggtcattttcctaataatcctgttatgcctggagttttaattatcgaaacattggcccaagctgcttcaattttgatattaaaatcaccgcatttttataagaaaacagcttatcttggcgcaattcataaagcaaggtttcgacaaatggtccgtcctggtgatgtattaaaactaaacgttgttatgaaaaaagttcgatcatcaatggggattgtagaaacacaagcgcttgtgaacggcaaattagcttgtagtgcggagcttgtctttatcgttgctgaacgagaagaaaagatttagcacggtgaatcattatcatttataatatattttgataatcaaattattcgggctatagaataaaattgaagggaagaaatatattatgagtaaagataatgattacgaaaaaattaataaaggattaatcaaagtttattccggaattctatggattgaagaaaatgaattgcggaaaagtacattcaatgatttaacaattaaagagatgcatgcaattgatgcaattacgatgtataaccatcaaacaatttctcaagtagcagaaaagcttcatctaactccaggaacaatgacttcaatggctgatcgtttaattcgaaaaggatatgtggaaagaattcgtgataaagatgatcgccgcattgttcggttatgtttaaccaaaagaggccgggtactatatcgtgcgcaccgggcctttcataacatgatggttgaacgttttcttaaaggaatggatgatgaagaaatgaaggttgttaaaaaagccttgcaaaacttagaagattttgtggatgagcatgcttagagataaggattgacaaacattgcaaaatttaagaattactagtactgcgagttatcatccaccccttaaaattactaaccagcaattatcaactattatgaatacttcagatgagtggattaagacgcggactggaattcatcaacgttatatcagcaacactgaaaatacttcagacttagctgtcaatgttggtacccagttattgactaatgccaatttaaaggcaactgaacttgatttaatcattattgcaacgatgtctcctgatgcctatactccttcaactgctgctattgttcaaggaagattaggtgcgaaaaatgcaattgcgtttgatatctcagcagcttgtactggttttatctatgccattaatacagctgaattaatgttgaaaagctctcattggaaaaatgcaatggtaattggtgcagaagtattatcaaaactgattgattggcaagatcgaagtactgccgtattgttcggcgatgggctggcggagtgttacttcaaaagacaactacaacaacccctttaattctcggccgtgatctccatacatttggtgacttaggagataaaattattgccggaaaaacaacgcctaaaactggcttccctaaacaactaacatccctttcaccatttacgatggccggccgtgacgtataccgctttgccactcatgaagtaccacgatcaatcacttctgccgttcaacaagctaatttgaaattagacgatatcgattattttttattacatcaagcaaatgaacggataattacccaaattgcaaagagactggcgcaaccaattacaaagtttccaatgaatattagtgaatatgggaatacagctgccgctagtgaaccaattttattgactcaagctattgctcatgaattgattaaaccaggtaacattattgcaatgagtggctttggtggcgggttaagtacaggaacaataattttaaactattaatagagaaagaagaatggatatgactaaagaggaaatttttaatactgtaaaaactattactgttgatgaattagatgttgacgaaaatcgtgtaacaatggatgcacgaatcaaagatgatcttgatgctgatagtcttgatgtttttgaaattatgaatgagcttgaagataagtttgagattgaattagatgccgatgaaggaatcgagactattagtgacgttgttgatttcgtaaagaaacagttggatgaaaaataatgtactacggaatattatttagcggtcaaggtgcacagcgatcgggaatgggagtcgagcttatggccgactcccttttttcaaggattgttagtcaggcaagcgctgtttgtgaacttgatttgctcaaaattatgaaaaacgagcataaggagttgaataaaacagcatatgttcaaccagcaattgtaacagttagttatggaatctaccgaatgttaaaacgggatttacctcaactaccaattaaagggatgattggcctatctttaggcgagtacgctgctttaattgctagtaatgcactttcatttgaagaaggaattaaattagttgcggaccgtgctcactttatgcaacaagacgcggatagagaaataagtacattagcagctgtccttgatcctcaacttcaagagataaaagaactaatcaccgctcaacaagaaaatggtcagcgagtttatattgctaactataattcaccacgacaaatcgtagtgggcggggcattaaacgctttaaaggctacccttaaaaagattgaagaggacaagcttgctaaaagaacgatcctgctcaaagttaatggtgcatttcatactcccttctttaacggtgcacgtcaacagatgcataaccggttacaagcagtcgacttccatgagccacagattgaagttattagcaatactactaacagcttgtttcattgtgaggatcttccaggaattcttgaaaaacaattagctgttccaacacactttggagctaatgttaaggaattagtcaagcacgcgaaaattgacacaatattggaaattggtcctggaaagacgttatctcgcttcgctcaccaagttgaccagcacttaaatacccaacacattgaaaatcttgctgattatgaaaaatttataaaggagcaaaaagatggaactgacagataaagtcgtttttataagcggaagcacacgcgggattggggcggctactgcattagagtttgctaaggctggtagtcggctaatcctcaatgggcggcaggataacttaccaaaaacgtttaaagaaaagctagatctactaggggcggattatcactatcttaagggcgatattgcaaatgaagaatcagttagtgaattagcagcagcggcttggcaaatatacgagaagatcgacattcttatcaataacgcgggaatcacgaatgataagttaatgatgggaatgaaagcgagcgattttgaccaggtcatcaatgttaatttacgcggaacatttatgttaacgcaacctatttttaagaagatgctcaaaaaaagagtcggttgcattatcaaccttgctagtattgtgggtctccatggtaatacgggacaagctaattatgcggcaagcaaggcaggtatcatcggccttactaaatctattgcccaagaaggagcacgccgtggaattcgttgcaatgcgattgctcccggaatgattactagtgatatgactgaaaaattatctgagcgagtaaaagaacaaattctcagtcgcatccccctcaaccgcttaggacagtcagaagaagttgctaagaccgcaaaatttttagcagaaaacgattatttgactggtcaaaccattgtagttgacggtggcatgacaatttaggaggaactaaatgacaagagttgtaataacaggaatgggtgctgttgctcctaatggtaacggtattcaagaatttataagtaatagttttgcaggcaaagttggaattaaagcgatcaagaaatttgatgccaagtcgacaggaattaccgtggcaggtgaaattgacgattttgaccctaatgatgtcattggaaggaaagccgcgcgccgaatggatctttattctcaatacgccttacaaagtgcgattgaagcaatggaaatggcggagattaacgaaacaaataccaagccagtcgacatgggtgttatctatggatctggaattggcggtttgacaactattcaagagcaaattatcaaaatgcatgataagggtcctagacgggtatcaccaatgtttgttccaatgtcaattgctaacatggcagccggtaatatttccattcactttaatgcgcaaaatatttgtacatcgattgtgactgcttgtgccactggaactaatgcaattggtgaagcctttcgtcaagttaaagcaggtcgcgctaaagtaatgatcgctggtggatcagaggcttcggtgaatgagatcgggattgctggttttgcggcattaacagcattatcacaagcaactgatccgcttaaagcttctttgccatttgataaggcacgtcaaggatttgttttgggtgaaggcggtgcaacacttgttttagaggatcttgagcatgcgcaaaaacgcagtgctaacattcttggtgagattgtcggttatggtgctacctcggacgcttaccatattacatcccctgatccaactggtgcaggggcggcaagagcaatggaactggcaattaaagaagctggaattagtcctagcgaaatttcctatattaatgcccacggaaccgctactcatgctaatgatgaaggcgaatccaaagcaatcaatcaggtatttggttccgatagtaatgttcgcgttagttcaacgaagggaatgaccggccatttgcttggggctgcgggcgcaattgaggcagtcttaacagtagccgctttacaaaagggacaattaccgttgaatataggttgctttaatcaagatccaaaatgctcggttaaccttgtgacggcagaaaataggaacgcctcaaccgcccgttacgcaataagcaattcttttggtttcggtggtcataatgctgttttagcctttaagaaatgggagtgatctatcttggaatttaaagaaattcaaacattaatgcaaaattttgaagattctgatattcgtgaattagaaataaatcaagattcctttcagctctatttaagcaaaaataagcaaacccacaagcatgaaaatcttataacaaccgaaaaaacagagcaaacgacttcagctaagaaaaaagcaaacgaacaactaactttaccttcgcaaaatataactgcgcccctagtcggaacagtctatctccaaccaacccccgatgcagatccctatgttaaaagtggcgaccacgtaaaaaagggggatgttgtttgtgtgattgaagcaatgaaaatgatgacagagataaaaagcccttttaacggaaccattacttcaatttgtgtaagcaatgaagaattagttgaagtagaacaaccgcttttctcagttcaggaggataaagacaatgcctaataaaactttagatataactgaaattcaaaaaatccttccgcatcgttacccgatgttgctaattgaccaagttgatgaattaatccctggtaagaaggccattacacaccgtaatgtcacgattaatgaagaggtttttaatggtcatttccccgaaaatccagttttaccaggagcattgattgttgaatcattggcgcaaacaggtgccgtcgctctcttatctcaagaagagttccaagggaaaacagcctattttggtggaattcggtcagcagaatttcgtaaggtagtccgtccgggcgatacgttaaggttagaagtcagactagaaaaagttcacaaaaatattggaattggtaaaggcattgcaacggtcgatggcaaaaaagcctgtacagccgaattaacttttatgattgggtaggtggtttaatgttttccaaagtactagtggctaatcgtggtgaaattgctgtcaggataatacggtcattacgggagctaggaattaagacagtagctatctattcaactgcagaccgcgaaagtcttcatgttcaactagcggatgaagctgtatgcgttggaactgcccgggcccaagattcatatttgaatgcgaaaaacattttagaagctgctcttggtacaggtgcccaggcaatccatcctggctttggctttctatcagaaaatgcggaatttgcgacaatgtgtgaagaatgcggaattacgttcatcggtccccaagcctcagtgattgacttaatgggaaataaggagcatgcacgggagcaaatgaaaaaatcaggggtgcctgtgattcctggaagcgatgactatattaccaatgttaatgacgctgttgaggtcgcaaacaagattgggtatccaattttgttaaaagcagctgctggtggtggcggtaaagggatccgacgaattaacgatcataaccagatgcggcaaatatttagcgaggcccaaaacgaagcccgactttcgtttaatgatgaccgaatgtaccttgaaaagattatggagaatgttaaacacattgaggtccaagtatttcgtgataattttggcaatgccgttttctttcctgaacgtgactgctcgattcaacggaataaacaaaaattgattgaagaaagtccttgtgtcctagtaaatgaacaagagcgaaaaacgctaggacaaattgcaatgcgagccattaatgcgattaactaccataatacggggacaatagaatttctaatggacaaggaccatcacttttactttatggaaatgaacactcgtatccaggttgaacatacagtgacggagatggtaactgggatcgacttagtgaaggcacaggttattgtcgctgcgaatgaaccacttccctttacccaacaggatattcaggttcatggacatgcaattgagtgtcggataaatgctgaaaatcctaaacaaaactttatgccagtgactgggacgattaattacttatatcttccagtcggtaatttggggatgcgcattgacactgctatttatcctggcagtaagatcactccttattatgattcaatgatcgctaaggtaattgcccttggtcaagatcgccaagaagctattgaaaaaatcaaacgacttttaaatgaaatggtaattacgggcgtaacgaccaatcaaaatttccatttagcaatcctaaacaatcctaaatttttggcaggaacagcttcaacaacgtttcttgaagacttcttcttgccacaatggaaaaaggagctgacagcgtgaaattatatgatcaaaataatactttaagtgaacggcacatcaaagcagataaaaatgctgatgaaagggtcccagatcaaatgtggttaaggtgtccacattgtcatcaattactattcgccaagcagttaacacaatatgctgtttgtcccaattgtgactatggattacgaatacctgcccgccatcgactctcgtggttagtagattcatttaaagaattcgataaggatctccagacaaagaatccgttacattttcctggataccaagaaaaaatcagcaaacttcaaagacaaactaagctgaatgattcagtcctaactggtgaagcttcaattaatgatcagctattttcactaggtattatggatccaacatttattatgggatccctcggaactgttaccggtgaaaagataacgcgcttatttgaatatgcaactatccatcgtcaagcagtagtactattcactgcttcaggcggggcacggatgcaagaaggaattatgtcgctaatgcagatggcgaagatttcacaagcaataaatgagcatgctgctgccggccttttatacattgtgatcttaaccgatccaacaactggtggagtaacagctagtttcgcaatggacggagatattattctcgctgagccccatgcacttgttggctttgcaggccgtcgagttattgaacagacgattcatcagcaaattcctgttgatctccaatcagctgaaaacatcctgcatcatggatttattgatcgaattgtaaaacgtcaagatgaaaaaaagctgcttgaatggctattaaaaacagggagcgttgctaatgaatgaacaattatcagcaagcgagattgttaaacgtgctcgcaatgacaataaaattacggggatggagattattcaaaatgttttcccagattttgttgagttgcacggcgaccgggcaggcggggatgatcctgcaatcgttggtggaatcgctactttccatcagcaagcagttaccgtcattaccactgatcgaggaaaaacaacagaagaaaaaatcataaagcattttggctcaccaatgcctagtggttatcgcaaggcactccgcttaattaagcaagcagttaaatttaagcgacctgtattctgttttgttaataccgcaggagcatttcctagcaaggaagccgaagaaaatgggcaaggaagtgcgattgcccaaaacattttacaaataagtcagcttgccattccaattatcacgattatttatggtgaaggaggtagtgggggagccttagcattggcatgtggagatgaagtatggatgttagaaaacagtacttattctattttatctcctgaagggtttgcctccatcatgtggaaagatagtacgaaagcagataaagcggcagaattaatgcaaatggtgccgcaagctttattaaaacaagggattatcgaaggaattattccagaaagcgaagagcatcgtaaaacttgcaaaaatatcgagcaggttttactaaagcgattaaacaagctgcaagaattaccgccaaaccaacttctagcaaacagaaaaaaacgttatcgaaagttttaaggaggataatatgggaaatatattaacaggaaaaaagatcgttgttatgggagtagctaataagcgttctattgcatggggatgtgcacaaatgatggctgaacaaggtgcccaagttatctatacttatcagaattccagaatgaaaaaaagcttacaacggttagtagatgatgaagatcaattaattgaatgtgatgtagcagatgatgaaagtattgaccaagcctttacgattatcaaggaacgttttacaaaagtagatggaattgtacacgcaattgcttttgcaaaaagagaagaattagctggttcaatccttggtgctagtcgcaaaggttatgcaattgcgcaagatatttcgtcttactcccttattgctgtcgctaaggttgctaatgagttaaatctattaaataatcctgcaagtattgttaccttaacttattttggctcagaacgtgctatccctaactataatgtaatgggaattgctaaagctgcccttgaagctagtgttcgctatttagcacgggatttaggacaaaaacgaatccgtgttaacgcaatctctgctggcgcggttaagacattagcagttacaggtattaaaggtcatgatgaacttttaaagatgtcccaagcaagaactgttgacggagaagatgtgactattagcgaaattgggaatgtgtgtgcattcttaatgagcgatttatcaactggagttactggcgacaccatttatgttgataagggggtacatttgatttaaaaatataaattttaaagactgagaaatgagtttttttcagtctttttgctgtcttttagaaggaaaacataacaatagcaagaactttttttaattttttctaaaaagttcttgctattttatagctcagttggtattattaataacgttgtgaaagacgagttaataattttgagaaaaattgtttaatatatttaaaaatggcccgttagtcaagtggttaagacaccagcctttcacgctggtatcgtgggttcaaatcccgcacgggtcacttttgcggaagtagttcagtggtagaacatcaccttgccatggtgggggtcgcgggttcgaatcccgtcttccgcttagccagttctattatgccggggtggcggaattggcagacgcacaggacttaaaatcctgcggttagtgataaccgtaccggttcgatcccggtcctcggcactatttcgggaaatagctcagcttggtagagcacctggtttgggaccagggggtcgcaggttcgaatcctgttttcccgatctggctaaacagctaagaggtaatcttcttagctgtttttttggatatctgtcaactggtatggttgactaaaaatttttttagtgcgcccggcatgggtattagctaggtggtgaaagtccgctatgggccgtagtagtcggaaccatgagctgaggacaagggtgtccaccgtgaggtggaatctgaaggaagtctaaggcaaagtactgcatcgatgaacaagaagtagctataaggctgaaattaactggataaggctgctagacaagttgaagtccaatactactcgaagttggtctcagtaaagctaacgatgacatggtacgaaagctaatattcttacccggggagatctggcctacacgtttccgacaagcagtactaacgcagccattaattagttttttgaaataaaataaacttttttgaaaaaagtattgctttttataggataacttgataatataatattcgtcgctgatatgcgggtatagttcagtggtaaaaccacagccttccaagctgttgtcgcgagttcgattctcgttacccgcttttaattaatttaatatggcccgttagtcaagtggttaagacaccagcctttcacgctggtatcgtgggttcaaatcccgcacgggtcatttttggaggattagctcagttgggagagcatctgccttacaagcagggggtcacaggttcgagccctgtatcctccattgaacaattttaatagttgttcaacattttaatataattgggctatagccaagcggtaaggcaacggtttttggtaccgtcatgcgctggttcgaatccagctagcccaattagtctaaaacagtgagtaaaaattactcgctgttttttattagaaaaaataggcaatctaataacgtaggctaaaagcgaacatcattggattgcccaataattagcataatttattcacgtcgtaacataatatgccagacaaagaagccgactccaataattaacgccaaccatgcccatagttgcaatggaagatagatagtacacgttaacaagcctaaaacccaactgattagcatcatcgataatccagcaatatcgccacctaaaccatcaataccacaagtagcgatatagacgattcctgatgccagatataatagcgaaacaattaattcggctgttccggcataaataggaattgcaggattaggattcccaaaacctcctcaagtcttattctacccggatgattagaatttataaagaattacagtttttctatttacaaatagagcagggggaacaaaaaatcaaaccagaaattatcttctgatttgattggatcttatttagttgtatcttttacttttttcaaggcttctaaaaatacttcataaggttgtgcaccagtgattgaatacttattattaatcacaaatagcggtgcacttggcattccaatcataaatgcgcgccgttcattttttcgaacttcatcttcatactgattagattcaagaacttttttaacatcagcaacaggtagtccgatttcattcattgcaacagttagagcttcatagtccgcgattgattcattatcattaaaataaagttgataaagacgtttaacagccttattaagtaatgcttggtcatttaaactctcaatgtattttattaagcgatgagcagctaatgtattaacaggaatcgcattttccatcttaattggtaaatcaatatcggcagcaagttgatcaattttatgtatttgtgtaacagcttcttgcttagttagttgatgagtttttgcataatattctgtcattgataaatcagttgttgttggcaacgttggatctagttgaaatgacataaattttaatggtgtctgatcggcaatctttaattctttaagcgcccgttgtaattgtgtaattcccatatagcaatatgggcacgcaatatcagcccagtattgaatttccatagatgatcgctccttataaatatatattaatttaatctaatcataaaataagcgaagttgctagtaaatacgtaaagagggaacgatttattttaaaattaacatgtcattaaaattatagttttaatgcgactgaatttaaaattccccccagaatatcaatttttagctttctcaaaagaatttatttaatgcttatttttaactttaataaaactttttctaaacaaaactaatacttttgatttatgttttaaaagatttatgtaatactattgatgaagtctatgtcaaaagtattttaaaaggagtttttatcatgaaacatacgcttaaagttgatcaagtacgtgacggtttatggctagattcagatattacgtatacgcaagttcctggatggcttggtaatacaacgcgagatttgaagctttcagtcattcggcattttcaaactaatgatgatacacgttacccagtaattttttggtttgctggtggcggctggatggatactgatcacaatgttcatctgccaaatttggttgattttgctcggcatggttatattgttgttggtgtcgaatatcgtgatagcaataaagttcagtttcctgggcaattggaagatgctaaggctgctattcgttatataagagctaatgccaagcgctttcaagctgatcctaatcggtttattgcgatgggagaatcagctggtggtcatatggcaagtatgttaggtgttactaatggtcttaaccaattcgacaaaggtgctaatttagattactccagtgatgttcaagtagcggttcctttttatggtgtagttgatcccttaaccgctaaaacaggaagtgcatcaaacgattttgattttgtttaccgtaacttgcttggcgctgaacctgaagctgcaaatcccctcacgtatgtaaattctacttctacgccctttcttatctttcatggtacggaggatgtcgttgttccaatcaaagatagtgaaaagctttatgatgtattagttgaaaacaacgttcctactgaattatacgaaattgaaggtgcaagtcacatggatgtaaaattccttcaaccacaggtatttaaaattgtgatggactttttagataagtatttaacccggccatagatcatttcttgcctttactactaaaaagcaaactaataacaatccaactttcacattattgacaaaatcaaaaagcaccatgaaattactttttcacggtgcaactattgttaatatttatctacttggcttccatcaatggagaaatagacaacttgatttgcaaacctaatccagcagcatatttatttaatgtaggcagcgtagggattgaatctaaattctcgattttagccaattgaggttgtgtcataccgattttttttgcaaattctgtttgcgaaatgccctgtttaattctttgtacttctaaaaaagacaaggtatcaacaatcgataattcttcttttgttgcagccgtctgatgcttatctatatcttcccattttctcattatttacccttcctttcgtaccaatcgtcaagtagcgacaaggctctttctatctgacgaggatctgtttcatcctttttcttagcataatgatttaataatacaaaattatttttcttccatactccataaaatactctttctggcataggtcgtaactcccatagttgatgcctataaccctttaatttttttgcttgtggtgtatgcaacacaggaccaagagcttgtaacatcttaatttgatggcgcatttttaaataaattgctttatcttgcttttgtttactttgcgaaattttctcgaaataatcaccaatttcactatttccatttttatcttcgtaaaaaactacttcatacatgcactatttcctttattatagcttatttattactaattatagcaagattgctatagctttactactattaaaatatattccataatcaaaaatttttctattgaaagcacccgcacactcaattaaatcaaataagtttacggcaatgattatttttggtgcacaaaaattgccgatgttccccagctttacaaactttcaccatttcataccattttaactaaacaaaagctccggtaaaatgccgttatatcagcattttatcggagcttcttttttatacaatcttaaaaaaatgcgtcccccgagcatagaagattgttgatatatcaacgttttgaaggagttagtgtgccaaacgtgtgcgaacttaactaaaataaaaaagagcgcctatcagggcgccccacgttataccggtacggatcaagtaacgaactgaatccaatttgcgtgccgatacttctcatctgatatttaaattataacataattattttatgatacaataattaatgcgtgtcgggagtaacgacccaaactatcacgcaaaaggggtgaattccccgtgacatttatacttctcattctgatcgtgccgaacgggctcgtcaagcaactgatgcattatgcaaaggttgtaatcaaaaaggctctcaaacatctaattgatgaattacttggttaattctgataggaaatgtgcgagatgctatttcagcggtagtgtttgctatcgctgttttttattatactttaagaatatagtcatggtatttttttaccttagtaactattcagtttaatgttactctattaaattttgaattcctttacatccttaacctctatcatattacaatctttatcttcaacgtagacgaattctgacatatcatcaatatcattgaaatctttattagctgcgagttctgcagcatccttaaaaaaattaggccttttttcttctattttgtccatcggtactaaacattttaatatgtaagttttgaaatgctcttgttcccatcttgtttgaagaggcccttttaatggcctacttctaatattatctaattcatatatggcactatcaaggtaccttaaaatttcaggacacttataaatgcaagaataatcaaaatgatatggtataaaaaagccactaattccgttatcattttccaaacgtgctgcaatatcattccaacttccaccagattcaatcaggtatgagtgcccattatatgaaataaaaggattattatttttacagtagaaacgaatattatttttttctaaaaatgatcctaacaaggtactagaatctgttataactttgctaagtggttttaatccatcatataaatagttcctttgctgtgaagaacaagttgtaaagtggtaaaaatatgcattttcaatttcatcttttttatatagttctaatgatctttcagccatttcatgagtcgataaatcaaatatcttgtattttttcttttcagacaccatggttttctcctttttatgatatatctaatttagattactatatgacaaaacaccctagcaaccattaatcggctactagggtgtttattactataatttttcacttaaatctgaattgtttgaccaatgaaaatcatattaggatttgcgatgccattcttttgagctaatgcttgccaggtcttgccaaacttagccgcaattccgcttagtgtgtcccctgcttggacagtataagcattggattgtccgttgccggctaagtagagtttttgcccaacgtagatcacattcgggttagtaatgtgattacggcttacgaggtctgagacagtggtgccgaatttggtggcaattccggataacgtatcgcctgattgaacaaagtaagtattttcgtttgatacttttccggtgactttgaggacttggccgacattgatctggtttggattaccaataccattgattgctgctaggttctgataagtagtcccgtatttttctgcaattccactcaatgtatcgcccggctgaacaatgtaggttccagtagctgggtgaccaatatgttgaaccggctgtggtgctggaataacagcttgcggaacattgcctgatactaaaccagtcgtaaaggcaccgtcaaagtcataactagtatcaattcccatgatgctatgatcagaatattgccaagcacttgcattatcaatccccagttcagtcacaccatagccagcaatccaaatcttccgggaatcaaagccatgtgaattaagaataccgccagtgaagaaggacttcatggagtaaatcccagtattcttgtaaccaagagcttctacttcttgaagaaaggctaaggatactgattgataatctgcagttgaatgaacttccgcatcatcaatcatcaaagtatcgtcatacataccaaattgcttagcgattttaacgaagaatcgagcttcattttgtgcatcagtcactgatgtataacgagcaaagtggtaacaggaaacgcgcaagccaaccgctaaggcattacgaatttgagcggccgcacgtggattaacataagccgaaccatcttcggacccttccgttaacttaacaacgacccctaatgcaccctgagctttagcagcttggaaaaaggcaacagtatctggttgataacttgaaacatcgatgaattgattacgcatattattcttcccctttcattggatcgattgctggtgcttggccggctggaatagtagcgggtttagttggttgtgatgtcggatcaacggttggtgtcaatgcagacttttcataagctgattgcacaagcgtttcaatagcgttaaggtcaatattcttaataccctgcttttgaagtgcttgttgaacaatacctgttgcttgatgaaatttctcatgaccagccatgtctttgccaactaaggaagcaacggcattatctgcaagtcgttcagcacaagaccaagcagcacgagaagtttctgttttggcatgagtgactttagcgtgaataaatgtctggctttgctttaaggcaaaataaaagaccgttgagataacggccgtcatgatataggttggaattgcattaatgattgtgctcatgttgtttctcttctttctctacataatctttgctgagcttgtaaatatgattctttacccaagatggtaatggtagtcccatttggccccagttttcgataatcgaaactaggtaaaataggaaaaagaaaaagacaagcgagtcgcctgcctgtcctaatccgcataattctaacattggataaacggttaaagtcaaaataataactgcagcatgtttcaaaagtccacccgttcctttactagaagttgttcggtgcgtaatgatacttttgaaaaaacctgtcacaatatcaatcacaatcgcccaaactaaccattcaatgagcacgttgtcaatcatgctggcaaaatattgaatgtatagaacgtgttgtggcggttgtgtcagtagtaagtgcatttatagtggtcacctccttataagggtaaaataaaaacgccctgtagaggacgtttaaaattatttaatttttattaaaagattcccaatttccatcttcattattattgattttaattattaagtcgttaagtttattatcaatatcattagctagcttatacgcacttgaatcacctcgtactctagtaatttcatatatttttccttgacgaagttcttcagtcaaagatttaatattatctatttcgttagaatatttctgtttgagattcactttatccccatctccttaccgctaaaataaaagtgttatattacgctgttactaaatatggaatattattatcagataaccattgaactgcattattattaatttttacaacatcatctaaaattttatctaaattaaatgaataaccaaatataaaaaattcatgcttatgaggatcgtatactcttaaagtttcatcttttgtaggagtgaacactttgtctccaacttttatagtttgatacaaattaccaattacttgtttttcaaccattatgtgctgacgtcggttaaatttaataagtccttcatcaatatgttctaaactattcctgaggcctcttaaattttcattttcgaccctattaatatagaaatcatcagttacattcaaaatttcccttaccttttttcgatcattactatttttagagttagaaccccatagaagcttagctatatctgacgtatacaatgcaatattttgtaaattataccaaatcatattatctttataatatttttcatgtttggtattatatatatttttaatagcaataatagtaaaattacataacagataagtttcactaattgcatactcctcgtctttactattcatagtataatgttgcataatatttaccattccccttattaattgtttagtaatattatcgtatcagtaattgaaataattatgatattattatgaaaaattgaagaaaaaccaaaggctaataattaatttacaaaagtatggaatcaaaattttaataaaatgccgcccataataaaagccccgctcgtttgagtgaggcttatttatgtattgtgtatttctaaggcgactataagtaattaccgttaagctaagaattcatttaattattagccattttctttagcttttctttatcttcaatctgatacaagcgaatcttagtaattaatccctgatacgcttttacttcattgggaaaattattagcaggagtaaatttatcatcttcttgggacaattcagaaacatagaatacatttcgataataacctatataaagtttatggttcagtttcctatataatttatttttttcaataatttttgcttctttaggagtataaccatcttgttttcggtcaaaataggcttcccatttaatgcactgttcctgtgaattaatccgatttgtttttaaaccaaatgctgctaatggatacccacctaatttgggattgttacacaattcattatcatattcgtaagcagtattaacaatatcttctaatgattttgaaatattcaagataaccacctctattttatagagtatcactaatttagactactaaacaataacttaaataattttattgtcatgatttatgctccttgaatttcgtaattctgaccggttaccgtcttatattcatctttagtgatcgctccgcatcgtacataaactttgtagaattctaaatcatggttaccccagtcattccaaaacatttgaagcatttgtaattgtgtcatcattatgccgttacctccttagatttttcgactgcctgttgctgattaacagtcataaacatttgttgtaattgcttgatttgagttgcttgttgagcattagtagcctgcaattttgcatttgattggttctgttgcataatcattgcttgcaaaatagtgatttgttgattggtattcccgtttgaatgcgaaataatcgtcacaggaatttgactatcattcataataaagctctccttagtatatttttgtttgataaaacactgttacagttacccctgtattttgtatagctgagttatcaatgtttctaacgattaatccatcgccgtttccagcttctgaaacactcaattgccagcggtcgccttctgcaacatagttaagtattttcccagtaactccaagttctttattgatagcatccattggaagtgtcttgacctcttgcgaattaagttgatcaatagctataaatttagtaatcgtctttaaattagaattgtaatctgtttgatgatcttgcaaactcattcctagcgatgtattgcctaaactaatgagcgaagttggcgttttatcagcataaattctcccagcttgaaaatgggctgagttttgatcgtaaaatttaactccagtattaaaagcctttccacctaacttaaagtgatccacatctagatatgttccagtcgtactaggcccaatatatagcccattcatgctttctctttcagctcctagcataatctcgtttgctgtaaactcagcgttagtttgcagagtgagcaactgatcgcattttcccccaagtgcaaggtaatcaatgtggccctttgatagcataatttttagattagcttgtccacttttatttgtaacaaaatggctttcccaatgtttgatagttagatcgtaagttgaatcaatcactgatccaatagcattgtctgctatatcaaacgtttcctcccacacaaaagaatattcaccgaaaccggattgttgcgataccgaatgctgatacaatgtttgatagcaattatagttccaaagtttgattttgatcccaatattatgtgtgttatctgcatctgtaccagttacgtttaatatacatcctctgaaataataccctttaatcgaaatattactatgaaacatggtatcaaaagaaaccccatacttagctgcacctcgctctagcataatttgaatatcattttgatttccatttctaaatttgaatgcaatttgatccacgtttagtgctggtgttaactttaacctcaacgaaatatgagactgcttcaagtcaatagctaatggactttcaattagataacatccattcccatgaacacttttattattatcaactgcataggttagaatgctttgaaggatttggtaatcatcatttttaccatctccatgtgctccccatgtttcaggtataatttcattttcaataagttccgctaatagtcctgatgctaattgttctgcatgtttagttgttgtatctgtgacataaaaaagccctcctcctccatcatttggttcataatagccaagagttcttgcgcacatatctttgcttaaagcactatcagctttcatggcagctacatttggatacacatgtgcatagttaatcgcaattgccttcatcatttcctgcttaaacgcctctgcttccgcctgggtaaacaaattgtcttgcttaatcttagcgtctagtgcatctaaaccattttgaacagtaacgccaagagctttgagcgtgttcattgattgggtaaacttatcaataaagtcactggtcatcttggtgagcttgtcggtactgtcttggtacttggcttcgatttcgttaattaactcttcgacgggtgaaatatatgttcgtggtaccaagccatcaatgaccttatcagcaaggacatcgaggtcgaattccaaggtagtgatagagttaccgtctttgaggatgcgaaaaaaggcttggcgataggaaccagcgactgtaaaggcatggccgggcatgtcgaaacggaaacgtccggcggttgggtcaagggcaacgtagcctttatcatcaattactcggaagtcgccggcggaattcttaggaagtaatccttcaaaccagacgttacagccagttaaatctagtggtgtcccgtcttcgttcttaatattgacaaaaacttgacgcatacttcgctcatactgacgcgcttggacccagttagtattactgccatcaaagttaaccttgaagtcttgtacattgtcaacatgcggacgccggtcttgaccgatcacataagttaaagtttgtgacattaaatcattcctttctctttcaaaatctgcatgacagcttgttcaaccgtttcatagtcggtccctaaagccacatgctcaattttctttttccacgcttcttgagcctcttttagttcatctcgcgtggctttttcttgttcgagttggtcgagacgagtattaatctcctcgagtagcctaatcaattctttatactgcctgtcacggagattatcgtctttgttatcctcattttgaattatcgttaaattatttattaattgctggcggaattttcttccactctgcaaatctagattagtttctaaatccaaatgaccatccccctcctcacttcaaactactccaatccgtcactctgtaacctagatttacctgggcccgtgatacgtatgctttatcagatccggaaacattaaaaaccagccttcctttagtagtgccggctggaattttaatgttaactgtgccggcggattgccaggcgccattattgacgatatagacaatattcgatttgccggcagaactaccatcgctcttcaagaactcgacataagtagtagcggaacttgaattatcggtcgcatttaacttagccatgagacgacttgagatggaggagacaccgttgaggttaaattcattgcttctgagagtgccttgctctacttcaattactggactgtcattatatttttcgctggaaacagttccgccagtcatcatagttaccaaattatgctggccattttgcatccagtaaatattctggtctactgcttccatattcttatccgtctctttcttatcattcacgacttgcgttaatgacttggattgctgatccatgctatttgctaatccactaatccggagtcgttgttcttcaagagcttcttgtagacgtttagctttctgattttgataatctaaaatagttgtttttgaattatttaacgttatttgattatcttgtcccttagcttccggatatatcgtataaccgacagtcttataactacctgaatatttatctcgcgcaagtactcttaaaatttcaccagcaacaggtacaaatgaatttccctgaagcgtgacttcaatatttaaatctggatcaggtttaagagtagttaaggcatatttacgcattgcatccgcatcttgaaatctttcatcaacaagatcaggtccaggatgttctccccattcgtcaattgacttttgatcaactaccataaacggttgaaaataatactcttcctgtgattctgtggttgtatcatttccgacagtatcattggtttctttaccaccgataccatcacgaataacttgtaagggatcaagccaggttccatcatttgaccaggcacttttcaatgcagtcattaaattcttttgcttagtaattccaatatgaagatggctagtatcacggtggccaataacatcccccgtcttaatttcttggccaacttgaacagtaatatctcctcgattcaaaaatgcttcttggtagcaaattaaataatcattcgacacaatagttatatagttttccagtccagcaatgtatccaatatcctggactttcccaccatgaatcgcatgaacatcgcgtccaggatgatcgacagaaccaaaatcaaggccatcgtgaaaaccattagttcgccccactccatcttgagggtgtgttccaaaagtttgccccaatgaaaaatgaccttctccaacatctgggaaaggccatccccacggattaggtggtgaaatgatcaatttatctttcgtgattggggcaccatgaggactccaaccagtaacaccattaatttgtcctagtgcattaggaatattgaagaacgcaattaactggtcaaaaccctttaagatatttgtatagggctcccgacaataagtattaaaagttcctcgcttaaattgaagtaacccaagcgccggaccagaaccatcgccatcgggatcagttcctggttgcggaatcgtctcgtttccacctgattccaaatgaatttgtgcccgtaaaacattaagctgctgagcattcggttttattccataaaagctagcagcatactgaataactggtgtccaatcaccgttgactggctcagttggtccattagcattagttccacctgtcccggtataaacagtgtgctgaacctcatgcttaccaccaacacaacgaatcatattaataattgattgactgtcccgagtagttttaactgactttgcatcccttggaaaatcaatcactcgaccattatccttatagaactcatcttctgaataaactcgtatcttcttgttatccggatatataactgcacttggccataactcagtaatcttactaagcatctctttaccactgccgctatcgaacttagaagttgactgcttatcaaaattaccatgaacttcatagctaaagcctagcttatttccatcaatccaagcctttagtaagtcctcaactccataagactgatccttactagcattactatcctgattgttatttaaggtaaggtcacttcgtttataaattcgactaatctcattatagacatgccaagcagtgatatttttcgtattagtatcaaagttttcaacacaattttttacaatatattcctgtccctcaagcacaatcgaactttcaacatctaatggatcatacaatgctgaatcgtaatcaaatactgaaaaagtcaattgaaaggtcgaattcttagaccactcgctttgcattgttggccagagaataatgtctccgattggctccttagttgtctctttatgaggtgtcatcaatactaaactaattgtcctcacctcctaaccaagatacataaatgggaaactgaatgtaatttccaaatcatctgcaccagtaacttcaaagtcattctttccaggcgctaaagtaatatacccataatcggtattggcactgtctgggttattattcttaaatgttcgccgtccctttagcaagagagtatcgtttccagatagattttgattgtacgtccaagaagtattagtagttgtattctttactgtgaattttccaccattatgcttaatagtaatttttagatcatgacgctgttcaacaggatcgattgtaatatcactatcgttaaggatagaaaacttattctggcctttgaaatggtatgaaggagtttcctcatccatgttcatattcaagtctaagaaatcctcatctttcatctcatccgtatgcaatttactaaatcgcattccgcttggattttcaaaaggcacttcaaacgtacaatagttaacttcttctggttcgcttttaatctcaaaggaactagcccgacagtaacggacaatatccggctcaactcctgttcttaacctaaagattcctttttgtgcaaaaacgcgataaatctcatgctttgccatcttaaaatcttttcgatcaaagaactgcaataagaacttagcggtgatcgtcgtttggccataacgggaataactccaaatttgaccatcttgttgggaatcatcacgataattattaataacactaggcgattcagttaagccaagaaaagttaagtggtcagtaatatctgtgctggccacttctttttgatcatcaattttaatgtaaagaatgtttacagcgattacgaccacctcctaaagatgttgattatcaaataatgcttgatcagttccttcaatcccataaaactggttcttgtcaaaggcgccggctttaattgcagaaagttgagctgcatttaagcccagcattgtcccaaagcggttgagcatctggtccatccgatctaacatctctttttgcattccattattactttggccaccgttattataattatttacaacttgggtgctttgcatattgggcttaaagttagttaataccgaaacagcatcatcagtaccattagcatattgtggtaagtgcttaaacatcttagcagtatcgctagctttaacaacttttgttcctcgtggtgctggaaagactacattacgcccataaggaataaatggtgtttcacctgggaattgaactaactcacgaaagacggggccaggttgatcattaacttccatcaatccacctttatggtaattagttccctgagcatgctttgatttatgaaaaataacattaactgttttctcaatagtattaggtatactcataaaattatggattgcatcaagtgctgttttaattggcccagaagctttatcattagcttttgcaatttttgctggtcccgtatttgatccagcaaaagcatttacaccattgcgagcactatacattggtccactagcattatcaacggctaaagcatttttaggagcgccaggatttgaactagcaaaaacatcgacaccaccagtagcatttctcatcggaccactagcattgtcaattgccagtgcattctttggttctcccggattagcatgacgccaagattgaagggcatcgttagcagcttttagatttccactagctttatcatcagcaatgatttgttttgcagcactaaccggcatccccttccacagcccataatcagtgatcagttgctgtagttcgggtccacccttagcatttacgattgcttgctgttgcttaggactaagttgattccagcgaccaattttattgagcgcatccattaatcgaccagcaccctgagtagtaaccaaagcacgttgttgcttaaacgaaagactattccaaacaccagcctgcactaatgcatcaactaattgtggagttcctttagcgtcaataatcgcttcctgttgttttaaggttaaattattccactgtccggaactttgcaaggcttcataaattggcttagttgccttattagtaattactgcttcctgttgttttaatgacaaggaattccatgcaccggattcaagcagaatgtcggccatttcttgcttacctttagccttaacaattgcttctttggtcttaagatcaagattattccattctcccgacttctctaaagcttgaacaattgtttcactaaagccgtctttcaaccaagctttctgctctttccaactcatgctatcccatttaccattttcgattaatgcagctgccaccatttgttgagcattggtacttaacttaccttcctttttcagaagtttaatttggttccattgatctttagaattaacagccttattaacttcctcctgggcattagtccgaactttaccagttttgggatcgaaaactaagtcattccacatatcagcggcttttttagcttttccacttaatttagtagtatttaccgctaagctttgtaggtttgtttcggcagctttggattgccgcttaatctcatcagtgccatctttataactaagtcccatactttttaggtcattcttaattaatttagtactttcaccattagcacgggccaggcgaatatattcggcagctgctttattcgtgtaatctgttaatgctttcttattagcacgcataccgaccttatacagttcagaatcttttccgtagcgtttagcaagcattgaagactgcttcttgtattggttttccatttcagcagtttcactggttatatctcccatttttgttgagcgttgttgcatagacatatgcttaatatcgttattaaggacagccattgctttagcacgcttacttccacttaatttaagaattttcatctcatcgttaagcatttcctgctggttgttctttaacatgactcgctgagtatcatttaattgtgaaacttttttattgtggtcagatagaattgtttgaacacgctgattggcattatctgcatcattcacatatgaattatattgttttttctccttttctaactcatcgtcaatagctttagatatttctggagacagacctttttctgcctttttggcattagcaaaatgttgttttgcactattttcaatattagtaaactcacggtcaaaatcagcagctaatttctttgttgaagttgaggcagcaacatccatatctgacagagtgttctgaattccttgagaagtcgactgcatttttgataaagccttatcagcagctgcaccaacatcagatccccaacgattcgttcgttgtgatgattcgacagctttcttaccccaaagctcccaaacagtaattccaccggcaatggctaaagtagcaactccagcaacagcggcaactgttcctaaagatactccagccgcagcagtagcagcacctgcttcacctgcagctacaccggtaccttgtaaagcgacagtcgcagcaccaacaccgttagcagcttgaaccgcccgaccacctgcaccagtcatggttgttccaaactttgcagcttgaaatgttgattttgaaaaagctgagccaataacgtctaacccactggctcctagtttcattgcagtttgtacacggccgattccggaagcaatttttccaaaggcaagaactgtcttcccagcaccactagtcagtttacccaatgctaggaacaacgggcctgcaccagcagtgaacagggtagttgttacaattgccttctggacagctggtgataaatcaccaaagccatgtgcaagtctggaaaggccttgaaccattggaataatagatggtaagacgtactttgccatatccattccagcgttagttaaagattccttgaagattgctaattgtgctttaggagattgaaggtttttctgtgacaagtcgccgatataatcccgcttggcagagttttgaacttctttattaagttcacgtaaccgattagcgttttcggttaaaattgccccagcttgttgtccagttgttccaaataacgcatgaaaaatatcattcttttggtgaccagataagcctttcatatggtcatttaacgtcttaaaaatagacgacattgattttaactttccactcttagtaagaaaatctttagtacttaaattaatacttgctaaagctttttgaccattagcggttggcgtaattagtgaattgataacctttcgtagaccggtaccagctttgtctgcttctagaccattattagacaagatccccattgcactagcagtttctgagagactaaaacctgcttgatgagcagttgagccaacatatgacattcccacaccaagtgattgaaaatcagttgatgtggcatcggcagcataggctaactcatttaatgttttagtagatctacgctgcatgactgtagcattcttgattggacgtcctgctttatcagtagccagtccaaaagattccattgtctgcgaagcaactttgattacatcattaaagtcatccccagtggccacagatgctttaagttcgttccgcattactccaattgctgctttagatgtataaccacgctttactagatcttgatacccggcagcaatcttttgctgactaacaccgtactcgtcagaatactttcgggcatctgcagtcatcgttttatatgcggcattcgtttctgcagctgattctccggaagttcgaataacgttcttgacctttgtcatttgatcttggaagtcaacgagtttcttagcggaatatgttaaccctgccgcaattggagctgttaaataagtcgacatccctcgaccgacacctgatattttagaaccaacattggtggccacatttccaaaatgttgagtgcgattagcaagttgtgtccacttattcgactgcaattcaatatcacgattaagagcttgcattcgtccacgtaattgttcaatttgcgcagacgttttattatattgggttgctgccttagttcgtgactgctcgctcctggtagtatcatccattgctttcttagcattctggagttgcgcattataattgcgcatttgttgactcatagtcgcataagcagcacgcatattattaatacttccaccggatgctttcagtgccgcttcttgcgcacgcaaagcattggctgttgatttaatttgcgcttttaaaacaccattagccgctttaaacggatcaatatccaaactaacagttgctgccaaatgtcctaatgattgggtcattatttaacctcctttcctagaataagaatgggaatgctttatcaatggttgtctgcttttcttcaaagacgtaattcattaatttcaaatctgaaagtgttagcttactgacctcattccatttatatccatcgcgcattttatttttaatgaaatctgtgagactcttgattgaatcatcaatcatctcaacagttatttttttggcttttcgttatcctttttctcttctaagtcatcttcacttagaggagaaccgagagtttcattaattgcattaacaatgctaattaacccttctatagccgtaactcctgaaagaacatcctttttagtcaatccattcttccaaaaattagcagcaaactcagcccgtaaacttaataatcgttcattatctttatctgttggcaattttttgggatcagagtacatcactatttcttgacgttgaacctttaatgcatctaatagattttcaagcattggggcttcttttcgttcatagattgtttctttgccatctgtttttacttttaatttatatggcatttccatatcctcctcatcgtctcacttaactcgtctctgtctgattaattagttaccctttatcaacaacagtttctggtttagtatcttttgtattggttggatcagttactggagcttcttccccaaagaccattgcatggaacttagcaaaatcaaatccttcattatcttcccgaccgattaaaaggatggttccagtatccgcatccccacgcggaacgaaattaccttcaatttcatctgcttctgggtctggagcaccgtcctgagtcttgattgaaatacctggtaaggagaacatccccttggttaaaccaacccaacaatgcttcccgtttgaaagctttgtccggaacatagtagcaacataatttggtacaagattcttagtataaacttctgtaccgttctgaatatcaattccatacaagtctttcttcatgatggaatcaatatcataaaggttgatggtttccttagcttcggtaattccaccagaaagaaccaagtaagggccgtcatcggctgctaacgtctttaattcgttagtcagctccagcttaacttcacttaacccgctcatctttcgggtatctttaatcttttcgttctcgtctagcaccccatattcaaaattagaggccccaaacttagcaacttttgcatttggtgttcccattaaatatcattcctttcttcaaacccttcaaagttagctgtgaccattatgcaattatccaagtccggatccggataagaattcttgtagtaacgttcaaagccatgactgtggagggtttcatatattcgttcttgaatatccattagccggtcatcattctcttcacgaatccaaaaatcaacttgtacacgtgggtattcaaaaaagcgagcattgtcactgtaaattgcatcatccccaggtaggggggtgatccgaatccacggtgctgaactcgctttaataaatggatcatcaggcgtactagtaaaaattggaacatatgataatttatcttgtcgcaactcatccatcatcacgactaggcactcatcattcgttagataatcggctacttgcatctctggagttttcatactttcaagtcctcaataaatttagccaatattttcttcttagcagcatttcttgaatcttcaacaaagtgttgtggatcttgcttagcagttccagcatcggggaagtgagcaatccatcctttatctttgtcataaccaacatcgacagaataatctccaccgttagtcttgagattaccgtgtttagtatgatcttttaaaggagtcttgcctgaatgatcagagcctttagaagccacaggcgtccattttcttaattcttcttgaaagactttagcaccatcacgggttgcttttcgtgccttcttttcttccgtcttttcaagtttagttaagttagcaatcagctctgcttcaccggtcactgccattttggacaacctcctgggcggtaatttttgttagatctcgctttgcataatcaggatccattcccgtgatttcataccattttcctcgccagttaattaaccaattcgattgaatttccttacgagttttgaaagcaattaaaaacgtgggtgattcttttctgaaccccacgtttgaactgttttggacaaattcacggactgacagcttaggaacttctgcccacacggtaaattccttaactttctgatccttgatcggtcgatgagtttccggattgatccccatttggactgagtaaaatgttatccgttccgtcatgttccgtagtttcatcggtattcacctcactacttatttgattaataaggccatcaattcctgatgaaagaactggtcggtaactatctgcagtaattcctcgctcgtagaagtcttctttcacctgcttcataagtgcaattttgaatcgcggttcgtcagagtagtcagcaggcttactctcccatttaattgcccttgcaatcatcaatgcggcagcatcaacaattattttaagaatttcatcatcaaagtcctggtcaattttgcagtagttttttagattggcaaagaattgttcatcactcgagaaagtgtggtctgtttccatcctactcacctagctttgctaacagttgatccttagtatcggttgaagtataggcaattccatgatcatctaagtactttttgattgtgtctaccgtatcagcactagtcggtttaacgtccgccccgttgtcggacggcgttattttgacgctgctgatggaccagtaacaaagtaaccagcgttttcatcggccttcttcacatctaaacgaagaaccgcttggagatattgaccataaatgtcattatcaacccaccgtacctggagatcttttcgatttgctaaaataattgcacggtatggatcaccaacaaaggcatgagcttcaccctttgctccaaataagtcatcttcaattacagcaacgttgattccagagaccgctttaccagacggactagaaatactatcttgaagtaagtaacgtccattcttatctttcaatgtatcaagccagttgtagaagctttggctggcaataatcatcttattgtaagccacatctaaatcgacattccaaattttctttaggtcatcgattgcattagagccatcaacggtcttagctgaaaatcccttaaaaattgtagcaatagccgcattctttgtatttaaggattgttcttttgcgtttcgagcaactaatccagttaaatcaattgctgaatcatcaatcgattcttgtgaaataggaattgctccacgataggtctcaatttgccatgctaccttaagaaattcaggtttttgaagatcaggattctttgcaagttcttcaacagtatgcatttgagcagttgcctttttaagaattggataactaccagaagcagttgtggcgttaaatacttgaacaaattggctaagatcagttactgttttaatttcattttcaggattgtaaatgattgattcaggaatagtaacgcttgcgtctgaagaagtaattccagtagtaccatcacgatgttcttggtgaagatatgcgttgaaattacgtttttcttcattttcgtcctcgttattttgagaacgcttatgaggatctggagctggattacccttagctgctttacggtaaagcttaaggtcatcttcaatactccgaacttccttttcagcagcttcaatttcagaacgtaatgacttagctcgtgttaagtcttcatcagttgcgtcttcatttgagagcagttggcgcatttcattagttttttcgttaattaatgctcgcttaccttctttttgagcaagcaattctttaattttttcgcgaaacatttaatttcctccttcgagtgtcttcaataattcttgtcgttctaattcacgtaacatctttttacgtttctgatcagtagcatctctattttcttggctctgcatttgcttaaccatattgatcgaacgctgaccgacctgaacttctgtatccggataagcaggtgttgtaactactgatacatcgaacaaacgatcaatttgtcgaatggttcgttcataatcaactccatcctggtcagattcttcccaatcttgggcctcatctgtgttggccaccgtaaaagcaaagctgcattgattaataacgcctgccttgatattggcgatcaaatcacgcgcaaatgatgtatcagttggcttaacagtgaatttcaagccaattgcatcagaagataaagtcatgttcacccctgatcgtcccagtacctggttaggatcgtgattgatcgtcgctactacatttgacatatcagcgtcatcgaacgctcctggagcaatttgctcaataaagcgagtaaagccacccaaaacttcggatggcttattgtatttggctgcataaccttcaattactggttcatcatcttcatcagttgccgttctcatctgaattggcatcattaactgtcgagtttccaagtcgcttgtcatcgattccacctcccttcgctgttgattgtttctgatactcttcttttttatcaaggaagacagtattcaaggttgattggaaacggtctaagtctggattgtcagatttgactaaccccatccgaactcgtccttcattaggagtaatgacgttatttgtaactcccttttgtacatcgtccattgacatccctgtttctttgcgtgtatcaaattcgatgtggcagttatgacgttgccgatcagtcagcattgtcatttccaaattacttgcaatcggcttgaagtaataaggtaaatcagaagtaataaagtcttcatttaattgtttaatcgattggttaggactattaactgctaatttatatgctggaatatgcaacgctttggcaatttgagcagttgaatagttgttgctgttaattagttgcaaaacattggtatcaatctcaattggcgagtaatcgaatgtatcatcagtaacgattgggctaccggcattgctattagcttgtgcatattcaaacgcctttctagttttaagacgcgcttccggacttaacttacctttagcctttagtaatccacctttaagacctgacttaaagaaccgccgtaaagtcttaattccatcatcctgtaatccaatctcatcagccaaagacaacaacggcgaccggccatgaataccatcgtaggtaaagaacataaaatgaattacatcttcagccggcacaacgattgtttgaccaccgcctttttgattgataggtgtaaattcgtatttaatattcgttacatcagaatcatcaatataggtttgcgatggtggaaaatattgtatttctaacggtgcttttgtatgaggatcacgaattattcttgaaaaaccatccccagttaaaattgcgttaactgtcattaggaaacgccaatgataagcagataacatatcgtttgggtgcttattcagcaaataatcaacacttttgatattctttactgcatttttaccatcatccaaaatgacaatcggaaatcgagctacattactagcaacatgagagactgcagtcagcacatcagagtttctaagagcaccgattccactataagatggcatattgctaaaccccggaagaatcccctggtcaatataatcttgtgcccattcgcgtttctcggtatgaaataacactcagcttcaccccctttcataagagataaaagccattacgaataattcaattgaaataattagtaagccaatcttcgtggaaaataaaaagcctgtaactgctaaacagatacaagctaatatgaacagcaaaaccggttcgtttagtttccagaatttcatggcatcacccggagaagaaatttcaagtcttcatccgtaatctcaaatttctcaatatacggggcaagctttctcagtcgttccaatcgtcccttgatttcattaagtgcgggagttacatcaatcttcattccttcttcaccgtcgtatagatacttagccatgtctattcctcctcgattcctttgataaattcaattgcttccttagttaaggccaatggatggaaatattctccatctgaacagtcattgatgatcttcccttttaataggtgctccacatcatttttagtgataaaggtggtccagttgctatcgttgccatattcatctagttgcttaatatcatggctaaatttatctttttcagaaaccaaagtcatcattaaacacatcctcatctgttaaataatcatcaatattttcacggaaacaaattgcatatgcgtctagcaatgcatcggcagcatcaatcttatttgaataacggttcttatcaatccggacaccattgttatctgatttaaggatcgcgttagccattgcgccagttaaaatttcattgtcagaatgtcgaactcgcttatctaaaatatcatccctaaattgcttggttggcattgaaagagttagtgttccttgacgaacagatatctgttgccattccggatgacctttttcaatctgagttaacaaggttccatattgtgcggggtcataacaaattgcctgaacatcaagattgtgttctctgacaaagccatccagccattcatatacccgttcaacatcaataactccagattcaagcttcgtaatttcacactggcccatcccttgcaaccggacataatctaaccggtctgctttaatctttgcttccaaaccatatttagtaccgacaaacgcataagaatccgcataccaatacccctcttgaggaattaaccaactaattgcataaaggtcagatgatttaccaacgtcaatcccaaaccaaactcgttgaccatcaatgtcaattggatcaatttgagctgcattccaagtatcaatatccatataactatcctcttcggcttgtcgccacatattaaagttcttaactagaacagaattctttgtccctttctgtttagcttctttccaccgctttgccaaatatccatatacctggtcttgcaaagccgggacactcagaattggattggatttaatccaggtacttttatcatcaacttctgatacattatcttgttcagcaatgtaggcaaaataagtatcatcagtaatttcacctttcaaaaccttcgttgcatagggatattcaattgtatgcattggaacattcaaatcaaagccggctgttgaaataatcatgataagtgagttatgaagtaaggcctgaccggattctagtaattccatcatttcagtggtcttacttgcagcatactcgtctaggattccaacatgaggttcaaaaccatcaaccgttcccgtttccttggaaagagaccgaacataagaataatcatcaaggttactaattaaatcacgattaacttttgtcccacgttttgtatcaccatcactggaacgtagagcattgaggcgtttcttaatcatattaaaaacgatgttcgcttgctttttatcgttagcggtacaaaatatttgccgggagaattcaggtgagttacccatcaagaactcatacaatgcaattccggaaataaggatcgacttaccattttttcgtgccattgataacattccttttcggaagcgccgttcagatggcttgtcttttttccaccagccatacatattagcaataatgaaacgctgaaaatctgctaaaggatacgcccgcatcgtttttggatccggaagtatttccatgaattgaataaccttattagcacgttcattatcataaaaatattcaaagtcatcgttatccgcctttttaagatcatttaagtacctttttgcagctaaaatcacctttttaccagcaactattttgccggccacgaccttttcagcataatctttagcgtaattcatcatgaaatcctaaacttatcacgcaaggattgatgctcttccttgtcagtttgtggcatgttcatctgcagccgcgaattaacattaagcccaagatcagatgcaagaccctttattgccctggttgctttatcaattgtcttaattgcgttattgattttagcaatgttctttcttttcttggactgttccttatttaattgaacggaagcatctttataaatgctgtaccaggtacaatacaattccaattcgctccggtccaagttacgcaatggcagctttccgatagaaccaataattcgccgatattccgctttggccaccttatctaaatgtgccggcggagtcttttgcaactcaggtaaaccatcggcagccataaattctgccttatattttgcttcttgttcaaccactctaaggtggcctgttgactgcgataataatttttgctttcttgccaa

SEQ ID NO: 12 - DNA, 12924 bp, synthetic construct
  misc_feature 1..12924: note = Description of sequence: Based on Lactobacillus reuteri sequence
  source 1..12924: mol_type = other DNA
ctatacaagcgcccgcaattgttgaatcatgcttaacacttggtaaggggtatttgacatgtcaatacctctattttgataccaaaattgcgccaacatctgaacagcgaacgtatattgttcatactgggttaagtctgctgtggggtttacagcgttttgtacataagccttagcgttctttagataatccttgattaattggtcttcttcttcaccgtcaagatataaggcttgcttaaagttgtctacttcccactgctctacatcaaatttcattaatttcaccttcttttagaacacatctaggtctaactttttagcaacatctactataaattgatcacgtaacttgtacgcttttgtagtgctacaccatatatcgccagttgctactaggttctttatcgtcttatgattagggttaaaatagcattctttaattatcttctgggtacagtccccagcgtcttgtaatgctaagtcaatagcttctttctcacgttttaagcggtgcatatatctatcttggtcaatggttattaacatatcatcgtaataagtccatggtcgattgatagcacgtccaccacctacattttcatctcttttgcggatagggtgcattagttcaaacttacggctttttagtttcttattaattgaaggataatctttcagtattactattactagactttttacttcttttcgcattaatacacctcataaaaaaagagtgggcgatattgcccaccctgtgctagtagctattatttactttcggtcgccttagttgtggttaaagagagattaaaagcagcgtcattactaattggttggtagtcattacggacgataacagataaaccttgactgtaactgtcgaacttgtcccattgggctgtaacttgattacgccggaatactgctaccgcttctgaaatatcacctaaaatcattggatgtgaaccgtctgtgttatctggtaagaacttattactaatcttaacaacgggttggccaagtaaagaaaatccactaggagcggttacatctggttgtaataagtaacgtccttcattgtctttcaaagtatctaatacttggaaagctgattgatttactaaccatgtagaagtactttgtaaggctgggtcaaggtcaacgttgaatacttgcttcaagtcgtcaacattagctacggttttcttagtaaagttatcgccttgtaataccttgataatttcagcgttatcagtgttatcaactaacttttgaagttggttctttacttcagatacaatatcaacttctgaatcgtcaacaacttcatttgatagtgcaatcttaccggcacgggtctttacatcaaacttcacatcttcaaacatattagcgtctacatcagcaatttctgcttgttcttccttagtagtaagtaccgctgtgttgtaacgggtagcgattggataatgaccggaaccagtccctacagtctttacagtagcgtactttgctaagttataagcactgttctttaagtctaggactggagtaacaacttcttctggaataacggcttctgcccccttagttgttaatccgtcacgggtttcaccacgtgaacgaatgaagctttcaaaggcgttagctggttgttctgtattgtttttggggtctaagattgtgtgttgcattttattatgttcctcactttcgataaattggttgtaactacgaatgcttgttttatctacatcaacatttgtctgatcatatgaaggcactgtaacggttgaaacctcaaataaatctttaatctgattaacggtgcgtgtaatttgtccgttgtcgtcacgtgtccaactgtcagaaccatcatcaacatcaaaacggaaagacatagaattaatgttcccattcttaacattattaagagtgtcacttgcgtaacttacacttgggtcaatagttgccttgaagtgaagccccttatcgtccacatcaagttgtaatgaaccagccttaacgcttgctagtggcttagtgtagtcgtgttgatcaagcataactacattggataggtctacgttatcaagggcgctagggtcaatcacttcaacaaaaccgcctaagtccttgctgggttgattaaagagtaacgcatagccttcaactgtattaggctgtgaggcttgcgtttcatcttggttatcttgctggtcttttgttgggtcttctttagggcttaccgtttgtaaacctgcgttagttgttagtcgtttctccattggctgtaactcctccttctttttcaataaaattatctccattagttaccggcactaaattaagtaaggctcgggcttcattgcgtgtgtagataccgttaacataaccatcaactgccatctgctgttgagcggacgggtcaaggcttaatagcttgctattatcaaagctaaaatcttgccctagcttgtagttcaattcagaagtaaagcaatcttcatagtgctgtaaagtaccttgtaaatactgaacgttactttgttgttggttgctatgctcgttttctacacctaaacgctcgggtggtagtccgaacgctttcgcaatttgacgggtattccagtcattagaatttactagctttaacacgtctgtattaagtggtaggcttttaacgtccataccatcatcagtaacaattgtgttaagtgaattattgccggtagttgcctcatcaaattgttttctaatgttctctttaccatccgaactaagatcagcttggtggacttggacaactgttgtgccatgaattccattattaaagaatccagtcattaacctgtttccagccgcctgtatcttcatttcatcttttagcgcatacagtggcgatagtccgactaaaccatcacgggtaaaatacttaaaatgtaagatgttagaagaggcaattactcgcttaatacgtccatctgggctgtatgtgtaagtcagcttttgcgttacatcatcttgctggacagttaatttattgttaggaataaaatttagcgtgtgtgaacttaaatcaatttcggcaaacgcattaccatttaatagcatttcaaccgctagggcgtacttgaaacttctgccgtccattaactcgttaggatgatcattaatcatcttgttatataggctagaactagacttaatcggattactggcaagatcaccagcgatagaactaattgcagcaaatatatcactattacgtaaagccgaagcactaacgtaactattagggtcattactagaaagacttactagagcgtctagaaaggggtcttgtgcgcttgtagtcgttgttctcttactaaataggcccattatctaccctcctttacttgtgattatcagaatagttaatcatgaaagctagaacaatcatttcaataccaagaaagccaatcccaaacaaccaaccggttaatcctatagcactgatctccattagaacgcctccggtgatcaacaaaaccggtttaatatagctagaaaagatttttagcagtttcatagtattcatttgtacgttcttgctcctttctcttagtgaagtaatcataaccagccgcccaagcatccataagagcagcaaggcaatctattttattttgctttctagtcttgtctaataacgcttgaccattaaagttatatttcaatattgcatttcctacgttgtaagctaacatctgattgttagtgtgaagaatgttaccagcaattaattcttccttaaaagtcccaattggtaccgataaattcttataggattgtgttaattgaagttgtggtagtcctgcccgttcaaaacgattaacaagatcagcccatttataagggtcaaatactaagaatttaacatttagattatacttaccgattaggtccatcacgaaatcatatacggaatcataatcaatcaagccactttctaatgaactgattgaacattctccacgtttagcagccatttcataatcaattccatcacgttttgatttagtaacaattccaccataacgagatacaaaggcgtgggaatcagcgtaaaactttccatcttctaacggaactaaccaactaatcgccgttaaatctcggctttttgatagatcagcaccaatataaacatctctattactaatatctggagctttttcaatcaatccatgttcccaatcttgcgtactaatatatgaattttcattctgctgtacaaacatattcatattctttacaagtacgttattaatattattttgttttgtggctacatctaaatcattttgaatgtgttccctcattgtcttagcaatcttaggattactcatcaaaggattagctttaatccacattgtggggtcgaatatttcttcttttttatcaagacaatacataaccgcaaagtaacgatcattcttaattttgccgttgagtatatcggctgcatagtcccattcatttttgaagggtgaattaagattaaaaccagcagtagaaataatcgctagaataccattgtcttgttgagccatacctgaattcaaggcgtttagaatagtgttatctttagcttggtgatattcatcgactacgcccagtgtcgggttataaccgtccgtactgtgggtatcagtagatagtggaattgcgaagctacctgtctttttgtcagtcacccgttgtttattgattttcactcgttgccttaaatactttgaattacgccgtaagttttctagttggtcactcataatatcaaacgcaaggtgtgcctgctgggaactgttagcagtaaatagtatttgtcgattacgggctggttgcttttccataagaagagacacaacgccaatcgctgccagtaagaagctctttccatttttacgggcttcacttaatagaatccgattgaagcgcaatgaattatcgtctacctgtcgccaaccatagatattagataagataaacacttcaaatggctctagcttcaatggttcaccgtttgtttgtggtaagctctcaacaaatttaataaccttaccagcatacgtttcagaataataataaggaaagtcatcggtcttttgctttttcatgtcgtctaagaagcgctggcaagcatgaataactaacttaccggcgattaactcacctttaacaactgaattagcatactgaatagctctatccattatgacaacatctcttcaaactcatcagtaggagcttcttcactcattgccttgtggacttctaggcgtacccgtgattgtgggctaagtgccatttgactatcaattgacttcatttctttaatagccttatcacgtgccatgtagtaaggtgactgctgtaatcctcgttttgtatcaataactaccccagtacgatcaatctctttagtacatttgataattgttgaatacaaagtacagtgagtagcgattaaactaacatctagttcgctaataggggcactcttctttaagagtgggacaattctacgccattccttttttgctacatcatcaagccatttaggggcgtgagatgttaagtttgggtattctgccaatgctttttcagcgtcttcacgttgtgctttttctgccttagtaatatgaccttctggacgggtcttcattggttgagccaactatttaacctccttctatttatttggtgacaccatgtcactatttgtttaaaaaaataaaaaaaacatcatttcatattcaattatcgtattatttaacccttattataccatatttgaatagtttgttaagttcaatgcctaaaagttataataaaaaatccttgacatttgaggctctaaattatttaataaagttgaaaagaaaaccatacaaaataaaaagaaacgtcttgaaatcttcggattttaaggcgtttttttgtaaagggagaatttttttaaaagaaacccccaccattacggctcctaccggtctataccaatagcgggggtattaaaattatttcagctattcatatttattttcctttctcgttgatgatcttttcttgtcttttctcggtgacaacgatagcaaagtgattgtagattagttgaatccaatcttttactccaatcttcacgtataggtacaatgtggtcaactaaatcagctttttttactattccctttcgtaaacacataacacaaactgggcttttctggagcgtttgacggcttaatttaacccacttactcgttttgtagaatctattatagtcatgaggagctttggcgcttctttccattcgctttttgtatacttttctattgtattcatgtttctgttctttggtcatctttttatagttatctcgttgccaatgtttacgcttgtcttcatattctttttgtagatattgatgttttttacaaaaatattgacctactggaatcacctctcgacaaccagcagcactacaaaaccttactacactcatttgataacctcctatgaaaaaagggcatcgcataacacgacacccttagcacttacttattaattaattccattgccttactatatgcttcaagcctgcttatgtgccagcttttaacatcgttaaccgttctcttaatgcaagcttcaagctccattgtatcggcgctgtaagggtcacactcgcttagtctataccttgccatcatcacccagtcttggcgactttcctctgcgtccttgcatactgccacttgtgcaatacactttactagctcttggttgtctggtagtcctagatcaatcgctaatggtaatggactgccatttactgagactaattttaaactataaagcgtagtatacatatgataattagaaggatcatcaccgcttgcctcaatcattacattaataaagttgttatactctgggtgcgtgttgactgttaatttactcatccccatttcacctcttatttatgactatcaagcttgcgtaaggttacaatatcatacgccaataactcatcatcaattgaccaaccagcaactttatagatactgccattgtattgtgcttctggatatttcttcatgtccgttaaatctttcttgtgacgaactaccaatataacatcatcagtaattgaactacctgcctcactaatagcttgactagcactgattgaatagttaccacaatgtactgtcacatctcctacaaaacctgtcatgatcataccagtattcgggttacgtttccccgtaccaccttcatgaccgaatgtgaccttatgctttaatcgtgtaattgcgtatttattacttcgcatttaattattcctttctgcctgtacttgcagatacaagcaatagtagaacgtttgcagccgttctagtgaaatctactgttactttatttttaactccttaataggcttttcacctatccggtggtatgtggtcactgcctcaaaataaaagtacggtaaaagcagttttaattgttaattttcatctcttcaattgaaaatagtttttacttttatcggttaccccgtttcaagtacccacaatcgcctttatatcaacatttatatggtaaccgataggatataaaggtaaccgatacattgcagttacttaatatagtaacccgaaactatcggttacctcaaaatgctttcggttacctttaaatagctttatatcaagctttataagaacttctgcaaaggtaaccgataaatataaaactataactaatttttaagagcaattcctttataacctttaacatttttaccgttaatcttaactgtagtgtgtttgtatcctcgtgctattagctctttcgcaaacttacgattactaagacaaggaacagcagtatttttacaatactctttgtaattatcatatagatatactgatttttctttatctttctcatttgtagtacagcaatcattaatccaattaccgatattgtcattagcatttacccagcttcgcttttgctgttccatgtattcagtcactggaaacttaccagaatcaagtgcttttttataagattttaagcattcataggcaaatgctggcatttctcttttaaagtcttgctctttgaattgctctttaaaatgtttaatcttatggaacgttacaataattggtcgtcttttaaatccgtctgtaaaatcattgaaggctggtaaatcattagcgctaaaaatcaatttagcttcattttcaagtttaaatggatctttatatttaaattgagctgttgtagtgtcgtcaccagttaatgttttaataatattagtctgttccataaatttggggcttatatcagcaaaaacattagcagctttatgatacagctgagaagtagcaaatttagcttctttttggtcggataatgcttctaaagaaacgtttgaaacgttaggcttccctattaactccattagtttatttagaaatgtagatttaccatccccacccaacccgtatagaatcatataaatttgaaagtccttaaaactacctgctaaagcaaaaccaataaaggcttttactgtttcaattgctgttaggtcatattcatcttgtagttttccagtatcatcacggatttctttagttgagggtactaatgattgttttaaccatttattccacgttgtagcttttccttttgtttctaattcatatggtctattttgtaaaagatagttctctggtgaattatcttctaaagtgtccgtcatcaagttgtaagtgtgtttaccaaacactgctttataaggtgctggttgatcaaatggattgtcttctacaatttcttgtgaatctaaggcaattaatttagaaaccgatgctaataagttcgttctccatattccatgaggtacaatttcatttttgattgatttttcaatgactgaatcagcgtaatttttccattgtctacttttaggatcgaaccaatagccgtgttctataaattggttggatgtaaagtgatactttttttctaaatatttagccaacttaatttcatcaaccagtttatttcctttatcacttacagttaaccattctggaaaactattgttatttactaagtcatcaatattagccatatttttcctctctttacatggtaatttagcgtttgtatttaagactgataatctctacttcgccttttgttttgatatattcaaagcttttcccgtctaaacttgtttcttcgtatatattgtcccaatcgactattgattgatcttcgttttgactagcattattccaatacttaaagagtggtgtacggtgcagtattcggttaacttgtttaacgtcatcccctgtgtagtagagaagataaaaacacattgcaccaatagcaggtttaacgctatgatatttatttaggtagtaggaaattatttcctcattatttttataaagagcaataaattttctactacttttactactaacagctcgttctataaccttgtaatcatcgttatagtattcaagtgctttctgaaactgtttctttgcgtactcattaaggggatgtgacgttgaattaataattttttccatcttgtcactatctaataaaggattaacggatagtttttcttcaaaacttaacactattaatcacgtcccacctattgcaattgatcatgaatttgatctaacttatccaacaaaatactagatagtgctaacaatgctttcttacgtgatactactgtttgagcaaccgtagacacttgttcatcactcattgctgcataattgtctactcgtacaattggagcaaaaacatcatttaactgatcatacagtgcttgggctattccggttgtttcttcaacattggaatctgcttcccacaactgattttcaaacgctagttcttggttcttatctttttccattgtttaaaccctccactcatgttagaatgaaagagaattaaatattagtaattcgtccaacatcccccgtaaagattgttggcttttttattagctaaatttaattctctaagttttatttagtcgttgttagtgtctgaattggcgttctcactatcaacggcttttttattaccatccataaagtcgttttcaaaatcagttaaagttttatattgaattgctaaatctttaatttctttagcgaccttaataatttcatctgctgaggaaatcggatcatctgcaattatttctccattttctttagtgatagtaccttcaaaacaatcttcagtaattgaatttatataactgccagctttaacaatcctgtcttctatatcaataattcttttacttaatatttcttattcaaattttttagtgtataaactcatttcttattactccttaaaataagttactaattgcaacaacaatacccagcaatggtaaaaacgttgctagataagcccaaaggcgttggtctggtgaagtccaccagtaagctaaaatcttaccatcaattttttgtaacatatgtctcacctcctcctatttatttctctacaatcacttgccagccagctgtgcgattattacttagaattttcttttacccaacggtcaacatcttcacgtttatatacatattttcctccaacctttgatgatggaaaatttcgggtcttaatccaacgatcaatagttgttcgagaaacacttagataatccgcagtttggcccttattcataaactctggttcggttttaacagtcttagcatcttgtaacttattgttctctagctttttagccacagcttcagctaactgatcaatgaactgatctaaaatagctgtactcattattatcacctctttatttcatatcgtaattacaatatataacataatgaaatattatgcaacaacaaaataaaatatattgtgttttgtattgttattttcggttatgtataatatattaactatacaaggaggaattaatgtgaaagaaatttcatctgaccagaaaataggttctaacattgcactaattcgtaaagctaaaaagatgactcaaaaagacttagcaagaaaaattggaatatcacaaccagccttggggaattatgaacgtggccagcggattatacctgcttcgatttttcacagtttacctcaaatattagatgttcctctagattttctagtctatggaattcccaacaatattacctttcatgatttagcaacgctcgattccgtatctctcaaaaagaaagctgctcatattgaaaatgatagaagtattattgaacgtgggttagggcgtgacgacataaaaagacatttattagctgctgttttgcttgataattatggttatcttgtgtacccagatgaatctagcaatgacaaagaacatgaacaacttaaaaaatttgtttggaatacattagatggattacttaccggcgtaccaattaccaatgaaataaattttaaacaaattaatgaacaatttgaattacttgcaaatacgtatttaagcctaattattgacgaagaggcaaatcgctatcctgctttccgcaaggatttaccacccttcaaagaaaaacttacacagtttttaaatgataacggatattttaaaattttaaaaaaataattttaaacctttctctacaatctcttgccagccagctgtgcgattgtaggaggaaagtaaaaatgtcagtatacaaggataaaagaaatggtacgtggtatgttaaataccgcattaatggtaaatcaaccagtaagcgaggtttcaaattaaaatcggacgctgtagcgtgggaacaagaaaaagctgtggagcttaaaaaatatggtcaaatgaataaccataacatgaccgttgaagagctatcaaaaaaatggttgccagtatatgaacagactgggatagaaaattctactattcataaaacaaaacaaatcattaaaaatcatattctaccccgcattggaaactataaagttgctgatcttagcattgagttattaactgaagttgctgctaaatggaaagaagaattagtaaagagtgacccttttaactatactaagcgaatgttagactacgcagtacaattaagagcgatcccaactaatccaatgaattctgtagtaaagcctcgtaaaaaacatgacaaaacatttactaacaataatttctttaatgaaaagcaacttcacaattttatcaagtgtataaaaaaagactacgaagaaaaaaatcctcgtgctttcatggttctatggttagccctttttactggcatgagaaaacaagaacttcaagccttgacgtggcaagatgttaaatttacaaaaagcggtggaataatccatattaacaaggctattaagaacgcaaaacatccctatcttggtggccctaaaaccgctaactcttatcgctggatttcaatagataaaaaaactgcttcctatctaaaaagatggaaacagcaacagatagatattttaactaaactaggttttaatccacaccaaaaagagcaattgattttctctacctacactaaaaataaaattgttttaggtgcagaattggacaagccgttaaataaagtaattgttagaaataatctaaagaaagttacattccatggattacgtcatacccatgctacacatttggcttctattggtacccagccaaaattaatagcagatagattaggcgatacaatggaaactgttcttgaagtttatattaatgcagatactgaaccagaacaaggaatcgccgacaagttcgccgatagtttagtataa

SEQ ID NO: 13 - DNA, 36497 bp, synthetic construct
  misc_feature 1..36497: note = Description of sequence: Based on Lactobacillus reuteri sequence
  source 1..36497: mol_type = other DNA
ttaaaattgactaagtgcctggacgattttgtcatcagttttacttttatattcatcaatcaagtaggcgtatgtgttcattgtggttgtgatatcattatgtccaagtctcttgctaattgcataaatatctattccgttacttaacagtaaagcaacgtgactatgacgtaaactatggaaatgaaaattcttctttgataattttgtatcactcataatttgtcgaagcgttttatttaatgctgtactagtagggatagtattaaattgatccataaaaacaaggttacttgatgaattattgcgcaattgtttaagtagtaatagtagtttttcattaacttttattttacgtgtagatgattcattcttggtgggtttaaagcgatgtgtattaaaatgttcgtctgtttcgtctcttccttttacctctctccatgctttatcaatattaatggtatgatgaatgaaatcaatatcattccaggtaagagcttgaatttcttcttttctcattccagtatatatcgcagtgactatcatataacggcttgtatatcttctattagtaattccattgattgtagtagttaataatttccttatttcagttacattagggtattctaccttcattgttttgctagtattggcagctaaagtaacacctttagtaaaatctttaagcagataatcatcaaggatagcagattgtacgcaagaccgtattattgaattaagcttctttacacttgctatagcatgagttgctccatacttgttaataaattcttgatatttagaacggttaatttctttaatagaagcttctttgaaataatcattaattagattaccgattataatataacgattcagagttacacttgatattttaggttgcttataagtattaacccattcttcgtaataatcactaaaagcaatttttttctcaatgtgtattccgcgattaagattagtttccatttcagctgcccactttttagctagtgcttttgttgcaaaaccagctttggactttgaaaaacgttttccttcagtatctttccactgaattcttacagaccattttccgcttcttttatatatttgagccatacttgtccctccattattaaattgtgtataatgaaagggttgatagaatttgtttggacgcaattaatatcaacccttggtccactgacagttgccgctgtcggtgggcttttttagtttaaatagctttttacgtcatcaatttagttggacgagaaatttagcttttgtttagcttctttaattgattaataatttcttgattttgcttaattgttatccaattttgctgtttgattattcttaattcatgaaaaagggcttttgtatcattacttgtaaaagtaggataggcacctgttaatttttcggcaactgctatatcttttacagattcgagaatttcgtcatctaaactttctaaacgattttctttaatatatttatctttgtctttagcaaatccatctttaccattgaaaatactcatttttagctttctcctatctttttaatttttacattgtaaagagcgttatttaaatcattacagttcctaagataggaaaaagcgttttttaaaagtttattcatcctttgactccttatattcctaaaatttgtttctttttagcatcaaattcttctttgttaatgattccatcatcaagtagttgttttaattctcttaattgcgaaaggtcattactagaattggattgtgctgatgtctgcttaaaatctgcagctgcctgtttaatagtctcagccatctttttagcagggtagggttgcatattttctatttgagttgtaatagctccgttggttactgcaatagatcccaacattaaacctttagaatatgaaacgccattaatcatatcaaggggaatgtcagttgatttactaccataaatcaggccgtggtcaagaaaaattacgcgcttattagtacaaaccgcaagaatggaattagtttcaacgaaagcattagcagcataaagtattttttctccaccatcgacatctattatgtctggcaaggctttgatttcctttttagtaccgaatagattttctacattggcatcctttaactgttgcttaataacatccaatttttcttgttcgcctttgttgatttcgtcaattgctgcttgtaaagtatcagcttttttaccagcgctatcagcaattgaatcaagtttctcagttactttagatagctttgatttatacaaatcaggatgttctttgtaaggtacggtagaatgaatccctaataaactttcaatttcactagcggtatgggaactaatgaaattgctaagtttaattggaacacggtaagtatttgctggacgtttgggatcaagtctgttggcatctttgccacaaattaaatatccatctttggtaatgcaagaagcctttaacattcccagggcatcattacatatgaagcatctctttttactcatttggatcaccttattccattgtcactgaatatttaactaatttaccaacaattcgacctggatgctcttcatcaagaattattggatcatagtttttattatctggcattagcataactaaattgccttgatgcttaatacgttttaaagtagcttcgttatcatcgtcaacaagaactgcagctatctcaccatcttcaacttcaggttgctctctaataatggctaatgcgccattaggaatggtaggctccatgctatcacctttacaacgaagaccaaaaagtgttccactaggaactggtttttcaaagatttcatcagtataaccttcgatattttcttcagcagtaatcggatcgccacaagcaatttcaccaatgattggaattgaaacaacttctaatccttctttaggataaataatattagaaggtttcttttctctggtagggaaaaagtcatcaacatttacattaaaaatttcagctaatttaaataaaacatcttgatttgcctttctagatcctaactcatatctagtcacagttgctcttgtcgtatcaagtttgtctgcaagttgttctactgaaaggccacgttgttcacgaaaatttctgattttttgaccaatgaatttacttagttccattttatgtacctctcttatgtaactaattatataacttgcgttaccgaaatggaatattttttatctttttcgaaaaaaatagttgcatttgttccagaatggttttaatatataattgttccaaattggaacggaaaggagagataatatgcagtcaagattatatgatttacgaaaacatgttaaaggaatgactcaacaacaaatggctgattatctaaatattagtgtcaaagcttatcgtgataaggaaaatggtaaaaatcaatttactcaagatgaaatgtttgctattagtaaattatttgatcttaatattgacactatttttttgcctcgtaagttccatattggaacaaagggctaagagaggtgacacaaaatgcaagcatcgctagatgaacaagattaccaagtaattactaatgaggttctcaggcgtattaaggaatgttacaacttagtgcctaaacaagatgtccaaactgacaaatgggtcggtatcaaggaattcacgagtaagttgcccgttataaaggacaaggaatgggtcagaatgtttctccttacacttccagtctttaagaactgggttatcaatttgaatgcaggtcaaggtcatcgaactaaggtaaatgtgaccaaggcattgccttggattatgtctcatcaagctgatattgattggaaccagtcattgccacgttaggaggtgaacagatggcacaaccgatatttgtaaaaggaatttttggaagacgaattatagtttctcaatttgaacaaacgtctgattttattttgcaaaagaaaaacgctgacctaataaaagaagccagcggattttcaaaaaacaagaaattgtttactcgttatcaaggtaaagactttcaaattatggagcaattaaagtggcaacccagtgttctttttgatgatgccagtaagaaccgtagtagccacagatttaaggacaccaatggaaactgactttagagtgttagtagctttctttgtttgtttccaaacctcatcatctcgaatggaatcaagtagttcatgcccagaccaagtgatgtcatagacagttaattggcgaaccattttattaataaatggtttggcactaatgaaaccgcctgagtagagctgataaagggtataaacaatatcatttttatcaaaggtctcattagcaataacatttgcaatttgagaatctgtaaaaccttggtaaagcatttgcaattgctgtttttctaatgataaaagaacatacctaacacagtcttgcttcaatttcacaattttcaccacctttattttgaattaatcaaagtatagcagaaagggagagcagttaaatggcgttggtaattggattaccaatattaattattgcaatgtgtttgttatatgcattggtgtacagcttattgtatgagcggaataaaccattgctattgaaagaaaagtatcggaagaaacattgaaaggaggtgatatgaactttggtaaatttatttcttttgttgttttttatctttgttataatgccgcttttgtatcaatttttgaattacatcagaaggcaaattgaaaatttcttttggaaaaaaagccataaaaataagaacgaatgaaatgaaagtagctgtaaattcaaattgttctttggtctgagttattaatagcccagtaatagtagaaagaataattgctatttttgaaagatcatccaagtatttttcgcttatttgaaatcctaaagtttctgtaagccagccaaggataaacaatggaaaagaaaagaagccagttagaaaaagaatagtaattaagcaatcaaatattctgagtttttggaattcaaaaattacagcatctggattagcagcatatgtaatatgtagagctaaataagtaaaacagatgaggcagaagatacaaaccataaagtcggttagtttcttactatagtaattaaatttggttaaaaacttaaaaattttacgcataatgattttccttttgtactgagttaatcaaagtatagcaaatgaaaggatggtagtttaatgagtaaattaattgcgctaatcgttggtgcttggatcatgtattgttcaatgattggctcttatgatggagcgatggcgatcctagcagtttatcttctgctagtggttcttgatccactaaataaaaaaggtacgactgccgcaaacagtcgtaccgataaataaataatctacgaggtaattatatatgaatgagtttaatttatcaaaattaaatgccagggttggcgataattgtgtatttgtatcgaatttggcagttcgctatcaaagcgctgctactcctgaagagcgaatggcaatggccatcaaaatggagaatgctgctacgatgttacgcatttcagctgagcgtttggccactgaaactaagaatgtttacggaggtaagaacaatgactaacgaagaaaagattaaggcaattaaacaaattcttggaccagaatacgaggaggtagccattttcgctgctaaaaaatcagaaattcgtgacgaacgtactaattcattagttgatgcggatcccggaacggtagcagccatgattatgaattggttacatactaatccgatagcagcttcaattgtcaaagcaactattgatactttggaaactgatccaactgctgatttatttgcacagatttttctaggaggtaagaactaatgaatttatttgaattaaacgacaactacaaaacacttgctagtcgggatgacttagatccaactattctaaaggatacattggaatcaattaaggatgatcggaagaccaaattggacaatcttgcttcatgggcagatcatttgaagtcagaaattgattttatgaccgacaaaaagaagtcatgggaagaagaaattacttaccgaaaaaataaacttacttggatcaagaaatatattactgaagttcttgatgatgccggtattaagaaaataactactgaaaatcacttacttagtgcccggaactttaaggcctcaaccattattgatagtgataagaagcttccggataagttcaaaattactgagacgactactaagccagataagcaggccatttaccaagcactcaaagctggagaagaagtaccaggagcacatttaaaagctaaccgtaacacggtgattaaataatgtttgaactccgtgattatcagcaagaaacgattgataacatcatgaattctataagtgctggtcaccgttctatcatggttcaacagccgccacgaacggggaagacagttattatggccgagattgctagacgagcaacggcaaagggtaaccgtatcttgttcgtggttcatcggcaagaaattgtccagcaggttatcaagactttcaaagctaatgatgtaaatatggatttagctaaaatcggcatggttcaaacgattacccgacacgttaataatttggacccaccggcgataatttttgttgatgaggcccatcatgttctggctaaatcatatcgaaggattcttgatgcttttccgaaagcttataagttactgtttaccgctactccttatcggttaggtggacagggttttactgatgtggctgatgatttaattattggcaaatcagtcccctggttaattgaccatcactttttagcaccagttgattattacgctccttcttacattgatactgccaagttaaaagtaaaacgaactggtgaatatgacactgattcaatcaaaaaagccatgaagcctaaaatctacgggaatgcggttaagcactatttgaaacttgctacgggaatgcaagccattgcctatacctataacgttgatagtgcaattaagttagccaatgcatttaatggctatgggataactgcaagcgccgtttccggaaaaacgcccaaagaagaacggaataaaatcattgaggactatcgccaaggaaaaattcaaattgtaaccaatgcagaattatttacagagggcctagatttaccaaatgttgattgtgtcattatgttacggccaactcaatcattatcgttgtacttacaatttgcaatgcgctcaatgaatccacgggaaggtaagactgcaataattatcgatcacgtgggaaatgttgaacgattcgggctgccgactgatgagcggcaatggacattagaaggtagcggaaaaaataaacaacaaccaggaacaacgcttaaacctgtatcagtatgtccgatatgttttgcttcgttttatcgtacaagtgatatttgcccttattgcggggcggcattaggagaagaaaaagaaattgaagtcgttgatgatgttcaactaaaaaaagttactaagtcacggctagcgattattaagaaaattcaatcgtcagcaattatgaataatgttgctggcaagcgtccaaacgaattgaagaatctgaaagaaatacaagcctatgccaaattaaaaggttacaaaccaggctgggcttaccactacgctaaacagcgtggatttattaagaagtgaggttgatattatgagtattttgccaccaaataagccacagaaggcacggcgagttccaagaaattactttatctacggagatacaatgtccggaaagtcatatctagctgaacgttttccaagtccgctatttcttaacaccgatggtaatagtgagatgaacaccgcaccaagtattcaattaaagaatgtccgaaagagcgatggaagcttgaaagagtcagtgattgatcaactagacaagattattcttgctcttggtactgaaaatcatggttacaaaacagtagttattgatgtgattgatgatgtagtaacactaattgaacaggccatctgttatgacaatggagtagaaacgctgggggatgttccttacggcaagggatatgcacaatttaataccgtctttcaagcatttgtcactgagctaaaagccttaccactgaatacggtttacattagccggttaatgatgctaactgatgaatcttctggccacaccgaagaccgaccatcactaaaacagaaatattacaacgtggttaacggaaattgtgatttagtgattgaaactaagcgctatggtgaccgttatatccggatggttaaagatcgacgaattcattatgtcaaagatgatattactgatccggcaatcttacgggtacttgaacatgtaaatggcgtctttgataagccaaagcagactactacaaaagagcagaatgaaattgttaacaaaattaaaaagcaaaatgtaaaggaaggttaatgaattatgagtttacgagatgcaatgaataaagctactgaaggttttgatccaaagaatgattcagttaataaatttaagggactggaaagtggtaaatataccgttgtagttgcaaaagtagaaaaccatgaaactccttggaatgctgaacagcttaactttgagttagaagttgtcgatggagaatcagccggccaaaaagaattcttacaaattggattagatgaattaacttctaagggtaatcccaatccaatgctagaaactaatttacgattggtttctaagttagcagcaattctaggtgttgaaattcccgatgaagtttgggatgacgatactttaatctacgagaacttggctaaagcatttgcaccggcagtaggaaagaccatgattatggatttgaaggttcgaccaaacaagaagaacccccaatatccataccgtaattatgactttgatgaagcggaacagccggaaacgccagaagttacagatgatgaaatgcccttttaagtaaatatttgagtcagtgaacttataacaccgtatggctgggaggccattaaggaggaaaaatgaaaaatctagttaattacgccttagcctatcaagcaaagggtttaagcgtcctcccaattgctggcaagcgtccactaattaagtttgctgatcgtgatccacttaccgccgaagaaataaagaccatctggatagaacatccatatgctcaaattgcgttgcggactgataagttcttcgttgttgatatagaccgcaaccatgctgataacattgatggttttgaatcaattaagcaattaccagcggaatattttccggaaactttaacccaaaccaccaagcatggtggccgacagttattttatttgaaacgttcagatatgcgagttaatcagttaattggttatttaccaggtgttgatgtaaaggcccaccaaaataattatgttgtggttgcgccatcagaaggttaccaatggctgaataagaagccgattgttacggctcctaagtcgttagtagtgaatattaaccaaatgcgagccagtaaccggcgaagttctccagatgatttagtttttaaaccacgtgaacgcaactcaactactgacttacttgagaccatcgcaaatggattgggagataaaggaataagaaataaaaacttagctggaatgattggtgctttattgtttcggggagtagaaccaaagtctgcttatcaattagcgatgatttgtaatgagaatacgcccgatccactaccagaagaagaagtgaaccggacatttcaatcaatgctaagacgtgatttgagaaacgggggtgaaatacgtggcggataatataattcgcaaaccaattgaatttgaattaaatactcaaggcaatcctaaaactaatagtttgaagaatattggtttaatccttgatggcgatccactactgcatggcaccttcaaatataacgagtttgcttattcaattgatgttgttaaggacattccacagctatttattgaaaaggggcaacttgatgatagctattcagcaattatgctccgttacattgaggatgagtatggggtgatgtttcaagaaaaattgttaaatatggcaatcactgttgaagcaaaaagccacccatataatccggttaaagagtatatggaaaagtgctataagaattgggaccacaaagaacgaatcaaagacttcctaccagtctatttaggagtacccagtggtgaagtaacaacgctgcagacaaaattattcttagtcggagcggtgatgaaagtctataagccggaaagtaaatttgattgggtgtttgatttagttggtggccaaggcgttggtaaaactactttacttaaaaagttagcccatggttggtatacagaccaatttacggacttcaaagacaaggataattttgccaatatgctgcgggcattgattgttaatgacgatgaaatgacggccacaaataattctgactttgaaaatttgaagaaatttatctcagctgaagaattagagttccggccaccatatggacgacatacaatccgccggccaaagaattttgttatggcccggactactaacgaatcaacctatttgaaagataaaaccggtgagcggcgtttcttacctaacatggctgataagtcccaagcaatggctaatccggtaactgatcttgatgatacgatggtcaatcacatttggggtgaagctgttggcctctacaaagaaggcttttctttcatattgacgaaggagcagcagaagctcattgaggataatcgaaagatatttatgtacattgatgaaactgaaaatcaaattgaacgggttctcagtacttgggacgatgactggattgaaagctcagaaattgctcatcaattaggtgaagataatctggttaagaatcgttcattagccaaaaagattaagtatgtaatggataaccggcatgattggaaagcaggacaacggcgaattaaaggaattagtcatcgtggttatagaaaagtgcatacagataatacactatgaatatagttaagtgtatgcagaaaaaacctattacatcaacgtttattaaggtttgcatacactactacactattttaataataaaaaataaatatatataaatactatatatgcgttataaaaagttgaaagttagtgtatgcgtgtatgcagtaactaaatccgttgagagagtaagaattaagctgtatacactactgtatgcatagtgtatgcaaggaggaaaatatgaatgaattaaaaaacatcagcacatcagaattacttgatgaactgattgaacgtaatgcattatttcgtgtagattgtggtctttatcggaattgggagttgaagggaaaatatcaatttagcgatattaaattgcctagcgcatatcctatttatgtaggaaattcaattattgaccgcatgattaagtgggagtgtgagcattgacgagtgaacataaaattcaaaacgatatccgggttgcactatcaaaacatcagtgtacagtgttccgggtaaatgtcggttcggtaaaaacaccagatggaagatttttttcagctggtgtacctagtggtcacccggacttatatggatttcgttggtcggatcatcaagtattttatattgaagtgaaaaacgaaaaaggtaagcctagagcggatcaaattagatttcatgaaatgctaactaaacgagaaattattcatggaatcgctaggtctgctggagatgcagtaaaaattgttgaggaaggattgattggttatggatatgaaaataaaaaagaaccaccacaacaagtaaaattagttgaagtaattcaggttataacctctcgtggagctggaacaaaggaggatccgataagaaagattattcagtattggagcaaagaaggcacattattagcagaaagttttggaaactaatttttctttgagtcacgtttctctatgtgctttagttgtttttcaggaagactattataaaacttatcaatttgatttggctgtgttattaaattgtctgtaattatatttaacagatttaaaagacttttcgccatttcttggttatcagttaagtctatctcaccaggatgaacagcattatttccaacaattcttacaatatcaaacgcttgttgtaatttagccggaagtcccttagatactagatcaccaatacgattatctaatgaatgtttattggaaaattcttttgttagtttatctattgccagacgagacaaagcagcagatgctcttggtgaatctctaagtacttttgccgcttcaatataaatttgtttaatatcatcgggcatatctggattaggagctgttaattttaaaggaaaactaggaaagataagtatatcttcattggattgccgattatgaaaccaaatagaatctaaataacatgaagtgcattttgcaactattaaattatattcgtattgttttgattttattttttctgcatatttccaatcaaattgagcatatacatgacaataaggacaaataataggagctttccctgtaaaatcattactaaaatatttagtcattaaattcacctcaaataagattatagcgtaaaggagtagatgtaatgctacacaaatatagaaagttaccaattgtggaagctgaacagtttgatgggtcagatgaaatgattgaaaggtattcagtgcatgtatttaatcctaatttagctaaaaacatcttctttataggtatgaacgttctagctattggtgattggattgttaaggatgaatatgggaattatcaagtggtagctgatgatatattccgtaaaagttatgagaggtgcgactaatgcacatttatgaagtaatcgttgtagctgtatttggcacagatattagccactttgttgttgctaagaatgccgataatgctaagaaaattattcttgattattacagcactcgtgatgatggtatcaggccaactgtgacaatgtatgacctaacaacaaaattaatcaatcttaataactacattgatgaggtgatgcttggatgagattaagtgacaaaattattatgacttcctttttactgttattaattgtttcagtcgtcttatcaatagttacgggaagtaaagtctggatatggatttttctttttctgatgacacttgagatgctttacaaaatttggcgttaggagcgtgactaatgaaagcaagatgtggcgattggagtactgatgtttatccagttgaaccgtttgtttatgaagtagcagctgatggtaagaaagaattttatcaatcagcatgggatgcttttagttctgaattgcatatgaaaaatgtgacggcgaagattatcaaaataccagttgttccaatgagtaatgatgaaattaaagcagagagtgttgcatttgaattaggcgaaaaaaggcattatccacatgtggaagaattcactaatagtaaaccgaggaggaaagttacatttcttcaggttgcggaagcttttgcaattataacgtggattattttaataattcttataataagtacggcgtttatgtaggaggtaagttactaatgacatttgaagaggcgttaaaacatgaagaaaataatgtgccagtattctatagcgatcgaaaatattatgtaatcgggcacaatgagttaaccgaacaatttacaattcgtgagctaagtggtaatccgttatttacggtgccggttgatgtacaagcggaggaattgtcatgagtattaaaattaatgcgcaaacagttgtatttaagggacaaagttttattcctagcaaaagtaactgtgagtattgtcaatttccatttaagaagttaatggttactaaagcatctccagatccatcagttaagacagaagtaccaatcaaagttgacggtgaggtatttaattattgtcctcattgtggacggaatttacaagggtgaggcaatgttacttttaattgtattaatgatgattgtctttattgctgggttcctattgggtaagaaaaatccatgacgaaaaaaggacccaccgtaaaagatgtgtcctcactaaaaatatttaaccataattattatatcagatagcgagggtacgtcatgcaaacaagtttgaatttagatattgattgtctgaaaactgcaagaaaggttactgactttcttgataagaagctggatcgctatctggctttatcggggaagcaacgttttgatttgaagtcaccagggatggacggaatgcctaaagctcccagccatggtaacggtagtgaaaatcgaatgctgaatatctggttggcagaagaggttgttgattgtgtgggttgcgctatgcggaatatgacaaaggaatcacaacggattttactaaatcggtattcggatcagatgttaacgtataacattgctagggagctaagtattagttcagcaacgtatagtcgaaagcaggaaaaagcgctgtgtgagtttgcggaccggtttgaatttcagttagttaagcacggtattcatactgaaattgatgatctacacgtttatcttgacgaggaagattgataaattgatgatagattattgagcgagcaattcatgatagaaatgtgataatagtattgtcgaatgattcgatattcatataataatcttcccaaatgaagtctagctattatggctaggcttttgtattatgtttaattgggtgatttttatatgattgaatacttaaaaacatttttggaggctttgagtatgaagccaaaaactaaatttgttggagttatttttggaatagtacttttatgcttaaaaccatttttaattcaatataatatgaaatggttttacaataagttttcttggattattattttaattactttattttttgtagcctcattaataattgaagtaatagttgaagtatacgaatggggtagaaacaagtataacaaacataaagttgaaagagattacgaaaaatatatcttaggcttgtctgataaaaagttggcaattgtaaagaaactttacgctaatgaacatcaccaaggatatttaagacaaaacgatactaatgttattgaattggttaatatgtacgtaattatgcaacttaataatgaaattatagtaagagaaagccaagttgaagatataaacgatcctgaatttctttttgtattacagccaccggctttacacatcatagaaaagaattcagaaaaatttaaataaatttaattagtttagcttaacggctgactttttactttttaggaggtgagtagcattactcaaaaattaacacagaagcaacaacgatttgtcgatgagtacattatttcgggtaatgctactcaggcggcaattaaagctggatattctaagaagacagctaagcagtctggtgctgaaaacctagcaaaaccttacttaaaagctgcaatcgaaaaacgcaatgctgaaattcaatccgagaaaacagctgatatgacagaggtgatggaatatcttacttcagttatgcgtggtgagcaaacagaatcggttgctactgctaagggtatttatgaagacgttgaagtgtcggcaaaagatcgtattaaagctgctgaattaattggaaagcgtcacggcgcctggactgataaaaaggttatttctggtgatgttcagattgatgtgggaatgggggattatgatgatgaagagtgaagaacaatggagaaaaatcaaagatcatcctcattacttggttagcaataaaggtaatgtttacagtgagtataaaggcggcttgcttaaacagatgaaagatgcttatggatattctcaggttaatttaaaccgccgctccaaaaaggtgcatcgtttagtagcggaagcttttatcccaaacccagacaaattgcctgaagttaatcataaagacgaagataaaaataataaccaggtggataacttggaatggtgtactagcaagtacaacatgaattatggtgacgtggagaaaaggtcaattctttcacaacaaagccatagtacttggaaaatttatcaatatgatttaaacggtaatttggtaaaagtatggaattcagcgagagaagccgacagacatggattcaaccgtagaagtgtgtatcgctgttgtgatggggaaataaaatctttcaaaggatacatatggtcaagacaaaagaaggtgataccatgccaaacatcaaactaaattttcctaaaccatacaacgttttcaataaacaaatttttgataacttgtttgattacagtcatttcgttgaggtttggtactgacttatggcggtgcatcttctggtaaatcgcatggtgtggtacagaaagttgtacttaaatcactccaacactggaaacatccccgcaaagtgctatggctgcggaaagttgatcgaacaattcaagaatctatcttcgctgacgtaattgactgtttatcaaattggcaattgttatcgttatgtagagtaaataaatcaaaccgtactgttcatttaccgaacggtgcggttttcctgtttaagggtatggatgacccagaaaagattaagtcaatcaaagggttatctgatgtggtaatggaagaagcttccgaatttacacaggacgacttcacgcagcttaccctacgtctccgtgaacctaagcataagaaacgacaattgttttgtatgtttaatccagttagcaaattgaactggacttataagcaatggtttgatccgaaagtgaaagttaatccggaacgagtatcaattcaccaatcaacttacaaggataatcactttttggacgctgataacattgcaacgattgagaacttaaaacaaaccaacccggcctactataaaatctatacgctgggcgagtttgctacattggataagctggtctttccagagtttgaaaaacgtcggttaagtattcgaaccttatcacagcttccctcgtacttcggcttggactttgggtacactaacgatgaaacagcctttatgcacgttaaagtggatgagagtacccgtaaaatttacgtgatggaagagtacgctaagcacggtatgttgaacgatgatattgcccgaataattaaacaaatgggttatagcaaggaagttattactgctgatgctgctgagcctaaatcgattgctgaaattaaacgcgatggtatctcgcggattcgtccagctaaaaaggggaaggacagcattatacagggactttcatttatgcagcaatatcacttagtcgttgatgaccggtgtgtgaaaacgattgaggaattggaaaattatacatacaagaaggacaaacaaactggtgaatacaccaatgagcctgtcgatagttacaaccacgaaatcgatgctatcaggtatgctttatctgaaatcaacggaatggctagtccaaaggcaactgtaatgaaaaatatttatatttaggtggtgattgaatggaaacagtaaacggtaaaggacaaattttagatggccatatttttatctatccagctgatgaagaagaacttgatccgcatgatttactgtcgttcatgagaagaaatattcagtatgctaaggattacaagcataatatgcaaatgtatctaggtaatcacgatatcttagatcaacagcggcggatgtatgggccagataatcggctagtagcaaatttaccgcattatattgttgatacttataatggattctttactggaatcccacctaagattactttagatgataagaatgagaacgaagcattacagcaatggaatgacactaattcgttccaggacaaattgagtgaaattagtaagcaaacggatatctacggacgttcgtttgcttttatttatcaagatgagaacgcagacacttgtattgcttatgcttctcctacagatgccttcatggtttacgatgatacggttgctagaaaaccttttgcttttgttcgttactggaaagatactgaaagcggattatggaccggaatggtttattacgctaataaaattaaaacctttaaaggtagtgttgttgaagattcagatcaaaataatatgtataatttagtgccagcagttgaattttatggaaatgaagagcggcaaggtgtttttgataatgtgaaaaccttaatcgacgaattagacagagtgttatcacagaaagctaaccaagtggaatattttgataatgcttaccttaaaattcttggtcttgatttagatgaggatggtgatggtagaccggatgctaatttaattggtaatcaaatgatttattcgcctaatgctgatgctgctaatgccgatgtcgaattcatttcaaaaccagatggtgataatatgcaagaacatattattgaccggcttgtttcaatgatttaccaggtaagtatggttgctaaccttaatgatgaagcgtttgctggtaatagttctggggtggctttgcaatataagttacttccaatgcgaaatatggcagctaataaagagcgtaaatttactcaggcactccggaagttatatcgaatagtgtttagtgctgatcaagtagtcaaagataaggaagcctggcaagacttgctctttgatttcaaacaaaacttaccgattgatgtttctgaagaagctgatactttacaaaaactatcaggggttgtgtcaaaagaaactgcattccgaaatagtcgtttaattgatgatcctaaaaaagaagttgagcgtatgcaaaaagagaagcaggaagaaataaaccaagcgcttcaacattctgcttctgctacagatcaaatgctaatggatgatcaaaaagaaaatgataaagagatagttggtttccggaagaacggtgaatccgatgacgaagaagaataattattgggctgatcgtattgctcgggaacgtaaatggcaagaagagcaattaagtaaagatgctcaatttaatcagcgccttcaacagtattatgatcaagcaattgtccagattaataaagacattgaagatcagataaattctttagctgtccggaataaagtttcttatgctgaagctcaaaaagaagtgtccactaccgatattgctgattatgaaacagaagctaagaaggtagttcaggaagctaatcgtttaagagcacaagggaagcatgttacttacaatgatttctctgatgaagttaatgaacgattgaggaattataatacggcgatgcgatataaccgattgaatttattgaaatctaaaattggtttatcgatggttgaagccggaatgaatattgatgctgatatgcaagctaaaattggtaaagattatactgatgagctaaaacgtcagtctggtattctagatcattctaccgaaaatagttcgttttggacttctaaagatgttgcgaaacaagtaatgaagcaaattaatggagcaacttttagtcaacgaatttgggctaatcaagatactttgaaagctcaacttgatacggttatcaccaacggaattttaactggtaagaatccgcgagttgtagcaagacaattaagagataaagtaaaagtcactgttaaaaatcacagttatgttactgaacgtattgccaggacagaatcagcacgggttcagtattctgctcagattgaattaatcaaaaagaatggttatcaatttgtccactggattgcggagccaagagcctgtgatgagtgtcgaaagattgcgacgcaagataatggctttggtgatggtatttatcgaattaataaagttcctaaaataccagacgatactcatcctaattgtcgctgttcaattagtgagacatgggtcgatggtcaacgcaatatagcattatctgatgatgaacaggcggcattgaataattatattagttcagattcatataagattaatgatgatttgaggcgtaataagatttctaaaaataagaaacaatttattgaaaacttagatgccgcattggctaaaatgccaatttatcatagtagcaagccactccagcgtgattatttctttgataaacaagaagcattggatgattttattagtaattttgaaattggtggagtcttcactgattcatcatacatttcaacttctaaaatttattatggacagggcaaagagacaattcatgttattattaaatcaagtaagacaggaagagatatctctgagtttaattttaatgagcaggaagtattattccccagaaatagtaagtttaggattgatgatgcatacgttgatgataacgggaagatgacaatggtttggagtgaattagatgagtaacaagccttttactgataaacgttggcgagataattctttggaaggcgttaaatttgataattcaaaagtaacgtcagaacaaaagaagaagacagaagaattccatgagttattcaaaaagacgtttgctaaacaattaaaagaaaaacactcacataaaaagtaggtgatccaatgggaaataatgatttctttacggtaacgtacaaaatactaagttatcttaagtattgttatgaaaatggaattaatcctgatcctaatattcttaatgctgatacatttaatattagtaaagttcaatttggaagaactctacaaatgttaagcgagcatggttatatttcaggagtgagatttacacaagccaaaatcgaaggtactgttgttggtggactccacaatacgtcaataacggttgaaggtctgcaatatttagctgaaaactcaatgatgaaaaaagcatatcgaatttttaaagaagtcagagattggcttccgggtttctaagcattcacaaattagtgagtgctttttgttttggactttttacttgttgcagtcgttaaagaacaacccggatattacagtccaccggactataaacgaggtgtatttatgtttgaaaaattaccaatgcgtttacaattctttgctgaagatccaacgccagatccagataatgatggtgcacctgaaggaactgatgatggagataacggtaaaagtgaaaagacatttactcaagcagaattaaacgatattgtcaaagcccgagtcaatcgagccttgaagaataagcaagaggaaattgaccaggctaagagtgaagctactaaacttgccaagatgaataaggatcaaaagcaagaatataagcttcaacaaactgaaaaacgtgcccaagatgctgaagcagaattggcccgttataaaatgcgtgatacagcgaagcaacaattaattgatggcggttatgacaatccaactgatgaagatatcgatttaattgttactgataaagcagaaacaactaaagaacgtggtgaagcatttcttaaagcttataaccgaattaaagaaaatgttcgtcaagaactattaaagggaaagtcaccacgaattaatggtgctcctgctactgcaatgactaaagaacaaattgcaaagatcaaggatcccgtcaaacgggtccaagccattcgggataacttatcccaatatgaaaaataaaaggaggaatataaaatggctgaaactaatttaacgacaagtacagacctagttgcacaatctatcgactttgtagaacaattctctggaggaatccaaactttattgaatgcattgggagttattcgtatgcagccaatgactactggttcacagattaagatttacaagtcagaagtaactaaggtcgatggtaatgttgctgaaggggaagttattccgttaagtaaggttactcgcaagctagctaatactttgacattaggatttaaaaagtatcgtaaggtaactaccattgaagctattcagtcagctggtggtgctacacctgctatcgtggatactgataataagctacttcgagaaattcaaaaggatgttaagaaggacttatttaattacattaccaagtctgatgcaaacaagactaccgcctctggtgacgattttcaaaaggcaatggcggcagctttaggacaactttcagttaagtgggaagactacgacacacaaactgtcgcctttgctaatccgcttgatctatatgcatggttaggtaaccaaactcttactgttcaatctgcctttggtttgcaatacattcagaatttccttggctttgacactattattctaagtgctgaagtaccacagggaacgattgctacaacagttgcagataatatcaattacttctacgctccaatttcatccgttggtcagttatttaacatgacttctgatgaaactggtttaattggtgtaactcatgatgcagttaataacaatttgtcatacgaaactgttgtaacaatggctaatgtattgactacagaacgtttggacggcattgtattgtcaacaatcagtggtgctaagtctgctggtaagtaggtgattgaatggaccagaatacggttttgcaaaacttaaaagtaatgcttgaaattaaaaatgatgaccgtgatgctttgctgaaactaatcattgataatacagaccaagcattgcgatttaagctagaactaactgaagacgaaaatttacctggagaactaggttatattgaattagaagtttcagttcgacgattcaatcgactgcaaaacgaagggatgagtcaatatagtcaagaaggggaaagtattacttttaattcttcagattttgatgatttccttgatgatattgatttgtggaaacgacgacaccagaaagatgttaaatctttaggtgccgtttcttttattaatccttatgcggggatgagtaaaaatgcgaaaaacacagataattaagttttattttcaagatgaaaatggctataatccatatgcagaagaagacacgattactagtcctaagctagtagcacaacggtatgccaatgttactgatgtagggacaaatcgcctagttgaattatttagcaggctagatcagaacgccaaggtaattcgattggagtctccagtaaatgactcttggtcatatctgactattgatgattgtcctatcaagtatcgtcttgaaacctgtcggaaaccattaaaaggcacaacgctgattgtaggtgaagccagtggctaattcttttaaggttgatgttaaaggtactaaagaactagcgaatttcttaaagaagaataaagatttgactccagttaaacggatagtcgcaaaacatggagcaggtctcaaaaagcaaacgcagcaaaatatgaacaatttgtataagggccactacgaatggaagaagggggctgggctaacaatggttagccctaccgggaatactagacggtccgtaacaaatacaatttctaataatggtttaacagcaacggttgctccacaaactgaatatttcccatatcttgaatatggaactcgctttatggcagcacgaccgacattacatccagcgtttgcaattgagtctatgaaatttgctaatgatttgaataagttatttaagtaggtgagaaaatgtcaccaagtattgaaatttatgatgcagtttttgctcaggtccaaaagcattatcagacctacgatcacccaccgcagttaaatgaaccagttacctatccattcgtggttgttgatgatagccagtcgattttgacgaactataaaacagccacaggaatgcgggtaactttaatagtccatgtgtgggggaagtctaaccaacgtaagactgttactaagatggttgatgaaattagtcgtctggggatgcaagcagttcggacgaaacattatgcttggcaaggacgacctaatgagcaagaacaacaattattaactgatacgagtgttccgaatactgtgttaaagcacggttatttaacactcgtttttgatttgaaataaaggaggataaatatggcaacatatccagtattggaagggaagaatgcagttctttttgaacgactattagaaaatgcaaggaaagagccggcacaattgatcccgtatcaaacatcactaagttatgatcctaaacgggatactgattcaacaactacgaagatggggaatgttcctactgcttctaatattgaaacagatttagaagtagagttcctaaatgcaatttccaaagctgcagatgatgtttatgattctttgtactttaataagaagattgaagtatggaaggttcatattgatcgagtccggtcagatggcaaagtttatgccgaatatatgcaaggaattgtgtcagaagactctaatgataacgacccagacgatcattcaactcgggatgtgacctttacgattgatggcgtggccaaacgtgggtgggtcactctaccgccagaaatcaaggaagaaattgactatgtattccgtggcttggcacagcttaaaggcgatgacgacaacggtgaaggtgaagcttttgctgatggcgatcgtggtgctggtgcaaatgaagcagtaacaactgaataggaggaagattatgaagttaaaaattaatggtcaagaccaatcatttgtattcggagttaagtttttgcgaaaacttgatgcttatcggggcgctgaacaagaaatccaaggagttaaggttaagctaggaatggggctagccatgatgcttccccaattaatgactaaggatgcggccgctttggcagacgtgttgtactgtgcggctaagtctagcattaagttagatacaattgatgattatattgataattgcaaggacttggattcattatttaatcgggtaatgaatgaaattaaggcaagtaatgccgctaagccgattgcaaaaaatctaaaagcctagatggtcctgagcttagttcagaacaaagctatcacgaaattcttttgaattcgttggcttatctaggctttcataatatttcagaaattgaagaaatgggattggctgaatatcagctccggatggaagcctataacctccaacgggttagccaggaacgagacttagcattgcaagctttccttaatcagtcggtacaagcgacgaaagggagcgaaaagcacccaattccgaagtataagaagtttagccaattttttgattatgataaatttgttgatgatgttcgtgggcactatgagcctgactatcagccaacaagcaaggccagccttgaaaagaaacgaaatgatctaatcgtcaagcggtggcgtgaattccggaagatgaaacaaaaacagagaggaggtaatggctagtgtcacaatcaatgagagttgaagcggtattatcagcatacgatgagagttttagcgcaaccttagataaggcgcttaaatcgattaataatttaggccgtgaaacccagtcaacctctcaaactgttagtgcaggtggttctagtatttccagtacctttaaatcgatggctggagcaatgggtgtagttgcgattgctggtaaagcatgggacgttgttaaagattcaatgagtggcgccattaaccggtttgatacattaaacaagtatccggtagtaatgaaggctttgaattattcaactaaggatgttgcaaagtcaaccgctatcttatctaagggaattgatggattacctacttctttgcaagacgttacaagtgttgcccaacaattagcgccattaactggtagtgcaactaaggcttctaagtcggcgattgccttgaataatgccttccttgcctccggtgctagtgttgccgatacctctcgtggacttcaacaatacacacaaatgctttcaactggtaaagtcgatttaatgtcttatcgaacattgatggaaaccatgccaattgcattacgtaaagtcgccaattcatttggttttactggtaagtctgctgaacaagacctttataaagctttgcagtcaggacaaattacggtagatcagttgaatgatcgttttatcaagctgaatggtggagttaatggttttgctcaattagcaaagaaaaatagtgaaggtatcggtacatcttttgcaaacttaaaaaatgccgttgtcaaaaatctggcaaatatgttatcggcaattgacaatggttttaagcaagcgggctttggaagtattgcacaagtcctagacaacatgaagggtagtattaattctgcttttcaagttattggaccagttgttactaatgctactgttgtagttcttaattttgcaaaggttataggcggagcgcttaaatctgctttcagtaatgatatttttaaaacagcagttgtgggaatattaggctttgtgggtgcagttatggcagcccataaggttatttcaatatttacaacattaagatctgcaatagttggtttaagtgtgattacaaaagctggtaatttggcaatggcgtttagtgaagcaatgtcaacacttgctaaaacttctaagattgctggtggagcgatgaaagcattcagtgcggtggcctcattaggtccctggggaattattgctgttgcaattgcagctgtggttgcagccttaacttatttctttacccaaacgaaaaccggtagggctttatggcaaagttttactacgtggttatctggagtatggcagagtttggttggagtggctactactgtttggaatgcaattggtaatgctattaatgcagtagttaattttattaaaccttattggcaaggattattaacattctttaccggaatctggacatcaattgtggcgggtgttgctccaatttggcaagggttagttaatgtctttaatagcattatcagtgcaattttagccgtttggcaggctttagctccaattattgttccgattgtagctggtgtagttgctatcattggggcaaccctaattacgattgttaccgtctttcaaactgtgtgtaatatgcttgtacccattgttcaagttgtatggcaattaatttcaacagttgtatctactgctattacgatgctaggtacaataatccaaacaggcttggcaattatcgttgctatttggaatgtggtctggaatacattcagtattgttgtaagtacggtatggaacgttattactactattatatctaccgtgctgaacgttattgcgggaataattcaagctatcactgctgcaatacaaggcgattggtcaggagcttggaatgcaattcagaatgttgtatcaactgtttggaatgcaattgccagtattacttctagtgtattgaatggaataaaaggaatctttgatggtgtaatgaatggtttaaagagtattacttctagtagttggaatggtattaaatcgctattcagtgaaggtgttaatttcattaaatcagttgttcatatagatttaggtgctgctggtagagctatcatgaattcactttggaatggaatgaaatccatttggaatagtattaagaattgggttagcggtattgctgattggattaaagaacataaaggaccaatcagttatgaccgtaagttacttattccagctgggcaagcaattatgaatggtcttaataacggattgattaatggattcagtgaagttcaatcaaacgttagtgatatggctaatcaaattcagcaagctattactaatccaggctttgatattggagcaagtattggtaacttgggttcaattaattcaaattatactggtagcctggcaattcaagatagtcagttacaaatgcagaataatgctttgcttcgtcaattacttaataaagacacgacaatggttcttgacgatggcactcttgttggctatacagcggatcaatacgattatcgcttgggtcaaaatacagcattgaaggataggtggagccgatgaaattcttaaataatgactattcttttcgtggattaggacccacaaaggatgatccagaatacttagaaaatgcagaatatatcgactttgccggttttaattcttctgattatgattggtggttgattgatcgaacagcaactacgccagaagaacaagaaattacagaaagcgtcccttacatgcaaggagaatatgatttctcaatgtatgatcaggaacgtttttttaagacccgtgagttgacttataagtttgtatattttggtgaagtttatcaggatcgtaaagcttacgaagaggagcttaaacggcaattactgccacatggtttcactaaactaattgattctcatgatcctgtttactactggtcagctaagtgtactagtgttgaggttgaggatgaccaagaaaagggaatgctcacagcaactattactttcaaggcttatccttttgcttatactaatcataacgagggcaccgattattgggatgatgtcgcatttgatcattggatttggcaaccagttaaattcaatgttaacggtgatcaggatgttaatgttaagaatatcggctcacgaccagtcgaatgctcatttcaattgacagggtccataactttgaagaacgattcaattggtgaagtaggtttaactcaagacaattttaaaacaaccacgattgtattagagatgggtgacaataaaatgcatctatccggaaacgggacaattgaatttcaatttaagcgtgaggagatgatttagtgtaccgaattattggttataatgaaccaacagataaagcaggatttattgtactggatccccgagttaatcgtcatattagttcgggaaaactcacgcttaaagaatctaatattgatgatttgactattacggttaatcaagcaagtccattatgggacaacgtaaggccttatcatactcatgttaacgtttatgatgataatgaacttatttttcgtggacgagctatcaaacctaaaaagtcgatggaagaaagcggacaattcattcgtgaatatgtttttgaagatattgaagcatatctcatggatagcacccaaagattttatgaaggtgttggtcaaacgcccaaagaatttttacaaactttaatcgatgttcataattcacaggttcctgactataaaaagtttcaagtccggaatgtaaatgtcactaataataaggatgaccaatatcgacaaattgattatcccaaaactagcgatgctattaatgataaattagttaaatctcttggtggttatattgtgactacttacaacgctaacggaataaactacattgactacttaacggatattggggttgatcataaagatgatactcctattcagttagctaaaaatatgaagtctgcaagtatgcaaattgatcctactaaggtgattacaagactgattccactgggaaagacactagaaccatcaaaagttgatgtaagtgatgatgatggagagggcggttctggatcattagatagccctgaagaattttgtaaatcagaaattaatgctacttggggtagtgatattaataatatgaaacaagattttgccgctcgttcttcgagagttcgggcttggggagtggacgttaatcgtttatatgatgtggtgaaaaatgctggagtaagtcctgaatggttctttgcttatgaacttcaagaacaaggaacttactatggatggcttaaccatacttatcgacacggtgatgcgtatagtgatgcgcaatctgtttgtgagtggattaaaaattgttcaaatagtaattccattaatccagcatggagcgcaccggaaggatcaatggcgccgaatcaagcattagcggataaatggaatcaagagtttggaaaaggtactattggccgcgtttatttacaagggactgccgctgctgtttgggatttagctggtcaaacgcctaatccagctattggaaagccaattagtggatgcatttcttgtattaaacgttggggtggtcattctaatgcagctggtggtacatggggatggccttttcctgatgttggggaaggtcatttttctcaagttcagagtttcggaaatgatggcggatatcgtcaaaatagttatcacgatggtgtggattttggatcaatagatcatcctggtagagaagtgcattgtattcatggtggaacggtaactatcaaatcagctatgggtggcttaggtaattttgtggttattcatacgccggaaggattcaatatcgtttatcaagaagcttttagttctccctctaatattattgttagtgttgggcaaaaagtaaaaactggtgatgtaattggatatcgtgatacagaccatgttcatattggcgtaactaagcaagatttttatcaagcagttcgaaattctttttctcctgcaggtggttggctagatccagtaaaactaattaaagaaggtggcgatgggtctaaaccacaagaaggaaagaaagatcaaactgttgataatagtaatgctgcacgtcctaaattaaccattactactgtcaataacggtagagactatattgatattcctgatttacaaaaagaattcggtattattgagggaactgttgaatttgataatgtagatgatccgaatgttttaatgcaacaagctcaaacatggataaaggctcaaagaatacctcaaagttgggaagttacagctttagaattacatatgacaaacttcaaatcttttaaggttgctgataggtacatgtttattaatccaaatgttgcaaaaccccaattattacgaattactcaaaaagaaattgatttactaaagccccatgcgtcttcattaacgattggtgataagacgatggggcttactgattatcagttagaaaatcaagtcaattttcaacaatttaaggaaattcgagtgatggttaatcaggttgtccaaacccaagagcaatctgctaataacaataataaggttatgcaaaattttgctagtagtgctgatcttgcacaaatgagacaggatctaagaaatcttcaagatgataacgatcgtgctcgcaaaggaatggtttccttagaagaattcaataaactaaaggaacaagtagaaaaactaacaacaggaggcgatgataatggcaagtgaaacctatgattatgagtcatttgataatacggatcatactatgaaacaaatcgctgacgctattcgtcacaagggttatggaaaagatgtgcgtgaggcaattgcacagggcttcgaaaacttagataaacatttaagtagtattgaagaagaactgaaacaacaagaaaagaaaaagtcatcgtctatggatgatatttttaattcttttggtaagaaggagtgatgataaatggcaaaagagattagtaatctgattacctttaacacctacaaatttgaacgaggcggccttttggttgatatgtttaaccaatttaatgctcgtgtaggagatcaaggaacggaattagccatccagtgggaaactagtaagactgaaactaaaattaatttaaaagaacgaggattacatttctttgggacaggttcagttggacagtaccttgaaaaattagaagatggaactggctttaagatgtctgcagatgcatctaccgttgaatgggaagataaagatgaagctggtagtttagacgatggaattacggttgttaagttgccaaaacaatttttccctcaaaaaggtattttctttggttactttggcctaaaagatagacaaggtaatatctttactagtgttaatgtttggttccgtgttcttggcggtgttccaacgatgggggctgctattccttactttgttactgaatttgatgaagtattagagcgatgcaatggtaagattattgacgctttagcagaattacgtgaaaagtaccaggcagaagttaagaagaatgaggatatgtctgcggaaacaagagcagcgttgagtaaacttgctgatgctgttggtgcaattcaagcgcagattgatgcaggtaatgtaattacacgtaaagaatataataatcttgctaatcagattgataatcgccttagtaaaatgacacagaacattgagagtttttcatcactcgatgacttaaaagctcaatatcctaatggaaaagatggactctttgttactaatgacaataatcataagtatcaatacaagaatggatcatgggtagatgaaggtatatggacagttactacctttgatccagaaacgcgacgacgccttacataccttgatacatctaattctattttgcaaaaatcactcagtgaattgactaaagaagtcgttgatataaattggtttttaggcgaaattgatgcaaatactggtaaaattacacctcacgaaagttttaatcgtgcatactcatcactcaaagtaattggtaaatcacatgcttttgaatttttgcttaatactgattatttgcaatatattagtatttttgaattcaattcaaatggggagattattaagcacgatagtgttggcgataaagcgatatatacatttgaaaaggatacgactgcggtaaggtttcagattactagtactgctgctactatgaagcaaaatgatttacgcgatactattcaaaatagtgggctaaaaattatcgatcgaggacaccgttcagtaattaatgatattgatcatttatatatccgtgaacatttaatgccgattgaagcaattgaaggctatacaattaacactaatgttgattatggtgaaactgttgatgtatcacatccaatagtaacgtctgcgtttcaatatattaatcaagtatgtaagccgggcgatattttcgtaattaataatctatctggaggctttaacgcaatggcttgggcctttattgatagcgagaaccggttaattcaaaaatctgaagtaaacttatctcaatctcaggttactttatatgcgccaagtaatgctgcaaaattaattatcaataatatggatagtaactgtacagcatatagatatactccggataaagaacatttagctaaaattaatattgctttaactaccaatattcatcgaaatatcaagttagcttatcaaacaggagaaacagtctcattaattcctgaaaaagtaagtaattacaaatatattatattagattgtaattttaatgatgctttcagaataaagggttatggtggattgaatccacgattatatggatttatttctgagcaaaacacattaatgaatgttgcgcctgcaaatgttaatgatcaattaactgatgtctttatcaaaacgccaaaaggcgccaaaaaattagtagttaatttcaatcttgatcaacaagtaaatcccaaacaaattgctgaactttataagcttccagacattaatgttattactgagcaaaaaataaaaaagaatttattgaatcaacaaacagtcgatacattaatttctagttatatgaatggtatattacttggtaaagatttagccaatcatcttgcggattttgctaaaagcggcgataaaatggttcatgtatcaacgttttataaagttaacgatactctttttatgtcttattatgctaatacaagatcggcttacgaagatccaacccagcatacagcacgattagtttatgcaccgtttgagaatctaaatcaacaaacgtacatcgatgtagccgacattggacaagaatataatggtcaaaagattgaagcaatatacgattctttgcttcttaaaacaagtgatgattcatatatgatctatgcctttactgctaaggtaggtggtaagttctatatgctttaccgtcgttttgatcctaagactaagctgttgagcgatattcatacaatgaattttaaagttggagtaatgacgtcgacatttgatacagtaagcgttcatgatttgttagctaaggcaggaattgactatgattatgaagatcgtgatatttcctttgtccaaaaattaagtccacggattgaagatggcgtagtgcaatactatgcaggtatcggaatcttacatttttgctttgtagtaaaatcaagcgacttaatcaactggacttttgtcagtactccagactttatgtacaagccagaatttgaaccttctgtatatgttaagggagacaatgtttattatttttgtcgtcagcgagggacagaaggtaatgcagttttagctaagtataatatccctaatggtcaatggtcgaatccgattcttgtaccagatactcaatcacgctatgactttttcgagaataatagtcagttgtaccttgttcattcaccgctagaccgtaatcatattagtttgatgcagattgatcagaatgttctggaaaagagctatgaagtagcaacagcagtagtacaagattgcttctacccatttactcaaaatattgatggtcagatgtatatgagctttacacaaagtaggcaacatatttggctaaataaatttaatccccacagtctgctagatagtgatgtagcaactattttcagtaatttaattgagtagcaaaacatagtcgccttataaatacacaatacatagaaagttgcattgcttaaatggaaacttaatgggcggcttatagacttttattatttaaaagagtatactggaattattctttttattgggaggaaatataaaagtgacaactaaagtgagagataaatcgcttgatattatccgggggagcatccttctagttgtcttgggtcatatatctgggataccttttgagttaaaaaagtacatatattcttttcatataccactgttcttttttgtttcgggatatttgttcaactttgctaaatacaggtatttttcttataaagagtttataaaatataaagctaaaaaatatatcctaccttatttcagaatggggttgatatgtttgcttctatttggcatagtttatccactatttgctgaagggtttagtaaacagtatatgcttcaatctacaaaatatgttttaggtttactatactcgcgtggaggtcctaattatatggcctggagttcgccactttggtttctaacagccttatttattgcagagattattttttttgtggttctaaaatttaattttaaatatccattaatagtgtttggaatcttagctatattgagttatatttactcgattacaattaaaattccgttaccgtggaatattgatgttgcaatgtttgcagtgttattcatgtacctaggctttattacacacaaatataatttaactaagcacattaacttaccggttttcttacttttgattgttatttttgtattatctgtagcttacaataatgagattgacatgaatttaagaaactatggcaatggatttttaacaattattagtggaacaataggtactgttatatgcttgcaaattgcgcgattgttaaaagagaataagatattagagttttatggtaaaaacacattatttatcatgggctacacgtatgctgtgtttaattgtattttggctttaagcagccattttagtactgtaaagaatgttgtagcttcgtttttgattcaaattataattttaactttattaatagtactgaagaatttattcaaacaaataaaaaagcccatttatgcatatactaggaaaattaacaattaaaaaaactaatacgtcctactcagggcgtttttattttaccttcaaaggagatgatcaacattgccctatcatttatttatgctgcaccaaatgcagacattagttgatgataaactaatgtgggcttttacaatcgtgatgattgtagatttaattacgggaatgattaaaccgtattatgcaaagaaaacaattaagaaaactaatagttcagttggaatccctgggataattaaacacacagtaatttatttagtggtagtaattgcttatccatatctttatacgattggagcaagcacgatggctaccacttttttaattgcttggatttatcaatatttaatttcaattgtagaaaattggacagagatggggtggtggttgcctaaaccaatcatggatttctttgaagccaaattagctaaggatcaagaagattatgacccatctaagtacaattttcttggtaaatataaaggaggtaaaaagtaa

SEQ ID NO: 14 - DNA, 300 bp, synthetic construct
  misc_feature 1..300: note = Description of sequence: Based on Lactobacillus reuteri sequence
  source 1..300: mol_type = other DNA
ttaaagtattaaaatagatgtaaaatttatttttttcaaaagaaattttaattgtacactgttggtattgaacggggttaaacaaaggtaaattagcatttctgcggattaagataaatagaaaaatgttaaagaacaccttaaaaagattaattttttataattggaccgtatcaatttgtaaaaaggttgactttttgaaaaaaaagtttatcattaacattgtaaatttaatgatttacgttatgttgttatagagcacaggacgtattgatttatatagaaggagtgtttattaga

SEQ ID NO: 15 - DNA, 300 bp, synthetic construct
  misc_feature 1..300: note = Description of sequence: Based on Lactobacillus reuteri sequence
  source 1..300: mol_type = other DNA
atgaatggacagatgttttaatcgctagaatagaaggaaagaaagtcgcaacaaatacggtttctagtacgtggcaggaacgactaggtaagcagattgacgaattaatagaaaaacattagtcaaatacatttacaaatgaacagatagttgatattatatttaagaattcttcttcagagcctaagattaaagctttcaattggcgaaaagaagttgtacaatatgtataaaggtatgtcagtcaccgaatcagatgatctggcattatacttgtaaattatcaggaggttttcatta

SEQ ID NO: 16 - DNA, 300 bp, synthetic construct
  misc_feature 1..300: note = Description of sequence: Based on Lactobacillus reuteri sequence
  source 1..300: mol_type = other DNA
atctcacgtgcgatccattacactaagggcgcgtcaacaaatattatactatcttaaataagaatgaattgcaagcattatttgaaaattttaattaaaataacgcttacatcagaaaaatgttgtgattgaatagacaatttttttgaagatggtatcataagtatcgtaggagttgtattattgcttagaccttaccactgcgtcacttacaatggttgagagttgcgatgctgatgtaatgtgataaactaagcaagtacactaattatgttttttcctaaaggaggaatttgcagt

SEQ ID NO: 17 - DNA, 300 bp, synthetic construct
  misc_feature 1..300: note = Description of sequence: Based on Lactobacillus reuteri sequence
  source 1..300: mol_type = other DNA
ttgtttaagatatctttcaaagctgcggaatttttcccagcttttttagttagttttgttttcataagctataattttaaccgattccaaatttcttttaaaagtttttttgatctagaccattaattgataaacgcttaccaaagactaatcaacaagccatttagcggtagtggtccattttaactttctaagacatcttctcagaaaacgtttcctttgatagtgcagattgtgctttaagagtatataattgtcacggtataagaattttctgaaatttcagaaggagtgaacatt

SEQ ID NO: 18 - DNA, 449 bp, synthetic construct
  misc_feature 1..449: note = Description of sequence: Based on Lactobacillus reuteri sequence
  source 1..449: mol_type = other DNA
ctcctctattattattcctgatcaattttaaattaatctccctagataggtatattttagcacaggtcaccaacgttccaaagtttaatctatgtttaaactttaattttcaaaaaaatgctatactatgttcacgatactttaaggaaaggtgattacaatagtgagtctcttaattgctattcttatctgctggttgctatggaagattgggggtttaacggttaagttcattggtctaatccttcttattctattaatcgggacattaattcatgttttactttggccagcgatccttttagcagttattatcttaggagcaggtttattcactaactaatttatctataaaatcttatagtaatttttctgcggaatgttataatcattactgtgagagaaatctcaaataatgtatacataagatgaaagggagactgtttatt

SEQ ID NO: 19 - DNA, 250 bp, synthetic construct
  misc_feature 1..250: note = Description of sequence: Based on Lactobacillus reuteri sequence
  source 1..250: mol_type = other DNA
acaaatacggtttctagtacgtggcaggaacgactaggtaagcagattgacgaattaatagaaaaacattagtcaaatacatttacaaatgaacagatagttgatattatatttaagaattcttcttcagagcctaagattaaagctttcaattggcgaaaagaagttgtacaatatgtataaaggtatgtcagtcaccgaatcagatgatctggcattatacttgtaaattatcaggaggttttcatta

SEQ ID NO: 20 - protein, 3017 aa, synthetic construct
  REGION 1..3017: note = Description of sequence: Based on Lactobacillus reuteri sequence
MVSKNNHQFYQQKHAERKQRWGIRKLSVGVASVLLGTTFMLYGNHAVLADTVTSPSDDVTRSTTTQGGNKDKVTEGTTEGTTSTPQTSGDSTDKQANGQNVNQQVPTTDTEEATNHQDTPQGQDTTQNTTNVDKKDTEVTPANDATTPTTQKITAKFTTAKFTTAKFTAAKFKVLAARPVMKVAGTASLPISNQDIKLDSQPMLTEIINKPTDNWVYNNLKWYQDTSTEKIKEILQNHTANDESGRYYFAGVANYNEHYHAIYLLARSNNLNDNSLYVTILHTGLGKNIQEAVVAPGESKKVEYSGTTHTPIFTNYDGTSASIDLDGIEKGDNIYGMVVGFAYGHNTGIKGDPASMGNGFVMTPIPTKMTTTIHYIDQATGDEIAVPKSFEGVAYQKYTITGEAPTIDGYTLKKSPETTGYISPYKVGESYDFRLDKHVVIKQTVIDAQGLVRVTAYYDGEVLNNTTRYLGNKLNVNDRMSFISHGKWYTYINQITSTNDGIVYYYAKDGSEDKSEVRVHYIDVTGSKNSIFVPGDGEEVATDKISGKLGENYNYDVNLPTDYNLATNQANTVNGTYTIDHHDEYVYVVKKTSAELDPTVPAKTKVDNPTSLTADEKKTIEDKIVEANKDKFPEGTGVTVANDGKATITYPDKSVDTIEGNQLVEEKTSAEKLDPTVPAKTKVDNPTSLTADEKKTIEDKIVEANKDKFPEGTGVTVANDGKATITYPDKSVDTIEGNQLVEEKTSAEKLDPTVPAKTKVDNPTSLTADEKKTIEDKIVEANKDKFPEGTGVTVANDGKATITYPDKSVDTIEGNQLVEEKTSAEKLDPTVPAKTKVDNPTSLTADEKKTIEDKIVEANKDKFPEGTGVTVANDGKATITYPDKSVDTIEGNQLVEEKTSAEKLDPTVPAKTKVDNPTSLTADEKKTIEDKIVEANKDKFPEGTGVTVANDGKATITYPDKSVDTIEGNQLVEEKTSAEKLDPTVPAKTKVDNPTSLTADEKKTIEDKIVEANKDKFPEGTGVTVANDGKATITYPDKSVDTIEGNQLVEEKTSAEKLDPTVPAKTKVDNPTSLTADEKKTIEDKIVEANKDKFPEGTGVTVANDGKATITYPDKSVDTIEGNQLVEEKTSAEKLDPTVPAKTKVDNPTSLTADEKKTIEDKIVEANKDKFPEGTGVTVANDGKATITYPDKSVDTIEGNQLVEEKTSAEKLDPTVPAKTKVDNPTSLTADEKKTIEDKIVEANKDKFPEGTGVTVANDGKATITYPDKSVDTIEGNQLVEEKTSAEKLDPTVPAKTKVDNPTSLTADEKKTIEDKIVEANKDKFPEGTGVTVANDGKATITYPDKSVDTIEGNQLVEEKTSAEKLDPTVPAKTKVDNPTSLTADEKKTIEDKIVEANKDKFPEGTGVTVANDGKATITYPDKSVDTIEGNQLVEEKTSAEKLDPTVPAKTKVDNPTSLTADEKKTIEDKIVEANKDKFPEGTGVTVANDGKATITYPDKSVDTIEGNQLVEEKTSAEKLDPTVPAKTKVDNPTSLTADEKKTIEDKIVEANKDKFPEGTGVTVANDGKATITYPDKSVDTIEGNQLVEEKTSAEKLDPTVPAKTKVDNPTSLTADEKKTIEDKIVEANKDKFPEGTGVTVANDGKATITYPDKSVDTIEGNQLVEEKTSAEKLDPTVPAKTKVDNPTSLTADEKKTIEDKIVEANKDKFPEGTGVTVANDGKATITYPDKSVDTIEGNQLVEEKTSAEKLDPTVPAKTKVDNPTSLTADEKKTIEDKIVEANKDKFPEGTGVTVANDGKATITYPDKSVDTIEGNQLVEEKTSAEKLDPTVPAKTKVDNPTSLTADEKKTIEDKIVEANKDKFPEGTGVTVANDGKATITYPDKSVDTIEGNQLVEEKTSAEKLDPTVPAKTKVDNPTSLTADEKKTIEDKIVEANKDKFPEGTGVTVANDGKATITYPDKSVDTIEGNQLVEEKTSAEKLDPTVPAKTKVDNPTSLTADEKKTIEDKIVEANKDKFPEGTGVTVANDGKATITYPDKSVDTIEGNQLVEEKTSAEKLDPTVPAKTKVDNPTSLTADEKKTIEDKIVEANKDKFPEGTGVTVANDGKATITYPDKSVDTIEGNQLVEEKTSAEKLDPTVPAKTKVDNPTSLTADEKKTIEDKIVEANKDKFPEGTGVTVANDGKATITYPDKSVDTIEGNQLVEEKTSAEKLDPTVPAKTKVDDPTKLTNDEKKEVEDNIRDHNTGLPEGTKIAVGDNGDTTITYPDKSVDTIEGNQLVEEKTSAEKLDPTVPAKTKVDDPTKLTNDEKKEVEDNIRDHNTGLPEGTKIAVGDNGDTTITYPDNSVDTIPGDKVVEGKSDAAKNEPKVPGDKVKVDDPNKLTEDEKSEVVKAVEDANKDENGKSTLPEGSKVTVGDNGDVTVTYPDGSKDTIPGDKVVEGKGTEGQTDADKNEPKVPGDKVKVDDPNKLTEDEKSEVVKAVEDANKDENGKSTLPEGSKVTVGDNGDVTVTYPDGSKDTIPGDKVVEGKGTEGQTDADKNEPKVPGDKVKVDDPNKLTEDEKSEVVKAVEDANKDENGKSTLPEGSKVTVGDNGDVTVTYPDGSKDTIPGDKVVEGRGTEGQTDADKNEPKVPGDKVKVDDPTKLTEDEKSDVEQAIKDANKDENGKSTLPEGSKVTVGDNDDVTVTYPDGSKDTIPGDKVVEGKGTEGQTDADKNEPKVPGDKVKVDDPNKLMEDEKSDVEQAIKDANKDENGKSTLPEGSKVTVSDNGDVTITYPDGSKDTIPGDQVIEGKSDADKNTPNVPGGDKVKVDDPTKLTDNEKNAVKDKVDEANSNLPDGTKVTVGDDGTTTITYPDGSTNTISGHDLVTGKTDADKYPLNPGQAVNVVDPNHLTQAEQDQVKEAIQTTNPTAPIATITVDTAGNVQVTFADGSTTTLQANLHKHVTEATTGSAIKPGVGTNGGQTKGATSTNQTATKQQAQQHLPQTGDQPATWAMLSGLGVAFLGLLGLKKKRED

SEQ ID NO: 21 - protein, 1472 aa, synthetic construct
  REGION 1..1472: note = Description of sequence: Based on Lactobacillus reuteri sequence
MEIKKHFKLYKDGKKWCCAAIATTVLGIGLAIGSPSVLADADTITSTSDANNSLVKNDNTSDTDSNSESTFTDTNKNSTNEKEINENKNIDSSQQINQEQTKSNNSEEQTTPVNVKAENTDIKDSIPEKSTPNSFKEINGSTYYYGENGDLYRNQFYNNWGRTYYFQANGARLDNGFYNNWGRTYYFGSDGARWDNRFYNNWGRTYYFQNDGSRLDNSFYNNWGRTYYFGVDGARWDNRYMVKWGRAYYFGNDGALLQNQLKSINGINYWINNEGIIPLKNQFLTANENQLFYFDGNGSLVVNKFYHNWGHTYYFGSDGARYTDQFLNRDGKVYYFDNQGIMYQDQYYKNWGHTYYFGSDGARYTDQFLNRDGKVYYFDNQGIMYQDQYYKNWGHTYYFGSDGARYTDQFLNRDGKVYYFDNQGIMYQDQYYKNWGHTYYFGSDGARYTDQFLNRDGKVYYFDNQGIMVTNQVRVIDGKGYEFNDNGEATETSDMGQTRDTVAKEVAQALTNQGIKGVKYDWRNTNNDYQELALHDIAQEVAQGDTNPDKNVIEKKLQANNLLSGKVLVVYSTDFTNDDPQKITNTFMNSYDFTNADNSVLGVGADLNKNKLVIILFKPGEKAEQPQATSTISASISDIFKKAGVNVDVDNGLTKGSVVNSADLGNALTNGTAELLKGDKGTIISQEVLKAIFAAFAGNTSAVEGTKNYYNGNDAYHYEFWLEGQSADDKLNNFLALNKGAKYGDQLKVNYTATLVFGQETGTNSNESKVPASERTDEQLDLAYKTGTDTGLRYDSVKVEKIPGMTDDMVRGVDVSSYQALINAGVKFYDFNGQESNLFKILKDSGVNWVRLRVWNDPYNAQGQPYAGGDNNEENLIKMAKEASDNGLKLLIDFQYSDFWTDPAQQILPKAWRNLSHGEMSQEVYLYTSKILNDLQKAGASVKMVQIGNEITNGAFGLYTGRNGGGNWASLWETSDGDQVAKYIQAGSSAVRRIDPTIKVAIQLETPEINKYRGIMNVLKKNNVDYDYLGTSYYPFWSTTQGNGWYDNVDLGYGANTPVNLEAIEKMAWNEFGKRTVILESGWLNNTNDADGTHNSVGENNETTNIDRYSADPQGQVDEIEDMYNAIIAQKGLGAFYWEPAWIPVKAGWNNWQYNKLMSNIYGSGWASQYAKGYAPDSVLYYDGKEAWGGSSWDNISLFDDHGHPLQSLNVYNGMLNGYESPKNVKSSLSTQLVKIWNETDVIPNDGLTEGTKLSTDLFGTTQLSGNDGQSIGNAELTKLAGRLKDGISSKVYTAANGARYHYIYWLEGGNNKVNTFVSANKDAKYGQPLIANYSATVVVDSEPGTQVATSPLQIKISQVWNTVNNEEIKIDNPLKQGDLITDKSDNAFSGILNSKDIKEALTGEKGKDVSESTVNDVKSLLPKEVKGSKTYTTADGNQYYYDFWLASVETSNVNYGEPIIVNYTASLKWLG

SEQ ID NO: 22 - protein, 1063 aa, synthetic construct
  REGION 1..1063: note = Description of sequence: Based on Lactobacillus reuteri sequence
MEKTMKKKALVATTAVAGITLVGEVTTVHAADNVQQPVNEQNVNQSSQEEKQAAQNLQNAQSDVNTATEANSNAQDNLASANNNLSNAKKAVSDQAAKVADATKAQSDASTKVDNDNKVVADAQQKADQATPANIENAKQAIEGQNKVIDQDNENIKYSNTDQDKAQNTLNNAQSNEDKANATLSNKKSSQASAQNNVKQAEDALNGTHLVEAQNAFNQAQSNVENAQSKYDQANNQLSDAQKKVTTNQNDLTAKNKALDNINNQVDTDQNNVNSNQATADSASSATQVAQNAVDQTKQSLDKVIEELNGFSENTIKVPAGAQEAYEAFIDAVDNNADQSQLDSLAKKMYDTLHQGQGTNGINHFNSSKYDQNQLVDVDHLTTDQLNELTQFAADMINSARKAWGSDKNAGTLIPTQGVSEMAQQIAKGYVSDNWHISQGHDVKRVTAAAGLIGLNDAGQFYEDASEGYVHAWPWEKDSYTMDNLKEAVYDSILGMLFADDNSGNGHMTDLLGLHVNRKEDHQYFGLSTNMCPGSYMGQLHFIIVENDPAYIKDPQTFNAKGGTTKIEYIDPKVQLNQQKDILTTTLSTQQADLATKQDALNKANQNLANAKKQLSEDQDLQTVAQQNRDSAQKALNDATAKVSNLQATVNSLSQDLNSAKATLDQAKKTLESYTADHKAKLDNYNNAKAALDDANKAVAEAQSAVDTAVNETKIAQNNLDQKKQAVTDAQNKLANDQEYLATLKQNLADLQNAPQNLQKAKDQLAKDQIALDNANKDLQNQKDSLDELNKKLEDAQVKVNEAQSAANVTKATLDQAQAKLSDAEATWKELHNDAHRYGNVVKVTPITMEAGTSLPDPVIENGFTVNTGTNQLFVSLAAIDSSNNNIPQGTKASWANRSKALTDSQNAGSYSEDILITFPDNSTVTVPVDLTVTAKKITEDQKATEGGYHIVNGSVVDKQNNLVSGWTVKNGQMVDPEGNVIKTTMSTAQGVTIEKNNSKSGNTKTNMIQTSLTIANNKATTNKDNQLPQTGNYNNNTKVLGLAGIALASALTMFGYKKRQHN

SEQ ID NO: 23 - protein, 212 aa, synthetic construct
  REGION 1..212: note = Description of sequence: Based on Lactobacillus reuteri sequence
MKSTTKKILASSLGVAGAMAMGTVTAKADTTVTVNAGDSLNGIAQKYNVSADDIATANHLQNKELIFVGQKLTIPTKDKNETPANNAEKKDQASKNSQSLQDSVNKAMSYLGTPYVWGGNKPGGFDCSGLVQYCYGIPQRTTYEQQALGPHIHDNVLNAPYGALVFYGSDDAPYHVAISLGDGRIIQAPNENETVKITDQQYFPGNYYVVMH

SEQ ID NO: 24 - DNA, 1029 bp, synthetic construct
  misc_feature 1..1029: note = Description of sequence: Based on Lactobacillus reuteri sequence
  source 1..1029: mol_type = other DNA
atggaaagccgtaataacaataacctgaagggcatcgatgtgagcaactggaagggcaacatcaattttcaaagcgtcaaaaatgacggtgttgaagttgtttacattaaggcaaccgaaggcaactacttcaaagacaaatatgctaagcaaaactacgagcgcgctaaagaacagggtctgcgtgtgggcttctaccactttttccgcgcaaacaaaggtgccaaagatcaggcgaacttcttcgtgaattacctgaacgaaatcggtgcggtcaattatgactgtaaactggcactggacatcgagactaccgaaggcgtcggtgcgcgtgacctgacctctatgtgcatcgagttcctggaagaggtgaagcgtattacgggtaaggaagttgtcgtgtacacctataccagcttcgcgaacaataatctggattcccgtctgtctagctatccggtgtggattgcgcactatggcgtcaacaccccgggtgcgaacaatatctggagcgagtgggtgggtttccagtacagcgagaatggctccgtcgccggtgtcagcggtggctgcgatatgaacgaatttaccaatggtatctttattgactcgaacaatttcacgttggacaatgcaacgaccaaaaatgttagcattaagctgaacattcgcgccaagggtacgaccaacagcaaagttattggtagcattccggcgaacgaaaagtttaagatcaaatgggttgatgaagattacctgggttggtattacgttgagtataacggtatcgtgggttacgttaacgccgattacgtcgagaaactgcaaatggcgaccacgcataatgttagcacctttctgaatgtacgcgaggagggttccttgaatagccgtattgtggacaagatcaacactggcgacatctttcgtattgactgggttgatagcgatttcattggttggtatcgtgtgacgacgaaaaacggcaaggtcggctttgttaatgcagagtttgtgaaaaagttgtaa

SEQ ID NO: 25 - DNA, 738 bp, synthetic construct
  misc_feature 1..738: note = Description of sequence: Based on Lactobacillus reuteri sequence
  source 1..738: mol_type = other DNA
atgacaacagtaaatgaagcattaaataatgtaagagctcaggttgggtccggtgcgtctgttggcaacggcgaatgctacgctttggctagttggtacgagcgcatgattagtccggatgcaactgtcggacttggcgctggtgtgggctgggtcagcggtgcaatcggcgatacaatctctgccaaaaacatcggctcatcatacaactggcaagctaacggctggacagtttccacatctggtccatttaaagcaggtcagattgtgacgcttggggcaacaccaggaaacccttacggacatgtggtaatcgtcgaagcagtggacggcgatagattgactattttggagcaaaactacggcgggaaacgttatcccgtccgtaattattacagcgctgcaagctatcgtcaacaggtcgtgcattacatcacaccgcctggcacggtcgcacagtcagcacccaaccttgcaggctctcgttcctatcgcgagacgggcactatgactgtcacggtcgatgctctcaatgttcgcagggcgccaaatacttcaggcgagattgtagcagtatacaagcgtggtgaatcatttgactatgatactgtcatcatcgatgtcaatggctatgtctgggtgtcttacataggcggcagcggcaaacgtaactacgttgcgacgggcgctaccaaagacggtaagcgtttcggcaatgcttggggtacatttaaataa

SEQ ID NO: 26 - DNA, 543 bp, synthetic construct
  misc_feature 1..543: note = Description of sequence: Based on Lactobacillus reuteri sequence
  source 1..543: mol_type = other DNA
atgtcgaagatttttggtttagatgcgggtcattgtacgagcggcgcagatacgggtgcgcagggcaatggttacaaagaacaagacttgacccgtcaagttgttacctatctgagcgaatacttggagaaagagggccacactaccaagtactgccattgcaatagcgcgagcacggttaacgaatccctgcgctatcgtgtgaacaaagccaactccatcggtgtcgactacttcgtgagcatccacctgaacgccggtggcggcgttggtaccgaaacgtacatctgcgcgcgtggcggcgaggccgagcgcgtggcgaaacgcgtcaattctaaactggtgcagtacggttatcgtgaccgtggtgtcaaggttggtaatctgtatgtgattaagaacaccaatgcaccggctatcctggttgagatctgtttcattgacagcagcagcgatgtggcaaagtttaacgcgaaggcaatcgcgaaagcgattgctgagggtctgctggataaaaccattggtgaagtcgagaataagtaa

SEQ ID NO: 27 - DNA, 1107 bp, synthetic construct
  misc_feature 1..1107: note = Description of sequence: Based on Lactobacillus reuteri sequence
  source 1..1107: mol_type = other DNA
atgcgtaatcaattcatcgatgtttcaagttatcaaccagatactgttgcctttttccaagctgctaaagctcagggtgcattaggggtcgttgttaagttaacggaagggtccgaagatggttcggcttatgttaatccacgtgcggccgctcaaattcgtaatgccttagcggttggcttgcgcgtttcctgttaccactttgctcgttatacatcagtgactgatgcacaaaatgaagctcgattcttcgttaaaatcgctaagcaatttggtatgtatgacgatactttgatgattgatgatgcggaagttcattcaactgcagattatcaatcagtatccttagcctttcttcaagaagtagaagctcttggttacaagaatactgggatttactccatgaagtccttcttcactggcggtattcttaattcacatggctttgattcccggaagatttggattgctggctatggtgtgactgaactggggattgataatgcaagtgcttggcaatattctgatcatagcatcatgggaattgatactagttatgactttgacggtgcctttacgactggtttagtatcaggcaatgttccgcaagctgttattccagcaccacagccggttcaacatattggtcacccagctactggaacctacattgttcagccgggcgatacattgagtggaattgcagaaaaatacgggactacttatcagaacctagcagcaatcaatggtattggtaatccaaaccagatcaatgtcggccaagtcctcaaagtcaccggaaaagtatcaaacgaaaatacttactttgttcaatcaggcgatacgttatccggaattgccaccaaattcggcaccactgtctcagacctcgtaagccgtaatcacattactaacccgaatgtgatctacgttgggcaaaaactctacttagccggcaacggacaatccaatgcttatactgtccaagcaggggacacactaagcggaattgcggctaagtttggcaagacctggcaagcattagctcaaaagaatggcatcgcaaatcctaatatgattttcattggtcaaacaattcagatttaa

SEQ ID NO: 28 - DNA, 2439 bp, synthetic construct
  misc_feature 1..2439: note = Description of sequence: Based on Lactobacillus reuteri sequence
  source 1..2439: mol_type = other DNA
gtgtaccgaattattggttataatgaaccaacagataaagcaggatttattgtactggatccccgagttaatcgtcatattagttcgggaaaactcacgcttaaagaatctaatattgatgatttgactattacggttaatcaagcaagtccattatgggacaacgtaaggccttatcatactcatgttaacgtttatgatgataatgaacttatttttcgtggacgagctatcaaacctaaaaagtcgatggaagaaagcggacaattcattcgtgaatatgtttttgaagatattgaagcatatctcatggatagcacccaaagattttatgaaggtgttggtcaaacgcccaaagaatttttacaaactttaatcgatgttcataattcacaggttcctgactataaaaagtttcaagtccggaatgtaaatgtcactaataataaggatgaccaatatcgacaaattgattatcccaaaactagcgatgctattaatgataaattagttaaatctcttggtggttatattgtgactacttacaacgctaacggaataaactacattgactacttaacggatattggggttgatcataaagatgatactcctattcagttagctaaaaatatgaagtctgcaagtatgcaaattgatcctactaaggtgattacaagactgattccactgggaaagacactagaaccatcaaaagttgatgtaagtgatgatgatggagagggcggttctggatcattagatagccctgaagaattttgtaaatcagaaattaatgctacttggggtagtgatattaataatatgaaacaagattttgccgctcgttcttcgagagttcgggcttggggagtggacgttaatcgtttatatgatgtggtgaaaaatgctggagtaagtcctgaatggttctttgcttatgaacttcaagaacaaggaacttactatggatggcttaaccatacttatcgacacggtgatgcgtatagtgatgcgcaatctgtttgtgagtggattaaaaattgttcaaatagtaattccattaatccagcatggagcgcaccggaaggatcaatggcgccgaatcaagcattagcggataaatggaatcaagagtttggaaaaggtactattggccgcgtttatttacaagggactgccgctgctgtttgggatttagctggtcaaacgcctaatccagctattggaaagccaattagtggatgcatttcttgtattaaacgttggggtggtcattctaatgcagctggtggtacatggggatggccttttcctgatgttggggaaggtcatttttctcaagttcagagtttcggaaatgatggcggatatcgtcaaaatagttatcacgatggtgtggattttggatcaatagatcatcctggtagagaagtgcattgtattcatggtggaacggtaactatcaaatcagctatgggtggcttaggtaattttgtggttattcatacgccggaaggattcaatatcgtttatcaagaagcttttagttctccctctaatattattgttagtgttgggcaaaaagtaaaaactggtgatgtaattggatatcgtgatacagaccatgttcatattggcgtaactaagcaagatttttatcaagcagttcgaaattctttttctcctgcaggtggttggctagatccagtaaaactaattaaagaaggtggcgatgggtctaaaccacaagaaggaaagaaagatcaaactgttgataatagtaatgctgcacgtcctaaattaaccattactactgtcaataacggtagagactatattgatattcctgatttacaaaaagaattcggtattattgagggaactgttgaatttgataatgtagatgatccgaatgttttaatgcaacaagctcaaacatggataaaggctcaaagaatacctcaaagttgggaagttacagctttagaattacatatgacaaacttcaaatcttttaaggttgctgataggtacatgtttattaatccaaatgttgcaaaaccccaattattacgaattactcaaaaagaaattgatttactaaagccccatgcgtcttcattaacgattggtgataagacgatggggcttactgattatcagttagaaaatcaagtcaattttcaacaatttaaggaaattcgagtgatggttaatcaggttgtccaaacccaagagcaatctgctaataacaataataaggttatgcaaaattttgctagtagtgctgatcttgcacaaatgagacaggatctaagaaatcttcaagatgataacgatcgtgctcgcaaaggaatggtttccttagaagaattcaataaactaaaggaacaagtagaaaaactaacaacaggaggcgatgataatggcaagtga

SEQ ID NO: 29 - protein, 154 aa, synthetic construct
  REGION 1..154: note = Description of sequence: Synthetic construct
EVQLVESGGGLVQAGGSLRLSCAGSGRTGSLYSMGWFRQAPGKEREFVAAITWRPSSTYYADSVKGRFTISRDDAKNTVYLQMNSLKPEDTAVYFCAARPRGGLSPTPQAYDYWGQGTQVTVSSAAASGSLEQKLISEEDLNGAAHHHHHHGAA

SEQ ID NO: 30 - protein, 141 aa, synthetic construct
  REGION 1..141: note = Description of sequence: Synthetic construct
EVQLVESGGGLVQPGGSLRLSCAASGSIATINDMGWFRQAPGKQRDWVATIVSDGSTAYADSVKGRFTISRDNAKNTVYLQMNSLKPEDTAVYYCSARRHYGQGTQVTVSSAAASGSLEQKLISEEDLNGAAHHHHHHGAA

SEQ ID NO: 31 - DNA, 462 bp, synthetic construct
  misc_feature 1..462: note = Description of sequence: Synthetic construct
  source 1..462: mol_type = other DNA
gaggtgcagctcgtggaaagtggcggaggtcttgttcaggctgggggatcgctccgtctgagctgtgcggggtctggcagaacaggtagtctctattccatgggttggtttcggcaggccccgggtaaggagcgggagttcgttgcagcgattacgtggaggcccagctctacctactacgcggacagcgtaaagggacgattcaccattagtagagacgacgcaaagaatactgtatatttgcagatgaattcgttgaagcctgaggacaccgctgtctatttttgcgcggcgcgaccgaggggcggtctctccccgacacctcaagcatatgattactggggacaagggacccaagtcactgtatccagtgcggccgcgagcggcagccttgaacaaaagctgataagcgaggaggatctcaatggtgctgcacatcatcatcaccatcacggggcagcg

SEQ ID NO: 32 - DNA, 423 bp, synthetic construct
  misc_feature 1..423: note = Description of sequence: Synthetic construct
  source 1..423: mol_type = other DNA
gaagttcagcttgtagagtccggtgggggtcttgtacagcccggcgggagcttgcgactctcatgcgctgcttccggaagcattgcgacaataaatgatatgggttggtttagacaagcccccgggaagcagcgtgactgggtcgcgactattgtgagtgacggcagcacggcttatgcggactcagtgaaagggagatttacgatttcgcgagataacgcgaaaaacactgtatacctgcagatgaattcactcaagccggaagatacagctgtgtattattgttctgcccgacggcactacggacaggggacccaggtcacagtctcgagcgctgccgccagtgggtcactcgagcagaagctgatatcagaggaggaccttaacggtgcggcgcaccatcaccaccatcatggtgcggcg

SEQ ID NO: 33 - protein, 148 aa, synthetic construct
  REGION 1..148: note = Description of sequence: Synthetic construct
EVQLVESGGGLVQAGGSLRLSCAASGSIFSTNVMGWYRQAPGKQREFVAGITIGGTARYPDSVKGRFTISRDNTQNTVYLQMNNLKPEDTAVYYCNAVLPSDQRRWSWGQGTQVTVSSAAASGSLEQKLISEEDLNGAAHHHHHHGAA

SEQ ID NO: 34 - protein, 143 aa, synthetic construct
  REGION 1..143: note = Description of sequence: Synthetic construct
EVQLVESGGGLVQTGGSLRLSCTASGTIDMTYGLIWYRQAPGKERELVASIRRDGRTNYADSVKGRFTISIDNAKNSIHLQMNSLKPDDTARYYCNSPYHALWGQGTQVTVSSAAASGSLEQKLISEEDLNGAAHHHHHHGAA

SEQ ID NO: 35 - DNA, 444 bp, synthetic construct
  misc_feature 1..444: note = Description of sequence: Synthetic construct
  source 1..444: mol_type = other DNA
gaggtacaactggttgagagtgggggtggtttggtgcaagccggaggttccttacgtttgtcttgcgcggctagtgggagcatcttttcaacaaacgtaatggggtggtaccgccaagccccaggtaagcagcgggaatttgtggccgggataacgatcggaggaactgcgaggtatcctgatagtgtgaaagggcgtttcacaattagtcgagataatacacagaatactgtctatctccaaatgaataatctcaagcccgaagacacagcagtttattattgtaatgccgttctcccctctgatcagcgtcgatggagctggggacaaggcacccaggttacggttagcagcgcggcagcgtctggttcgctcgagcaaaagctcatatctgaggaggacctgaacggggcagcccaccatcaccaccatcacggagcagct

SEQ ID NO: 36 - DNA, 429 bp, synthetic construct
  misc_feature 1..429: note = Description of sequence: Synthetic construct
  source 1..429: mol_type = other DNA
gaggtacagctggtggagtccggcggtggtttggtgcaaaccgggggtagtctgcggcttagttgcacggcgtctgggacaatagacatgacttatggtctcatatggtacaggcaagcgcctgggaaagagagggaactcgttgcgagtatcagaagggacggccgcacaaattacgctgattcagtgaaagggcgcttcactatctcgatcgataatgcgaaaaacagtattcaccttcaaatgaactcccttaagcccgatgataccgccaggtattattgcaacagcccatatcacgcactttggggtcagggtacgcaggtaacagtgtctagtgcggcagcctctggtagtttggagcaaaagttgataagtgaggaggacttaaatggggcggcacatcaccaccaccatcatggggcggct

SEQ ID NO: 37 - DNA, 1001 bp, synthetic construct
  misc_feature 1..1001: note = Description of sequence: Synthetic construct
  source 1..1001: mol_type = other DNA
aaattaaaaggctggattttttcggcctttttttagtgcaaataattattttttacgtatttatattatagggctaatcactaaactaataattagtggttgaagcgctgaaaattttctgctattttattaatagtttgataataaaataatgatatttaatataaagagggataaacgaaataatgaaatcaacaacaaagaaaattcttgcatcgtcgttaggggtagctggcgcaatggcaatgggcacggtaactgcaaaggctgatacgaccgttacggtcaatgctggcgatagtttgaatgggattgctcaaaagtataatgttagtgcggatgatattgcaaccgctaatcacttgcaaaataaagagttgatttttgtgggacaaaagttgacaattccaaccaaagataaaaatgaaacagaagttcaattagttgaaagtggtggtggtttagttcaaccaggtggtagtttacgtttatcatgtgctgcaagtggttcaattgcaactattaatgatatgggttggtttcgtcaagcaccaggaaagcaacgtgattgggttgctactattgttagtgatggttcaactgcttatgctgatagtgttaaaggtcgttttactatttcacgtgataatgctaagaatactgtttaccttcaaatgaatagtcttaagccagaagatactgcagtttactattgttcagctcgtcgtcattatggtcaaggtactcaagttactgttagttcagctgcagctagtggttcattagaacaaaaattaatttcagaagaagatttaaatggtgcagctcatcatcatcatcatcatggtgcagcttacttccctggaaattactatgttgtgatgcattaatattccctttcacctcacctttaataatttaaattagtaattatcttgcgcatcacaaaagagtgctatatactatttcagattagaaagttttatgagggagacaaattg

SEQ ID NO: 38 - DNA, 1393 bp, synthetic construct
  misc_feature 1..1393: note = Description of sequence: Synthetic construct
  source 1..1393: mol_type = other DNA
gaagtacaaagttactttaactataatgaaaaacaagacaatataaagaaaacaacatataaggttcagttcataactgattagatttataataaatattgtaaatcggacaaaaataaattaattttcaattaattcaaaaaaaccatatttttttcgttttggcatatttggatttgctacactaaagatgatcaagaaaggggaaaagataatcttcaatcttgtgtacttagtttgttaattaatttataaatttagggaggaaacctatcatggaaaagactatgaaaaagaaagctttagttgcaactactgctgtagccggtattactttagtaggagaggttactactgttcatgccgctgacaatgtacaacaagaagttcaattagttgaaagtggtggtggtttagttcaaccaggtggtagtttacgtttatcatgtgctgcaagtggttcaattgcaactattaatgatatgggttggtttcgtcaagcaccaggaaagcaacgtgattgggttgctactattgttagtgatggttcaactgcttatgctgatagtgttaaaggtcgttttactatttcacgtgataatgctaagaatactgtttaccttcaaatgaatagtcttaagccagaagatactgcagtttactattgttcagctcgtcgtcattatggtcaaggtactcaagttactgttagttcagctgcagctagtggttcattagaacaaaaattaatttcagaagaagatttaaatggtgcagctcatcatcatcatcatcatggtgcagctaagaagattacagaagatcaaaaagcaacagaaggcggttatcatattgttaatggatctgttgtagataagcagaataacttggttagtggttggactgttaagaatggtcaaatggttgatcctgaaggtaacgttatcaaaacaacaatgtctacagcccaaggtgttactattgaaaaaaataatagcaagtccgggaatacaaagacaaacatgattcaaacttctttaactattgctaacaacaaggcaacaacaaacaaagacaaccagttaccacaaactggcaattacaacaacaatacaaaggtattaggattagctggtattgcacttgcatctgctttaactatgtttggatacaagaagcgccaacataactaattttcttacttgatgggtttctaaataaaaaatggactacttcagctcaaggtagcccatttttattattatagtgaagcagttattcttacgtataaccagactaaaatataataaaaatctattatatattaatcaacatctcggtttaatcgttaaaactcctctgagagctaattgttaatattgagttgtatagt

SEQ ID NO: 39 - DNA, 1419 bp, synthetic construct
  misc_feature 1..1419: note = Description of sequence: Synthetic construct
  source 1..1419: mol_type = other DNA
gcaatgcacaagatgctgaaacaaaggcacaacaaaatgcagatcaagcttcaccagctaatattcaaaaggcacaagatgctattgctaatcaagaaactcaaattagtaaagacaccgatgctattaatgacgctaacaaagccgttagcgatgcacaaagcacagttgatgcagcgcaaaaaaagttaatgatgcaactactgctcgtgacaatcaacaaaagaatgttgatactgctagtgatgcagttaagaatgctcaagctattcttgacaacagtgatcaggctaaaaaggaagcccaagatgctttgaacaaggctaaccaaaaagttgctgatgatactactgccgttaacaacaaacaaactgatgttaacaatgcagcagaagctaagaagaatgcagatgaggcattgaagaacgccaatgatgcgcaaacttctgcacaaaagaataaagatgctaagcaagcaattgctgatgaggcaagtgtagaagttcaattagttgaaagtggtggtggtttagttcaaccaggtggtagtttacgtttatcatgtgctgcaagtggttcaattgcaactattaatgatatgggttggtttcgtcaagcaccaggaaagcaacgtgattgggttgctactattgttagtgatggttcaactgcttatgctgatagtgttaaaggtcgttttactatttcacgtgataatgctaagaatactgtttaccttcaaatgaatagtcttaagccagaagatactgcagtttactattgttcagctcgtcgtcattatggtcaaggtactcaagttactgttagttcagctgcagctagtggttcattagaacaaaaattaatttcagaagaagatttaaatggtgcagctcatcatcatcatcatcatggtgcagctgctgaaaaggaagctaaagataatggctaccatatcgaaaataaccaagttgttgacgctaaaggtaatagtgtcaatggctggacagttaagggcaaccaaattgttagtccaactaatgctactgtagatcccgctgtttctgtaaccaccaatgtcaatgttgatagtaaaggtcaagtacaaccacaaactagtgttactgctaatagtgttaagactgtagctgcaactgaatcagcaaatccagtagcaactactactgtgcaaacccgcgaacaatacaagcaacaattgaagagcaataatcaattaccacaaactggtaataatgatagtgctgttctttcacttgctggagtagcacttgcagcaatgttgagtttgttcggtattaagaaacgtgaatactaatttagaaaatgtaagtattattatgtaaaaaggttcaaccaaattggctgaacctttttgtctaaaatttaaggagaagtttt

SEQ ID NO: 40 - protein, 739 aa, synthetic construct
  REGION 1..739: note = Description of sequence: Based on Lactobacillus reuteri sequence
MNKANQKVADDTTAVNNKQTDVNNAAEAKKNADEALKNANDAQTSAQKNKDAKQAIADEASVALADANTAVKDAQAKVDAINDKLANFNTITLPAGYKDDLIAYYNYFGNSNYNQDEANNLAQDLLKYRDQAMSQNKFKDNLSDDRVVDIDNLNSTDRAELSQFVASLINQVRTQMGTNLVISSPAADDYAEQVSQNYNKDNWNSADNGKHDQSALNNATDQLNISWNGENMGLDQSIFTTDYTVLTDGTKLPTGNKQTINDLKHLIYDDFISMMFDDADSAWGHATNFAGIDNFAAEKQAVGFSLDKFYNTHYDLVEANQKVEENSYTLPSINALTQKLADAKDDLSIKQTDQASKQKANDDAQNALSSANQVLVAAQNDVKDKTATAQEANDNLTTAQNDLATLQNQLSADQANQKQAQTTFDSFDADLATKQANLQKATDSLKAEQGRLAIAQADLDNANKALSDANNNLAQKKQVVENDNETLKVDNDKLVQLQNNLSDLQNAPKLLAAAKEQVATAQKALADAQEAYNVANDKLTSLKQTAAGTTTNVSKAQQALAEAKNNEDAAKEVLDQAQQALTELRQKEALAKQVAEEQAKLAAEKEAKDNGYHIENNQVVDAKGNSVNGWTVKGNQIVSPTNATVDPAVSVTTNVNVDSKGQVQPQTSVTANSVKTVAATESANPVATTTVQTREQYKQQLKSNNQLPQTGNNDSAVLSLAGVALAAMLSLFGIKKREY

SEQ ID NO: 41 - DNA, 185 bp, synthetic construct
  misc_feature 1..185: note = Description of sequence: Based on Lactobacillus reuteri sequence
  source 1..185: mol_type = other DNA
aaattaaaaggctggattttttcggcctttttttagtgcaaataattattttttacgtatttatattatagggctaatcactaaactaataattagtggttgaagcgctgaaaattttctgctattttattaatagtttgataataaaataatgatatttaatataaagagggataaacgaaata

SEQ ID NO: 42 - DNA, 275 bp, synthetic construct
  misc_feature 1..275: note = Description of sequence: Based on Lactobacillus reuteri sequence
  source 1..275: mol_type = other DNA
gaagtacaaagttactttaactataatgaaaaacaagacaatataaagaaaacaacatataaggttcagttcataactgattagatttataataaatattgtaaatcggacaaaaataaattaattttcaattaattcaaaaaaaccatatttttttcgttttggcatatttggatttgctacactaaagatgatcaagaaaggggaaaagataatcttcaatcttgtgtacttagtttgttaattaatttataaatttagggaggaaacctatc

SEQ ID NO: 43 - DNA, 313 bp, synthetic construct
  misc_feature 1..313: note = Description of sequence: Based on Lactobacillus reuteri sequence
  source 1..313: mol_type = other DNA
gcaatgcacaagatgctgaaacaaaggcacaacaaaatgcagatcaagcttcaccagctaatattcaaaaggcacaagatgctattgctaatcaagaaactcaaattagtaaagacaccgatgctattaatgacgctaacaaagccgttagcgatgcacaaagcacagttgatgcagcgcaaaaaaagttaatgatgcaactactgctcgtgacaatcaacaaaagaatgttgatactgctagtgatgcagttaagaatgctcaagctattcttgacaacagtgatcaggctaaaaaggaagcccaagatgct

SEQ ID NO: 44 - protein, 46 aa, synthetic construct
  REGION 1..46: note = Description of sequence: Based on Lactobacillus reuteri sequence
AIKPVPSPNGIFAASFELNGTTWIFKYKYYDSSKGYWVGIYESVDK

SEQ ID NO: 45 - protein, 64 aa, synthetic construct
  REGION 1..64: note = Description of sequence: Based on Lactobacillus reuteri sequence
LASTLGISTAAAKKAIDIIDAASTIASIISLIGIVTGAGAISYAIVATAKTMIKKYGKKYAAAW

SEQ ID NO: 46 - protein, 38 aa, synthetic construct
  REGION 1..38: note = Description of sequence: Based on Lactobacillus reuteri sequence
EFSGGGGAEQRGISQGNDGKLCTLTWECGLCPTHTCWC

SEQ ID NO: 47 - protein, 35 aa, synthetic construct
  REGION 1..35: note = Description of sequence: Based on Lactobacillus reuteri sequence
SDATPMTVTPTTITIPISLAGCPTTKCASIVSPCN

SEQ ID NO: 48 - protein, 36 aa, synthetic construct
  REGION 1..36: note = Description of sequence: Based on Lactobacillus reuteri sequence
SEATPMTVTPTTITIPISLAGCPTTKCASIVSPCND

SEQ ID NO: 49 - protein, 154 aa, synthetic construct
  REGION 1..154: note = Description of sequence: Synthetic construct
EVQLVESGGGLVQAGGSLRLSCAGSGHTGSLYSMGWFRQAPGKEREFVAAITWRPSSTYYADSVKGRFTISRDDAKNTVYLQMNSLKPEDTAVYFCAARPRGGLSPTPQAYDYWGQGTQVTVSSAAASGSLEQKLISEEDLNGAAHHHHHHGAA

SEQ ID NO: 50 - protein, 143 aa, synthetic construct
  REGION 1..143: note = Description of sequence: Synthetic construct
EVQLVESGGGLVQTGGSLRLSCTASGTIDMTYGLIWYRQAPGKERELVASIRRDGHTNYADSVKGRFTISIDNAKNSIHLQMNSLKPDDTARYYCNSPYHALWGQGTQVTVSSAAASGSLEQKLISEEDLNGAAHHHHHHGAA

(19) United States
(12) Patent Application Publication
(10) Pub. No.: US 2026/0207686 A1
(43) Pub. Date: Jul. 23, 2026

(54) GENETICALLY MODIFIED LACTOBACILLUS AND USES THEREOF

(71) Applicant: BiomEdit, Inc., Fishers, IN (US)

(72) Inventors: Dharanesh Mahimapura GANGAIAH, Fishers, IN (US); Arvind KUMAR, Fishers, IN (US); Lin LIU, Fishers, IN (US); Shrinivasrao Peerajirao MANE, Fishers, IN (US); Valerie Elyse RYAN, Fishers, IN (US)

(21) Appl. No.: 19/565,034

(22) Filed: Mar. 12, 2026

Related U.S. Application Data
(62) Division of application No. 17/428,506, filed on Feb. 4, 2020, now Pat. No. 12,599,637.
(60) Provisional application No. 62/801,307, filed on Feb. 5, 2019.

(51) Int. Cl. A61K 35/747; A23K 10/18; A23K 20/163; A23K 50/75; A23L 33/135; A61K 35/742; A61K 38/00; A61P 1/00; A61P 31/04; C07K 14/32; C07K 14/335; C07K 16/1282; C12N 1/205; C12N 15/74; C12R 1/225

(52) U.S. Cl. CPC A61K 35/747; A23K 10/18; A23K 20/163; A23K 50/75; A23L 33/135; A61K 35/742; A61P 1/00; A61P 31/04; C07K 16/1282; C12N 1/205; C12N 15/746; A23V 2002/00; A23V 2400/173; A61K 38/00; C07K 14/32; C07K 14/335; C07K 2317/22; C07K 2317/76; C07K 2319/02; C07K 2319/035; C12R 2001/225

(57) ABSTRACT

The present invention relates to efficient delivery of antibiotic activity, immunomodulatory factors, or growth-promoting biomolecules directly to the digestive tract of an animal via a live delivery platform. The live delivery platform can be a genetically modified microorganism. Delivery can be accomplished with a Lactobacillus sp which colonizes the gastrointestinal tract. The anti-infective activity can be a bacteriocidal or bacteriostatic peptide, an antibody or fragment thereof which specifically recognizes a pathogen, or a phage, or a lytic peptide from a phage which specifically targets a certain pathogen.

### CROSS-REFERENCE TO RELATED APPLICATIONS

The present application is a divisional of U.S. application Ser. No. 17/428,506, filed Aug. 4, 2021, which is a National Stage Application claiming priority from copending PCT Application No. PCT/US2020/016522 filed Feb. 4, 2020, which in turn claims benefit of priority to U.S. Provisional Patent Application Ser. No. 62/801,307, filed Feb. 5, 2019, all of which are hereby incorporated by reference in their entirety.

### SEQUENCE LISTING XML

The instant application contains a Sequence Listing, conforming to the rules of WIPO Standard ST.26, submitted in XML format which was filed electronically by EFS-web and is hereby incorporated by reference in its entirety. Said XML format Sequence Listing, created on Mar. 16, 2026, is named “2950-3_PCT-US-DIV_ST26.xml” and is 190,305 bytes in size.

### FIELD OF THE INVENTION

The present invention relates to a live delivery platform, such as a genetically-modified bacterium, to deliver preventative or therapeutic anti-infective activity, immunomodulatory factors, or growth-promoting biomolecules directly to the mucosa of an animal in need thereof.

### BACKGROUND OF THE INVENTION

Direct fed microbials (DFMs), often also called probiotics, are microorganisms which colonize the gastrointestinal tract of an animal and provide some beneficial effect to that animal. The microorganisms can be bacterial species, for example those from the genera Bacillus, Lactobacillus, Lactococcus, and Entercoccus. The microorganisms can also be yeast or even molds. The microorganisms can be provided to an animal orally or mucosally or, in the case of birds, provided to a fertilized egg, i.e. in ovo.

The beneficial activity provided by a DFM can be the synthesis of vitamins or other nutritional molecules needed for a healthy metabolism of the host animal. A DFM can also protect the host animal from disease, disorders, or clinical symptoms caused by other, pathogenic microorganisms. For example, the DFM may naturally produce factors having inhibitory or cytotoxic activity against certain species of pathogens, such as deleterious or disease-causing bacteria. However, the DFM may not be able to produce such factors in sufficient quantity to reduce infection of the host with the pathogen, or the factors may affect only a limited set of pathogens, leaving the host vulnerable to other pathogens.

Stronger or more broad-based antibiotics can be administered to the host animal, for example orally or parenterally, but these would have a limited duration in the host, and thus may require repeated administration. Oral delivery may also result in the degradation of the antibiotics or failure to deliver the antibiotic to the particular anatomical site where the therapeutic effect is most needed. Development of antibiotic resistance by pathogens is another important concern.

What is needed is a delivery system which can constantly deliver anti-infective molecules directly to the gastrointestinal or respiratory tract where pathogenic bacteria are replicating in the host. The gastrointestinal and respiratory systems are also often a point of entry of the pathogen into the host. Preferably, the delivery system is a live genetically modified microorganism, such as a bacterium, which can colonize the gastrointestinal or respiratory tract of a host and directly deliver antibiotic factors to reduce the number of, or block the entry of, a pathogen. For example, in ovo delivery of a live delivery platform could prevent early colonization of an embryo by pathogens, possibly through competitive exclusion or direct or indirect anti-infective effects. In ovo delivery has the further advantage of bypassing any limitations of colonization by the genetically modified microorganism due to maternal antibody interference. Preferably, the live bacterial delivery system synthesizes the anti-infective factor in sufficient quantity to have the desired effect on a pathogen. A targeted pathogen may be, without limitation, a bacterium of the genera Salmonella, Clostridium, Campylobacter, Fusobacterium, Staphylococcus, or Streptococcus, or an E. coli bacterium, or a parasite such as an Eimeria species. Preferably, the live bacterial system persists in the host gastrointestinal tract for a period of time. Preferably, the live bacterial delivery system produces a broad-spectrum anti-infective factor or multiple anti-infective factors, such that a variety of pathogens are targeted. Alternatively, a combination of live delivery systems could be administered to a single animal, with genetically modified bacteria producing multiple anti-infective factors, immunomodulatory molecules, or growth-promoting biomolecules, or any combination thereof. Thus, more than one disease state is prevented or reduced, or diseases and syndromes having multiple causes can be effectively treated.

Provided herein is disclosure of anti-infective peptides, including new mersacidin-like peptides, which target multiple bacterial species. Also disclosed are antibodies, including single chain antibodies, which target specific pathogens and pathogenic molecules. Also disclosed are phage or phage lytic peptides which target pathogenic species. Provided also is a Lactobacillus expression system which can produce high levels of at least one or a multiplicity of the above molecules, preferably as surface-displayed or secreted molecules.

### SUMMARY OF THE INVENTION

The present invention provides a live delivery platform comprising a genetically-modified microorganism. The genetically-modified microorganism comprises an expression cassette containing one or more of: a promoter for transcriptional expression, a nucleic acid sequence encoding a signal sequence for secretion, a nucleic acid sequence encoding a cell-wall anchor, at least one heterologous coding region encoding a desired biomolecule, a nucleic acid sequence encoding an expressed peptide tag for detection, and terminators for translation and transcription termination. The genetically modified microorganism may be a bacterium, a yeast, or a fungus. A genetically modified bacterium is preferably a Bacillus, Lactobacillus, Lactococcus, or an Entercoccus. The genetically modified bacterium may also preferably be an E. coli bacterium. The genetically modified bacterium may also preferably be a Lactobacillus reuteri strain.

The present invention provides an expression cassette within a genetically modified microorganism that may include a promoter for transcriptional expression. The promoter for transcriptional expression may comprise a nucleic acid sequence of SEQ ID NO: 14, SEQ ID NO: 15, SEQ ID NO: 16, SEQ ID NO: 17, SEQ ID NO: 18, SEQ ID NO: 19, SEQ ID NO: 41, SEQ ID NO: 42, and SEQ ID NO: 43.

The present invention provides an expression cassette within a genetically modified microorganism that may include a nucleic acid sequence encoding a signal sequence for secretion. The signal sequence for secretion may be at least 20 amino acids, at least 25 amino acids, at least 30 amino acids, at least 35 amino acids, at least 40 amino acids, at least 44 amino acids, at least 50 amino acids, at least 55 amino acids, at least 60 amino acids, or at least 65 amino acids. The signal sequence for secretion may also be 20-65 amino acids, 20-60 amino acids; 20-55 amino acids; 20-50 amino acids, 20-45 amino acids, 20-40 amino acids, 20-35 amino acids, 20-30 amino acids, 25-65 amino acids, 25-60 amino acids; 25-55 amino acids; 25-50 amino acids, 25-45 amino acids, 25-40 amino acids, 25-35 amino acids, 25-30 amino acids, 30-65 amino acids, 30-60 amino acids; 30-55 amino acids; 30-50 amino acids, 30-45 amino acids, 30-40 amino acids, or 30-35 amino acids. The signal sequence for secretion may comprise an amino acid sequence that is a fragment of SEQ ID NO: 20, SEQ ID NO: 21, SEQ ID NO: 22, SEQ ID NO: 23, and SEQ ID NO: 40, where the fragment has a length as given herein. The signal sequence for secretion may comprise an amino acid sequence that is an amino-terminal fragment of SEQ ID NO: 20, SEQ ID NO: 21, SEQ ID NO: 22, SEQ ID NO: 23, and SEQ ID NO: 40, where the fragment has a length as given herein.

The present invention provides an expression cassette within a genetically modified microorganism that may include a nucleic acid sequence encoding a cell-wall anchor peptide. The cell-wall anchor peptide may have a length of 100-250 amino acids, 100-225 amino acids, 100-200 amino acids, 100-175 amino acids, 100-150 amino acids, 125-250 amino acids, 125-225 amino acids, 125-200 amino acids, 125-175 amino acids, or 125-150 amino acids. The cell wall anchor may comprise an amino acid sequence that is a fragment of SEQ ID NO: 20, SEQ ID NO: 21, SEQ ID NO: 22, SEQ ID NO: 23, and SEQ ID NO: 40, where the fragment has a length as given herein. The cell wall anchor may comprise an amino acid sequence that is a carboxy-terminal fragment of SEQ ID NO: 20, SEQ ID NO: 21, SEQ ID NO: 22, SEQ ID NO: 23, and SEQ ID NO: 40, where the fragment has a length as given herein.

The present invention provides an expression cassette within a genetically modified microorganism that includes a heterologous coding region encoding a desired biomolecule. The desired biomolecule may be a biomolecule having anti-infective activity, a probiotic factor, an immunomodulatory factor, or a growth-promoting biomolecule. The biomolecule may have anti-infective activity active against a pathogenic bacterium or a parasite. The parasite may preferably be an Eimeria species. The pathogenic bacterium may preferably be a Salmonella, Clostridium, Campylobacter, Staphylococcus, Streptococcus, Fusobacterium, and an E. coli bacterium.

The present invention provides an expression cassette within a genetically modified microorganism that includes a heterologous coding region encoding a desired biomolecule having anti-infective activity. The anti-infective biomolecule may be a bactericidal peptide, an enzyme, a lysin, a phage, or an antibody. The bactericidal peptide may be a mersacidin-like molecule. The mersacidin-like molecule may comprise a sequence disclosed herein as SEQ ID NO: 2 or SEQ ID NO: 4. The desired biomolecule may be an enzyme. The enzyme may comprise a sequence disclosed herein as SEQ ID NO: 5 or SEQ ID NO: 6. The desired biomolecule may be a lysin. The lysin may comprise a sequence disclosed herein as SEQ ID NO: 24, SEQ ID NO: 25, SEQ ID NO: 26, SEQ ID NO: 27, or SEQ ID NO: 28. The desired biomolecule may be a phage, or a phage in a pro-phage form. The phage genetic material may comprise a sequence disclosed herein as SEQ ID NO: 11, SEQ ID NO: 12, or SEQ ID NO: 13. The desired biomolecule may be derived from a Bacillus species. The desired biomolecule may be a Bacillus bacteriocin. A Bacillus anti-infective molecule may comprise a sequence disclosed herein as SEQ ID NO: 44, SEQ ID NO: 45, SEQ ID NO: 46, SEQ ID NO: 47, or SEQ ID NO: 48.

The present invention provides an expression cassette within a genetically-modified microorganism that includes a heterologous coding region encoding a desired biomolecule that is an antibody. The antibody may be a single chain antibody. The single chain antibody may be from a camelid. The single chain antibody may specifically recognize a pathogenic microorganism or a molecule produced by a pathogen. The single chain antibody may specifically recognize a bacterial protein, such as for example a toxin or an attachment molecule. The single chain antibody may specifically recognize a bacterial protein from Clostridium perfringens. The bacterial protein may be a protein produced by C. perfringens. The bacterial protein may be C. perfringens alpha toxin or C. perfringens NetB toxin.

The present invention provides an expression cassette within a genetically modified microorganism that includes a heterologous coding region encoding a desired biomolecule that is an antibody that recognizes C. perfringens alpha toxin or C. perfringens NetB toxin. The single chain antibody may comprise an amino acid sequence as disclosed herein as SEQ ID NO: 29, SEQ ID NO: 30, SEQ ID NO: 33, or SEQ ID NO: 34.

The present invention provides an expression cassette within a genetically modified microorganism that includes a heterologous coding region encoding a desired biomolecule that is a probiotic factor. The probiotic factor may be an agglutinin receptor. The agglutinin receptor may comprise a sequence disclosed herein as SEQ ID NO: 9 or SEQ ID NO: 10.

The present invention provides a genetically-modified microorganism comprising an expression cassette. The expression cassette may comprise a promoter for transcriptional expression, and at least one heterologous coding region encoding a desired biomolecule. The expression cassette may also optionally comprise one or more of a nucleic acid sequence encoding a signal sequence for secretion, a nucleic acid sequence encoding a cell-wall anchor, a nucleic acid sequence encoding an expressed peptide tag for detection, and terminators for translation and transcription termination. The expression cassette may comprise a promoter having a sequence disclosed herein as SEQ ID NO: 14, SEQ ID NO: 15, SEQ ID NO: 16, SEQ ID NO: 17, SEQ ID NO: 18, SEQ ID NO: 19, SEQ ID NO: 41, SEQ ID NO: 42, or SEQ ID NO: 43. The at least one heterologous coding region encoding a desired biomolecule may encode a biomolecule such as a bactericidal peptide, an enzyme, a lysin, a phage, and an antibody. The at least one heterologous coding region encoding a desired biomolecule may encode a biomolecule having anti-infective activity. The at least one heterologous coding region encoding a desired biomolecule may encode a biomolecule having a sequence disclosed herein as SEQ ID NO: 2, SEQ ID NO: 4, SEQ ID NO: 5, SEQ ID NO: 6, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 33, SEQ ID NO:34, SEQ ID NO: 44, SEQ ID NO: 45, SEQ ID NO: 46, SEQ ID NO: 47, SEQ ID NO: 48, SEQ ID NO: 49, or SEQ ID NO: 50.

The present invention provides a genetically modified microorganism comprising an expression cassette, where the genetically modified bacterium is a Bacillus, Lactobacillus, Lactococcus, or an Entercoccus. The genetically modified bacterium may be a Lactobacillus reuteri strain.

The present invention provides a genetically modified microorganism comprising an expression cassette, where the expression cassette is located on a plasmid. The plasmid may comprise a sequence disclosed herein as SEQ ID NO: 8. The present invention provides a genetically modified microorganism comprising an expression cassette, where the expression cassette is located on a bacterial chromosome. The expression cassette located on a bacterial chromosome may be inserted into a transposase locus. The expression cassette located on a bacterial chromosome may be inserted into a Uracil phosphoribosyl (UPP) transferase locus. The expression cassette located on a bacterial chromosome may be inserted into a pyrE locus.

The present invention provides a method of reducing colonization of an animal by a pathogenic bacterium. The method may comprise treating an animal in need thereof with a live delivery platform. The live delivery platform comprises a genetically modified microorganism. The genetically modified microorganism comprises an expression cassette containing one or more of: a promoter for transcriptional expression, a nucleic acid sequence encoding a signal sequence for secretion, a nucleic acid sequence encoding a cell-wall anchor, at least one heterologous coding region encoding a desired biomolecule, a nucleic acid sequence encoding an expressed peptide tag for detection, and terminators for translation and transcription termination. The genetically modified microorganism may be a bacterium, a yeast, or a fungus. A genetically modified bacterium is preferably a Bacillus, Lactobacillus, Lactococcus, or an Entercoccus, The genetically modified bacterium may also preferably be an E. coli bacterium. The genetically modified bacterium may also preferably be a Lactobacillus reuteri strain.

The present invention provides a method of reducing colonization of an animal by a pathogenic bacterium, where the method comprises treating an animal in need thereof with a genetically modified microorganism. The genetically modified microorganism is modified to contain an expression cassette. The expression cassette may comprise a promoter for transcriptional expression having a sequence disclosed herein as SEQ ID NO: 14, SEQ ID NO: 15, SEQ ID NO: 16, SEQ ID NO: 17, SEQ ID NO: 18, SEQ ID NO: 19, SEQ ID NO: 41, SEQ ID NO: 42, or SEQ ID NO: 43. The expression cassette may further comprise a signal sequence for secretion having an amino acid sequence that is a fragment of SEQ ID NO: 20, SEQ ID NO: 21, SEQ ID NO: 22, SEQ ID NO: 23, and SEQ ID NO: 40, where the fragment has a length as given herein. A signal sequence for secretion may comprise an amino acid sequence that is an amino-terminal fragment of SEQ ID NO: 20, SEQ ID NO: 21, SEQ ID NO: 22, SEQ ID NO: 23, and SEQ ID NO: 40, where the fragment has a length as given herein. The expression cassette preferably also includes a heterologous coding region encoding a desired biomolecule. The desired biomolecule may be a biomolecule having anti-infective activity, a probiotic factor, an immunomodulatory factor, or a growth-promoting biomolecule. The biomolecule may have anti-infective activity active against a pathogenic bacterium or a parasite. The parasite may preferably be an Eimeria species. The pathogenic bacterium may preferably be a Salmonella, Clostridium, Campylobacter, Staphylococcus, Streptococcus, Fusobacterium, and an E. coli bacterium. The anti-infective biomolecule may be a bactericidal peptide, an enzyme, a lysin, a phage, or an antibody. The expression cassette may optionally contain a nucleic acid sequence encoding a cell-wall anchor, a nucleic acid sequence encoding an expressed peptide tag for detection, and/or terminators for translation and transcription termination.

The present invention provides a method of reducing colonization of an animal by a pathogenic bacterium, where the method comprises treating an animal in need thereof with a genetically modified microorganism. The animal may be a bird, a human, or a non-human mammal. The treatment may be administered orally, parentally, nasally, or mucosally. When the animal is a bird the treatment may be administered in ovo.

The present invention provides a use of any genetically modified microorganism disclosed herein in therapy. The present invention provides a use in therapy of a bacterium genetically modified to contain any expression cassette as disclosed herein. A therapy may be reducing colonization of an animal by a pathogenic bacterium.

The present invention provides a use of any expression cassette disclosed herein in therapy. The present invention provides a use in therapy of any expression cassette as disclosed herein. A therapy may be reducing colonization of an animal by a pathogenic bacterium. The expression cassette should comprise at least one heterologous coding region encoding a desired biomolecule. The at least one heterologous coding region encoding a desired biomolecule may encode a biomolecule such as a bactericidal peptide, an enzyme, a lysin, a phage, and an antibody. The at least one heterologous coding region encoding a desired biomolecule may encode a biomolecule having anti-infective activity. The at least one heterologous coding region encoding a desired biomolecule may encode a biomolecule having a sequence disclosed herein as SEQ ID NO: 2, SEQ ID NO: 4, SEQ ID NO: 5, SEQ ID NO: 6, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 33, SEQ ID NO:34, SEQ ID NO: 44, SEQ ID NO: 45, SEQ ID NO: 46, SEQ ID NO: 47, SEQ ID NO: 48, SEQ ID NO: 49, or SEQ ID NO: 50.

The present invention provides a use of any genetically modified microorganism disclosed herein in the manufacture of a medicament for reducing colonization of an animal by a pathogenic bacterium. The present invention provides for use of a bacterium genetically modified to contain any expression cassette as disclosed herein in the manufacture of a medicament for reducing colonization of an animal by a pathogenic bacterium.

The present invention provides a use of any expression cassette disclosed herein in the manufacture of a medicament for reducing colonization of an animal by a pathogenic bacterium. The present invention provides a use in manufacture of a medicament of any expression cassette as disclosed herein. The medicament may be for reducing colonization of an animal by a pathogenic bacterium. The expression cassette should comprise at least one heterologous coding region encoding a desired biomolecule. The at least one heterologous coding region encoding a desired biomolecule may encode a biomolecule such as a bactericidal peptide, an enzyme, a lysin, a phage, and an antibody. The at least one heterologous coding region encoding a desired biomolecule may encode a biomolecule having anti-infective activity. The at least one heterologous coding region encoding a desired biomolecule may encode a biomolecule having a sequence disclosed herein as SEQ ID NO: 2, SEQ ID NO: 4, SEQ ID NO: 5, SEQ ID NO: 6, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 33, SEQ ID NO:34, SEQ ID NO: 44, SEQ ID NO: 45, SEQ ID NO: 46, SEQ ID NO: 47, SEQ ID NO: 48, SEQ ID NO: 49, or SEQ ID NO: 50.

The present invention provides an antibody comprising an amino acid sequence of SEQ ID NO: 29, SEQ ID NO: 49, SEQ ID NO: 30, SEQ ID NO: 33, SEQ ID NO: 50 or SEQ ID NO: 34. The antibody preferably binds a toxin produced by Clostridium perfringens. The antibody preferably binds C. perfringens alpha toxin or a C. perfringens NetB toxin. The present invention provides use of an antibody comprising an amino acid sequence of SEQ ID NO: 29, SEQ ID NO: 49, SEQ ID NO: 30, SEQ ID NO: 33, SEQ ID NO: 50 or SEQ ID NO: 34 in therapy. The present invention provides use of an antibody comprising an amino acid sequence of SEQ ID NO: 29, SEQ ID NO: 49, SEQ ID NO: 30, SEQ ID NO: 33, SEQ ID NO: 50 or SEQ ID NO: 34 in reducing colonization of an animal by a C. perfringens bacterium. The present invention provides use of an antibody comprising an amino acid sequence of SEQ ID NO: 29, SEQ ID NO: 49, SEQ ID NO: 30, SEQ ID NO: 33, SEQ ID NO: 50 or SEQ ID NO: 34 in the manufacture of medicament to reduce colonization of an animal by a C. perfringens bacterium. The present invention provides a method of treating an animal for C. perfringens infection or colonization, where the method comprises administering an antibody comprising an amino acid sequence of SEQ ID NO: 29, SEQ ID NO: 49, SEQ ID NO: 30, SEQ ID NO: 33, SEQ ID NO: 50 or SEQ ID NO: 34 to an animal in need thereof.

### BRIEF DESCRIPTION OF THE DRAWINGS

FIG. 1. Antibiotic resistance or sensitivity of Lactobacillus reuteri strains, as determined with an AVIPRO® Plate.

FIG. 2. Pathogenic bacteria, but not nonpathogenic Lactobacillus acidophilus are killed by L. reuteri strain 3632, as evidenced by a clear “halo” surrounding strain 3632.

FIG. 3. Chromosomal integration of an expression cassette. A. Example of a suicide vector map with a representative expression cassette. B. Map of possible chromosomal integration sites.

### DETAILED DESCRIPTION OF THE INVENTION

As used herein, a “genetically-modified microorganism” means any microorganism which has been altered from the natural state using molecular biological techniques. A genetic modification could be the deletion of a portion of the bacterial chromosome or a naturally-occurring plasmid. The genetic modification could also be the introduction of an artificial or exogenous nucleic acid into a portion of the chromosome. The introduction may or may not disturb or perturb the expression of a bacterial gene. The genetic modification could also be the introduction of an artificial plasmid. The genetically-modified microorganism may be a bacterium, a virus, a yeast, a mold, or a single-celled organism.

An “artificial nucleic acid” or “artificial plasmid” is any nucleic acid or plasmid which does not occur naturally, but rather has been constructed using molecular biological techniques. Portions of the nucleic acid or plasmid may occur naturally, but the those portions are in an artificial relationship or organization.

As used herein, an “expression cassette” is an artificial nucleic acid constructed to result in the expression of a desired biomolecule by the genetically-modified microorganism. An expression cassette comprises one or more of a promoter for transcriptional expression, a nucleic acid sequence encoding a signal sequence for secretion, a nucleic acid sequence encoding a cell-wall anchor, at least one heterologous coding region encoding a desired biomolecule, a nucleic acid sequence encoding an expressed peptide tag for detection, and terminators for translation and transcription termination. A promoter directs the initiation of transcription of the coding regions into a messenger RNA and the translation of the mRNA into a peptide. A signal sequence for secretion, or a secretion signal sequence, directs the peptide to be located outside the cell membrane. The extracellular peptide could be a soluble, secreted protein or it may be cell-associated, particularly if the expression cassette contains a cell wall anchor sequence which attaches the extracellular peptide to a bacterial cell wall. An expressed peptide tag is any amino acid sequence which may be recognized by an antibody or other binding protein. The expressed peptide tag may also bind an inorganic substance, such as a six-histidine tag which binds to nickel molecules. Terminators for translation may be a stop codon or a spacer open reading frame containing a stop codon.

As used herein, a “heterologous coding region” is a nucleic acid sequence containing an open reading frame which encodes a peptide. The coding region is heterologous to the associated promoter, meaning the coding region and the promoter are not associated in their natural states.

As used herein, a “protein” is a sequence of amino acids which assumes a three-dimensional structure. A “peptide” can be used interchangeably with protein but may also be a short linear sequence of amino acids without a defined three-dimensional structure.

As used herein, a “desired biomolecule” is any peptide which may be advantageous to a host when administered via a live delivery platform. The desired biomolecule may be a peptide with anti-infective activity, a probiotic factor, an immunomodulatory factor, an anti-antinutritional factor, or a growth-promoting biomolecule. The desired biomolecule may also be an enzyme which produces a substance with anti-infective activity or a probiotic factor such as a vitamin.

As used herein, “anti-infective activity” includes any activity which prevents infection of a host with a pathogenic organism. The following molecules are examples of biomolecules possessing anti-infective activity: an antibacterial peptide; a lysin or lytic enzyme; a prophage, phage or virus; an enzyme, for example one that cleaves or disables a protein made by a pathogen; and an antibody which blocks, inhibits, or clears a pathogenic molecule. An anti-infective may have bactiostatic activity, which slows, reduces, or prevents the growth of a pathogenic species. A non-limiting example of an antibacterial peptide is a member of the mersacidin family or a mersacidin-like molecule, such as those described in EP0700998. A non-limiting example of lysins are lytic molecules produced by phage. Lysins may have specificity for certain pathogenic species of bacteria and have been suggested for use in substitution for traditional antibiotics. V. A. Fischetti, Viruses, vol. 10, no. 310 (2018); and R. Vazquez et al. Frontiers in Immunology, vol. 9, article 2252 (2018).

As used herein, a “probiotic factor” is a substance which, when produced by a genetically-modified microorganism, proves beneficial to a host. The probiotic factor may be an attachment molecule or an agglutinizing molecule which promotes colonization of the host with the genetically modified microorganism and/or prolongs the period of time where the genetically modified microorganism colonizes the host. The longer the genetically-modified microorganism persists in the host the longer the beneficial effect is provided.

As used herein, an “immunomodulatory factor” could be a cytokine, lymphokine, chemokine, interleukin, interferon, a colony stimulating factor, or a growth factor. The immunomodulatory factor could provide nonspecific enhancement of an immune response or the immunomodulatory factor could increase the number or tissue distribution of immune cells present in the host. The immunomodulatory factor may also reduce an inappropriate immune response, such as without limitation an autoimmune response.

As used herein, a “growth-promoting biomolecule” could be a growth factor, a transfer factor (such as an iron-chelating molecule), a hormone, or any other factor which promotes healthy metabolic activity.

As used herein, an “anti-nutritional factor” could include protease inhibitors for example, a trypsin inhibitors.

As used herein, “delivery” or “administration” means the act of providing a beneficial activity to a host. The delivery may be direct or indirect. An administration could be by an oral, nasal, or mucosal route. For example without limitation, an oral route may be an administration through drinking water, a nasal route of administration may be through a spray or vapor, and a mucosal route of administration may be through direct contact with mucosal tissue. Mucosal tissue is a membrane rich in mucous glands such as those that line the inside surface of the nose, mouth, esophagus, trachea, lungs, stomach, gut, intestines, and anus. In the case of birds, administration may be in ovo, i.e. administration to a fertilized egg. In ovo administration can be via a liquid which is sprayed onto the egg shell surface, or an injected through the shell.

As used herein, the terms “treating”, “to treat”, or “treatment”, include restraining, slowing, stopping, reducing, ameliorating, or reversing the progression or severity of an existing symptom, disorder, condition, or disease. A treatment may also be applied prophylactically to prevent or reduce the incidence, occurrence, risk, or severity of a clinical symptom, disorder, condition, or disease. As used herein, the term “reducing” may apply to both prophylactic (e.g. preventative) treatments or therapeutic treatments.

The following experimental examples are illustrative of a live delivery system comprising Lactobacillus expression cassettes which can be delivered by the disclosed live delivery platform. It will be appreciated that other embodiments and uses will be apparent to those skilled in the art and that the invention is not limited to these specific illustrative examples or preferred embodiments. The descriptive headings of these Examples are for convenience only and should not influence interpretation of any of the results presented therein.

### Example 1. L. reuteri Strain Identification

Seven Lactobacillus reuteri (L. reuteri) strains are isolated from older birds at the Research Center, Hannover from the cecal contents received from the Poultry Clinic, University of Hannover. All the seven strains are identified to be L. reuteri by 16S rRNA sequencing.

While only limited growth is observed for most strains under aerobic conditions in MRS broth and agar (de Man, J. D.; Rogosa, M.; and Sharpe, M. E. “A Medium for the Cultivation of Lactobacilli”. J Appl Bact. 23:130-135 (1960)), all isolates show very good growth on MRS agar and MRS broth under anaerobic conditions at 39° C. Culturing the bacterial strains on blood agar under anaerobic conditions results mostly in limited growth. None of the strains is able to grow in Mueller Hinton broth under anaerobic conditions. For all further analysis, bacterial strains are grown in MRS medium under anaerobic conditions at 39° C.

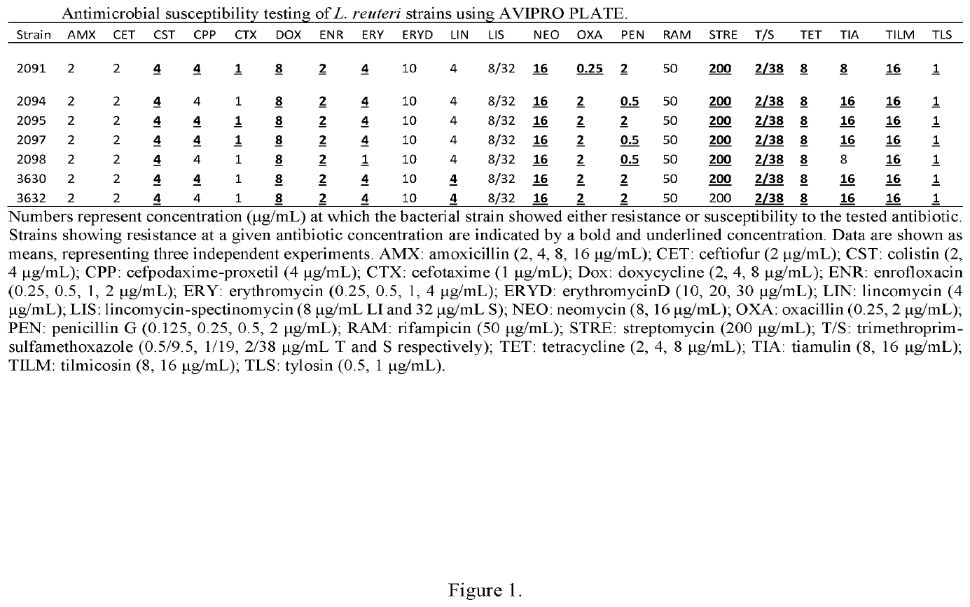

Antimicrobial susceptibility of bacterial isolates are tested using the AVIPRO® PLATE (FIG. 1). All strains are resistant against colistin, doxycycline, enrofloxacin, erythromycin, neomycin, oxacillin, penicillin G, trimethroprim-sulfamethoxazole, tetracycline, tilmicosin and tylosin. All strains are resistant to streptomycin except strain 3632, and to tiamulin except strain 2098. In addition, resistance to cefpodaxime-proxetil is observed with strains 2091, 2095, 2097 and 3630; resistance to cefotaxime is observed with strains 2091, 2095 and 2097; and resistance to lincomycin is observed with strains 3630 and 3632. No strain is found to be resistant against amoxicillin, ceftiofur, erythromycin D, lincomycin-spectinomycin and rifampicin under tested concentrations. Lactobacillus reuteri strain 3632 was deposited on 19 Jun. 2020 according to the Budapest Treaty in the American Type Culture Collection (ATCC), ATCC Patent Depository, 10801 University Boulevard, Manassas, Va., 20110, USA. The deposit was assigned ATCC Patent Deposit Number PTA-126788. Lactobacillus reuteri strain 3630 was deposited on 19 Jun. 2020 according to the Budapest Treaty in the American Type Culture Collection (ATCC), ATCC Patent Depository, 10801 University Boulevard, Manassas, Va., 20110, USA. The deposit was assigned ATCC Patent Deposit Number PTA-126787.

### Example 2. L. reuteri Strain Selection and Anti-Infective Activity

To select the best strain for further engineering, the L. reuteri isolates are tested for various desirable probiotic anti-infective properties, such as growth kinetics, ability to produce hydrogen peroxide, autoaggregation, enzyme profile, survival in the presence of ox bile and pancreatic enzymes, and sensitivity to heat shock and pH changes. The L. reuteri strains are also tested for safety using a haemolytic assay.

In general, all strains behave very similar in terms of probiotic properties, including growth kinetics and ability to produce hydrogen peroxide, except for strain 3632, which shows some unique properties, including the ability to auto-aggregate in liquid media (comparable to that of the well-characterized human probiotic strain L. reuteri ATCC 23272). None of the strains is found to be hemolytic on blood agar plates, suggesting that these isolates are less likely to be pathogenic to humans.

Whole-genome sequencing is performed for L. reuteri strains 2091 and 3632, and an independently isolated L. reuteri strain 170331 of European origin, using PACBIO® sequencing (Amplicon Express). Sequencing, assembly and annotation statistics are summarized in TABLE 1. Genomic structures and organization differ among the tested strains.

TABLE 1
Genome sequencing, assembly and annotation statistics.

| Strain | 170331 | 2091 | 3632 |
|---|---|---|---|
| Genome size | 2,090,596 bp | 2,231,245 bp | 2,482,713 bp |
| Contigs | 9 | 11 | 7 |
| Coding Sequences | 2,154 | 2,280 | 2,595 |
| (ORFs) | | | |
| Ribosomal | 2,231 | 2.354 | 2,680 |
| Binding Sites | | | |
| Transcription | 1,123 | 1,123 | 1,375 |
| Terminators | | | |
| Operons | 452 | 472 | 541 |
| tRNA | 75 | 61 | 77 |
| rRNA | 18 | 19 | 13 |
| Other RNAs | 39 | 99 | 89 |
| CRISPR | 1 | 0 | 0 |
| Prophages | 7 | 1 | 8 |
| Islands | 30 | 22 | 24 |
| Bacteriocins | 0 | 1 | 4 |

Based on the genome sequencing data, strain 3632 encodes for two bacteriocins belonging to mersacidin family based on homology to the mersacidin conserved domain. These bacteriocins appear to be unique to strain 3632. A cDNA encoding one mersacidin (mersacidin-E1) could be:

(SEQ ID NO: 1)

| | |
|---|---|
| 1 | atggacaaag aagaattaga aaaaattgta |
| | ggtaataact ttgaggaaat gagtttacaa |
| 61 | aaaatgacag aaattcaagg tatgggtgaa |
| | taccaagtgg attcaacacc agcagcttct |
| 121 | gcgatttcac gggcaacaat tcaagtatca |
| | cgtgcatctt ctggaaaatg tctaagttgg |
| 181 | ggtagtggtg cagcatttag tgcttatttt |
| | actcataaaa gatggtgcta g. |

This novel open reading frame would encode a polypeptide of mersacidin-E1:

(SEQ ID NO: 2)
MDKEELEKIVGNNFEEMSLQKMTEIQGMGEYQVDSTPAASAISRATIQVS
RASSGKCLSWGSGAAFSAYFTHKRWC.

Another cDNA encoding the second form of mersacidin (mersacidin-E2) could be:

| | |
|---|---|
| 1 | atggaagaaa aagaattaga aggtgtaata gggaattcgt ttgaaagtat gactgtagag |
| 61 | gaaatgacaa aaattcaagg tatgggtgaa tatcaagtag attcgacgcc tggatatttt |
| 121 | atggaaagtg ctgccttttc agctcttaca gccaatataa caagacatgc tatgcatcat |
| 181 | cattaa. |

(SEQ ID NO: 3). This novel open reading frame would encode a polypeptide of mersacidin-E2: MEEKELEGVIGNSFESMTVEEMTKIQGMGEYQVDSTPGYFMESAAFSALTANITR HAMHHH (SEQ ID NO: 4).

L. reuteri strain 3632 encodes other polypeptides which contribute anti-infective activity to this strain. Capreomycidine synthase, involved in capreomycin synthesis, is identified to be expressed in both the culture supernatant and pelleted cells.

(SEQ ID NO: 5)
MVEIAHFGVEAWLNKWEKSATYDISQSTIASLSMHDLLNLDGNNGEEFYE
MLDKQQMNYGWIEGSPEFKEEVAKLYHHVDPENILQTNGATGANILALYA
LINPGDHVIAEYPSYQQLYDIPKSLGADVDYWHIHEEDNWYPRIDDLKAM
VKPNTKMICLNNANNPTGTVLDKEFLEQVVEIAKSVDAYVLVDEVYLPLD
HPEKFAQIIDLYDKGISTNSLSKTYSVPGVRIGWTATNAEVADIFRKFRD
YTMICGGVFNDQLATYVLRHRDQVLARNRKLVLGNLAIYKDWIDHEDRAS
VIMPQAVSTSFPKLDVPVDIHTFCENLLHDEGVLLVPGDAFDTPGHVRLG
YCAPEATLKEGLKRLSKYMHQYD.

Colicin V production protein is also expressed in pelleted cells of strain 3632: MILTTFIILILMGCFINGHRRGLLTMTLMLGTYIVAWIVARQGAQLIGGWLKSLLP SIGTPATFSESLLANVNSNLFFYNGIAFMIIFTIVSILCHWGIRQLNWIKRIPVVGTV DKIAGGLISFLIGYLIIYVVLLIMQLFPAGWWQMQIANSELARFMINQTPGIAHLVI DTLVQGG (SEQ ID NO:6). A person of skill in the art would recognize that, because of the redundancy of the genetic code, multiple nucleic acid sequences could encode the above peptides.

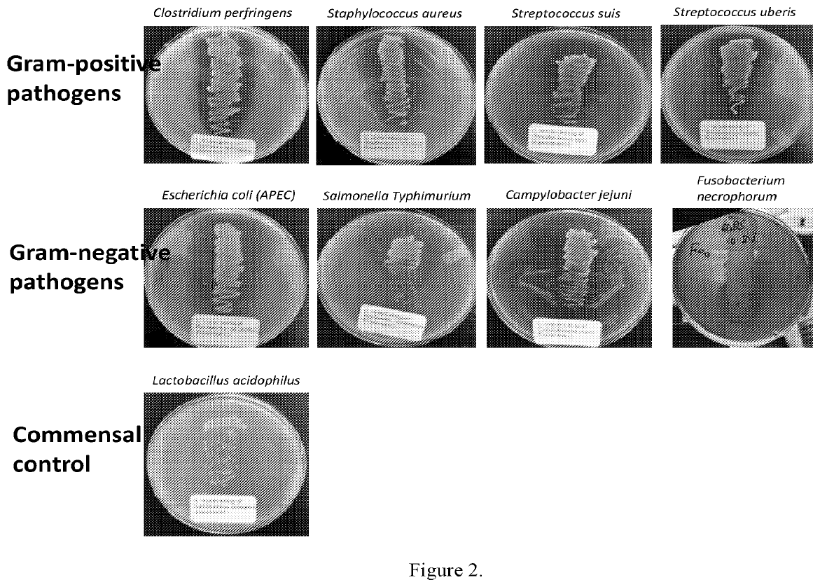

The functionality of these putative bacterocins is tested by co-plating pathogenic bacteria with strain 3632. As shown in FIG. 2, the pathogenic bacteria Clostridium perfringens, Staphylococcus aureus, Streptococcus suis, Streptococcus uberis, Escherichia coli (APEC), Salmonella Typhimurium, Fusobacterium necrophorum, and Campylobacter jejuni are killed by L. reuteri 3632. A non-pathogenic control, Lactobacillus acidophilus, is not killed. DFM strains such as 3632 may be used to control, limit, prevent, or eliminate infection by pathogenic bacteria in swine (S. suis), poultry (C. perfringens, E. coli, S. typhimirium, and C. jejuni), and cattle (S. aureus, S. uberis, and F. necrophorum). DFM strains such as 3632 may be used to control, limit, prevent, or eliminate infection by food-borne pathogenic bacteria in humans.

### Example 3. Other Genetic Elements of L. reuteri Strain 3632

In order to identify any potential plasmids containing antimicrobial resistance genes, native plasmids are isolated and identified by high throughput sequencing (ACGT, Inc). The 3632 strain contains three native plasmids but none with any known antimicrobial resistance genes. However, one of the plasmids is maintained in high copy numbers:

| (SEQ ID NO: 7) | |
|---|---|
| 1 | TTGGCAATCT TTCACCTATC AGCAAAAATC ATTAGTCGAG GAAAAGGGCA ATCAGCAATT |
| 61 | GCCTCGGCTG CTTATCGTTC AGGGAACAAA CTTCACGATG AACGATACGA CGAAACACAA |
| 121 | GATTACACAA ACAAACGTTT CATCGAACAC TCAGAAATCC AACTTCCAGA GAATGCCCCA |
| 181 | GCTAAGTATC AAGATAGGGC AACCCTTTGG AACAGCGTAG AAAAGGCAGA AAAAGCTAAG |
| 241 | AATTCGCAGT TAGCTAGAGA AATTGAAATA GCCTTACCAC GAGAGCTAAC ACCAGAACAA |
| 301 | CGAGTTAAAT TAGTCCACGA TTACGTTCAG AAAACCTTTG TCGATAAGGG AATGGTAGCC |
| 361 | GATTGGTCTA TTCACAACCC ACAACCAGAT AAGGATAATC CAGAAAAGCC AGCAAACCCG |
| 421 | CACGCTCATA TCATGCTCAC ACTGCGTAGC TTGCGTTCTA ACGGCTCTTG GGCACCAAAG |
| 481 | AAGACAAGTC ACTATGAACT AGACGAAAAC GGCCAGAAGG TGCCTGTAAT CGATCCTGAG |
| 541 | ACAGGCAAAC AGAAGTTAGG GGCGAAAAAC CAAAAAATCT GGAAACGAGT AATTACCCCA |
| 601 | ACTAATGACT GGAATAACCC CAAAAACGTT GAAAAATGGC GGGCTGAATG GGCGAAGACT |
| 661 | TGTAATAAGT ACTTGGCTCC TGACCACCAG ATTGACCACC GCAGTTATAA ACGGCAGGGC |
| 721 | AAAAAACAAA TTCCAACAAT CCATGAGGGC TATGTTGCTC GTAAAATGGA ACGAGAAGCC |
| 781 | ACAGGAAGCT CAGAGAGGGC TTCTTTTAAC CAAATCGTAA AATATATCAA CAACGAGTTA |
| 841 | AAATCGCTTA GAAAGCAAAT TAGGGGCATT ATACGCGAAA TACGGAATCT AGAAAAAGGA |
| 901 | CGTGACCAAG ATGAGAAAGT TCGACTTCAG CAAGACACCC GAGCAAATCA AACAAGAACA |
| 961 | AGCAGAGATC AAGCGCTGGG AGGAGGAGTT CAACGCACGA TTACAGACAA TCGGGAGCAA |
| 1021 | TCCGGAAACG GTAGAGCCAA CGGAACTGCC GAAGAAGCCC AACGAACAGA TCCTAATCAA |
| 1081 | CTTCATCAAC AGTCTTTCGG AGAAACAGCA AGAAGAATTT TCCGAGATTT CCGGCTATTT |
| 1141 | GCGGAGCGTC AACGAGAAGC TCGACAGCGA CAACGCCGAC TTAGCCAAGG CTTACAACAT |
| 1201 | CATCAAGAAA CAGAAAAACG ACTTGAAGCA GGGCAACAAC AACTTACTCG AACAAAACAT |
| 1261 | ACAGCTGACG AACGAAAACA ACAGCTTACA GAACGACAAA TTCGAGTTAG AAAAGCTTTT |
| 1321 | AGCGACGCTT CTAAGTCCAA AGTCTTGGAA CGATACTACA CTCACTTCCG TCAAACAAAA |
| 1381 | ACTCAAGCAA ATCATGCAAA AGGATTAGAC TACCAAGGAC CAATTCTGGG ACATTCCCGA |
| 1441 | GGACGCTCAA GGTAGTTATG CTAGAATGAT TAAGCCGTCC GTGGCAACTC CTACGTAAAG |
| 1501 | GAGGTGGCAA CATGGCACAT ATACTCCTCT CAGCAGTTTT GAGCCTTCTG GGTTCCTTGA |
| 1561 | TAACTGCACT GTTCGCTGAT TGGCTTCGTC GGCGAAAATA GTTTTTGACG GCATGAAGCG |
| 1621 | CAAATTAAAA GCCACTCAAC TATTCGCACT AGTTGGGTGG CTTTTTTGCA ACGTGGCAAT |
| 1681 | ATACTTCTCT CGGTTAAATT ATAACATGGT TGTTCCAGAA GTGCATTAGG TAGGCGGTTT |
| 1741 | GTCCCCCCCC ATGGGTTTTG GCTTCGAAAA TCAATCGTTG AAGTGACCAG CGAAGCTCAG |
| 1801 | CTGATGGAGC CCTCATATTC AAGAAGCAGG TCAACGAATG CCAATTCCAA CATGAGGGCC |
| 1861 | ATGACTTAGG CCACTGATAC TCGCAAATGA CTGTCACACC AACGGTATTA GCCATTCTGA |
| 1921 | TAAGCGAGTC TAACGACTTT TGACGCATAA TCGACCACCT ACCTTATTTT CAAAGCGCCT |
| 1981 | CAGAGCTAAT TAGAGTATTC TTCAAGGAGC TTAGCGAGGA TTATAGACGC TTGGCGGACT |
| 2041 | TCACTTTCGG CTTGATTGAC CCGTTTAGCG ATCTTGTCAT CATGGCGAGC CACCTTTTCC |
| 2101 | AAGTCATGGA GATTATCAGC GATTTGGTCA GTCATGACGG AGTAGTTCTT AATACCCGAC |
| 2161 | AGCAGGCTTT CAACGTTAGT ATTCTCAAGG TTTTCGTTGA ATATCATGGT TATTCCTCCT |
| 2221 | TATCCTCAAA CTTAGTCATC TTCTGTCTCC TGTTTTAGAG AATTTTCAAT CATGTCTAAT |
| 2281 | AATGTTGACA TGTCAATGTT AAATGAATCA AGGCTACCAT ACCGCTTGAT TTGAGTTTTA |
| 2341 | ACACTATCCA ATCCATATTC TTCAAGTAAT GCTTTTAATT TACCAGTAAC TTTTTCACCT |
| 2401 | AAATGCCCAA AATACTGAAG CCAGCTCTTA AGTAATTCTC GGTACTGGTC AAATTTTTCT |
| 2461 | TTTTCAGTAA GCTGTTTCTC AACTTCTTCT TGGTCCTCAG AACCGTTAAC TACTTTCCAG |
| 2521 | AGGTCAGGGT CAGTAGTACC CAACTTATAA CCAAGTACCC GATCATATGC TCTGGCCTTT |
| 2581 | TCCTTTGGAG TCAAATCAGG ATTAAGGTCA ATATTATCTA GCTTCATACG TTGGTCAGCA |
| 2641 | TAACGACCTT TTTCAAAGTC ATCGGCATTC TTGGCTTCAG GTTGCCAAGT GAACTCATAG |
| 2701 | CCAACAACAG GACGTCCTCT ACCAGCTCCT CGAATAGTTT TTACGGTTAA GCCACGAATA |
| 2761 | ACAGGGGTTA GCTCTTCTTT AATTGGACGT AACACTCTTT TTCTTACGTT ACCCTGGTCT |
| 2821 | TTCTTGTAGC TCTTGGGTAG ATCTAGTTGC TTAAATAATT CGTCACTGGT TAACTTCAAC |
| 2881 | CGCCCTACTG TTCGATATTG TTTAATTAAC ACTCTTTTTC TTACGTTACC CTGGTCTTTC |
| 2941 | TTGTAGCTCT TGGGTAGATC TAGTTGCTTA AATAATTCGT CACTGGTTAA CTTCAACCGC |
| 3001 | CCTACTGTTC GATATTGTTT AATCAAGCGA AACATGTTCT TAGCATAAGC AGATTGAAGA |
| 3061 | CTATTAAACT GAGCTAGTTG AAATCTTGTC CAATGACTTA AATCATTGAA TAGCTTCTGA |
| 3121 | AATAGAGGGT TAACTTGAAC AGTCAGAATC TGTTTACTCC TCTGAATCTG AAATACATTC |
| 3181 | CAACAAGCAA ATTGAGTAAT GGTGTCTCCA TCATCGGTGT AGGCATTAAT CTCTAATAGC |
| 3241 | TTTTTATTAG TTTTCATCAA ATCTTTTACA AAATTAGATG TACTCCGTTC ATACTTACTA |
| 3301 | AGACTTTGTA GTTGCGTAAA TGAAAACTTT AATTTCTGAG TACCCTTTTG ATACGACTGC |
| 3361 | TGAACTAGTG TAATAAATAG ATTTAGCTCA TTAGAGTTCA GTCGGCCAAG TGGAATAGTA |
| 3421 | TTAAGCCGAT TACTATACTT CACAATTTCA TTACTCAATT TTCTCACCTC AAATATATTA |
| 3481 | TAACATTTTA TCGTACACAA TCAAGAGTAC GTACTTATAG CACACGTACA GTATTAGCGC |
| 3541 | GTATATTAAG ACAAAAATTT AGACAAAATG TACCTTATAA CGGACAAAAT GTACCTTATA |
| 3601 | AACCGGACAA AATGTACCTT ATAACGGACA AAATGTACCT TATAAAAAAC GAAACTCTTA |
| 3661 | CTCTCCCAAG GAGTTTCAAA CCCCTAAAGA GTATTTAAAG AATATATATA AAGATATTTA |
| 3721 | AAGAGGCACC ATACGGAAAA TCTCCTTTCT GATTTCAATT TCAGAGAGGA GATTATCACT |
| 3781 | AAATTTCAAA ATTCAATTTT TCGCCAAAAA CTTTTTTAGT AGTTTTCGGT AACTAGCAAA |
| 3841 | ATCAACTTCG TTGATTATTT TGACTATTTC GAAATTCATC AAAAACTGAA TGCCCGTCTT |
| 3901 | TATAGTGAAT TTGTTTAGTG AAGTTCACTA ACTGCTTCAG CATATTCAGT TGACCACGTA |
| 3961 | ACCATTGTTC ACTATCAGCA GGACTAATTT GTTCTGATAA TTGCTCTTTT TGGTGGTTAT |
| 4021 | AAGCCTTATA AGCCTGATAT TTAGCATAAC CTTGTGGGTC ATCTGATTTA GCCACTCTAG |
| 4081 | CCACTTTAGG TACTTTTCTA AGCCCCTTAG CTTGAGCTTG AATATCTTCT AAAACCGCCC |
| 4141 | CTTTTTCGAT GAGACGCTTA TTTCGTTGCT TTCTCTTTTC AGTATTGATG CGAGCCATTT |
| 4201 | GACGCTCTCG ACGAGCCTTA AACACTGCTT CCTTTTCTTG AAGGCGTTCA AGTTCTGCTT |
| 4261 | TCATTTTTTC AATATCAGCC ATAACAAATC CTCCTAATGT GAATAATGTT TTCTGAAATT |
| 4321 | CAAATGTTAT CTAGATTATA TGATGTTACA ATTGATTTAA CCACTACGTT TGGTCTCCCT |
| 4381 | GTTAAGGGCG CACTTATACA CATTCAAAGA ATGTAATTAC GCCTGACGGC GACGTGCGCT |
| 4441 | CTCCGAGGGG ACTTACCCAG TCGGGGCGAC TTCATCGAGA CCATGTCATG GAGATTCAGT |
| 4501 | TTCACCGAAA CGGACCACTA ACCCCGTGGA GGGGAGAAGT TTCACTTCTA GGGACCATTC |
| 4561 | CATGGAGGGT CGCTTTTTAC AAAAGCAACG CAAAACAAAA GACTAGCTCC CAAGCTTACG |
| 4621 | CAAGGTCGTT AGTCCCTAAG GAGGCAGCAC AGA. |

A putative L. reuteri plasmid origin of replication identified based on BLAST analysis could be:

| (SEQ ID NO: 8) | |
|---|---|
| 1 | TTAGTCATCT TCTGTCTCCT GTTTTAGAGA ATTTTCAATC ATGTCTAATA ATGTTGACAT |
| 61 | GTCAATGTTA AATGAATCAA GGCTACCATA CCGCTTGATT TGAGTTTTAA CACTATCCAA |
| 121 | TCCATATTCT TCAAGTAATG CTTTTAATTT ACCAGTAACT TTTTCACCTA AATGCCCAAA |
| 181 | ATACTGAAGC CAGCTCTTAA GTAATTCTCG GTACTGGTCA AATTTTTCTT TTTCAGTAAG |
| 241 | CTGTTTCTCA ACTTCTTCTT GGTCCTCAGA ACCGTTAACT ACTTTCCAGA GGTCAGGGTC |
| 301 | AGTAGTACCC AACTTATAAC CAAGTACCCG ATCATATGCT CTGGCCTTTT CCTTTGGAGT |
| 361 | CAAATCAGGA TTAAGGTCAA TATTATCTAG CTTCATACGT TGGTCAGCAT AACGACCTTT |
| 421 | TTCAAAGTCA TCGGCATTCT TGGCTTCAGG TTGCCAAGTG AACTCATAGC CAACAACAGG |
| 481 | ACGTCCTCTA CCAGCTCCTC GAATAGTTTT TACGGTTAAG CCACGAATAA CAGGGGTTAG |
| 541 | CTCTTCTTTA ATTGGACGTA ACACTCTTTT TCTTACGTTA CCCTGGTCTT TCTTGTAGCT |
| 601 | CTTGGGTAGA TCTAGTTGCT TAAATAATTC GTCACTGGTT AACTTCAACC GCCCTACTGT |
| 661 | TCGATATTGT TTAATTAACA CTCTTTTTCT TACGTTACCC TGGTCTTTCT TGTAGCTCTT |
| 721 | GGGTAGATCT AGTTGCTTAA ATAATTCGTC ACTGGTTAAC TTCAACCGCC CTACTGTTCG |
| 781 | ATATTGTTTA ATCAAGCGAA ACATGTTCTT AGCATAAGCA GATTGAAGAC TATTAAACTG |
| 841 | AGCTAGTTGA AATCTTGTCC AATGACTTAA ATCATTGAAT AGCTTCTGAA ATAGAGGGTT |
| 901 | AACTTGAACA GTCAGAATCT GTTTACTCCT CTGAATCTGA AATACATTCC AACAAGCAAA |
| 961 | TTGAGTAATG GTGTCTCCAT CATCGGTGTA GGCATTAATC TCTAATAGCT TTTTATTAGT |
| 1021 | TTTCAT. |

Analysis of the strain 3632 sequence reveals two agglutinin receptor precursors (ARP) that are uniquely found in L. reuteri strain 3632 compared to L. reuteri 2091, strain 170331 and L. reuteri ATCC 23272 (human). These agglutinin receptor precursors likely result in the increased level of auto-aggregation observed with this strain, and these receptors would contribute to the probiotic potential of a DFM genetically modified to express them. The amino acid sequence of ARP-1 is: MNEYNAEMAKLNQGANAPVITTNSVNQALSLKPENNATVDIEALNPRITFKRVE EGTKYAGYQIFDKNNAYVNNIDGEFLRVTYTNLKNSTYKGSKISKIVVTYSDSTP TGNRITQSGLNAVTEGANDNFLVVFEDPVRGDMHSTTVTATYQYYDANGNLIDF SGTNNAWLSVGSLNFDQGNDYQGGKNEGNPTSGISEGVKLISGAQIKQLAGSSIS VHDDGWAYAGFNNYSGTGMNNGINTDNGGSGWDMDGSPNAYYGAIVFQLTGS SVSLRQGLVSWGGADIASQYNNQFLNNAWFTAGTTLPETQIKQPIRKTSETHYHY NPSVIRL (SEQ ID NO: 9). The amino acid sequence of ARP-2 is: MAQKLMSANSTDKNFKMYKSKKSWVFAYSTTLALAAVAGITLSTTNVHADTTN GGDNQVNATAVTQNTTSNTVDQIAANTAQTDNTSTSINIRSLMDDLASGDDTSSS QNGQEQSQNYASSNQNSQTQQENGTTGQSTASQNGTTSDQTNSDQSDKNYYVIS TRDLDKNGNVNYLTQKNYTSIKGQEVADGTVVTWPLSVSALPANRAQDLKSHVI SETLDPHLEYLHYRAYLTNTDGTVTDVTNHVNLNRSGQTLIFTDDNYLLSIYNNN RYRVQNLPVIKLVTKANGNGYIIPNAFKSSYVFNDGSHDVSFTTTSNNVQIKTFNP GNSKDVEIGGNVQGDPSGTINGQVVADGSVVTWPMSVGDLPANRAQDVLSHIET DTLYNGLNYEGYHAYLPQADGSFQDVSSHINVQQNGQDLTFIADDYLIGLYNQD KSTAFKMPIIDLITSVHGTSIIAPNKFNSQLAFKDGNGQTVINNTSNQVQISTYHPT NTKDVELGGNVQGDTPNSINDKVVANGAIVTWPMASSELPANRVQDLQSRVISE TLDSHLQYQGYKAWLQNADGKYTDVTSHVKLTQDGQNLTFADDEYLLNLYNS NKGTAYKLPIIDLVTKVNGAGITAPNSYTTKYVYSDGDGNTTINVTSNTVKISTEN PTTNKDVELGDNIHGDTESSIAGKLVSEGTIVTWPLSTSDLPANRAQDVVSHTAV DALEPTLQYISYTAWLPDSNGQLQDVTSHVKMTRDGQKLTFTDDDYLIGLYNQN KDIALKMPIIDLVTKATGNTKLLPNSFDSQFVYNDVDGNTIINVSSNKPTVETFDP TVHKDVELGGNNVQGDTPNSIDGKIVAQGTVVTWPMSTSDLPANRTQDVVSHST SETLNQNLQYVGYHAYMPDANGKLQDVTSHVQLQQNGQNLVFTDDSYLINLYN QDKSIAFKMPIIDLMTKAISDSATIPNTFESQYVFNDGNGNTTFKSTSNTVQIITYK PKTTKDVELGDNIHGDTNASIAGQMITDGTVVTWPMSTSDLPANRTQDLQQHVV TDNLNDNLIFQGYTAWLPTANGLVDVTNHIELTRDGQNLTFTDDAYLLNLYNQN KDTAYKLPIIDLVTKANGNTKLIPNNFDSMFVYNDGDQQTTVNVTSNTVNISTYD PTATKDVELGDDIEGDTADTINNLMVQIGTKMTYPLTVSDLPANRADEITAHQSV DTLSDYLEYQGYKAYLPDADGKLQDITEHVNLKREGQKLSFNDDDYLINLYNNS KATKQALPVIDLVAKVTGSNDGKKVHIIPNHFDSTITTKDGKINTTSNTVVINSND PEAVKDVELGDNVVGDTPNSVTGTTVADGTIVTWPMSVGSLGANRAQNVIKHT ETENLDSGLTYLSFKAYLPDADGKMQDITEHINIQQDGQKLVFTDDDYLISLYNK DKSQRFALPVIDLVTRVNGDNKIIPNTFVSQFTFNDGKGNTITSVTSNQVNVSTFK SNPEKHVTLGTDIEGDDAENADGTVVAQGSEVTWPLSDKSPLPANRSQDVKSHT LVDKLDDNLQYNSYKAYLKGTDGKLQDVTDHIKLTRDGQNLTFIDDDYLLDLY NKDKSTAFNLPIIDLVTTVVGNDKLIPNKFDSNFVFSDGNKDTSMKTTSNEVSIST YTPVTNKDAELGDNVVGDTSDSIANETVPDGTIVTWPLSVSSLPANRSQDVFKHV IEDILDGNLTYNSFKAYLKDAAGNLQEVTDHVKLAQEGQHLTFTDDDYLINLYN SSKNKEQSLPIIDLVTTVHGDSKLIPNEFDNVFVFKDGKGQTTVKTTSNKVTIKTA SLPTPTKEETDDQGNNINGNEVKAGEHVNYTLNWDLSNDKDVKATPEMIKKGFF FIDPIDSRALSVDDLSKAKVVDQNGNKVDGISFHLYNSLSEVPEFIQEQVKANNLQ DKITGPFVVAQADDLQAFFDKYVKTGAKLKVTIPTIVKSGFTGEFSNTAYQFGFG KATPTNTVTNYVKPMHKPASPETPAAIAPQVISATAQPMTSDAPVTPSEKTAKLP QTGNADEGALLGLAAVSLVGSLGLAALGLKQNRNDD (SEQ ID NO: 10). A person of skill in the art would recognize that, because of the redundancy of the genetic code, multiple nucleic acid sequences could encode the above peptides.

L. reuteri strain 3632 contains eight bacteriophage-encoding loci, but only three of these loci appear to encode productive prophages. Bacteriophage loci 1 has a sequence of:

| (SEQ ID NO: 11) | |
|---|---|
| 1 | CTATTCAAAT TTGAAATTTT GAACAACTTT ATCAAGGTCT TCTTGTTCAA TCCCTAGATA |
| 61 | GTGGCGCGTA TTTCGTTCAG AGGAGTGATT AAATAACTGC GAAATGATTT CAACGTTAAC |
| 121 | ACCTTTCTTA TAGAGTTGTC GACCAAAAGT TTTTCTAAAA GAGTGTGTTC CAATTTTAGC |
| 181 | AACGATTTTA TTATTTTCCG TTTTTCTAGC CATTCGTTGC AACATTTCGT AAAAGCCATG |
| 241 | TACTGAAAAA TGACCTTCTT GTTTCCCAGG GAAAAGATAA TCATTCTCGT CTTTATAATT |
| 301 | CAAGTCATTA AGATAATCAA TAATTTCGGC AAGACAATTA TTCCAAAAGA GCGTTTTAGC |
| 361 | CTTACCTGTT TTTTGCTCAA TAACACGGGT TTTAGTTTTA TTTAGAACAT GACCAACCCT |
| 421 | TAACGTTACA ACGTCTGATG CTCGCAATCC GTTATTAAGG GCAATTGCAA TTAGTAATAG |
| 481 | GTTTCGGTTA ACCAATTCAG GACGCGAATG CAGCTTAATT GTCTCACGTA ACTGATCATA |
| 541 | TTCTTCGAGA GTCAAGAATT TAACATTTTC TACCCATATT TTCCGTTTAC CCTTGATTTG |
| 601 | ACCATTAGTT TTATTTTTGG GAATATAAAC TCGTTTTTTA GTTGACATAA TATTTCTTCC |
| 661 | TTTGTTAATT TGCGAGGTAA TCAACTAAAG CAGGGACGAT TTCCTCTCCT TCTTTCCGCC |
| 721 | CAGTTTCATA AAGTGCTCGA ATTTTCTTAA TATTTCCTTC AATCCGCTTA ATTTTAATTG |
| 781 | GTTTAGAGGG AGCAATATTA AAGATCTTTC CTTCACAAGA CAAACGATTA ATTTCAGCAG |
| 841 | TCTGTTGGTT GTAGAGAAGG GGACGATCAA TCCCCGCTTT AGCAAATTCG GGATAATCTT |
| 901 | TATATACCAT GTCGTACAAC TTCTTTACAG CAGAACTAGA TGGCTTCTTT CGGTAGTTAA |
| 961 | TATCCCTGGT CCGTACAACA ACAATCTTGT CATAGCCTTG TCGTTGTGCA ATATCAAATG |
| 1021 | GAATTGAATC TGTGATCCCA CCATCAAGGC AAGGCCCTTG AGAAGTTTCC TGTGGATCTG |
| 1081 | AAAGAAATGG CATTGATGAG GAAGCTTTGA GGGCATTGGT TAGCTCCTCA CCAACAGGAT |
| 1141 | CCGTAAATAA AACTGTTTTT CCTGTTTTCA CAGAAGTAGC AACTGCTGTG AAATGAGATG |
| 1201 | CTGACCGTCG ATATGCAGCT TCATTAAAAT TCTGCCAAGA ATATCCGTGA TCTTCAAAAA |
| 1261 | GATAATCAAG ATTAATTATT TGCTTTTTAA ATACGCGAGC CATCGAGATA TATTGGCGAT |
| 1321 | CGTGCCGATG ATCAATATTA ATATTGGCAG CGCGTCCGTA TTGCTTAGAG ACGAAATTAA |
| 1381 | CTCCACAAAG TGAACCAGCA GAAACGCCAA TTACACTTCG AAATTCAATA TGGTGAGCGA |
| 1441 | GAAATGTATC AACAATTCCG GCAGTATATT GTCCACGGAA TGCGCCACCT TCTAAAACTA |
| 1501 | ATGCTGCATT ATATAACATA AAATGAAATA ACTCCTTTTT GTCCGCCAAA AATAGCAAAA |
| 1561 | ATGACCTAAT TTTTAGTGAG ACACTTTTTC AATAAAATTA TACAACAGTC TCACACGAAA |
| 1621 | ATATAGGATT TTAAAAGCTT GTTATATCAA CGTTTATTCT CGGTTACCGT AAGACTCTTT |
| 1681 | TATCAAAAGA GTCTTACTGA TTTTGAAATA ACGCAAAAGT AGGGGCAAAA ACGGATTTTG |
| 1741 | CCTCTCAGAC GAACTGAAAT TCATTTTGAT GAGTAGTAGG TCGTTACAAT TCGTCTCAGA |
| 1801 | AGAAAAACGC ATATTGATAT CAAAAAATGA GTAAATAGAC AGTAAATATT TAAATATGAA |
| 1861 | CTTTTTATGA AGAAACTATT GATAATTTAA GATAATCATA ATAAAATAGT GCACAGTTAA |
| 1921 | TTGTTAAACG TTTAGCAAAA AAGGTAAAGA AACGGGGTTA TTTCTATGCT AGAACGCAAG |
| 1981 | GAACATAAGA AAATGTATAA AAGTGGTAAA AATTGGGCAG TTGTTACACT TTCTACGGCT |
| 2041 | GCACTGGTGT TTGGTGTAAC AACTGTAAAC GCATCCGCAG ACACGAATAC TGAAAACAAT |
| 2101 | GATTCTTCTA CTGTACAGGT TACAACAGGG GATAATGATA TTGCTGTTAA AAGTGTGACA |
| 2161 | CTTGGTAGTG GTCAGGTTAG TGCAGCTAGT GATGCGACTA AAAATTCTGC TAATGCAAAT |
| 2221 | AGTGCTTCTT CTGCCGCTAA TACACAAAAT TCTAACAGTC AAGTAGCAAG TTCTGCTGCA |
| 2281 | ACAGCTTCAT CTACAAGTTC AGCATCTTCA TCAACTAACA CAGATAGTAA AGCAGCTACA |
| 2341 | GAAAATACTA ATGTAGCCAA AGAGGATGAT ACACAAAAAG CTGCACCCGC TAACGAATCC |
| 2401 | TCTGAAGCCA AAAAAGAACC AGCTGTAAAC ACTAATGATT CTTCAGCTGC CAAAAACGAT |
| 2461 | GATCAACAAT CCAGTAAAAA GAATACTGCC GCTAAGTTAA ACAAGGATGC TGAAAACGTT |
| 2521 | GTAAAAAAGG CGTGGATTGA TCCTAATAGT TTAACTGATG ACCAGATTAA AGCATTACAT |
| 2581 | TAAATAAGAT GAACTTCTCG AAAGCTGCAA AGTCTGGTAC ACAAATGACT TATAATGACC |
| 2641 | TTAAAAAGAT TGGTGAGGCT TTAGTTGATC AAGATCCTAA ATACGCTATT CCTTATTTTA |
| 2701 | ATGCAAGTCA AATAAAGAAT ATGCCCGCTG CATATACTAG GGATGCTGAA ACAGGTGAAT |
| 2761 | ATGCTGATTT AGATATTTGG GATTCATGGC CAGTTCAAGA TCCAGTGACT GGTTATGTTT |
| 2821 | CTAATTGGAA TGGCTATCAA CTTGTGATCG CTATGATGGG ACGGCCTCAT CACGAAGATA |
| 2881 | ATCATATTTA TTTACTTTAT AATAAGTATG GTGATAATGA TTTTTCACAC TGGCGAAATG |
| 2941 | CAGGATCTAT ATTTGGATAT AATGAATCTC CACTAACACA AGAATGGTCC GGGTCTGCTA |
| 3001 | TTGTTAATTC TGACAACTCT ATTCAACTCT TTTATACTAT CAATGATACA AATAATGCTA |
| 3061 | TTAACCATCA AAAATTAGCA AGTGCTACTA TGTACTTAAC AGCCGACAAT GATGGTGTCC |
| 3121 | ATATTAATAA TGTAGAGAAT AATCATGTGG TATTTGCAGG TGATGGTTAT CATTACCAAA |
| 3181 | CTTATGATCA ATGGAAAGCT GCAAATAGTT TTGCTGACAA CTACACTTTG CGGGATGGAC |
| 3241 | ATGTTGTACA AATGCCAAAT GGTGATCGGT ATTTAGTATT TGAAGGAAAT ACTGGAACTG |
| 3301 | AAAATTATCA AGGTGAAGAT CAATTATATA ATTGGTCAAA TTATGGTGGT AACGATCGCT |
| 3361 | TTAATATTGA AAGCTTGTTT CATCTTTTAA GTAGTGATGT TGATTATAAA AAAGCTATCT |
| 3421 | TTGCTAACGG GGCACTTGGA ATTATTAAAC TAACGAATGA TGAAAAGAAC CCGCAAGTAG |
| 3481 | AAGAAGTATA TACTCCATTA GTTACATCAA ATATGGTTAG TGATGAACTT GAACGTCCTA |
| 3541 | ATGTTGTTAA ACTTGGAGAT AAGTATTATC TATTCTCGGC TACACGTTTA AGCAGAGGAA |
| 3601 | CTAATATCGA TACTCTTAAT AAAGCTAATA AAGTTGTCGG AGATAATGTT GCAATGATTG |
| 3661 | GTTACGTAGC CGATAGTCTT ACAGGTCCAT ATAAACCATT AAATGGTTCA GGGGTTGTAG |
| 3721 | TGACAGCTTC TGTTCCTGCC AATTGGCGTA CCGCTACTTA TTCTTACTAT GCTGTTCCAG |
| 3781 | TAGAAGGAAA AGAAAATCAA TTACTCATTA CTTCGTATAT GACAAATAGA GGCGAAGTTG |
| 3841 | CTGGAAAAGG TATGAATTCA ACATGGGCAC CAAGTTTTAT TGTGCAAATT AATCCTGATG |
| 3901 | ATACAACTAT GGTATTAGCT AAAGTAACTA ACCAAGGGGA TTGGATTTGG GACGAATCAA |
| 3961 | GTAACAATAA TAATATGTTA GGAAACATTC AAACAGCTGC TTTACCTGGG GAATTTGGTA |
| 4021 | AACCAATTGA TTGGGATTTA ATTGGTGGTT ATGGATTAAA ACCGCATGAT CCTGCTACAC |
| 4081 | CAAATGATCC TGAAACGCCA ACTACACCAG AAACCCCTGA GACACCTAAT ACTCCCGAAA |
| 4141 | CACCAAAGAC TCCTGAAAAT CCTGGGACAC CTCAAACTCC CGATACACCT AATACTCCGG |
| 4201 | AAGTTCCTTT AACTCCAGAA ACGCCTAAGC AACCTGAAAC CCAAACTAAT AATCGTTTAC |
| 4261 | CACAAACTGG AAATAACGCC AATAAAGCCA TGATTGGCCT AGGTATGGGA ACATTGCTTA |
| 4321 | GTATGTTTGG TCTTGCAGGA ATTAACAAAC GTCGAGTTAA CTAAATACTT TAAAATAAAA |
| 4381 | CCGCTAAGCC TTAAATTCAG CTTAACGGTT TTTTATTTTG AAAGTTTTTA TTATCGAAAA |
| 4441 | AAACAAATCC TCGTTAATCC TTTAATGCAA TTGTTGTAAA ACCTTGCGAC AGTAATAACA |
| 4501 | GTGGATTTGC CCATCTTTGT CAGCTAACTT CCGTGCATGC ATTGCAGAAA AAGTATAGTG |
| 4561 | CTCATGACAA AATGGACAAA CATATTGCTT TTTCCCAAAT AGTGAGGTAA TAAAGCCCAA |
| 4621 | AATTTTCTCC TCCATAAAAA AATTATACAC CCCTTAACAT TATAGCGAAG ATTATCTTTA |
| 4681 | TAGAAAAGGC TTTTATTCAT TTGTTTTTTT ATATGAAGTT TCACTAATTT CTTTAAGAGC |
| 4741 | ATCACGCTGC TGTTTCTGGT CAACGTGAGT ATATAAATCG GTAGCAGATG TTCCTTTTTG |
| 4801 | TCCCAACTGT TGGGCCACTA ATACTTGATC TTTCGTTACC TCATACATTT CAGAAGCAAG |
| 4861 | GGTGTGCCGT AATTTATGGG GAGTAAGAGG ATGGCCAAAT GCGGTTGAAT ACTTTTTGAC |
| 4921 | CATTTTTTCA ATTGCATTAG CTGTCATCCG TCGAGTTTGC TTGTGATAAA CGGTTAGGAA |
| 4981 | GAAAGCAGTA TCCTTTTTTA AAGCGTGGTA TCGTTGTGCA CGAATCGCTT GGTAAGTTTG |
| 5041 | GATATAAGTA ATAGCCCAAG GAGCAATTGG TACCGAATCC CTCTGGCCAC CTTTTCTTGT |
| 5101 | TACATCTAGC AGCGATTGTT TTAAGTTTAA ATCACCAAGG TTTACATTTG CCGCCTCGGA |
| 5161 | TACCCGGACG CCTGTTCCTA AAATAAGAGC AATAATTGCA ATATCTCGTT CCTTATTGAC |
| 5221 | CTTATAAGAG GGGAGAGCTC TTTTATCACA TTTATTAGGG TATTCTTTTT CAATGAAAGT |
| 5281 | AATAAAATCA AACTTCATTT GTCCCCGGTA CATGTGTGAA GCCAGCGTAT GAGCCCGATA |
| 5341 | ATTTAATGTT TTTGTATCAT TAAGGGAGTC AATTTTCAGC ATTACATTAC GGTCAAAATA |
| 5401 | AGATTCACCA TTATTGTTAT CTGCGGTAAC TGTTAAAAAT TTATACAATG ACCTTAATGC |
| 5461 | ATTAATAGAT CGATTGATTG AAGTAGGAGA ATTTAACCGT CCTTGTGCAT TGGTAGTATG |
| 5521 | CTGCAAATAA TCGATATACA ACATTACATC GCTACGCCGT AGATTCGCCA GTGTATCAAT |
| 5581 | TGGTAAGTCC TTATTAGAAT TAACAGAGAC GAGTCCACTT GACCGTAACC AATCAAAGAA |
| 5641 | TCGACGGATT TCAGTTAAGT ACTGATAGGT TGTTGTGACT GCATGGTTGG TCCCTAAATA |
| 5701 | ATACTCGTTA ACATAATCAG GAAGGTTCTG AAGCTCCTCT TGAATTAGCT TTAAATATTT |
| 5761 | ATCTGCTTCC ATTTTTATAT CCTCCTTGAT ACTAAAAAGA GTCGCTTGAA AGAAAACTTT |
| 5821 | CAACGACTCT CTAAACCTAT CGGGAAAACA GGATTCGAAC CTGCGACCCC CTGGTCCCAA |
| 5881 | ACCAGGTGCT CTACCAAGCT GAGCTATTTC CCGTTAATAA CGAACAAATA TTATTCTACC |
| 5941 | AATTCTCAAT TAATTAGTAA AGGAATATTT TAATTTAAAG TGATTAATAG TTAATGATTA |
| 6001 | AATGACATAA TAGTAAAATT CCTATTAATT GCAAAAAAAT CATGATTAAC CGAAATCCTT |
| 6061 | TTCTAGCATC TAATGAAGAT GATAAATAAT TTTATTTTCG ACTAGTTATA AAAGATACCT |
| 6121 | TTCCGAAAAT AATTTGACAT TCAAAATACT TTTGAATATA ATTTGATTAT CGAATATTTT |
| 6181 | GATACTCGAA ATATTTTCCG AAGGCAGGTG AATCTTTTTG GCAATAATGA ATGCGCAGGA |
| 6241 | GATTATGGAA TTAATTCCTA ATCGATATCC GATTTGCTAT ATCGATTATG TAGATAAATT |
| 6301 | AGTTCCTGAA GAAAAGATTA CCGCAACAAA AAATGTCACA ATTAATGAAT CATTTTTTCG |
| 6361 | CGGTCATTTT CCTAATAATC CTGTTATGCC TGGAGTTTTA ATTATCGAAA CATTGGCCCA |
| 6421 | AGCTGCTTCA ATTTTGATAT TAAAATCACC GCATTTTTAT AAGAAAACAG CTTATCTTGG |
| 6481 | CGCAATTCAT AAAGCAAGGT TTCGACAAAT GGTCCGTCCT GGTGATGTAT TAAAACTAAA |
| 6541 | CGTTGTTATG AAAAAAGTTC GATCATCAAT GGGGATTGTA GAAACACAAG CGCTTGTGAA |
| 6601 | CGGCAAATTA GCTTGTAGTG CGGAGCTTGT CTTTATCGTT GCTGAACGAG AAGAAAAGAT |
| 6661 | TTAGCACGGT GAATCATTAT CATTTATAAT ATATTTTGAT AATCAAATTA TTCGGGCTAT |
| 6721 | AGAATAAAAT TGAAGGGAAG AAATATATTA TGAGTAAAGA TAATGATTAC GAAAAAATTA |
| 6781 | ATAAAGGATT AATCAAAGTT TATTCCGGAA TTCTATGGAT TGAAGAAAAT GAATTGCGGA |
| 6841 | AAAGTACATT CAATGATTTA ACAATTAAAG AGATGCATGC AATTGATGCA ATTACGATGT |
| 6901 | ATAACCATCA AACAATTTCT CAAGTAGCAG AAAAGCTTCA TCTAACTCCA GGAACAATGA |
| 6961 | CTTCAATGGC TGATCGTTTA ATTCGAAAAG GATATGTGGA AAGAATTCGT GATAAAGATG |
| 7021 | ATCGCCGCAT TGTTCGGTTA TGTTTAACCA AAAGAGGCCG GGTACTATAT CGTGCGCACC |
| 7081 | GGGCCTTTCA TAACATGATG GTTGAACGTT TTCTTAAAGG AATGGATGAT GAAGAAATGA |
| 7141 | AGGTTGTTAA AAAAGCCTTG CAAAACTTAG AAGATTTTGT GGATGAGCAT GCTTAGAGAT |
| 7201 | AAGGATTGAC AAACATTGCA AAATTTAAGA ATTACTAGTA CTGCGAGTTA TCATCCACCC |
| 7261 | CTTAAAATTA CTAACCAGCA ATTATCAACT ATTATGAATA CTTCAGATGA GTGGATTAAG |
| 7321 | ACGCGGACTG GAATTCATCA ACGTTATATC AGCAACACTG AAAATACTTC AGACTTAGCT |
| 7381 | GTCAATGTTG GTACCCAGTT ATTGACTAAT GCCAATTTAA AGGCAACTGA ACTTGATTTA |
| 7441 | ATCATTATTG CAACGATGTC TCCTGATGCC TATACTCCTT CAACTGCTGC TATTGTTCAA |
| 7501 | GGAAGATTAG GTGCGAAAAA TGCAATTGCG TTTGATATCT CAGCAGCTTG TACTGGTTTT |
| 7561 | ATCTATGCCA TTAATACAGC TGAATTAATG TTGAAAAGCT CTCATTGGAA AAATGCAATG |
| 7621 | GTAATTGGTG CAGAAGTATT ATCAAAACTG ATTGATTGGC AAGATCGAAG TACTGCCGTA |
| 7681 | TTGTTCGGCG ATGGGCTGGC GGAGTGTTAC TTCAAAAGAC AACTACAACA ACCCCTTTAA |
| 7741 | TTCTCGGCCG TGATCTCCAT ACATTTGGTG ACTTAGGAGA TAAAATTATT GCCGGAAAAA |
| 7801 | CAACGCCTAA AACTGGCTTC CCTAAACAAC TAACATCCCT TTCACCATTT ACGATGGCCG |
| 7861 | GCCGTGACGT ATACCGCTTT GCCACTCATG AAGTACCACG ATCAATCACT TCTGCCGTTC |
| 7921 | AACAAGCTAA TTTGAAATTA GACGATATCG ATTATTTTTT ATTACATCAA GCAAATGAAC |
| 7981 | GGATAATTAC CCAAATTGCA AAGAGACTGG CGCAACCAAT TACAAAGTTT CCAATGAATA |
| 8041 | TTAGTGAATA TGGGAATACA GCTGCCGCTA GTGAACCAAT TTTATTGACT CAAGCTATTG |
| 8101 | CTCATGAATT GATTAAACCA GGTAACATTA TTGCAATGAG TGGCTTTGGT GGCGGGTTAA |
| 8161 | GTACAGGAAC AATAATTTTA AACTATTAAT AGAGAAAGAA GAATGGATAT GACTAAAGAG |
| 8221 | GAAATTTTTA ATACTGTAAA AACTATTACT GTTGATGAAT TAGATGTTGA CGAAAATCGT |
| 8281 | GTAACAATGG ATGCACGAAT CAAAGATGAT CTTGATGCTG ATAGTCTTGA TGTTTTTGAA |
| 8341 | ATTATGAATG AGCTTGAAGA TAAGTTTGAG ATTGAATTAG ATGCCGATGA AGGAATCGAG |
| 8401 | ACTATTAGTG ACGTTGTTGA TTTCGTAAAG AAACAGTTGG ATGAAAAATA ATGTACTACG |
| 8461 | GAATATTATT TAGCGGTCAA GGTGCACAGC GATCGGGAAT GGGAGTCGAG CTTATGGCCG |
| 8521 | ACTCCCTTTT TTCAAGGATT GTTAGTCAGG CAAGCGCTGT TTGTGAACTT GATTTGCTCA |
| 8581 | AAATTATGAA AAACGAGCAT AAGGAGTTGA ATAAAACAGC ATATGTTCAA CCAGCAATTG |
| 8641 | TAACAGTTAG TTATGGAATC TACCGAATGT TAAAACGGGA TTTACCTCAA CTACCAATTA |
| 8701 | AAGGGATGAT TGGCCTATCT TTAGGCGAGT ACGCTGCTTT AATTGCTAGT AATGCACTTT |
| 8761 | CATTTGAAGA AGGAATTAAA TTAGTTGCGG ACCGTGCTCA CTTTATGCAA CAAGACGCGG |
| 8821 | ATAGAGAAAT AAGTACATTA GCAGCTGTCC TTGATCCTCA ACTTCAAGAG ATAAAAGAAC |
| 8881 | TAATCACCGC TCAACAAGAA AATGGTCAGC GAGTTTATAT TGCTAACTAT AATTCACCAC |
| 8941 | GACAAATCGT AGTGGGCGGG GCATTAAACG CTTTAAAGGC TACCCTTAAA AAGATTGAAG |
| 9001 | AGGACAAGCT TGCTAAAAGA ACGATCCTGC TCAAAGTTAA TGGTGCATTT CATACTCCCT |
| 9061 | TCTTTAACGG TGCACGTCAA CAGATGCATA ACCGGTTACA AGCAGTCGAC TTCCATGAGC |
| 9121 | CACAGATTGA AGTTATTAGC AATACTACTA ACAGCTTGTT TCATTGTGAG GATCTTCCAG |
| 9181 | GAATTCTTGA AAAACAATTA GCTGTTCCAA CACACTTTGG AGCTAATGTT AAGGAATTAG |
| 9241 | TCAAGCACGC GAAAATTGAC ACAATATTGG AAATTGGTCC TGGAAAGACG TTATCTCGCT |
| 9301 | TCGCTCACCA AGTTGACCAG CACTTAAATA CCCAACACAT TGAAAATCTT GCTGATTATG |
| 9361 | AAAAATTTAT AAAGGAGCAA AAAGATGGAA CTGACAGATA AAGTCGTTTT TATAAGCGGA |
| 9421 | AGCACACGCG GGATTGGGGC GGCTACTGCA TTAGAGTTTG CTAAGGCTGG TAGTCGGCTA |
| 9481 | ATCCTCAATG GGCGGCAGGA TAACTTACCA AAAACGTTTA AAGAAAAGCT AGATCTACTA |
| 9541 | GGGGCGGATT ATCACTATCT TAAGGGCGAT ATTGCAAATG AAGAATCAGT TAGTGAATTA |
| 9601 | GCAGCAGCGG CTTGGCAAAT ATACGAGAAG ATCGACATTC TTATCAATAA CGCGGGAATC |
| 9661 | ACGAATGATA AGTTAATGAT GGGAATGAAA GCGAGCGATT TTGACCAGGT CATCAATGTT |
| 9721 | AATTTACGCG GAACATTTAT GTTAACGCAA CCTATTTTTA AGAAGATGCT CAAAAAAAGA |
| 9781 | GTCGGTTGCA TTATCAACCT TGCTAGTATT GTGGGTCTCC ATGGTAATAC GGGACAAGCT |
| 9841 | AATTATGCGG CAAGCAAGGC AGGTATCATC GGCCTTACTA AATCTATTGC CCAAGAAGGA |
| 9901 | GCACGCCGTG GAATTCGTTG CAATGCGATT GCTCCCGGAA TGATTACTAG TGATATGACT |
| 9961 | GAAAAATTAT CTGAGCGAGT AAAAGAACAA ATTCTCAGTC GCATCCCCCT CAACCGCTTA |
| 10021 | GGACAGTCAG AAGAAGTTGC TAAGACCGCA AAATTTTTAG CAGAAAACGA TTATTTGACT |
| 10081 | GGTCAAACCA TTGTAGTTGA CGGTGGCATG ACAATTTAGG AGGAACTAAA TGACAAGAGT |
| 10141 | TGTAATAACA GGAATGGGTG CTGTTGCTCC TAATGGTAAC GGTATTCAAG AATTTATAAG |
| 10201 | TAATAGTTTT GCAGGCAAAG TTGGAATTAA AGCGATCAAG AAATTTGATG CCAAGTCGAC |
| 10261 | AGGAATTACC GTGGCAGGTG AAATTGACGA TTTTGACCCT AATGATGTCA TTGGAAGGAA |
| 10321 | AGCCGCGCGC CGAATGGATC TTTATTCTCA ATACGCCTTA CAAAGTGCGA TTGAAGCAAT |
| 10381 | GGAAATGGCG GAGATTAACG AAACAAATAC CAAGCCAGTC GACATGGGTG TTATCTATGG |
| 10441 | ATCTGGAATT GGCGGTTTGA CAACTATTCA AGAGCAAATT ATCAAAATGC ATGATAAGGG |
| 10501 | TCCTAGACGG GTATCACCAA TGTTTGTTCC AATGTCAATT GCTAACATGG CAGCCGGTAA |
| 10561 | TATTTCCATT CACTTTAATG CGCAAAATAT TTGTACATCG ATTGTGACTG CTTGTGCCAC |
| 10621 | TGGAACTAAT GCAATTGGTG AAGCCTTTCG TCAAGTTAAA GCAGGTCGCG CTAAAGTAAT |
| 10681 | GATCGCTGGT GGATCAGAGG CTTCGGTGAA TGAGATCGGG ATTGCTGGTT TTGCGGCATT |
| 10741 | AACAGCATTA TCACAAGCAA CTGATCCGCT TAAAGCTTCT TTGCCATTTG ATAAGGCACG |
| 10801 | TCAAGGATTT GTTTTGGGTG AAGGCGGTGC AACACTTGTT TTAGAGGATC TTGAGCATGC |
| 10861 | GCAAAAACGC AGTGCTAACA TTCTTGGTGA GATTGTCGGT TATGGTGCTA CCTCGGACGC |
| 10921 | TTACCATATT ACATCCCCTG ATCCAACTGG TGCAGGGGCG GCAAGAGCAA TGGAACTGGC |
| 10981 | AATTAAAGAA GCTGGAATTA GTCCTAGCGA AATTTCCTAT ATTAATGCCC ACGGAACCGC |
| 11041 | TACTCATGCT AATGATGAAG GCGAATCCAA AGCAATCAAT CAGGTATTTG GTTCCGATAG |
| 11101 | TAATGTTCGC GTTAGTTCAA CGAAGGGAAT GACCGGCCAT TTGCTTGGGG CTGCGGGCGC |
| 11161 | AATTGAGGCA GTCTTAACAG TAGCCGCTTT ACAAAAGGGA CAATTACCGT TGAATATAGG |
| 11221 | TTGCTTTAAT CAAGATCCAA AATGCTCGGT TAACCTTGTG ACGGCAGAAA ATAGGAACGC |
| 11281 | CTCAACCGCC CGTTACGCAA TAAGCAATTC TTTTGGTTTC GGTGGTCATA ATGCTGTTTT |
| 11341 | AGCCTTTAAG AAATGGGAGT GATCTATCTT GGAATTTAAA GAAATTCAAA CATTAATGCA |
| 11401 | AAATTTTGAA GATTCTGATA TTCGTGAATT AGAAATAAAT CAAGATTCCT TTCAGCTCTA |
| 11461 | TTTAAGCAAA AATAAGCAAA CCCACAAGCA TGAAAATCTT ATAACAACCG AAAAAACAGA |
| 11521 | GCAAACGACT TCAGCTAAGA AAAAAGCAAA CGAACAACTA ACTTTACCTT CGCAAAATAT |
| 11581 | AACTGCGCCC CTAGTCGGAA CAGTCTATCT CCAACCAACC CCCGATGCAG ATCCCTATGT |
| 11641 | TAAAAGTGGC GACCACGTAA AAAAGGGGGA TGTTGTTTGT GTGATTGAAG CAATGAAAAT |
| 11701 | GATGACAGAG ATAAAAAGCC CTTTTAACGG AACCATTACT TCAATTTGTG TAAGCAATGA |
| 11761 | AGAATTAGTT GAAGTAGAAC AACCGCTTTT CTCAGTTCAG GAGGATAAAG ACAATGCCTA |
| 11821 | ATAAAACTTT AGATATAACT GAAATTCAAA AAATCCTTCC GCATCGTTAC CCGATGTTGC |
| 11881 | TAATTGACCA AGTTGATGAA TTAATCCCTG GTAAGAAGGC CATTACACAC CGTAATGTCA |
| 11941 | CGATTAATGA AGAGGTTTTT AATGGTCATT TCCCCGAAAA TCCAGTTTTA CCAGGAGCAT |
| 12001 | TGATTGTTGA ATCATTGGCG CAAACAGGTG CCGTCGCTCT CTTATCTCAA GAAGAGTTCC |
| 12061 | AAGGGAAAAC AGCCTATTTT GGTGGAATTC GGTCAGCAGA ATTTCGTAAG GTAGTCCGTC |
| 12121 | CGGGCGATAC GTTAAGGTTA GAAGTCAGAC TAGAAAAAGT TCACAAAAAT ATTGGAATTG |
| 12181 | GTAAAGGCAT TGCAACGGTC GATGGCAAAA AAGCCTGTAC AGCCGAATTA ACTTTTATGA |
| 12241 | TTGGGTAGGT GGTTTAATGT TTTCCAAAGT ACTAGTGGCT AATCGTGGTG AAATTGCTGT |
| 12301 | CAGGATAATA CGGTCATTAC GGGAGCTAGG AATTAAGACA GTAGCTATCT ATTCAACTGC |
| 12361 | AGACCGCGAA AGTCTTCATG TTCAACTAGC GGATGAAGCT GTATGCGTTG GAACTGCCCG |
| 12421 | GGCCCAAGAT TCATATTTGA ATGCGAAAAA CATTTTAGAA GCTGCTCTTG GTACAGGTGC |
| 12481 | CCAGGCAATC CATCCTGGCT TTGGCTTTCT ATCAGAAAAT GCGGAATTTG CGACAATGTG |
| 12541 | TGAAGAATGC GGAATTACGT TCATCGGTCC CCAAGCCTCA GTGATTGACT TAATGGGAAA |
| 12601 | TAAGGAGCAT GCACGGGAGC AAATGAAAAA ATCAGGGGTG CCTGTGATTC CTGGAAGCGA |
| 12661 | TGACTATATT ACCAATGTTA ATGACGCTGT TGAGGTCGCA AACAAGATTG GGTATCCAAT |
| 12721 | TTTGTTAAAA GCAGCTGCTG GTGGTGGCGG TAAAGGGATC CGACGAATTA ACGATCATAA |
| 12781 | CCAGATGCGG CAAATATTTA GCGAGGCCCA AAACGAAGCC CGACTTTCGT TTAATGATGA |
| 12841 | CCGAATGTAC CTTGAAAAGA TTATGGAGAA TGTTAAACAC ATTGAGGTCC AAGTATTTCG |
| 12901 | TGATAATTTT GGCAATGCCG TTTTCTTTCC TGAACGTGAC TGCTCGATTC AACGGAATAA |
| 12961 | ACAAAAATTG ATTGAAGAAA GTCCTTGTGT CCTAGTAAAT GAACAAGAGC GAAAAACGCT |
| 13021 | AGGACAAATT GCAATGCGAG CCATTAATGC GATTAACTAC CATAATACGG GGACAATAGA |
| 13081 | ATTTCTAATG GACAAGGACC ATCACTTTTA CTTTATGGAA ATGAACACTC GTATCCAGGT |
| 13141 | TGAACATACA GTGACGGAGA TGGTAACTGG GATCGACTTA GTGAAGGCAC AGGTTATTGT |
| 13201 | CGCTGCGAAT GAACCACTTC CCTTTACCCA ACAGGATATT CAGGTTCATG GACATGCAAT |
| 13261 | TGAGTGTCGG ATAAATGCTG AAAATCCTAA ACAAAACTTT ATGCCAGTGA CTGGGACGAT |
| 13321 | TAATTACTTA TATCTTCCAG TCGGTAATTT GGGGATGCGC ATTGACACTG CTATTTATCC |
| 13381 | TGGCAGTAAG ATCACTCCTT ATTATGATTC AATGATCGCT AAGGTAATTG CCCTTGGTCA |
| 13441 | AGATCGCCAA GAAGCTATTG AAAAAATCAA ACGACTTTTA AATGAAATGG TAATTACGGG |
| 13501 | CGTAACGACC AATCAAAATT TCCATTTAGC AATCCTAAAC AATCCTAAAT TTTTGGCAGG |
| 13561 | AACAGCTTCA ACAACGTTTC TTGAAGACTT CTTCTTGCCA CAATGGAAAA AGGAGCTGAC |
| 13621 | AGCGTGAAAT TATATGATCA AAATAATACT TTAAGTGAAC GGCACATCAA AGCAGATAAA |
| 13681 | AATGCTGATG AAAGGGTCCC AGATCAAATG TGGTTAAGGT GTCCACATTG TCATCAATTA |
| 13741 | CTATTCGCCA AGCAGTTAAC ACAATATGCT GTTTGTCCCA ATTGTGACTA TGGATTACGA |
| 13801 | ATACCTGCCC GCCATCGACT CTCGTGGTTA GTAGATTCAT TTAAAGAATT CGATAAGGAT |
| 13861 | CTCCAGACAA AGAATCCGTT ACATTTTCCT GGATACCAAG AAAAAATCAG CAAACTTCAA |
| 13921 | AGACAAACTA AGCTGAATGA TTCAGTCCTA ACTGGTGAAG CTTCAATTAA TGATCAGCTA |
| 13981 | TTTTCACTAG GTATTATGGA TCCAACATTT ATTATGGGAT CCCTCGGAAC TGTTACCGGT |
| 14041 | GAAAAGATAA CGCGCTTATT TGAATATGCA ACTATCCATC GTCAAGCAGT AGTACTATTC |
| 14101 | ACTGCTTCAG GCGGGGCACG GATGCAAGAA GGAATTATGT CGCTAATGCA GATGGCGAAG |
| 14161 | ATTTCACAAG CAATAAATGA GCATGCTGCT GCCGGCCTTT TATACATTGT GATCTTAACC |
| 14221 | GATCCAACAA CTGGTGGAGT AACAGCTAGT TTCGCAATGG ACGGAGATAT TATTCTCGCT |
| 14281 | GAGCCCCATG CACTTGTTGG CTTTGCAGGC CGTCGAGTTA TTGAACAGAC GATTCATCAG |
| 14341 | CAAATTCCTG TTGATCTCCA ATCAGCTGAA AACATCCTGC ATCATGGATT TATTGATCGA |
| 14401 | ATTGTAAAAC GTCAAGATGA AAAAAAGCTG CTTGAATGGC TATTAAAAAC AGGGAGCGTT |
| 14461 | GCTAATGAAT GAACAATTAT CAGCAAGCGA GATTGTTAAA CGTGCTCGCA ATGACAATAA |
| 14521 | AATTACGGGG ATGGAGATTA TTCAAAATGT TTTCCCAGAT TTTGTTGAGT TGCACGGCGA |
| 14581 | CCGGGCAGGC GGGGATGATC CTGCAATCGT TGGTGGAATC GCTACTTTCC ATCAGCAAGC |
| 14641 | AGTTACCGTC ATTACCACTG ATCGAGGAAA AACAACAGAA GAAAAAATCA TAAAGCATTT |
| 14701 | TGGCTCACCA ATGCCTAGTG GTTATCGCAA GGCACTCCGC TTAATTAAGC AAGCAGTTAA |
| 14761 | ATTTAAGCGA CCTGTATTCT GTTTTGTTAA TACCGCAGGA GCATTTCCTA GCAAGGAAGC |
| 14821 | CGAAGAAAAT GGGCAAGGAA GTGCGATTGC CCAAAACATT TTACAAATAA GTCAGCTTGC |
| 14881 | CATTCCAATT ATCACGATTA TTTATGGTGA AGGAGGTAGT GGGGGAGCCT TAGCATTGGC |
| 14941 | ATGTGGAGAT GAAGTATGGA TGTTAGAAAA CAGTACTTAT TCTATTTTAT CTCCTGAAGG |
| 15001 | GTTTGCCTCC ATCATGTGGA AAGATAGTAC GAAAGCAGAT AAAGCGGCAG AATTAATGCA |
| 15061 | AATGGTGCCG CAAGCTTTAT TAAAACAAGG GATTATCGAA GGAATTATTC CAGAAAGCGA |
| 15121 | AGAGCATCGT AAAACTTGCA AAAATATCGA GCAGGTTTTA CTAAAGCGAT TAAACAAGCT |
| 15181 | GCAAGAATTA CCGCCAAACC AACTTCTAGC AAACAGAAAA AAACGTTATC GAAAGTTTTA |
| 15241 | AGGAGGATAA TATGGGAAAT ATATTAACAG GAAAAAAGAT CGTTGTTATG GGAGTAGCTA |
| 15301 | ATAAGCGTTC TATTGCATGG GGATGTGCAC AAATGATGGC TGAACAAGGT GCCCAAGTTA |
| 15361 | TCTATACTTA TCAGAATTCC AGAATGAAAA AAAGCTTACA ACGGTTAGTA GATGATGAAG |
| 15421 | ATCAATTAAT TGAATGTGAT GTAGCAGATG ATGAAAGTAT TGACCAAGCC TTTACGATTA |
| 15481 | TCAAGGAACG TTTTACAAAA GTAGATGGAA TTGTACACGC AATTGCTTTT GCAAAAAGAG |
| 15541 | AAGAATTAGC TGGTTCAATC CTTGGTGCTA GTCGCAAAGG TTATGCAATT GCGCAAGATA |
| 15601 | TTTCGTCTTA CTCCCTTATT GCTGTCGCTA AGGTTGCTAA TGAGTTAAAT CTATTAAATA |
| 15661 | ATCCTGCAAG TATTGTTACC TTAACTTATT TTGGCTCAGA ACGTGCTATC CCTAACTATA |
| 15721 | ATGTAATGGG AATTGCTAAA GCTGCCCTTG AAGCTAGTGT TCGCTATTTA GCACGGGATT |
| 15781 | TAGGACAAAA ACGAATCCGT GTTAACGCAA TCTCTGCTGG CGCGGTTAAG ACATTAGCAG |
| 15841 | TTACAGGTAT TAAAGGTCAT GATGAACTTT TAAAGATGTC CCAAGCAAGA ACTGTTGACG |
| 15901 | GAGAAGATGT GACTATTAGC GAAATTGGGA ATGTGTGTGC ATTCTTAATG AGCGATTTAT |
| 15961 | CAACTGGAGT TACTGGCGAC ACCATTTATG TTGATAAGGG GGTACATTTG ATTTAAAAAT |
| 16021 | ATAAATTTTA AAGACTGAGA AATGAGTTTT TTTCAGTCTT TTTGCTGTCT TTTAGAAGGA |
| 16081 | AAACATAACA ATAGCAAGAA CTTTTTTTAA TTTTTTCTAA AAAGTTCTTG CTATTTTATA |
| 16141 | GCTCAGTTGG TATTATTAAT AACGTTGTGA AAGACGAGTT AATAATTTTG AGAAAAATTG |
| 16201 | TTTAATATAT TTAAAAATGG CCCGTTAGTC AAGTGGTTAA GACACCAGCC TTTCACGCTG |
| 16261 | GTATCGTGGG TTCAAATCCC GCACGGGTCA CTTTTGCGGA AGTAGTTCAG TGGTAGAACA |
| 16321 | TCACCTTGCC ATGGTGGGGG TCGCGGGTTC GAATCCCGTC TTCCGCTTAG CCAGTTCTAT |
| 16381 | TATGCCGGGG TGGCGGAATT GGCAGACGCA CAGGACTTAA AATCCTGCGG TTAGTGATAA |
| 16441 | CCGTACCGGT TCGATCCCGG TCCTCGGCAC TATTTCGGGA AATAGCTCAG CTTGGTAGAG |
| 16501 | CACCTGGTTT GGGACCAGGG GGTCGCAGGT TCGAATCCTG TTTTCCCGAT CTGGCTAAAC |
| 16561 | AGCTAAGAGG TAATCTTCTT AGCTGTTTTT TTGGATATCT GTCAACTGGT ATGGTTGACT |
| 16621 | AAAAATTTTT TTAGTGCGCC CGGCATGGGT ATTAGCTAGG TGGTGAAAGT CCGCTATGGG |
| 16681 | CCGTAGTAGT CGGAACCATG AGCTGAGGAC AAGGGTGTCC ACCGTGAGGT GGAATCTGAA |
| 16741 | GGAAGTCTAA GGCAAAGTAC TGCATCGATG AACAAGAAGT AGCTATAAGG CTGAAATTAA |
| 16801 | CTGGATAAGG CTGCTAGACA AGTTGAAGTC CAATACTACT CGAAGTTGGT CTCAGTAAAG |
| 16861 | CTAACGATGA CATGGTACGA AAGCTAATAT TCTTACCCGG GGAGATCTGG CCTACACGTT |
| 16921 | TCCGACAAGC AGTACTAACG CAGCCATTAA TTAGTTTTTT GAAATAAAAT AAACTTTTTT |
| 16981 | GAAAAAAGTA TTGCTTTTTA TAGGATAACT TGATAATATA ATATTCGTCG CTGATATGCG |
| 17041 | GGTATAGTTC AGTGGTAAAA CCACAGCCTT CCAAGCTGTT GTCGCGAGTT CGATTCTCGT |
| 17101 | TACCCGCTTT TAATTAATTT AATATGGCCC GTTAGTCAAG TGGTTAAGAC ACCAGCCTTT |
| 17161 | CACGCTGGTA TCGTGGGTTC AAATCCCGCA CGGGTCATTT TTGGAGGATT AGCTCAGTTG |
| 17221 | GGAGAGCATC TGCCTTACAA GCAGGGGGTC ACAGGTTCGA GCCCTGTATC CTCCATTGAA |
| 17281 | CAATTTTAAT AGTTGTTCAA CATTTTAATA TAATTGGGCT ATAGCCAAGC GGTAAGGCAA |
| 17341 | CGGTTTTTGG TACCGTCATG CGCTGGTTCG AATCCAGCTA GCCCAATTAG TCTAAAACAG |
| 17401 | TGAGTAAAAA TTACTCGCTG TTTTTTATTA GAAAAAATAG GCAATCTAAT AACGTAGGCT |
| 17461 | AAAAGCGAAC ATCATTGGAT TGCCCAATAA TTAGCATAAT TTATTCACGT CGTAACATAA |
| 17521 | TATGCCAGAC AAAGAAGCCG ACTCCAATAA TTAACGCCAA CCATGCCCAT AGTTGCAATG |
| 17581 | GAAGATAGAT AGTACACGTT AACAAGCCTA AAACCCAACT GATTAGCATC ATCGATAATC |
| 17641 | CAGCAATATC GCCACCTAAA CCATCAATAC CACAAGTAGC GATATAGACG ATTCCTGATG |
| 17701 | CCAGATATAA TAGCGAAACA ATTAATTCGG CTGTTCCGGC ATAAATAGGA ATTGCAGGAT |
| 17761 | TAGGATTCCC AAAACCTCCT CAAGTCTTAT TCTACCCGGA TGATTAGAAT TTATAAAGAA |
| 17821 | TTACAGTTTT TCTATTTACA AATAGAGCAG GGGGAACAAA AAATCAAACC AGAAATTATC |
| 17881 | TTCTGATTTG ATTGGATCTT ATTTAGTTGT ATCTTTTACT TTTTTCAAGG CTTCTAAAAA |
| 17941 | TACTTCATAA GGTTGTGCAC CAGTGATTGA ATACTTATTA TTAATCACAA ATAGCGGTGC |
| 18001 | ACTTGGCATT CCAATCATAA ATGCGCGCCG TTCATTTTTT CGAACTTCAT CTTCATACTG |
| 18061 | ATTAGATTCA AGAACTTTTT TAACATCAGC AACAGGTAGT CCGATTTCAT TCATTGCAAC |
| 18121 | AGTTAGAGCT TCATAGTCCG CGATTGATTC ATTATCATTA AAATAAAGTT GATAAAGACG |
| 18181 | TTTAACAGCC TTATTAAGTA ATGCTTGGTC ATTTAAACTC TCAATGTATT TTATTAAGCG |
| 18241 | ATGAGCAGCT AATGTATTAA CAGGAATCGC ATTTTCCATC TTAATTGGTA AATCAATATC |
| 18301 | GGCAGCAAGT TGATCAATTT TATGTATTTG TGTAACAGCT TCTTGCTTAG TTAGTTGATG |
| 18361 | AGTTTTTGCA TAATATTCTG TCATTGATAA ATCAGTTGTT GTTGGCAACG TTGGATCTAG |
| 18421 | TTGAAATGAC ATAAATTTTA ATGGTGTCTG ATCGGCAATC TTTAATTCTT TAAGCGCCCG |
| 18481 | TTGTAATTGT GTAATTCCCA TATAGCAATA TGGGCACGCA ATATCAGCCC AGTATTGAAT |
| 18541 | TTCCATAGAT GATCGCTCCT TATAAATATA TATTAATTTA ATCTAATCAT AAAATAAGCG |
| 18601 | AAGTTGCTAG TAAATACGTA AAGAGGGAAC GATTTATTTT AAAATTAACA TGTCATTAAA |
| 18661 | ATTATAGTTT TAATGCGACT GAATTTAAAA TTCCCCCCAG AATATCAATT TTTAGCTTTC |
| 18721 | TCAAAAGAAT TTATTTAATG CTTATTTTTA ACTTTAATAA AACTTTTTCT AAACAAAACT |
| 18781 | AATACTTTTG ATTTATGTTT TAAAAGATTT ATGTAATACT ATTGATGAAG TCTATGTCAA |
| 18841 | AAGTATTTTA AAAGGAGTTT TTATCATGAA ACATACGCTT AAAGTTGATC AAGTACGTGA |
| 18901 | CGGTTTATGG CTAGATTCAG ATATTACGTA TACGCAAGTT CCTGGATGGC TTGGTAATAC |
| 18961 | AACGCGAGAT TTGAAGCTTT CAGTCATTCG GCATTTTCAA ACTAATGATG ATACACGTTA |
| 19021 | CCCAGTAATT TTTTGGTTTG CTGGTGGCGG CTGGATGGAT ACTGATCACA ATGTTCATCT |
| 19081 | GCCAAATTTG GTTGATTTTG CTCGGCATGG TTATATTGTT GTTGGTGTCG AATATCGTGA |
| 19141 | TAGCAATAAA GTTCAGTTTC CTGGGCAATT GGAAGATGCT AAGGCTGCTA TTCGTTATAT |
| 19201 | AAGAGCTAAT GCCAAGCGCT TTCAAGCTGA TCCTAATCGG TTTATTGCGA TGGGAGAATC |
| 19261 | AGCTGGTGGT CATATGGCAA GTATGTTAGG TGTTACTAAT GGTCTTAACC AATTCGACAA |
| 19321 | AGGTGCTAAT TTAGATTACT CCAGTGATGT TCAAGTAGCG GTTCCTTTTT ATGGTGTAGT |
| 19381 | TGATCCCTTA ACCGCTAAAA CAGGAAGTGC ATCAAACGAT TTTGATTTTG TTTACCGTAA |
| 19441 | CTTGCTTGGC GCTGAACCTG AAGCTGCAAA TCCCCTCACG TATGTAAATT CTACTTCTAC |
| 19501 | GCCCTTTCTT ATCTTTCATG GTACGGAGGA TGTCGTTGTT CCAATCAAAG ATAGTGAAAA |
| 19561 | GCTTTATGAT GTATTAGTTG AAAACAACGT TCCTACTGAA TTATACGAAA TTGAAGGTGC |
| 19621 | AAGTCACATG GATGTAAAAT TCCTTCAACC ACAGGTATTT AAAATTGTGA TGGACTTTTT |
| 19681 | AGATAAGTAT TTAACCCGGC CATAGATCAT TTCTTGCCTT TACTACTAAA AAGCAAACTA |
| 19741 | ATAACAATCC AACTTTCACA TTATTGACAA AATCAAAAAG CACCATGAAA TTACTTTTTC |
| 19801 | ACGGTGCAAC TATTGTTAAT ATTTATCTAC TTGGCTTCCA TCAATGGAGA AATAGACAAC |
| 19861 | TTGATTTGCA AACCTAATCC AGCAGCATAT TTATTTAATG TAGGCAGCGT AGGGATTGAA |
| 19921 | TCTAAATTCT CGATTTTAGC CAATTGAGGT TGTGTCATAC CGATTTTTTT TGCAAATTCT |
| 19981 | GTTTGCGAAA TGCCCTGTTT AATTCTTTGT ACTTCTAAAA AAGACAAGGT ATCAACAATC |
| 20041 | GATAATTCTT CTTTTGTTGC AGCCGTCTGA TGCTTATCTA TATCTTCCCA TTTTCTCATT |
| 20101 | ATTTACCCTT CCTTTCGTAC CAATCGTCAA GTAGCGACAA GGCTCTTTCT ATCTGACGAG |
| 20161 | GATCTGTTTC ATCCTTTTTC TTAGCATAAT GATTTAATAA TACAAAATTA TTTTTCTTCC |
| 20221 | ATACTCCATA AAATACTCTT TCTGGCATAG GTCGTAACTC CCATAGTTGA TGCCTATAAC |
| 20281 | CCTTTAATTT TTTTGCTTGT GGTGTATGCA ACACAGGACC AAGAGCTTGT AACATCTTAA |
| 20341 | TTTGATGGCG CATTTTTAAA TAAATTGCTT TATCTTGCTT TTGTTTACTT TGCGAAATTT |
| 20401 | TCTCGAAATA ATCACCAATT TCACTATTTC CATTTTTATC TTCGTAAAAA ACTACTTCAT |
| 20461 | ACATGCACTA TTTCCTTTAT TATAGCTTAT TTATTACTAA TTATAGCAAG ATTGCTATAG |
| 20521 | CTTTACTACT ATTAAAATAT ATTCCATAAT CAAAAATTTT TCTATTGAAA GCACCCGCAC |
| 20581 | ACTCAATTAA ATCAAATAAG TTTACGGCAA TGATTATTTT TGGTGCACAA AAATTGCCGA |
| 20641 | TGTTCCCCAG CTTTACAAAC TTTCACCATT TCATACCATT TTAACTAAAC AAAAGCTCCG |
| 20701 | GTAAAATGCC GTTATATCAG CATTTTATCG GAGCTTCTTT TTTATACAAT CTTAAAAAAA |
| 20761 | TGCGTCCCCC GAGCATAGAA GATTGTTGAT ATATCAACGT TTTGAAGGAG TTAGTGTGCC |
| 20821 | AAACGTGTGC GAACTTAACT AAAATAAAAA AGAGCGCCTA TCAGGGCGCC CCACGTTATA |
| 20881 | CCGGTACGGA TCAAGTAACG AACTGAATCC AATTTGCGTG CCGATACTTC TCATCTGATA |
| 20941 | TTTAAATTAT AACATAATTA TTTTATGATA CAATAATTAA TGCGTGTCGG GAGTAACGAC |
| 21001 | CCAAACTATC ACGCAAAAGG GGTGAATTCC CCGTGACATT TATACTTCTC ATTCTGATCG |
| 21061 | TGCCGAACGG GCTCGTCAAG CAACTGATGC ATTATGCAAA GGTTGTAATC AAAAAGGCTC |
| 21121 | TCAAACATCT AATTGATGAA TTACTTGGTT AATTCTGATA GGAAATGTGC GAGATGCTAT |
| 21181 | TTCAGCGGTA GTGTTTGCTA TCGCTGTTTT TTATTATACT TTAAGAATAT AGTCATGGTA |
| 21241 | TTTTTTTACC TTAGTAACTA TTCAGTTTAA TGTTACTCTA TTAAATTTTG AATTCCTTTA |
| 21301 | CATCCTTAAC CTCTATCATA TTACAATCTT TATCTTCAAC GTAGACGAAT TCTGACATAT |
| 21361 | CATCAATATC ATTGAAATCT TTATTAGCTG CGAGTTCTGC AGCATCCTTA AAAAAATTAG |
| 21421 | GCCTTTTTTC TTCTATTTTG TCCATCGGTA CTAAACATTT TAATATGTAA GTTTTGAAAT |
| 21481 | GCTCTTGTTC CCATCTTGTT TGAAGAGGCC CTTTTAATGG CCTACTTCTA ATATTATCTA |
| 21541 | ATTCATATAT GGCACTATCA AGGTACCTTA AAATTTCAGG ACACTTATAA ATGCAAGAAT |
| 21601 | AATCAAAATG ATATGGTATA AAAAAGCCAC TAATTCCGTT ATCATTTTCC AAACGTGCTG |
| 21661 | CAATATCATT CCAACTTCCA CCAGATTCAA TCAGGTATGA GTGCCCATTA TATGAAATAA |
| 21721 | AAGGATTATT ATTTTTACAG TAGAAACGAA TATTATTTTT TTCTAAAAAT GATCCTAACA |
| 21781 | AGGTACTAGA ATCTGTTATA ACTTTGCTAA GTGGTTTTAA TCCATCATAT AAATAGTTCC |
| 21841 | TTTGCTGTGA AGAACAAGTT GTAAAGTGGT AAAAATATGC ATTTTCAATT TCATCTTTTT |
| 21901 | TATATAGTTC TAATGATCTT TCAGCCATTT CATGAGTCGA TAAATCAAAT ATCTTGTATT |
| 21961 | TTTTCTTTTC AGACACCATG GTTTTCTCCT TTTTATGATA TATCTAATTT AGATTACTAT |
| 22021 | ATGACAAAAC ACCCTAGCAA CCATTAATCG GCTACTAGGG TGTTTATTAC TATAATTTTT |
| 22081 | CACTTAAATC TGAATTGTTT GACCAATGAA AATCATATTA GGATTTGCGA TGCCATTCTT |
| 22141 | TTGAGCTAAT GCTTGCCAGG TCTTGCCAAA CTTAGCCGCA ATTCCGCTTA GTGTGTCCCC |
| 22201 | TGCTTGGACA GTATAAGCAT TGGATTGTCC GTTGCCGGCT AAGTAGAGTT TTTGCCCAAC |
| 22261 | GTAGATCACA TTCGGGTTAG TAATGTGATT ACGGCTTACG AGGTCTGAGA CAGTGGTGCC |
| 22321 | GAATTTGGTG GCAATTCCGG ATAACGTATC GCCTGATTGA ACAAAGTAAG TATTTTCGTT |
| 22381 | TGATACTTTT CCGGTGACTT TGAGGACTTG GCCGACATTG ATCTGGTTTG GATTACCAAT |
| 22441 | ACCATTGATT GCTGCTAGGT TCTGATAAGT AGTCCCGTAT TTTTCTGCAA TTCCACTCAA |
| 22501 | TGTATCGCCC GGCTGAACAA TGTAGGTTCC AGTAGCTGGG TGACCAATAT GTTGAACCGG |
| 22561 | CTGTGGTGCT GGAATAACAG CTTGCGGAAC ATTGCCTGAT ACTAAACCAG TCGTAAAGGC |
| 22621 | ACCGTCAAAG TCATAACTAG TATCAATTCC CATGATGCTA TGATCAGAAT ATTGCCAAGC |
| 22681 | ACTTGCATTA TCAATCCCCA GTTCAGTCAC ACCATAGCCA GCAATCCAAA TCTTCCGGGA |
| 22741 | ATCAAAGCCA TGTGAATTAA GAATACCGCC AGTGAAGAAG GACTTCATGG AGTAAATCCC |
| 22801 | AGTATTCTTG TAACCAAGAG CTTCTACTTC TTGAAGAAAG GCTAAGGATA CTGATTGATA |
| 22861 | ATCTGCAGTT GAATGAACTT CCGCATCATC AATCATCAAA GTATCGTCAT ACATACCAAA |
| 22921 | TTGCTTAGCG ATTTTAACGA AGAATCGAGC TTCATTTTGT GCATCAGTCA CTGATGTATA |
| 22981 | ACGAGCAAAG TGGTAACAGG AAACGCGCAA GCCAACCGCT AAGGCATTAC GAATTTGAGC |
| 23041 | GGCCGCACGT GGATTAACAT AAGCCGAACC ATCTTCGGAC CCTTCCGTTA ACTTAACAAC |
| 23101 | GACCCCTAAT GCACCCTGAG CTTTAGCAGC TTGGAAAAAG GCAACAGTAT CTGGTTGATA |
| 23161 | ACTTGAAACA TCGATGAATT GATTACGCAT ATTATTCTTC CCCTTTCATT GGATCGATTG |
| 23221 | CTGGTGCTTG GCCGGCTGGA ATAGTAGCGG GTTTAGTTGG TTGTGATGTC GGATCAACGG |
| 23281 | TTGGTGTCAA TGCAGACTTT TCATAAGCTG ATTGCACAAG CGTTTCAATA GCGTTAAGGT |
| 23341 | CAATATTCTT AATACCCTGC TTTTGAAGTG CTTGTTGAAC AATACCTGTT GCTTGATGAA |
| 23401 | ATTTCTCATG ACCAGCCATG TCTTTGCCAA CTAAGGAAGC AACGGCATTA TCTGCAAGTC |
| 23461 | GTTCAGCACA AGACCAAGCA GCACGAGAAG TTTCTGTTTT GGCATGAGTG ACTTTAGCGT |
| 23521 | GAATAAATGT CTGGCTTTGC TTTAAGGCAA AATAAAAGAC CGTTGAGATA ACGGCCGTCA |
| 23581 | TGATATAGGT TGGAATTGCA TTAATGATTG TGCTCATGTT GTTTCTCTTC TTTCTCTACA |
| 23641 | TAATCTTTGC TGAGCTTGTA AATATGATTC TTTACCCAAG ATGGTAATGG TAGTCCCATT |
| 23701 | TGGCCCCAGT TTTCGATAAT CGAAACTAGG TAAAATAGGA AAAAGAAAAA GACAAGCGAG |
| 23761 | TCGCCTGCCT GTCCTAATCC GCATAATTCT AACATTGGAT AAACGGTTAA AGTCAAAATA |
| 23821 | ATAACTGCAG CATGTTTCAA AAGTCCACCC GTTCCTTTAC TAGAAGTTGT TCGGTGCGTA |
| 23881 | ATGATACTTT TGAAAAAACC TGTCACAATA TCAATCACAA TCGCCCAAAC TAACCATTCA |
| 23941 | ATGAGCACGT TGTCAATCAT GCTGGCAAAA TATTGAATGT ATAGAACGTG TTGTGGCGGT |
| 24001 | TGTGTCAGTA GTAAGTGCAT TTATAGTGGT CACCTCCTTA TAAGGGTAAA ATAAAAACGC |
| 24061 | CCTGTAGAGG ACGTTTAAAA TTATTTAATT TTTATTAAAA GATTCCCAAT TTCCATCTTC |
| 24121 | ATTATTATTG ATTTTAATTA TTAAGTCGTT AAGTTTATTA TCAATATCAT TAGCTAGCTT |
| 24181 | ATACGCACTT GAATCACCTC GTACTCTAGT AATTTCATAT ATTTTTCCTT GACGAAGTTC |
| 24241 | TTCAGTCAAA GATTTAATAT TATCTATTTC GTTAGAATAT TTCTGTTTGA GATTCACTTT |
| 24301 | ATCCCCATCT CCTTACCGCT AAAATAAAAG TGTTATATTA CGCTGTTACT AAATATGGAA |
| 24361 | TATTATTATC AGATAACCAT TGAACTGCAT TATTATTAAT TTTTACAACA TCATCTAAAA |
| 24421 | TTTTATCTAA ATTAAATGAA TAACCAAATA TAAAAAATTC ATGCTTATGA GGATCGTATA |
| 24481 | CTCTTAAAGT TTCATCTTTT GTAGGAGTGA ACACTTTGTC TCCAACTTTT ATAGTTTGAT |
| 24541 | ACAAATTACC AATTACTTGT TTTTCAACCA TTATGTGCTG ACGTCGGTTA AATTTAATAA |
| 24601 | GTCCTTCATC AATATGTTCT AAACTATTCC TGAGGCCTCT TAAATTTTCA TTTTCGACCC |
| 24661 | TATTAATATA GAAATCATCA GTTACATTCA AAATTTCCCT TACCTTTTTT CGATCATTAC |
| 24721 | TATTTTTAGA GTTAGAACCC CATAGAAGCT TAGCTATATC TGACGTATAC AATGCAATAT |
| 24781 | TTTGTAAATT ATACCAAATC ATATTATCTT TATAATATTT TTCATGTTTG GTATTATATA |
| 24841 | TATTTTTAAT AGCAATAATA GTAAAATTAC ATAACAGATA AGTTTCACTA ATTGCATACT |
| 24901 | CCTCGTCTTT ACTATTCATA GTATAATGTT GCATAATATT TACCATTCCC CTTATTAATT |
| 24961 | GTTTAGTAAT ATTATCGTAT CAGTAATTGA AATAATTATG ATATTATTAT GAAAAATTGA |
| 25021 | AGAAAAACCA AAGGCTAATA ATTAATTTAC AAAAGTATGG AATCAAAATT TTAATAAAAT |
| 25081 | GCCGCCCATA ATAAAAGCCC CGCTCGTTTG AGTGAGGCTT ATTTATGTAT TGTGTATTTC |
| 25141 | TAAGGCGACT ATAAGTAATT ACCGTTAAGC TAAGAATTCA TTTAATTATT AGCCATTTTC |
| 25201 | TTTAGCTTTT CTTTATCTTC AATCTGATAC AAGCGAATCT TAGTAATTAA TCCCTGATAC |
| 25261 | GCTTTTACTT CATTGGGAAA ATTATTAGCA GGAGTAAATT TATCATCTTC TTGGGACAAT |
| 25321 | TCAGAAACAT AGAATACATT TCGATAATAA CCTATATAAA GTTTATGGTT CAGTTTCCTA |
| 25381 | TATAATTTAT TTTTTTCAAT AATTTTTGCT TCTTTAGGAG TATAACCATC TTGTTTTCGG |
| 25441 | TCAAAATAGG CTTCCCATTT AATGCACTGT TCCTGTGAAT TAATCCGATT TGTTTTTAAA |
| 25501 | CCAAATGCTG CTAATGGATA CCCACCTAAT TTGGGATTGT TACACAATTC ATTATCATAT |
| 25561 | TCGTAAGCAG TATTAACAAT ATCTTCTAAT GATTTTGAAA TATTCAAGAT AACCACCTCT |
| 25621 | ATTTTATAGA GTATCACTAA TTTAGACTAC TAAACAATAA CTTAAATAAT TTTATTGTCA |
| 25681 | TGATTTATGC TCCTTGAATT TCGTAATTCT GACCGGTTAC CGTCTTATAT TCATCTTTAG |
| 25741 | TGATCGCTCC GCATCGTACA TAAACTTTGT AGAATTCTAA ATCATGGTTA CCCCAGTCAT |
| 25801 | TCCAAAACAT TTGAAGCATT TGTAATTGTG TCATCATTAT GCCGTTACCT CCTTAGATTT |
| 25861 | TTCGACTGCC TGTTGCTGAT TAACAGTCAT AAACATTTGT TGTAATTGCT TGATTTGAGT |
| 25921 | TGCTTGTTGA GCATTAGTAG CCTGCAATTT TGCATTTGAT TGGTTCTGTT GCATAATCAT |
| 25981 | TGCTTGCAAA ATAGTGATTT GTTGATTGGT ATTCCCGTTT GAATGCGAAA TAATCGTCAC |
| 26041 | AGGAATTTGA CTATCATTCA TAATAAAGCT CTCCTTAGTA TATTTTTGTT TGATAAAACA |
| 26101 | CTGTTACAGT TACCCCTGTA TTTTGTATAG CTGAGTTATC AATGTTTCTA ACGATTAATC |
| 26161 | CATCGCCGTT TCCAGCTTCT GAAACACTCA ATTGCCAGCG GTCGCCTTCT GCAACATAGT |
| 26221 | TAAGTATTTT CCCAGTAACT CCAAGTTCTT TATTGATAGC ATCCATTGGA AGTGTCTTGA |
| 26281 | CCTCTTGCGA ATTAAGTTGA TCAATAGCTA TAAATTTAGT AATCGTCTTT AAATTAGAAT |
| 26341 | TGTAATCTGT TTGATGATCT TGCAAACTCA TTCCTAGCGA TGTATTGCCT AAACTAATGA |
| 26401 | GCGAAGTTGG CGTTTTATCA GCATAAATTC TCCCAGCTTG AAAATGGGCT GAGTTTTGAT |
| 26461 | CGTAAAATTT AACTCCAGTA TTAAAAGCCT TTCCACCTAA CTTAAAGTGA TCCACATCTA |
| 26521 | GATATGTTCC AGTCGTACTA GGCCCAATAT ATAGCCCATT CATGCTTTCT CTTTCAGCTC |
| 26581 | CTAGCATAAT CTCGTTTGCT GTAAACTCAG CGTTAGTTTG CAGAGTGAGC AACTGATCGC |
| 26641 | ATTTTCCCCC AAGTGCAAGG TAATCAATGT GGCCCTTTGA TAGCATAATT TTTAGATTAG |
| 26701 | CTTGTCCACT TTTATTTGTA ACAAAATGGC TTTCCCAATG TTTGATAGTT AGATCGTAAG |
| 26761 | TTGAATCAAT CACTGATCCA ATAGCATTGT CTGCTATATC AAACGTTTCC TCCCACACAA |
| 26821 | AAGAATATTC ACCGAAACCG GATTGTTGCG ATACCGAATG CTGATACAAT GTTTGATAGC |
| 26881 | AATTATAGTT CCAAAGTTTG ATTTTGATCC CAATATTATG TGTGTTATCT GCATCTGTAC |
| 26941 | CAGTTACGTT TAATATACAT CCTCTGAAAT AATACCCTTT AATCGAAATA TTACTATGAA |
| 27001 | ACATGGTATC AAAAGAAACC CCATACTTAG CTGCACCTCG CTCTAGCATA ATTTGAATAT |
| 27061 | CATTTTGATT TCCATTTCTA AATTTGAATG CAATTTGATC CACGTTTAGT GCTGGTGTTA |
| 27121 | ACTTTAACCT CAACGAAATA TGAGACTGCT TCAAGTCAAT AGCTAATGGA CTTTCAATTA |
| 27181 | GATAACATCC ATTCCCATGA ACACTTTTAT TATTATCAAC TGCATAGGTT AGAATGCTTT |
| 27241 | GAAGGATTTG GTAATCATCA TTTTTACCAT CTCCATGTGC TCCCCATGTT TCAGGTATAA |
| 27301 | TTTCATTTTC AATAAGTTCC GCTAATAGTC CTGATGCTAA TTGTTCTGCA TGTTTAGTTG |
| 27361 | TTGTATCTGT GACATAAAAA AGCCCTCCTC CTCCATCATT TGGTTCATAA TAGCCAAGAG |
| 27421 | TTCTTGCGCA CATATCTTTG CTTAAAGCAC TATCAGCTTT CATGGCAGCT ACATTTGGAT |
| 27481 | ACACATGTGC ATAGTTAATC GCAATTGCCT TCATCATTTC CTGCTTAAAC GCCTCTGCTT |
| 27541 | CCGCCTGGGT AAACAAATTG TCTTGCTTAA TCTTAGCGTC TAGTGCATCT AAACCATTTT |
| 27601 | GAACAGTAAC GCCAAGAGCT TTGAGCGTGT TCATTGATTG GGTAAACTTA TCAATAAAGT |
| 27661 | CACTGGTCAT CTTGGTGAGC TTGTCGGTAC TGTCTTGGTA CTTGGCTTCG ATTTCGTTAA |
| 27721 | TTAACTCTTC GACGGGTGAA ATATATGTTC GTGGTACCAA GCCATCAATG ACCTTATCAG |
| 27781 | CAAGGACATC GAGGTCGAAT TCCAAGGTAG TGATAGAGTT ACCGTCTTTG AGGATGCGAA |
| 27841 | AAAAGGCTTG GCGATAGGAA CCAGCGACTG TAAAGGCATG GCCGGGCATG TCGAAACGGA |
| 27901 | AACGTCCGGC GGTTGGGTCA AGGGCAACGT AGCCTTTATC ATCAATTACT CGGAAGTCGC |
| 27961 | CGGCGGAATT CTTAGGAAGT AATCCTTCAA ACCAGACGTT ACAGCCAGTT AAATCTAGTG |
| 28021 | GTGTCCCGTC TTCGTTCTTA ATATTGACAA AAACTTGACG CATACTTCGC TCATACTGAC |
| 28081 | GCGCTTGGAC CCAGTTAGTA TTACTGCCAT CAAAGTTAAC CTTGAAGTCT TGTACATTGT |
| 28141 | CAACATGCGG ACGCCGGTCT TGACCGATCA CATAAGTTAA AGTTTGTGAC ATTAAATCAT |
| 28201 | TCCTTTCTCT TTCAAAATCT GCATGACAGC TTGTTCAACC GTTTCATAGT CGGTCCCTAA |
| 28261 | AGCCACATGC TCAATTTTCT TTTTCCACGC TTCTTGAGCC TCTTTTAGTT CATCTCGCGT |
| 28321 | GGCTTTTTCT TGTTCGAGTT GGTCGAGACG AGTATTAATC TCCTCGAGTA GCCTAATCAA |
| 28381 | TTCTTTATAC TGCCTGTCAC GGAGATTATC GTCTTTGTTA TCCTCATTTT GAATTATCGT |
| 28441 | TAAATTATTT ATTAATTGCT GGCGGAATTT TCTTCCACTC TGCAAATCTA GATTAGTTTC |
| 28501 | TAAATCCAAA TGACCATCCC CCTCCTCACT TCAAACTACT CCAATCCGTC ACTCTGTAAC |
| 28561 | CTAGATTTAC CTGGGCCCGT GATACGTATG CTTTATCAGA TCCGGAAACA TTAAAAACCA |
| 28621 | GCCTTCCTTT AGTAGTGCCG GCTGGAATTT TAATGTTAAC TGTGCCGGCG GATTGCCAGG |
| 28681 | CGCCATTATT GACGATATAG ACAATATTCG ATTTGCCGGC AGAACTACCA TCGCTCTTCA |
| 28741 | AGAACTCGAC ATAAGTAGTA GCGGAACTTG AATTATCGGT CGCATTTAAC TTAGCCATGA |
| 28801 | GACGACTTGA GATGGAGGAG ACACCGTTGA GGTTAAATTC ATTGCTTCTG AGAGTGCCTT |
| 28861 | GCTCTACTTC AATTACTGGA CTGTCATTAT ATTTTTCGCT GGAAACAGTT CCGCCAGTCA |
| 28921 | TCATAGTTAC CAAATTATGC TGGCCATTTT GCATCCAGTA AATATTCTGG TCTACTGCTT |
| 28981 | CCATATTCTT ATCCGTCTCT TTCTTATCAT TCACGACTTG CGTTAATGAC TTGGATTGCT |
| 29041 | GATCCATGCT ATTTGCTAAT CCACTAATCC GGAGTCGTTG TTCTTCAAGA GCTTCTTGTA |
| 29101 | GACGTTTAGC TTTCTGATTT TGATAATCTA AAATAGTTGT TTTTGAATTA TTTAACGTTA |
| 29161 | TTTGATTATC TTGTCCCTTA GCTTCCGGAT ATATCGTATA ACCGACAGTC TTATAACTAC |
| 29221 | CTGAATATTT ATCTCGCGCA AGTACTCTTA AAATTTCACC AGCAACAGGT ACAAATGAAT |
| 29281 | TTCCCTGAAG CGTGACTTCA ATATTTAAAT CTGGATCAGG TTTAAGAGTA GTTAAGGCAT |
| 29341 | ATTTACGCAT TGCATCCGCA TCTTGAAATC TTTCATCAAC AAGATCAGGT CCAGGATGTT |
| 29401 | CTCCCCATTC GTCAATTGAC TTTTGATCAA CTACCATAAA CGGTTGAAAA TAATACTCTT |
| 29461 | CCTGTGATTC TGTGGTTGTA TCATTTCCGA CAGTATCATT GGTTTCTTTA CCACCGATAC |
| 29521 | CATCACGAAT AACTTGTAAG GGATCAAGCC AGGTTCCATC ATTTGACCAG GCACTTTTCA |
| 29581 | ATGCAGTCAT TAAATTCTTT TGCTTAGTAA TTCCAATATG AAGATGGCTA GTATCACGGT |
| 29641 | GGCCAATAAC ATCCCCCGTC TTAATTTCTT GGCCAACTTG AACAGTAATA TCTCCTCGAT |
| 29701 | TCAAAAATGC TTCTTGGTAG CAAATTAAAT AATCATTCGA CACAATAGTT ATATAGTTTT |
| 29761 | CCAGTCCAGC AATGTATCCA ATATCCTGGA CTTTCCCACC ATGAATCGCA TGAACATCGC |
| 29821 | GTCCAGGATG ATCGACAGAA CCAAAATCAA GGCCATCGTG AAAACCATTA GTTCGCCCCA |
| 29881 | CTCCATCTTG AGGGTGTGTT CCAAAAGTTT GCCCCAATGA AAAATGACCT TCTCCAACAT |
| 29941 | CTGGGAAAGG CCATCCCCAC GGATTAGGTG GTGAAATGAT CAATTTATCT TTCGTGATTG |
| 30001 | GGGCACCATG AGGACTCCAA CCAGTAACAC CATTAATTTG TCCTAGTGCA TTAGGAATAT |
| 30061 | TGAAGAACGC AATTAACTGG TCAAAACCCT TTAAGATATT TGTATAGGGC TCCCGACAAT |
| 30121 | AAGTATTAAA AGTTCCTCGC TTAAATTGAA GTAACCCAAG CGCCGGACCA GAACCATCGC |
| 30181 | CATCGGGATC AGTTCCTGGT TGCGGAATCG TCTCGTTTCC ACCTGATTCC AAATGAATTT |
| 30241 | GTGCCCGTAA AACATTAAGC TGCTGAGCAT TCGGTTTTAT TCCATAAAAG CTAGCAGCAT |
| 30301 | ACTGAATAAC TGGTGTCCAA TCACCGTTGA CTGGCTCAGT TGGTCCATTA GCATTAGTTC |
| 30361 | CACCTGTCCC GGTATAAACA GTGTGCTGAA CCTCATGCTT ACCACCAACA CAACGAATCA |
| 30421 | TATTAATAAT TGATTGACTG TCCCGAGTAG TTTTAACTGA CTTTGCATCC CTTGGAAAAT |
| 30481 | CAATCACTCG ACCATTATCC TTATAGAACT CATCTTCTGA ATAAACTCGT ATCTTCTTGT |
| 30541 | TATCCGGATA TATAACTGCA CTTGGCCATA ACTCAGTAAT CTTACTAAGC ATCTCTTTAC |
| 30601 | CACTGCCGCT ATCGAACTTA GAAGTTGACT GCTTATCAAA ATTACCATGA ACTTCATAGC |
| 30661 | TAAAGCCTAG CTTATTTCCA TCAATCCAAG CCTTTAGTAA GTCCTCAACT CCATAAGACT |
| 30721 | GATCCTTACT AGCATTACTA TCCTGATTGT TATTTAAGGT AAGGTCACTT CGTTTATAAA |
| 30781 | TTCGACTAAT CTCATTATAG ACATGCCAAG CAGTGATATT TTTCGTATTA GTATCAAAGT |
| 30841 | TTTCAACACA ATTTTTTACA ATATATTCCT GTCCCTCAAG CACAATCGAA CTTTCAACAT |
| 30901 | CTAATGGATC ATACAATGCT GAATCGTAAT CAAATACTGA AAAAGTCAAT TGAAAGGTCG |
| 30961 | AATTCTTAGA CCACTCGCTT TGCATTGTTG GCCAGAGAAT AATGTCTCCG ATTGGCTCCT |
| 31021 | TAGTTGTCTC TTTATGAGGT GTCATCAATA CTAAACTAAT TGTCCTCACC TCCTAACCAA |
| 31081 | GATACATAAA TGGGAAACTG AATGTAATTT CCAAATCATC TGCACCAGTA ACTTCAAAGT |
| 31141 | CATTCTTTCC AGGCGCTAAA GTAATATACC CATAATCGGT ATTGGCACTG TCTGGGTTAT |
| 31201 | TATTCTTAAA TGTTCGCCGT CCCTTTAGCA AGAGAGTATC GTTTCCAGAT AGATTTTGAT |
| 31261 | TGTACGTCCA AGAAGTATTA GTAGTTGTAT TCTTTACTGT GAATTTTCCA CCATTATGCT |
| 31321 | TAATAGTAAT TTTTAGATCA TGACGCTGTT CAACAGGATC GATTGTAATA TCACTATCGT |
| 31381 | TAAGGATAGA AAACTTATTC TGGCCTTTGA AATGGTATGA AGGAGTTTCC TCATCCATGT |
| 31441 | TCATATTCAA GTCTAAGAAA TCCTCATCTT TCATCTCATC CGTATGCAAT TTACTAAATC |
| 31501 | GCATTCCGCT TGGATTTTCA AAAGGCACTT CAAACGTACA ATAGTTAACT TCTTCTGGTT |
| 31561 | CGCTTTTAAT CTCAAAGGAA CTAGCCCGAC AGTAACGGAC AATATCCGGC TCAACTCCTG |
| 31621 | TTCTTAACCT AAAGATTCCT TTTTGTGCAA AAACGCGATA AATCTCATGC TTTGCCATCT |
| 31681 | TAAAATCTTT TCGATCAAAG AACTGCAATA AGAACTTAGC GGTGATCGTC GTTTGGCCAT |
| 31741 | AACGGGAATA ACTCCAAATT TGACCATCTT GTTGGGAATC ATCACGATAA TTATTAATAA |
| 31801 | CACTAGGCGA TTCAGTTAAG CCAAGAAAAG TTAAGTGGTC AGTAATATCT GTGCTGGCCA |
| 31861 | CTTCTTTTTG ATCATCAATT TTAATGTAAA GAATGTTTAC AGCGATTACG ACCACCTCCT |
| 31921 | AAAGATGTTG ATTATCAAAT AATGCTTGAT CAGTTCCTTC AATCCCATAA AACTGGTTCT |
| 31981 | TGTCAAAGGC GCCGGCTTTA ATTGCAGAAA GTTGAGCTGC ATTTAAGCCC AGCATTGTCC |
| 32041 | CAAAGCGGTT GAGCATCTGG TCCATCCGAT CTAACATCTC TTTTTGCATT CCATTATTAC |
| 32101 | TTTGGCCACC GTTATTATAA TTATTTACAA CTTGGGTGCT TTGCATATTG GGCTTAAAGT |
| 32161 | TAGTTAATAC CGAAACAGCA TCATCAGTAC CATTAGCATA TTGTGGTAAG TGCTTAAACA |
| 32221 | TCTTAGCAGT ATCGCTAGCT TTAACAACTT TTGTTCCTCG TGGTGCTGGA AAGACTACAT |
| 32281 | TACGCCCATA AGGAATAAAT GGTGTTTCAC CTGGGAATTG AACTAACTCA CGAAAGACGG |
| 32341 | GGCCAGGTTG ATCATTAACT TCCATCAATC CACCTTTATG GTAATTAGTT CCCTGAGCAT |
| 32401 | GCTTTGATTT ATGAAAAATA ACATTAACTG TTTTCTCAAT AGTATTAGGT ATACTCATAA |
| 32461 | AATTATGGAT TGCATCAAGT GCTGTTTTAA TTGGCCCAGA AGCTTTATCA TTAGCTTTTG |
| 32521 | CAATTTTTGC TGGTCCCGTA TTTGATCCAG CAAAAGCATT TACACCATTG CGAGCACTAT |
| 32581 | ACATTGGTCC ACTAGCATTA TCAACGGCTA AAGCATTTTT AGGAGCGCCA GGATTTGAAC |
| 32641 | TAGCAAAAAC ATCGACACCA CCAGTAGCAT TTCTCATCGG ACCACTAGCA TTGTCAATTG |
| 32701 | CCAGTGCATT CTTTGGTTCT CCCGGATTAG CATGACGCCA AGATTGAAGG GCATCGTTAG |
| 32761 | CAGCTTTTAG ATTTCCACTA GCTTTATCAT CAGCAATGAT TTGTTTTGCA GCACTAACCG |
| 32821 | GCATCCCCTT CCACAGCCCA TAATCAGTGA TCAGTTGCTG TAGTTCGGGT CCACCCTTAG |
| 32881 | CATTTACGAT TGCTTGCTGT TGCTTAGGAC TAAGTTGATT CCAGCGACCA ATTTTATTGA |
| 32941 | GCGCATCCAT TAATCGACCA GCACCCTGAG TAGTAACCAA AGCACGTTGT TGCTTAAACG |
| 33001 | AAAGACTATT CCAAACACCA GCCTGCACTA ATGCATCAAC TAATTGTGGA GTTCCTTTAG |
| 33061 | CGTCAATAAT CGCTTCCTGT TGTTTTAAGG TTAAATTATT CCACTGTCCG GAACTTTGCA |
| 33121 | AGGCTTCATA AATTGGCTTA GTTGCCTTAT TAGTAATTAC TGCTTCCTGT TGTTTTAATG |
| 33181 | ACAAGGAATT CCATGCACCG GATTCAAGCA GAATGTCGGC CATTTCTTGC TTACCTTTAG |
| 33241 | CCTTAACAAT TGCTTCTTTG GTCTTAAGAT CAAGATTATT CCATTCTCCC GACTTCTCTA |
| 33301 | AAGCTTGAAC AATTGTTTCA CTAAAGCCGT CTTTCAACCA AGCTTTCTGC TCTTTCCAAC |
| 33361 | TCATGCTATC CCATTTACCA TTTTCGATTA ATGCAGCTGC CACCATTTGT TGAGCATTGG |
| 33421 | TACTTAACTT ACCTTCCTTT TTCAGAAGTT TAATTTGGTT CCATTGATCT TTAGAATTAA |
| 33481 | CAGCCTTATT AACTTCCTCC TGGGCATTAG TCCGAACTTT ACCAGTTTTG GGATCGAAAA |
| 33541 | CTAAGTCATT CCACATATCA GCGGCTTTTT TAGCTTTTCC ACTTAATTTA GTAGTATTTA |
| 33601 | CCGCTAAGCT TTGTAGGTTT GTTTCGGCAG CTTTGGATTG CCGCTTAATC TCATCAGTGC |
| 33661 | CATCTTTATA ACTAAGTCCC ATACTTTTTA GGTCATTCTT AATTAATTTA GTACTTTCAC |
| 33721 | CATTAGCACG GGCCAGGCGA ATATATTCGG CAGCTGCTTT ATTCGTGTAA TCTGTTAATG |
| 33781 | CTTTCTTATT AGCACGCATA CCGACCTTAT ACAGTTCAGA ATCTTTTCCG TAGCGTTTAG |
| 33841 | CAAGCATTGA AGACTGCTTC TTGTATTGGT TTTCCATTTC AGCAGTTTCA CTGGTTATAT |
| 33901 | CTCCCATTTT TGTTGAGCGT TGTTGCATAG ACATATGCTT AATATCGTTA TTAAGGACAG |
| 33961 | CCATTGCTTT AGCACGCTTA CTTCCACTTA ATTTAAGAAT TTTCATCTCA TCGTTAAGCA |
| 34021 | TTTCCTGCTG GTTGTTCTTT AACATGACTC GCTGAGTATC ATTTAATTGT GAAACTTTTT |
| 34081 | TATTGTGGTC AGATAGAATT GTTTGAACAC GCTGATTGGC ATTATCTGCA TCATTCACAT |
| 34141 | ATGAATTATA TTGTTTTTTC TCCTTTTCTA ACTCATCGTC AATAGCTTTA GATATTTCTG |
| 34201 | GAGACAGACC TTTTTCTGCC TTTTTGGCAT TAGCAAAATG TTGTTTTGCA CTATTTTCAA |
| 34261 | TATTAGTAAA CTCACGGTCA AAATCAGCAG CTAATTTCTT TGTTGAAGTT GAGGCAGCAA |
| 34321 | CATCCATATC TGACAGAGTG TTCTGAATTC CTTGAGAAGT CGACTGCATT TTTGATAAAG |
| 34381 | CCTTATCAGC AGCTGCACCA ACATCAGATC CCCAACGATT CGTTCGTTGT GATGATTCGA |
| 34441 | CAGCTTTCTT ACCCCAAAGC TCCCAAACAG TAATTCCACC GGCAATGGCT AAAGTAGCAA |
| 34501 | CTCCAGCAAC AGCGGCAACT GTTCCTAAAG ATACTCCAGC CGCAGCAGTA GCAGCACCTG |
| 34561 | CTTCACCTGC AGCTACACCG GTACCTTGTA AAGCGACAGT CGCAGCACCA ACACCGTTAG |
| 34621 | CAGCTTGAAC CGCCCGACCA CCTGCACCAG TCATGGTTGT TCCAAACTTT GCAGCTTGAA |
| 34681 | ATGTTGATTT TGAAAAAGCT GAGCCAATAA CGTCTAACCC ACTGGCTCCT AGTTTCATTG |
| 34741 | CAGTTTGTAC ACGGCCGATT CCGGAAGCAA TTTTTCCAAA GGCAAGAACT GTCTTCCCAG |
| 34801 | CACCACTAGT CAGTTTACCC AATGCTAGGA ACAACGGGCC TGCACCAGCA GTGAACAGGG |
| 34861 | TAGTTGTTAC AATTGCCTTC TGGACAGCTG GTGATAAATC ACCAAAGCCA TGTGCAAGTC |
| 34921 | TGGAAAGGCC TTGAACCATT GGAATAATAG ATGGTAAGAC GTACTTTGCC ATATCCATTC |
| 34981 | CAGCGTTAGT TAAAGATTCC TTGAAGATTG CTAATTGTGC TTTAGGAGAT TGAAGGTTTT |
| 35041 | TCTGTGACAA GTCGCCGATA TAATCCCGCT TGGCAGAGTT TTGAACTTCT TTATTAAGTT |
| 35101 | CACGTAACCG ATTAGCGTTT TCGGTTAAAA TTGCCCCAGC TTGTTGTCCA GTTGTTCCAA |
| 35161 | ATAACGCATG AAAAATATCA TTCTTTTGGT GACCAGATAA GCCTTTCATA TGGTCATTTA |
| 35221 | ACGTCTTAAA AATAGACGAC ATTGATTTTA ACTTTCCACT CTTAGTAAGA AAATCTTTAG |
| 35281 | TACTTAAATT AATACTTGCT AAAGCTTTTT GACCATTAGC GGTTGGCGTA ATTAGTGAAT |
| 35341 | TGATAACCTT TCGTAGACCG GTACCAGCTT TGTCTGCTTC TAGACCATTA TTAGACAAGA |
| 35401 | TCCCCATTGC ACTAGCAGTT TCTGAGAGAC TAAAACCTGC TTGATGAGCA GTTGAGCCAA |
| 35461 | CATATGACAT TCCCACACCA AGTGATTGAA AATCAGTTGA TGTGGCATCG GCAGCATAGG |
| 35521 | CTAACTCATT TAATGTTTTA GTAGATCTAC GCTGCATGAC TGTAGCATTC TTGATTGGAC |
| 35581 | GTCCTGCTTT ATCAGTAGCC AGTCCAAAAG ATTCCATTGT CTGCGAAGCA ACTTTGATTA |
| 35641 | CATCATTAAA GTCATCCCCA GTGGCCACAG ATGCTTTAAG TTCGTTCCGC ATTACTCCAA |
| 35701 | TTGCTGCTTT AGATGTATAA CCACGCTTTA CTAGATCTTG ATACCCGGCA GCAATCTTTT |
| 35761 | GCTGACTAAC ACCGTACTCG TCAGAATACT TTCGGGCATC TGCAGTCATC GTTTTATATG |
| 35821 | CGGCATTCGT TTCTGCAGCT GATTCTCCGG AAGTTCGAAT AACGTTCTTG ACCTTTGTCA |
| 35881 | TTTGATCTTG GAAGTCAACG AGTTTCTTAG CGGAATATGT TAACCCTGCC GCAATTGGAG |
| 35941 | CTGTTAAATA AGTCGACATC CCTCGACCGA CACCTGATAT TTTAGAACCA ACATTGGTGG |
| 36001 | CCACATTTCC AAAATGTTGA GTGCGATTAG CAAGTTGTGT CCACTTATTC GACTGCAATT |
| 36061 | CAATATCACG ATTAAGAGCT TGCATTCGTC CACGTAATTG TTCAATTTGC GCAGACGTTT |
| 36121 | TATTATATTG GGTTGCTGCC TTAGTTCGTG ACTGCTCGCT CCTGGTAGTA TCATCCATTG |
| 36181 | CTTTCTTAGC ATTCTGGAGT TGCGCATTAT AATTGCGCAT TTGTTGACTC ATAGTCGCAT |
| 36241 | AAGCAGCACG CATATTATTA ATACTTCCAC CGGATGCTTT CAGTGCCGCT TCTTGCGCAC |
| 36301 | GCAAAGCATT GGCTGTTGAT TTAATTTGCG CTTTTAAAAC ACCATTAGCC GCTTTAAACG |
| 36361 | GATCAATATC CAAACTAACA GTTGCTGCCA AATGTCCTAA TGATTGGGTC ATTATTTAAC |
| 36421 | CTCCTTTCCT AGAATAAGAA TGGGAATGCT TTATCAATGG TTGTCTGCTT TTCTTCAAAG |
| 36481 | ACGTAATTCA TTAATTTCAA ATCTGAAAGT GTTAGCTTAC TGACCTCATT CCATTTATAT |
| 36541 | CCATCGCGCA TTTTATTTTT AATGAAATCT GTGAGACTCT TGATTGAATC ATCAATCATC |
| 36601 | TCAACAGTTA TTTTTTTGGC TTTTCGTTAT CCTTTTTCTC TTCTAAGTCA TCTTCACTTA |
| 36661 | GAGGAGAACC GAGAGTTTCA TTAATTGCAT TAACAATGCT AATTAACCCT TCTATAGCCG |
| 36721 | TAACTCCTGA AAGAACATCC TTTTTAGTCA ATCCATTCTT CCAAAAATTA GCAGCAAACT |
| 36781 | CAGCCCGTAA ACTTAATAAT CGTTCATTAT CTTTATCTGT TGGCAATTTT TTGGGATCAG |
| 36841 | AGTACATCAC TATTTCTTGA CGTTGAACCT TTAATGCATC TAATAGATTT TCAAGCATTG |
| 36901 | GGGCTTCTTT TCGTTCATAG ATTGTTTCTT TGCCATCTGT TTTTACTTTT AATTTATATG |
| 36961 | GCATTTCCAT ATCCTCCTCA TCGTCTCACT TAACTCGTCT CTGTCTGATT AATTAGTTAC |
| 37021 | CCTTTATCAA CAACAGTTTC TGGTTTAGTA TCTTTTGTAT TGGTTGGATC AGTTACTGGA |
| 37081 | GCTTCTTCCC CAAAGACCAT TGCATGGAAC TTAGCAAAAT CAAATCCTTC ATTATCTTCC |
| 37141 | CGACCGATTA AAAGGATGGT TCCAGTATCC GCATCCCCAC GCGGAACGAA ATTACCTTCA |
| 37201 | ATTTCATCTG CTTCTGGGTC TGGAGCACCG TCCTGAGTCT TGATTGAAAT ACCTGGTAAG |
| 37261 | GAGAACATCC CCTTGGTTAA ACCAACCCAA CAATGCTTCC CGTTTGAAAG CTTTGTCCGG |
| 37321 | AACATAGTAG CAACATAATT TGGTACAAGA TTCTTAGTAT AAACTTCTGT ACCGTTCTGA |
| 37381 | ATATCAATTC CATACAAGTC TTTCTTCATG ATGGAATCAA TATCATAAAG GTTGATGGTT |
| 37441 | TCCTTAGCTT CGGTAATTCC ACCAGAAAGA ACCAAGTAAG GGCCGTCATC GGCTGCTAAC |
| 37501 | GTCTTTAATT CGTTAGTCAG CTCCAGCTTA ACTTCACTTA ACCCGCTCAT CTTTCGGGTA |
| 37561 | TCTTTAATCT TTTCGTTCTC GTCTAGCACC CCATATTCAA AATTAGAGGC CCCAAACTTA |
| 37621 | GCAACTTTTG CATTTGGTGT TCCCATTAAA TATCATTCCT TTCTTCAAAC CCTTCAAAGT |
| 37681 | TAGCTGTGAC CATTATGCAA TTATCCAAGT CCGGATCCGG ATAAGAATTC TTGTAGTAAC |
| 37741 | GTTCAAAGCC ATGACTGTGG AGGGTTTCAT ATATTCGTTC TTGAATATCC ATTAGCCGGT |
| 37801 | CATCATTCTC TTCACGAATC CAAAAATCAA CTTGTACACG TGGGTATTCA AAAAAGCGAG |
| 37861 | CATTGTCACT GTAAATTGCA TCATCCCCAG GTAGGGGGGT GATCCGAATC CACGGTGCTG |
| 37921 | AACTCGCTTT AATAAATGGA TCATCAGGCG TACTAGTAAA AATTGGAACA TATGATAATT |
| 37981 | TATCTTGTCG CAACTCATCC ATCATCACGA CTAGGCACTC ATCATTCGTT AGATAATCGG |
| 38041 | CTACTTGCAT CTCTGGAGTT TTCATACTTT CAAGTCCTCA ATAAATTTAG CCAATATTTT |
| 38101 | CTTCTTAGCA GCATTTCTTG AATCTTCAAC AAAGTGTTGT GGATCTTGCT TAGCAGTTCC |
| 38161 | AGCATCGGGG AAGTGAGCAA TCCATCCTTT ATCTTTGTCA TAACCAACAT CGACAGAATA |
| 38221 | ATCTCCACCG TTAGTCTTGA GATTACCGTG TTTAGTATGA TCTTTTAAAG GAGTCTTGCC |
| 38281 | TGAATGATCA GAGCCTTTAG AAGCCACAGG CGTCCATTTT CTTAATTCTT CTTGAAAGAC |
| 38341 | TTTAGCACCA TCACGGGTTG CTTTTCGTGC CTTCTTTTCT TCCGTCTTTT CAAGTTTAGT |
| 38401 | TAAGTTAGCA ATCAGCTCTG CTTCACCGGT CACTGCCATT TTGGACAACC TCCTGGGCGG |
| 38461 | TAATTTTTGT TAGATCTCGC TTTGCATAAT CAGGATCCAT TCCCGTGATT TCATACCATT |
| 38521 | TTCCTCGCCA GTTAATTAAC CAATTCGATT GAATTTCCTT ACGAGTTTTG AAAGCAATTA |
| 38581 | AAAACGTGGG TGATTCTTTT CTGAACCCCA CGTTTGAACT GTTTTGGACA AATTCACGGA |
| 38641 | CTGACAGCTT AGGAACTTCT GCCCACACGG TAAATTCCTT AACTTTCTGA TCCTTGATCG |
| 38701 | GTCGATGAGT TTCCGGATTG ATCCCCATTT GGACTGAGTA AAATGTTATC CGTTCCGTCA |
| 38761 | TGTTCCGTAG TTTCATCGGT ATTCACCTCA CTACTTATTT GATTAATAAG GCCATCAATT |
| 38821 | CCTGATGAAA GAACTGGTCG GTAACTATCT GCAGTAATTC CTCGCTCGTA GAAGTCTTCT |
| 38881 | TTCACCTGCT TCATAAGTGC AATTTTGAAT CGCGGTTCGT CAGAGTAGTC AGCAGGCTTA |
| 38941 | CTCTCCCATT TAATTGCCCT TGCAATCATC AATGCGGCAG CATCAACAAT TATTTTAAGA |
| 39001 | ATTTCATCAT CAAAGTCCTG GTCAATTTTG CAGTAGTTTT TTAGATTGGC AAAGAATTGT |
| 39061 | TCATCACTCG AGAAAGTGTG GTCTGTTTCC ATCCTACTCA CCTAGCTTTG CTAACAGTTG |
| 39121 | ATCCTTAGTA TCGGTTGAAG TATAGGCAAT TCCATGATCA TCTAAGTACT TTTTGATTGT |
| 39181 | GTCTACCGTA TCAGCACTAG TCGGTTTAAC GTCCGCCCCG TTGTCGGACG GCGTTATTTT |
| 39241 | GACGCTGCTG ATGGACCAGT AACAAAGTAA CCAGCGTTTT CATCGGCCTT CTTCACATCT |
| 39301 | AAACGAAGAA CCGCTTGGAG ATATTGACCA TAAATGTCAT TATCAACCCA CCGTACCTGG |
| 39361 | AGATCTTTTC GATTTGCTAA AATAATTGCA CGGTATGGAT CACCAACAAA GGCATGAGCT |
| 39421 | TCACCCTTTG CTCCAAATAA GTCATCTTCA ATTACAGCAA CGTTGATTCC AGAGACCGCT |
| 39481 | TTACCAGACG GACTAGAAAT ACTATCTTGA AGTAAGTAAC GTCCATTCTT ATCTTTCAAT |
| 39541 | GTATCAAGCC AGTTGTAGAA GCTTTGGCTG GCAATAATCA TCTTATTGTA AGCCACATCT |
| 39601 | AAATCGACAT TCCAAATTTT CTTTAGGTCA TCGATTGCAT TAGAGCCATC AACGGTCTTA |
| 39661 | GCTGAAAATC CCTTAAAAAT TGTAGCAATA GCCGCATTCT TTGTATTTAA GGATTGTTCT |
| 39721 | TTTGCGTTTC GAGCAACTAA TCCAGTTAAA TCAATTGCTG AATCATCAAT CGATTCTTGT |
| 39781 | GAAATAGGAA TTGCTCCACG ATAGGTCTCA ATTTGCCATG CTACCTTAAG AAATTCAGGT |
| 39841 | TTTTGAAGAT CAGGATTCTT TGCAAGTTCT TCAACAGTAT GCATTTGAGC AGTTGCCTTT |
| 39901 | TTAAGAATTG GATAACTACC AGAAGCAGTT GTGGCGTTAA ATACTTGAAC AAATTGGCTA |
| 39961 | AGATCAGTTA CTGTTTTAAT TTCATTTTCA GGATTGTAAA TGATTGATTC AGGAATAGTA |
| 40021 | ACGCTTGCGT CTGAAGAAGT AATTCCAGTA GTACCATCAC GATGTTCTTG GTGAAGATAT |
| 40081 | GCGTTGAAAT TACGTTTTTC TTCATTTTCG TCCTCGTTAT TTTGAGAACG CTTATGAGGA |
| 40141 | TCTGGAGCTG GATTACCCTT AGCTGCTTTA CGGTAAAGCT TAAGGTCATC TTCAATACTC |
| 40201 | CGAACTTCCT TTTCAGCAGC TTCAATTTCA GAACGTAATG ACTTAGCTCG TGTTAAGTCT |
| 40261 | TCATCAGTTG CGTCTTCATT TGAGAGCAGT TGGCGCATTT CATTAGTTTT TTCGTTAATT |
| 40321 | AATGCTCGCT TACCTTCTTT TTGAGCAAGC AATTCTTTAA TTTTTTCGCG AAACATTTAA |
| 40381 | TTTCCTCCTT CGAGTGTCTT CAATAATTCT TGTCGTTCTA ATTCACGTAA CATCTTTTTA |
| 40441 | CGTTTCTGAT CAGTAGCATC TCTATTTTCT TGGCTCTGCA TTTGCTTAAC CATATTGATC |
| 40501 | GAACGCTGAC CGACCTGAAC TTCTGTATCC GGATAAGCAG GTGTTGTAAC TACTGATACA |
| 40561 | TCGAACAAAC GATCAATTTG TCGAATGGTT CGTTCATAAT CAACTCCATC CTGGTCAGAT |
| 40621 | TCTTCCCAAT CTTGGGCCTC ATCTGTGTTG GCCACCGTAA AAGCAAAGCT GCATTGATTA |
| 40681 | ATAACGCCTG CCTTGATATT GGCGATCAAA TCACGCGCAA ATGATGTATC AGTTGGCTTA |
| 40741 | ACAGTGAATT TCAAGCCAAT TGCATCAGAA GATAAAGTCA TGTTCACCCC TGATCGTCCC |
| 40801 | AGTACCTGGT TAGGATCGTG ATTGATCGTC GCTACTACAT TTGACATATC AGCGTCATCG |
| 40861 | AACGCTCCTG GAGCAATTTG CTCAATAAAG CGAGTAAAGC CACCCAAAAC TTCGGATGGC |
| 40921 | TTATTGTATT TGGCTGCATA ACCTTCAATT ACTGGTTCAT CATCTTCATC AGTTGCCGTT |
| 40981 | CTCATCTGAA TTGGCATCAT TAACTGTCGA GTTTCCAAGT CGCTTGTCAT CGATTCCACC |
| 41041 | TCCCTTCGCT GTTGATTGTT TCTGATACTC TTCTTTTTTA TCAAGGAAGA CAGTATTCAA |
| 41101 | GGTTGATTGG AAACGGTCTA AGTCTGGATT GTCAGATTTG ACTAACCCCA TCCGAACTCG |
| 41161 | TCCTTCATTA GGAGTAATGA CGTTATTTGT AACTCCCTTT TGTACATCGT CCATTGACAT |
| 41221 | CCCTGTTTCT TTGCGTGTAT CAAATTCGAT GTGGCAGTTA TGACGTTGCC GATCAGTCAG |
| 41281 | CATTGTCATT TCCAAATTAC TTGCAATCGG CTTGAAGTAA TAAGGTAAAT CAGAAGTAAT |
| 41341 | AAAGTCTTCA TTTAATTGTT TAATCGATTG GTTAGGACTA TTAACTGCTA ATTTATATGC |
| 41401 | TGGAATATGC AACGCTTTGG CAATTTGAGC AGTTGAATAG TTGTTGCTGT TAATTAGTTG |
| 41461 | CAAAACATTG GTATCAATCT CAATTGGCGA GTAATCGAAT GTATCATCAG TAACGATTGG |
| 41521 | GCTACCGGCA TTGCTATTAG CTTGTGCATA TTCAAACGCC TTTCTAGTTT TAAGACGCGC |
| 41581 | TTCCGGACTT AACTTACCTT TAGCCTTTAG TAATCCACCT TTAAGACCTG ACTTAAAGAA |
| 41641 | CCGCCGTAAA GTCTTAATTC CATCATCCTG TAATCCAATC TCATCAGCCA AAGACAACAA |
| 41701 | CGGCGACCGG CCATGAATAC CATCGTAGGT AAAGAACATA AAATGAATTA CATCTTCAGC |
| 41761 | CGGCACAACG ATTGTTTGAC CACCGCCTTT TTGATTGATA GGTGTAAATT CGTATTTAAT |
| 41821 | ATTCGTTACA TCAGAATCAT CAATATAGGT TTGCGATGGT GGAAAATATT GTATTTCTAA |
| 41881 | CGGTGCTTTT GTATGAGGAT CACGAATTAT TCTTGAAAAA CCATCCCCAG TTAAAATTGC |
| 41941 | GTTAACTGTC ATTAGGAAAC GCCAATGATA AGCAGATAAC ATATCGTTTG GGTGCTTATT |
| 42001 | CAGCAAATAA TCAACACTTT TGATATTCTT TACTGCATTT TTACCATCAT CCAAAATGAC |
| 42061 | AATCGGAAAT CGAGCTACAT TACTAGCAAC ATGAGAGACT GCAGTCAGCA CATCAGAGTT |
| 42121 | TCTAAGAGCA CCGATTCCAC TATAAGATGG CATATTGCTA AACCCCGGAA GAATCCCCTG |
| 42181 | GTCAATATAA TCTTGTGCCC ATTCGCGTTT CTCGGTATGA AATAACACTC AGCTTCACCC |
| 42241 | CCTTTCATAA GAGATAAAAG CCATTACGAA TAATTCAATT GAAATAATTA GTAAGCCAAT |
| 42301 | CTTCGTGGAA AATAAAAAGC CTGTAACTGC TAAACAGATA CAAGCTAATA TGAACAGCAA |
| 42361 | AACCGGTTCG TTTAGTTTCC AGAATTTCAT GGCATCACCC GGAGAAGAAA TTTCAAGTCT |
| 42421 | TCATCCGTAA TCTCAAATTT CTCAATATAC GGGGCAAGCT TTCTCAGTCG TTCCAATCGT |
| 42481 | CCCTTGATTT CATTAAGTGC GGGAGTTACA TCAATCTTCA TTCCTTCTTC ACCGTCGTAT |
| 42541 | AGATACTTAG CCATGTCTAT TCCTCCTCGA TTCCTTTGAT AAATTCAATT GCTTCCTTAG |
| 42601 | TTAAGGCCAA TGGATGGAAA TATTCTCCAT CTGAACAGTC ATTGATGATC TTCCCTTTTA |
| 42661 | ATAGGTGCTC CACATCATTT TTAGTGATAA AGGTGGTCCA GTTGCTATCG TTGCCATATT |
| 42721 | CATCTAGTTG CTTAATATCA TGGCTAAATT TATCTTTTTC AGAAACCAAA GTCATCATTA |
| 42781 | AACACATCCT CATCTGTTAA ATAATCATCA ATATTTTCAC GGAAACAAAT TGCATATGCG |
| 42841 | TCTAGCAATG CATCGGCAGC ATCAATCTTA TTTGAATAAC GGTTCTTATC AATCCGGACA |
| 42901 | CCATTGTTAT CTGATTTAAG GATCGCGTTA GCCATTGCGC CAGTTAAAAT TTCATTGTCA |
| 42961 | GAATGTCGAA CTCGCTTATC TAAAATATCA TCCCTAAATT GCTTGGTTGG CATTGAAAGA |
| 43021 | GTTAGTGTTC CTTGACGAAC AGATATCTGT TGCCATTCCG GATGACCTTT TTCAATCTGA |
| 43081 | GTTAACAAGG TTCCATATTG TGCGGGGTCA TAACAAATTG CCTGAACATC AAGATTGTGT |
| 43141 | TCTCTGACAA AGCCATCCAG CCATTCATAT ACCCGTTCAA CATCAATAAC TCCAGATTCA |
| 43201 | AGCTTCGTAA TTTCACACTG GCCCATCCCT TGCAACCGGA CATAATCTAA CCGGTCTGCT |
| 43261 | TTAATCTTTG CTTCCAAACC ATATTTAGTA CCGACAAACG CATAAGAATC CGCATACCAA |
| 43321 | TACCCCTCTT GAGGAATTAA CCAACTAATT GCATAAAGGT CAGATGATTT ACCAACGTCA |
| 43381 | ATCCCAAACC AAACTCGTTG ACCATCAATG TCAATTGGAT CAATTTGAGC TGCATTCCAA |
| 43441 | GTATCAATAT CCATATAACT ATCCTCTTCG GCTTGTCGCC ACATATTAAA GTTCTTAACT |
| 43501 | AGAACAGAAT TCTTTGTCCC TTTCTGTTTA GCTTCTTTCC ACCGCTTTGC CAAATATCCA |
| 43561 | TATACCTGGT CTTGCAAAGC CGGGACACTC AGAATTGGAT TGGATTTAAT CCAGGTACTT |
| 43621 | TTATCATCAA CTTCTGATAC ATTATCTTGT TCAGCAATGT AGGCAAAATA AGTATCATCA |
| 43681 | GTAATTTCAC CTTTCAAAAC CTTCGTTGCA TAGGGATATT CAATTGTATG CATTGGAACA |
| 43741 | TTCAAATCAA AGCCGGCTGT TGAAATAATC ATGATAAGTG AGTTATGAAG TAAGGCCTGA |
| 43801 | CCGGATTCTA GTAATTCCAT CATTTCAGTG GTCTTACTTG CAGCATACTC GTCTAGGATT |
| 43861 | CCAACATGAG GTTCAAAACC ATCAACCGTT CCCGTTTCCT TGGAAAGAGA CCGAACATAA |
| 43921 | GAATAATCAT CAAGGTTACT AATTAAATCA CGATTAACTT TTGTCCCACG TTTTGTATCA |
| 43981 | CCATCACTGG AACGTAGAGC ATTGAGGCGT TTCTTAATCA TATTAAAAAC GATGTTCGCT |
| 44041 | TGCTTTTTAT CGTTAGCGGT ACAAAATATT TGCCGGGAGA ATTCAGGTGA GTTACCCATC |
| 44101 | AAGAACTCAT ACAATGCAAT TCCGGAAATA AGGATCGACT TACCATTTTT TCGTGCCATT |
| 44161 | GATAACATTC CTTTTCGGAA GCGCCGTTCA GATGGCTTGT CTTTTTTCCA CCAGCCATAC |
| 44221 | ATATTAGCAA TAATGAAACG CTGAAAATCT GCTAAAGGAT ACGCCCGCAT CGTTTTTGGA |
| 44281 | TCCGGAAGTA TTTCCATGAA TTGAATAACC TTATTAGCAC GTTCATTATC ATAAAAATAT |
| 44341 | TCAAAGTCAT CGTTATCCGC CTTTTTAAGA TCATTTAAGT ACCTTTTTGC AGCTAAAATC |
| 44401 | ACCTTTTTAC CAGCAACTAT TTTGCCGGCC ACGACCTTTT CAGCATAATC TTTAGCGTAA |
| 44461 | TTCATCATGA AATCCTAAAC TTATCACGCA AGGATTGATG CTCTTCCTTG TCAGTTTGTG |
| 44521 | GCATGTTCAT CTGCAGCCGC GAATTAACAT TAAGCCCAAG ATCAGATGCA AGACCCTTTA |
| 44581 | TTGCCCTGGT TGCTTTATCA ATTGTCTTAA TTGCGTTATT GATTTTAGCA ATGTTCTTTC |
| 44641 | TTTTCTTGGA CTGTTCCTTA TTTAATTGAA CGGAAGCATC TTTATAAATG CTGTACCAGG |
| 44701 | TACAATACAA TTCCAATTCG CTCCGGTCCA AGTTACGCAA TGGCAGCTTT CCGATAGAAC |
| 44761 | CAATAATTCG CCGATATTCC GCTTTGGCCA CCTTATCTAA ATGTGCCGGC GGAGTCTTTT |
| 44821 | GCAACTCAGG TAAACCATCG GCAGCCATAA ATTCTGCCTT ATATTTTGCT TCTTGTTCAA |
| 44881 | CCACTCTAAG GTGGCCTGTT GACTGCGATA ATAATTTTTG CTTTCTTGCC AA. |

(SEQ ID NO: 11). Bacteriophage loci 3 has a sequence of:

| | |
|---|---|
| 1 | CTATACAAGC GCCCGCAATT GTTGAATCAT GCTTAACACT TGGTAAGGGG TATTTGACAT |
| 61 | GTCAATACCT CTATTTTGAT ACCAAAATTG CGCCAACATC TGAACAGCGA ACGTATATTG |
| 121 | TTCATACTGG GTTAAGTCTG CTGTGGGGTT TACAGCGTTT TGTACATAAG CCTTAGCGTT |
| 181 | CTTTAGATAA TCCTTGATTA ATTGGTCTTC TTCTTCACCG TCAAGATATA AGGCTTGCTT |
| 241 | AAAGTTGTCT ACTTCCCACT GCTCTACATC AAATTTCATT AATTTCACCT TCTTTTAGAA |
| 301 | CACATCTAGG TCTAACTTTT TAGCAACATC TACTATAAAT TGATCACGTA ACTTGTACGC |
| 361 | TTTTGTAGTG CTACACCATA TATCGCCAGT TGCTACTAGG TTCTTTATCG TCTTATGATT |
| 421 | AGGGTTAAAA TAGCATTCTT TAATTATCTT CTGGGTACAG TCCCCAGCGT CTTGTAATGC |
| 481 | TAAGTCAATA GCTTCTTTCT CACGTTTTAA GCGGTGCATA TATCTATCTT GGTCAATGGT |
| 541 | TATTAACATA TCATCGTAAT AAGTCCATGG TCGATTGATA GCACGTCCAC CACCTACATT |
| 601 | TTCATCTCTT TTGCGGATAG GGTGCATTAG TTCAAACTTA CGGCTTTTTA GTTTCTTATT |
| 661 | AATTGAAGGA TAATCTTTCA GTATTACTAT TACTAGACTT TTTACTTCTT TTCGCATTAA |
| 721 | TACACCTCAT AAAAAAAGAG TGGGCGATAT TGCCCACCCT GTGCTAGTAG CTATTATTTA |
| 781 | CTTTCGGTCG CCTTAGTTGT GGTTAAAGAG AGATTAAAAG CAGCGTCATT ACTAATTGGT |
| 841 | TGGTAGTCAT TACGGACGAT AACAGATAAA CCTTGACTGT AACTGTCGAA CTTGTCCCAT |
| 901 | TGGGCTGTAA CTTGATTACG CCGGAATACT GCTACCGCTT CTGAAATATC ACCTAAAATC |
| 961 | ATTGGATGTG AACCGTCTGT GTTATCTGGT AAGAACTTAT TACTAATCTT AACAACGGGT |
| 1021 | TGGCCAAGTA AAGAAAATCC ACTAGGAGCG GTTACATCTG GTTGTAATAA GTAACGTCCT |
| 1081 | TCATTGTCTT TCAAAGTATC TAATACTTGG AAAGCTGATT GATTTACTAA CCATGTAGAA |
| 1141 | GTACTTTGTA AGGCTGGGTC AAGGTCAACG TTGAATACTT GCTTCAAGTC GTCAACATTA |
| 1201 | GCTACGGTTT TCTTAGTAAA GTTATCGCCT TGTAATACCT TGATAATTTC AGCGTTATCA |
| 1261 | GTGTTATCAA CTAACTTTTG AAGTTGGTTC TTTACTTCAG ATACAATATC AACTTCTGAA |
| 1321 | TCGTCAACAA CTTCATTTGA TAGTGCAATC TTACCGGCAC GGGTCTTTAC ATCAAACTTC |
| 1381 | ACATCTTCAA ACATATTAGC GTCTACATCA GCAATTTCTG CTTGTTCTTC CTTAGTAGTA |
| 1441 | AGTACCGCTG TGTTGTAACG GGTAGCGATT GGATAATGAC CGGAACCAGT CCCTACAGTC |
| 1501 | TTTACAGTAG CGTACTTTGC TAAGTTATAA GCACTGTTCT TTAAGTCTAG GACTGGAGTA |
| 1561 | ACAACTTCTT CTGGAATAAC GGCTTCTGCC CCCTTAGTTG TTAATCCGTC ACGGGTTTCA |
| 1621 | CCACGTGAAC GAATGAAGCT TTCAAAGGCG TTAGCTGGTT GTTCTGTATT GTTTTTGGGG |
| 1681 | TCTAAGATTG TGTGTTGCAT TTTATTATGT TCCTCACTTT CGATAAATTG GTTGTAACTA |
| 1741 | CGAATGCTTG TTTTATCTAC ATCAACATTT GTCTGATCAT ATGAAGGCAC TGTAACGGTT |
| 1801 | GAAACCTCAA ATAAATCTTT AATCTGATTA ACGGTGCGTG TAATTTGTCC GTTGTCGTCA |
| 1861 | CGTGTCCAAC TGTCAGAACC ATCATCAACA TCAAAACGGA AAGACATAGA ATTAATGTTC |
| 1921 | CCATTCTTAA CATTATTAAG AGTGTCACTT GCGTAACTTA CACTTGGGTC AATAGTTGCC |
| 1981 | TTGAAGTGAA GCCCCTTATC GTCCACATCA AGTTGTAATG AACCAGCCTT AACGCTTGCT |
| 2041 | AGTGGCTTAG TGTAGTCGTG TTGATCAAGC ATAACTACAT TGGATAGGTC TACGTTATCA |
| 2101 | AGGGCGCTAG GGTCAATCAC TTCAACAAAA CCGCCTAAGT CCTTGCTGGG TTGATTAAAG |
| 2161 | AGTAACGCAT AGCCTTCAAC TGTATTAGGC TGTGAGGCTT GCGTTTCATC TTGGTTATCT |
| 2221 | TGCTGGTCTT TTGTTGGGTC TTCTTTAGGG CTTACCGTTT GTAAACCTGC GTTAGTTGTT |
| 2281 | AGTCGTTTCT CCATTGGCTG TAACTCCTCC TTCTTTTTCA ATAAAATTAT CTCCATTAGT |
| 2341 | TACCGGCACT AAATTAAGTA AGGCTCGGGC TTCATTGCGT GTGTAGATAC CGTTAACATA |
| 2401 | ACCATCAACT GCCATCTGCT GTTGAGCGGA CGGGTCAAGG CTTAATAGCT TGCTATTATC |
| 2461 | AAAGCTAAAA TCTTGCCCTA GCTTGTAGTT CAATTCAGAA GTAAAGCAAT CTTCATAGTG |
| 2521 | CTGTAAAGTA CCTTGTAAAT ACTGAACGTT ACTTTGTTGT TGGTTGCTAT GCTCGTTTTC |
| 2581 | TACACCTAAA CGCTCGGGTG GTAGTCCGAA CGCTTTCGCA ATTTGACGGG TATTCCAGTC |
| 2641 | ATTAGAATTT ACTAGCTTTA ACACGTCTGT ATTAAGTGGT AGGCTTTTAA CGTCCATACC |
| 2701 | ATCATCAGTA ACAATTGTGT TAAGTGAATT ATTGCCGGTA GTTGCCTCAT CAAATTGTTT |
| 2761 | TCTAATGTTC TCTTTACCAT CCGAACTAAG ATCAGCTTGG TGGACTTGGA CAACTGTTGT |
| 2821 | GCCATGAATT CCATTATTAA AGAATCCAGT CATTAACCTG TTTCCAGCCG CCTGTATCTT |
| 2881 | CATTTCATCT TTTAGCGCAT ACAGTGGCGA TAGTCCGACT AAACCATCAC GGGTAAAATA |
| 2941 | CTTAAAATGT AAGATGTTAG AAGAGGCAAT TACTCGCTTA ATACGTCCAT CTGGGCTGTA |
| 3001 | TGTGTAAGTC AGCTTTTGCG TTACATCATC TTGCTGGACA GTTAATTTAT TGTTAGGAAT |
| 3061 | AAAATTTAGC GTGTGTGAAC TTAAATCAAT TTCGGCAAAC GCATTACCAT TTAATAGCAT |
| 3121 | TTCAACCGCT AGGGCGTACT TGAAACTTCT GCCGTCCATT AACTCGTTAG GATGATCATT |
| 3181 | AATCATCTTG TTATATAGGC TAGAACTAGA CTTAATCGGA TTACTGGCAA GATCACCAGC |
| 3241 | GATAGAACTA ATTGCAGCAA ATATATCACT ATTACGTAAA GCCGAAGCAC TAACGTAACT |
| 3301 | ATTAGGGTCA TTACTAGAAA GACTTACTAG AGCGTCTAGA AAGGGGTCTT GTGCGCTTGT |
| 3361 | AGTCGTTGTT CTCTTACTAA ATAGGCCCAT TATCTACCCT CCTTTACTTG TGATTATCAG |
| 3421 | AATAGTTAAT CATGAAAGCT AGAACAATCA TTTCAATACC AAGAAAGCCA ATCCCAAACA |
| 3481 | ACCAACCGGT TAATCCTATA GCACTGATCT CCATTAGAAC GCCTCCGGTG ATCAACAAAA |
| 3541 | CCGGTTTAAT ATAGCTAGAA AAGATTTTTA GCAGTTTCAT AGTATTCATT TGTACGTTCT |
| 3601 | TGCTCCTTTC TCTTAGTGAA GTAATCATAA CCAGCCGCCC AAGCATCCAT AAGAGCAGCA |
| 3661 | AGGCAATCTA TTTTATTTTG CTTTCTAGTC TTGTCTAATA ACGCTTGACC ATTAAAGTTA |
| 3721 | TATTTCAATA TTGCATTTCC TACGTTGTAA GCTAACATCT GATTGTTAGT GTGAAGAATG |
| 3781 | TTACCAGCAA TTAATTCTTC CTTAAAAGTC CCAATTGGTA CCGATAAATT CTTATAGGAT |
| 3841 | TGTGTTAATT GAAGTTGTGG TAGTCCTGCC CGTTCAAAAC GATTAACAAG ATCAGCCCAT |
| 3901 | TTATAAGGGT CAAATACTAA GAATTTAACA TTTAGATTAT ACTTACCGAT TAGGTCCATC |
| 3961 | ACGAAATCAT ATACGGAATC ATAATCAATC AAGCCACTTT CTAATGAACT GATTGAACAT |
| 4021 | TCTCCACGTT TAGCAGCCAT TTCATAATCA ATTCCATCAC GTTTTGATTT AGTAACAATT |
| 4081 | CCACCATAAC GAGATACAAA GGCGTGGGAA TCAGCGTAAA ACTTTCCATC TTCTAACGGA |
| 4141 | ACTAACCAAC TAATCGCCGT TAAATCTCGG CTTTTTGATA GATCAGCACC AATATAAACA |
| 4201 | TCTCTATTAC TAATATCTGG AGCTTTTTCA ATCAATCCAT GTTCCCAATC TTGCGTACTA |
| 4261 | ATATATGAAT TTTCATTCTG CTGTACAAAC ATATTCATAT TCTTTACAAG TACGTTATTA |
| 4321 | ATATTATTTT GTTTTGTGGC TACATCTAAA TCATTTTGAA TGTGTTCCCT CATTGTCTTA |
| 4381 | GCAATCTTAG GATTACTCAT CAAAGGATTA GCTTTAATCC ACATTGTGGG GTCGAATATT |
| 4441 | TCTTCTTTTT TATCAAGACA ATACATAACC GCAAAGTAAC GATCATTCTT AATTTTGCCG |
| 4501 | TTGAGTATAT CGGCTGCATA GTCCCATTCA TTTTTGAAGG GTGAATTAAG ATTAAAACCA |
| 4561 | GCAGTAGAAA TAATCGCTAG AATACCATTG TCTTGTTGAG CCATACCTGA ATTCAAGGCG |
| 4621 | TTTAGAATAG TGTTATCTTT AGCTTGGTGA TATTCATCGA CTACGCCCAG TGTCGGGTTA |
| 4681 | TAACCGTCCG TACTGTGGGT ATCAGTAGAT AGTGGAATTG CGAAGCTACC TGTCTTTTTG |
| 4741 | TCAGTCACCC GTTGTTTATT GATTTTCACT CGTTGCCTTA AATACTTTGA ATTACGCCGT |
| 4801 | AAGTTTTCTA GTTGGTCACT CATAATATCA AACGCAAGGT GTGCCTGCTG GGAACTGTTA |
| 4861 | GCAGTAAATA GTATTTGTCG ATTACGGGCT GGTTGCTTTT CCATAAGAAG AGACACAACG |
| 4921 | CCAATCGCTG CCAGTAAGAA GCTCTTTCCA TTTTTACGGG CTTCACTTAA TAGAATCCGA |
| 4981 | TTGAAGCGCA ATGAATTATC GTCTACCTGT CGCCAACCAT AGATATTAGA TAAGATAAAC |
| 5041 | ACTTCAAATG GCTCTAGCTT CAATGGTTCA CCGTTTGTTT GTGGTAAGCT CTCAACAAAT |
| 5101 | TTAATAACCT TACCAGCATA CGTTTCAGAA TAATAATAAG GAAAGTCATC GGTCTTTTGC |
| 5161 | TTTTTCATGT CGTCTAAGAA GCGCTGGCAA GCATGAATAA CTAACTTACC GGCGATTAAC |
| 5221 | TCACCTTTAA CAACTGAATT AGCATACTGA ATAGCTCTAT CCATTATGAC AACATCTCTT |
| 5281 | CAAACTCATC AGTAGGAGCT TCTTCACTCA TTGCCTTGTG GACTTCTAGG CGTACCCGTG |
| 5341 | ATTGTGGGCT AAGTGCCATT TGACTATCAA TTGACTTCAT TTCTTTAATA GCCTTATCAC |
| 5401 | GTGCCATGTA GTAAGGTGAC TGCTGTAATC CTCGTTTTGT ATCAATAACT ACCCCAGTAC |
| 5461 | GATCAATCTC TTTAGTACAT TTGATAATTG TTGAATACAA AGTACAGTGA GTAGCGATTA |
| 5521 | AACTAACATC TAGTTCGCTA ATAGGGGCAC TCTTCTTTAA GAGTGGGACA ATTCTACGCC |
| 5581 | ATTCCTTTTT TGCTACATCA TCAAGCCATT TAGGGGCGTG AGATGTTAAG TTTGGGTATT |
| 5641 | CTGCCAATGC TTTTTCAGCG TCTTCACGTT GTGCTTTTTC TGCCTTAGTA ATATGACCTT |
| 5701 | CTGGACGGGT CTTCATTGGT TGAGCCAACT ATTTAACCTC CTTCTATTTA TTTGGTGACA |
| 5761 | CCATGTCACT ATTTGTTTAA AAAAATAAAA AAAACATCAT TTCATATTCA ATTATCGTAT |
| 5821 | TATTTAACCC TTATTATACC ATATTTGAAT AGTTTGTTAA GTTCAATGCC TAAAAGTTAT |
| 5881 | AATAAAAAAT CCTTGACATT TGAGGCTCTA AATTATTTAA TAAAGTTGAA AAGAAAACCA |
| 5941 | TACAAAATAA AAAGAAACGT CTTGAAATCT TCGGATTTTA AGGCGTTTTT TTGTAAAGGG |
| 6001 | AGAATTTTTT TAAAAGAAAC CCCCACCATT ACGGCTCCTA CCGGTCTATA CCAATAGCGG |
| 6061 | GGGTATTAAA ATTATTTCAG CTATTCATAT TTATTTTCCT TTCTCGTTGA TGATCTTTTC |
| 6121 | TTGTCTTTTC TCGGTGACAA CGATAGCAAA GTGATTGTAG ATTAGTTGAA TCCAATCTTT |
| 6181 | TACTCCAATC TTCACGTATA GGTACAATGT GGTCAACTAA ATCAGCTTTT TTTACTATTC |
| 6241 | CCTTTCGTAA ACACATAACA CAAACTGGGC TTTTCTGGAG CGTTTGACGG CTTAATTTAA |
| 6301 | CCCACTTACT CGTTTTGTAG AATCTATTAT AGTCATGAGG AGCTTTGGCG CTTCTTTCCA |
| 6361 | TTCGCTTTTT GTATACTTTT CTATTGTATT CATGTTTCTG TTCTTTGGTC ATCTTTTTAT |
| 6421 | AGTTATCTCG TTGCCAATGT TTACGCTTGT CTTCATATTC TTTTTGTAGA TATTGATGTT |
| 6481 | TTTTACAAAA ATATTGACCT ACTGGAATCA CCTCTCGACA ACCAGCAGCA CTACAAAACC |
| 6541 | TTACTACACT CATTTGATAA CCTCCTATGA AAAAAGGGCA TCGCATAACA CGACACCCTT |
| 6601 | AGCACTTACT TATTAATTAA TTCCATTGCC TTACTATATG CTTCAAGCCT GCTTATGTGC |
| 6661 | CAGCTTTTAA CATCGTTAAC CGTTCTCTTA ATGCAAGCTT CAAGCTCCAT TGTATCGGCG |
| 6721 | CTGTAAGGGT CACACTCGCT TAGTCTATAC CTTGCCATCA TCACCCAGTC TTGGCGACTT |
| 6781 | TCCTCTGCGT CCTTGCATAC TGCCACTTGT GCAATACACT TTACTAGCTC TTGGTTGTCT |
| 6841 | GGTAGTCCTA GATCAATCGC TAATGGTAAT GGACTGCCAT TTACTGAGAC TAATTTTAAA |
| 6901 | CTATAAAGCG TAGTATACAT ATGATAATTA GAAGGATCAT CACCGCTTGC CTCAATCATT |
| 6961 | ACATTAATAA AGTTGTTATA CTCTGGGTGC GTGTTGACTG TTAATTTACT CATCCCCATT |
| 7021 | TCACCTCTTA TTTATGACTA TCAAGCTTGC GTAAGGTTAC AATATCATAC GCCAATAACT |
| 7081 | CATCATCAAT TGACCAACCA GCAACTTTAT AGATACTGCC ATTGTATTGT GCTTCTGGAT |
| 7141 | ATTTCTTCAT GTCCGTTAAA TCTTTCTTGT GACGAACTAC CAATATAACA TCATCAGTAA |
| 7201 | TTGAACTACC TGCCTCACTA ATAGCTTGAC TAGCACTGAT TGAATAGTTA CCACAATGTA |
| 7261 | CTGTCACATC TCCTACAAAA CCTGTCATGA TCATACCAGT ATTCGGGTTA CGTTTCCCCG |
| 7321 | TACCACCTTC ATGACCGAAT GTGACCTTAT GCTTTAATCG TGTAATTGCG TATTTATTAC |
| 7381 | TTCGCATTTA ATTATTCCTT TCTGCCTGTA CTTGCAGATA CAAGCAATAG TAGAACGTTT |
| 7441 | GCAGCCGTTC TAGTGAAATC TACTGTTACT TTATTTTTAA CTCCTTAATA GGCTTTTCAC |
| 7501 | CTATCCGGTG GTATGTGGTC ACTGCCTCAA AATAAAAGTA CGGTAAAAGC AGTTTTAATT |
| 7561 | GTTAATTTTC ATCTCTTCAA TTGAAAATAG TTTTTACTTT TATCGGTTAC CCCGTTTCAA |
| 7621 | GTACCCACAA TCGCCTTTAT ATCAACATTT ATATGGTAAC CGATAGGATA TAAAGGTAAC |
| 7681 | CGATACATTG CAGTTACTTA ATATAGTAAC CCGAAACTAT CGGTTACCTC AAAATGCTTT |
| 7741 | CGGTTACCTT TAAATAGCTT TATATCAAGC TTTATAAGAA CTTCTGCAAA GGTAACCGAT |
| 7801 | AAATATAAAA CTATAACTAA TTTTTAAGAG CAATTCCTTT ATAACCTTTA ACATTTTTAC |
| 7861 | CGTTAATCTT AACTGTAGTG TGTTTGTATC CTCGTGCTAT TAGCTCTTTC GCAAACTTAC |
| 7921 | GATTACTAAG ACAAGGAACA GCAGTATTTT TACAATACTC TTTGTAATTA TCATATAGAT |
| 7981 | ATACTGATTT TTCTTTATCT TTCTCATTTG TAGTACAGCA ATCATTAATC CAATTACCGA |
| 8041 | TATTGTCATT AGCATTTACC CAGCTTCGCT TTTGCTGTTC CATGTATTCA GTCACTGGAA |
| 8101 | ACTTACCAGA ATCAAGTGCT TTTTTATAAG ATTTTAAGCA TTCATAGGCA AATGCTGGCA |
| 8161 | TTTCTCTTTT AAAGTCTTGC TCTTTGAATT GCTCTTTAAA ATGTTTAATC TTATGGAACG |
| 8221 | TTACAATAAT TGGTCGTCTT TTAAATCCGT CTGTAAAATC ATTGAAGGCT GGTAAATCAT |
| 8281 | TAGCGCTAAA AATCAATTTA GCTTCATTTT CAAGTTTAAA TGGATCTTTA TATTTAAATT |
| 8341 | GAGCTGTTGT AGTGTCGTCA CCAGTTAATG TTTTAATAAT ATTAGTCTGT TCCATAAATT |
| 8401 | TGGGGCTTAT ATCAGCAAAA ACATTAGCAG CTTTATGATA CAGCTGAGAA GTAGCAAATT |
| 8461 | TAGCTTCTTT TTGGTCGGAT AATGCTTCTA AAGAAACGTT TGAAACGTTA GGCTTCCCTA |
| 8521 | TTAACTCCAT TAGTTTATTT AGAAATGTAG ATTTACCATC CCCACCCAAC CCGTATAGAA |
| 8581 | TCATATAAAT TTGAAAGTCC TTAAAACTAC CTGCTAAAGC AAAACCAATA AAGGCTTTTA |
| 8641 | CTGTTTCAAT TGCTGTTAGG TCATATTCAT CTTGTAGTTT TCCAGTATCA TCACGGATTT |
| 8701 | CTTTAGTTGA GGGTACTAAT GATTGTTTTA ACCATTTATT CCACGTTGTA GCTTTTCCTT |
| 8761 | TTGTTTCTAA TTCATATGGT CTATTTTGTA AAAGATAGTT CTCTGGTGAA TTATCTTCTA |
| 8821 | AAGTGTCCGT CATCAAGTTG TAAGTGTGTT TACCAAACAC TGCTTTATAA GGTGCTGGTT |
| 8881 | GATCAAATGG ATTGTCTTCT ACAATTTCTT GTGAATCTAA GGCAATTAAT TTAGAAACCG |
| 8941 | ATGCTAATAA GTTCGTTCTC CATATTCCAT GAGGTACAAT TTCATTTTTG ATTGATTTTT |
| 9001 | CAATGACTGA ATCAGCGTAA TTTTTCCATT GTCTACTTTT AGGATCGAAC CAATAGCCGT |
| 9061 | GTTCTATAAA TTGGTTGGAT GTAAAGTGAT ACTTTTTTTC TAAATATTTA GCCAACTTAA |
| 9121 | TTTCATCAAC CAGTTTATTT CCTTTATCAC TTACAGTTAA CCATTCTGGA AAACTATTGT |
| 9181 | TATTTACTAA GTCATCAATA TTAGCCATAT TTTTCCTCTC TTTACATGGT AATTTAGCGT |
| 9241 | TTGTATTTAA GACTGATAAT CTCTACTTCG CCTTTTGTTT TGATATATTC AAAGCTTTTC |
| 9301 | CCGTCTAAAC TTGTTTCTTC GTATATATTG TCCCAATCGA CTATTGATTG ATCTTCGTTT |
| 9361 | TGACTAGCAT TATTCCAATA CTTAAAGAGT GGTGTACGGT GCAGTATTCG GTTAACTTGT |
| 9421 | TTAACGTCAT CCCCTGTGTA GTAGAGAAGA TAAAAACACA TTGCACCAAT AGCAGGTTTA |
| 9481 | ACGCTATGAT ATTTATTTAG GTAGTAGGAA ATTATTTCCT CATTATTTTT ATAAAGAGCA |
| 9541 | ATAAATTTTC TACTACTTTT ACTACTAACA GCTCGTTCTA TAACCTTGTA ATCATCGTTA |
| 9601 | TAGTATTCAA GTGCTTTCTG AAACTGTTTC TTTGCGTACT CATTAAGGGG ATGTGACGTT |
| 9661 | GAATTAATAA TTTTTTCCAT CTTGTCACTA TCTAATAAAG GATTAACGGA TAGTTTTTCT |
| 9721 | TCAAAACTTA ACACTATTAA TCACGTCCCA CCTATTGCAA TTGATCATGA ATTTGATCTA |
| 9781 | ACTTATCCAA CAAAATACTA GATAGTGCTA ACAATGCTTT CTTACGTGAT ACTACTGTTT |
| 9841 | GAGCAACCGT AGACACTTGT TCATCACTCA TTGCTGCATA ATTGTCTACT CGTACAATTG |
| 9901 | GAGCAAAAAC ATCATTTAAC TGATCATACA GTGCTTGGGC TATTCCGGTT GTTTCTTCAA |
| 9961 | CATTGGAATC TGCTTCCCAC AACTGATTTT CAAACGCTAG TTCTTGGTTC TTATCTTTTT |
| 10021 | CCATTGTTTA AACCCTCCAC TCATGTTAGA ATGAAAGAGA ATTAAATATT AGTAATTCGT |
| 10081 | CCAACATCCC CCGTAAAGAT TGTTGGCTTT TTTATTAGCT AAATTTAATT CTCTAAGTTT |
| 10141 | TATTTAGTCG TTGTTAGTGT CTGAATTGGC GTTCTCACTA TCAACGGCTT TTTTATTACC |
| 10201 | ATCCATAAAG TCGTTTTCAA AATCAGTTAA AGTTTTATAT TGAATTGCTA AATCTTTAAT |
| 10261 | TTCTTTAGCG ACCTTAATAA TTTCATCTGC TGAGGAAATC GGATCATCTG CAATTATTTC |
| 10321 | TCCATTTTCT TTAGTGATAG TACCTTCAAA ACAATCTTCA GTAATTGAAT TTATATAACT |
| 10381 | GCCAGCTTTA ACAATCCTGT CTTCTATATC AATAATTCTT TTACTTAATA TTTCTTATTC |
| 10441 | AAATTTTTTA GTGTATAAAC TCATTTCTTA TTACTCCTTA AAATAAGTTA CTAATTGCAA |
| 10501 | CAACAATACC CAGCAATGGT AAAAACGTTG CTAGATAAGC CCAAAGGCGT TGGTCTGGTG |
| 10561 | AAGTCCACCA GTAAGCTAAA ATCTTACCAT CAATTTTTTG TAACATATGT CTCACCTCCT |
| 10621 | CCTATTTATT TCTCTACAAT CACTTGCCAG CCAGCTGTGC GATTATTACT TAGAATTTTC |
| 10681 | TTTTACCCAA CGGTCAACAT CTTCACGTTT ATATACATAT TTTCCTCCAA CCTTTGATGA |
| 10741 | TGGAAAATTT CGGGTCTTAA TCCAACGATC AATAGTTGTT CGAGAAACAC TTAGATAATC |
| 10801 | CGCAGTTTGG CCCTTATTCA TAAACTCTGG TTCGGTTTTA ACAGTCTTAG CATCTTGTAA |
| 10861 | CTTATTGTTC TCTAGCTTTT TAGCCACAGC TTCAGCTAAC TGATCAATGA ACTGATCTAA |
| 10921 | AATAGCTGTA CTCATTATTA TCACCTCTTT ATTTCATATC GTAATTACAA TATATAACAT |
| 10981 | AATGAAATAT TATGCAACAA CAAAATAAAA TATATTGTGT TTTGTATTGT TATTTTCGGT |
| 11041 | TATGTATAAT ATATTAACTA TACAAGGAGG AATTAATGTG AAAGAAATTT CATCTGACCA |
| 11101 | GAAAATAGGT TCTAACATTG CACTAATTCG TAAAGCTAAA AAGATGACTC AAAAAGACTT |
| 11161 | AGCAAGAAAA ATTGGAATAT CACAACCAGC CTTGGGGAAT TATGAACGTG GCCAGCGGAT |
| 11221 | TATACCTGCT TCGATTTTTC ACAGTTTACC TCAAATATTA GATGTTCCTC TAGATTTTCT |
| 11281 | AGTCTATGGA ATTCCCAACA ATATTACCTT TCATGATTTA GCAACGCTCG ATTCCGTATC |
| 11341 | TCTCAAAAAG AAAGCTGCTC ATATTGAAAA TGATAGAAGT ATTATTGAAC GTGGGTTAGG |
| 11401 | GCGTGACGAC ATAAAAAGAC ATTTATTAGC TGCTGTTTTG CTTGATAATT ATGGTTATCT |
| 11461 | TGTGTACCCA GATGAATCTA GCAATGACAA AGAACATGAA CAACTTAAAA AATTTGTTTG |
| 11521 | GAATACATTA GATGGATTAC TTACCGGCGT ACCAATTACC AATGAAATAA ATTTTAAACA |
| 11581 | AATTAATGAA CAATTTGAAT TACTTGCAAA TACGTATTTA AGCCTAATTA TTGACGAAGA |
| 11641 | GGCAAATCGC TATCCTGCTT TCCGCAAGGA TTTACCACCC TTCAAAGAAA AACTTACACA |
| 11701 | GTTTTTAAAT GATAACGGAT ATTTTAAAAT TTTAAAAAAA TAATTTTAAA CCTTTCTCTA |
| 11761 | CAATCTCTTG CCAGCCAGCT GTGCGATTGT AGGAGGAAAG TAAAAATGTC AGTATACAAG |
| 11821 | GATAAAAGAA ATGGTACGTG GTATGTTAAA TACCGCATTA ATGGTAAATC AACCAGTAAG |
| 11881 | CGAGGTTTCA AATTAAAATC GGACGCTGTA GCGTGGGAAC AAGAAAAAGC TGTGGAGCTT |
| 11941 | AAAAAATATG GTCAAATGAA TAACCATAAC ATGACCGTTG AAGAGCTATC AAAAAAATGG |
| 12001 | TTGCCAGTAT ATGAACAGAC TGGGATAGAA AATTCTACTA TTCATAAAAC AAAACAAATC |
| 12061 | ATTAAAAATC ATATTCTACC CCGCATTGGA AACTATAAAG TTGCTGATCT TAGCATTGAG |
| 12121 | TTATTAACTG AAGTTGCTGC TAAATGGAAA GAAGAATTAG TAAAGAGTGA CCCTTTTAAC |
| 12181 | TATACTAAGC GAATGTTAGA CTACGCAGTA CAATTAAGAG CGATCCCAAC TAATCCAATG |
| 12241 | AATTCTGTAG TAAAGCCTCG TAAAAAACAT GACAAAACAT TTACTAACAA TAATTTCTTT |
| 12301 | AATGAAAAGC AACTTCACAA TTTTATCAAG TGTATAAAAA AAGACTACGA AGAAAAAAAT |
| 12361 | CCTCGTGCTT TCATGGTTCT ATGGTTAGCC CTTTTTACTG GCATGAGAAA ACAAGAACTT |
| 12421 | CAAGCCTTGA CGTGGCAAGA TGTTAAATTT ACAAAAAGCG GTGGAATAAT CCATATTAAC |
| 12481 | AAGGCTATTA AGAACGCAAA ACATCCCTAT CTTGGTGGCC CTAAAACCGC TAACTCTTAT |
| 12541 | CGCTGGATTT CAATAGATAA AAAAACTGCT TCCTATCTAA AAAGATGGAA ACAGCAACAG |
| 12601 | ATAGATATTT TAACTAAACT AGGTTTTAAT CCACACCAAA AAGAGCAATT GATTTTCTCT |
| 12661 | ACCTACACTA AAAATAAAAT TGTTTTAGGT GCAGAATTGG ACAAGCCGTT AAATAAAGTA |
| 12721 | ATTGTTAGAA ATAATCTAAA GAAAGTTACA TTCCATGGAT TACGTCATAC CCATGCTACA |
| 12781 | CATTTGGCTT CTATTGGTAC CCAGCCAAAA TTAATAGCAG ATAGATTAGG CGATACAATG |
| 12841 | GAAACTGTTC TTGAAGTTTA TATTAATGCA GATACTGAAC CAGAACAAGG AATCGCCGAC |
| 12901 | AAGTTCGCCG ATAGTTTAGT ATAA |

(SEQ ID NO: 12). Bacteriophage loci 6 has a sequence of:

| (SEQ ID NO: 13) | |
|---|---|
| 1 | TTAAAATTGA CTAAGTGCCT GGACGATTTT GTCATCAGTT TTACTTTTAT ATTCATCAAT |
| 61 | CAAGTAGGCG TATGTGTTCA TTGTGGTTGT GATATCATTA TGTCCAAGTC TCTTGCTAAT |
| 121 | TGCATAAATA TCTATTCCGT TACTTAACAG TAAAGCAACG TGACTATGAC GTAAACTATG |
| 181 | GAAATGAAAA TTCTTCTTTG ATAATTTTGT ATCACTCATA ATTTGTCGAA GCGTTTTATT |
| 241 | TAATGCTGTA CTAGTAGGGA TAGTATTAAA TTGATCCATA AAAACAAGGT TACTTGATGA |
| 301 | ATTATTGCGC AATTGTTTAA GTAGTAATAG TAGTTTTTCA TTAACTTTTA TTTTACGTGT |
| 361 | AGATGATTCA TTCTTGGTGG GTTTAAAGCG ATGTGTATTA AAATGTTCGT CTGTTTCGTC |
| 421 | TCTTCCTTTT ACCTCTCTCC ATGCTTTATC AATATTAATG GTATGATGAA TGAAATCAAT |
| 481 | ATCATTCCAG GTAAGAGCTT GAATTTCTTC TTTTCTCATT CCAGTATATA TCGCAGTGAC |
| 541 | TATCATATAA CGGCTTGTAT ATCTTCTATT AGTAATTCCA TTGATTGTAG TAGTTAATAA |
| 601 | TTTCCTTATT TCAGTTACAT TAGGGTATTC TACCTTCATT GTTTTGCTAG TATTGGCAGC |
| 661 | TAAAGTAACA CCTTTAGTAA AATCTTTAAG CAGATAATCA TCAAGGATAG CAGATTGTAC |
| 721 | GCAAGACCGT ATTATTGAAT TAAGCTTCTT TACACTTGCT ATAGCATGAG TTGCTCCATA |
| 781 | CTTGTTAATA AATTCTTGAT ATTTAGAACG GTTAATTTCT TTAATAGAAG CTTCTTTGAA |
| 841 | ATAATCATTA ATTAGATTAC CGATTATAAT ATAACGATTC AGAGTTACAC TTGATATTTT |
| 901 | AGGTTGCTTA TAAGTATTAA CCCATTCTTC GTAATAATCA CTAAAAGCAA TTTTTTTCTC |
| 961 | AATGTGTATT CCGCGATTAA GATTAGTTTC CATTTCAGCT GCCCACTTTT TAGCTAGTGC |
| 1021 | TTTTGTTGCA AAACCAGCTT TGGACTTTGA AAAACGTTTT CCTTCAGTAT CTTTCCACTG |
| 1081 | AATTCTTACA GACCATTTTC CGCTTCTTTT ATATATTTGA GCCATACTTG TCCCTCCATT |
| 1141 | ATTAAATTGT GTATAATGAA AGGGTTGATA GAATTTGTTT GGACGCAATT AATATCAACC |
| 1201 | CTTGGTCCAC TGACAGTTGC CGCTGTCGGT GGGCTTTTTT AGTTTAAATA GCTTTTTACG |
| 1261 | TCATCAATTT AGTTGGACGA GAAATTTAGC TTTTGTTTAG CTTCTTTAAT TGATTAATAA |
| 1321 | TTTCTTGATT TTGCTTAATT GTTATCCAAT TTTGCTGTTT GATTATTCTT AATTCATGAA |
| 1381 | AAAGGGCTTT TGTATCATTA CTTGTAAAAG TAGGATAGGC ACCTGTTAAT TTTTCGGCAA |
| 1441 | CTGCTATATC TTTTACAGAT TCGAGAATTT CGTCATCTAA ACTTTCTAAA CGATTTTCTT |
| 1501 | TAATATATTT ATCTTTGTCT TTAGCAAATC CATCTTTACC ATTGAAAATA CTCATTTTTA |
| 1561 | GCTTTCTCCT ATCTTTTTAA TTTTTACATT GTAAAGAGCG TTATTTAAAT CATTACAGTT |
| 1621 | CCTAAGATAG GAAAAAGCGT TTTTTAAAAG TTTATTCATC CTTTGACTCC TTATATTCCT |
| 1681 | AAAATTTGTT TCTTTTTAGC ATCAAATTCT TCTTTGTTAA TGATTCCATC ATCAAGTAGT |
| 1741 | TGTTTTAATT CTCTTAATTG CGAAAGGTCA TTACTAGAAT TGGATTGTGC TGATGTCTGC |
| 1801 | TTAAAATCTG CAGCTGCCTG TTTAATAGTC TCAGCCATCT TTTTAGCAGG GTAGGGTTGC |
| 1861 | ATATTTTCTA TTTGAGTTGT AATAGCTCCG TTGGTTACTG CAATAGATCC CAACATTAAA |
| 1921 | CCTTTAGAAT ATGAAACGCC ATTAATCATA TCAAGGGGAA TGTCAGTTGA TTTACTACCA |
| 1981 | TAAATCAGGC CGTGGTCAAG AAAAATTACG CGCTTATTAG TACAAACCGC AAGAATGGAA |
| 2041 | TTAGTTTCAA CGAAAGCATT AGCAGCATAA AGTATTTTTT CTCCACCATC GACATCTATT |
| 2101 | ATGTCTGGCA AGGCTTTGAT TTCCTTTTTA GTACCGAATA GATTTTCTAC ATTGGCATCC |
| 2161 | TTTAACTGTT GCTTAATAAC ATCCAATTTT TCTTGTTCGC CTTTGTTGAT TTCGTCAATT |
| 2221 | GCTGCTTGTA AAGTATCAGC TTTTTTACCA GCGCTATCAG CAATTGAATC AAGTTTCTCA |
| 2281 | GTTACTTTAG ATAGCTTTGA TTTATACAAA TCAGGATGTT CTTTGTAAGG TACGGTAGAA |
| 2341 | TGAATCCCTA ATAAACTTTC AATTTCACTA GCGGTATGGG AACTAATGAA ATTGCTAAGT |
| 2401 | TTAATTGGAA CACGGTAAGT ATTTGCTGGA CGTTTGGGAT CAAGTCTGTT GGCATCTTTG |
| 2461 | CCACAAATTA AATATCCATC TTTGGTAATG CAAGAAGCCT TTAACATTCC CAGGGCATCA |
| 2521 | TTACATATGA AGCATCTCTT TTTACTCATT TGGATCACCT TATTCCATTG TCACTGAATA |
| 2581 | TTTAACTAAT TTACCAACAA TTCGACCTGG ATGCTCTTCA TCAAGAATTA TTGGATCATA |
| 2641 | GTTTTTATTA TCTGGCATTA GCATAACTAA ATTGCCTTGA TGCTTAATAC GTTTTAAAGT |
| 2701 | AGCTTCGTTA TCATCGTCAA CAAGAACTGC AGCTATCTCA CCATCTTCAA CTTCAGGTTG |
| 2761 | CTCTCTAATA ATGGCTAATG CGCCATTAGG AATGGTAGGC TCCATGCTAT CACCTTTACA |
| 2821 | ACGAAGACCA AAAAGTGTTC CACTAGGAAC TGGTTTTTCA AAGATTTCAT CAGTATAACC |
| 2881 | TTCGATATTT TCTTCAGCAG TAATCGGATC GCCACAAGCA ATTTCACCAA TGATTGGAAT |
| 2941 | TGAAACAACT TCTAATCCTT CTTTAGGATA AATAATATTA GAAGGTTTCT TTTCTCTGGT |
| 3001 | AGGGAAAAAG TCATCAACAT TTACATTAAA AATTTCAGCT AATTTAAATA AAACATCTTG |
| 3061 | ATTTGCCTTT CTAGATCCTA ACTCATATCT AGTCACAGTT GCTCTTGTCG TATCAAGTTT |
| 3121 | GTCTGCAAGT TGTTCTACTG AAAGGCCACG TTGTTCACGA AAATTTCTGA TTTTTTGACC |
| 3181 | AATGAATTTA CTTAGTTCCA TTTTATGTAC CTCTCTTATG TAACTAATTA TATAACTTGC |
| 3241 | GTTACCGAAA TGGAATATTT TTTATCTTTT TCGAAAAAAA TAGTTGCATT TGTTCCAGAA |
| 3301 | TGGTTTTAAT ATATAATTGT TCCAAATTGG AACGGAAAGG AGAGATAATA TGCAGTCAAG |
| 3361 | ATTATATGAT TTACGAAAAC ATGTTAAAGG AATGACTCAA CAACAAATGG CTGATTATCT |
| 3421 | AAATATTAGT GTCAAAGCTT ATCGTGATAA GGAAAATGGT AAAAATCAAT TTACTCAAGA |
| 3481 | TGAAATGTTT GCTATTAGTA AATTATTTGA TCTTAATATT GACACTATTT TTTTGCCTCG |
| 3541 | TAAGTTCCAT ATTGGAACAA AGGGCTAAGA GAGGTGACAC AAAATGCAAG CATCGCTAGA |
| 3601 | TGAACAAGAT TACCAAGTAA TTACTAATGA GGTTCTCAGG CGTATTAAGG AATGTTACAA |
| 3661 | CTTAGTGCCT AAACAAGATG TCCAAACTGA CAAATGGGTC GGTATCAAGG AATTCACGAG |
| 3721 | TAAGTTGCCC GTTATAAAGG ACAAGGAATG GGTCAGAATG TTTCTCCTTA CACTTCCAGT |
| 3781 | CTTTAAGAAC TGGGTTATCA ATTTGAATGC AGGTCAAGGT CATCGAACTA AGGTAAATGT |
| 3841 | GACCAAGGCA TTGCCTTGGA TTATGTCTCA TCAAGCTGAT ATTGATTGGA ACCAGTCATT |
| 3901 | GCCACGTTAG GAGGTGAACA GATGGCACAA CCGATATTTG TAAAAGGAAT TTTTGGAAGA |
| 3961 | CGAATTATAG TTTCTCAATT TGAACAAACG TCTGATTTTA TTTTGCAAAA GAAAAACGCT |
| 4021 | GACCTAATAA AAGAAGCCAG CGGATTTTCA AAAAACAAGA AATTGTTTAC TCGTTATCAA |
| 4081 | GGTAAAGACT TTCAAATTAT GGAGCAATTA AAGTGGCAAC CCAGTGTTCT TTTTGATGAT |
| 4141 | GCCAGTAAGA ACCGTAGTAG CCACAGATTT AAGGACACCA ATGGAAACTG ACTTTAGAGT |
| 4201 | GTTAGTAGCT TTCTTTGTTT GTTTCCAAAC CTCATCATCT CGAATGGAAT CAAGTAGTTC |
| 4261 | ATGCCCAGAC CAAGTGATGT CATAGACAGT TAATTGGCGA ACCATTTTAT TAATAAATGG |
| 4321 | TTTGGCACTA ATGAAACCGC CTGAGTAGAG CTGATAAAGG GTATAAACAA TATCATTTTT |
| 4381 | ATCAAAGGTC TCATTAGCAA TAACATTTGC AATTTGAGAA TCTGTAAAAC CTTGGTAAAG |
| 4441 | CATTTGCAAT TGCTGTTTTT CTAATGATAA AAGAACATAC CTAACACAGT CTTGCTTCAA |
| 4501 | TTTCACAATT TTCACCACCT TTATTTTGAA TTAATCAAAG TATAGCAGAA AGGGAGAGCA |
| 4561 | GTTAAATGGC GTTGGTAATT GGATTACCAA TATTAATTAT TGCAATGTGT TTGTTATATG |
| 4621 | CATTGGTGTA CAGCTTATTG TATGAGCGGA ATAAACCATT GCTATTGAAA GAAAAGTATC |
| 4681 | GGAAGAAACA TTGAAAGGAG GTGATATGAA CTTTGGTAAA TTTATTTCTT TTGTTGTTTT |
| 4741 | TTATCTTTGT TATAATGCCG CTTTTGTATC AATTTTTGAA TTACATCAGA AGGCAAATTG |
| 4801 | AAAATTTCTT TTGGAAAAAA AGCCATAAAA ATAAGAACGA ATGAAATGAA AGTAGCTGTA |
| 4861 | AATTCAAATT GTTCTTTGGT CTGAGTTATT AATAGCCCAG TAATAGTAGA AAGAATAATT |
| 4921 | GCTATTTTTG AAAGATCATC CAAGTATTTT TCGCTTATTT GAAATCCTAA AGTTTCTGTA |
| 4981 | AGCCAGCCAA GGATAAACAA TGGAAAAGAA AAGAAGCCAG TTAGAAAAAG AATAGTAATT |
| 5041 | AAGCAATCAA ATATTCTGAG TTTTTGGAAT TCAAAAATTA CAGCATCTGG ATTAGCAGCA |
| 5101 | TATGTAATAT GTAGAGCTAA ATAAGTAAAA CAGATGAGGC AGAAGATACA AACCATAAAG |
| 5161 | TCGGTTAGTT TCTTACTATA GTAATTAAAT TTGGTTAAAA ACTTAAAAAT TTTACGCATA |
| 5221 | ATGATTTTCC TTTTGTACTG AGTTAATCAA AGTATAGCAA ATGAAAGGAT GGTAGTTTAA |
| 5281 | TGAGTAAATT AATTGCGCTA ATCGTTGGTG CTTGGATCAT GTATTGTTCA ATGATTGGCT |
| 5341 | CTTATGATGG AGCGATGGCG ATCCTAGCAG TTTATCTTCT GCTAGTGGTT CTTGATCCAC |
| 5401 | TAAATAAAAA AGGTACGACT GCCGCAAACA GTCGTACCGA TAAATAAATA ATCTACGAGG |
| 5461 | TAATTATATA TGAATGAGTT TAATTTATCA AAATTAAATG CCAGGGTTGG CGATAATTGT |
| 5521 | GTATTTGTAT CGAATTTGGC AGTTCGCTAT CAAAGCGCTG CTACTCCTGA AGAGCGAATG |
| 5581 | GCAATGGCCA TCAAAATGGA GAATGCTGCT ACGATGTTAC GCATTTCAGC TGAGCGTTTG |
| 5641 | GCCACTGAAA CTAAGAATGT TTACGGAGGT AAGAACAATG ACTAACGAAG AAAAGATTAA |
| 5701 | GGCAATTAAA CAAATTCTTG GACCAGAATA CGAGGAGGTA GCCATTTTCG CTGCTAAAAA |
| 5761 | ATCAGAAATT CGTGACGAAC GTACTAATTC ATTAGTTGAT GCGGATCCCG GAACGGTAGC |
| 5821 | AGCCATGATT ATGAATTGGT TACATACTAA TCCGATAGCA GCTTCAATTG TCAAAGCAAC |
| 5881 | TATTGATACT TTGGAAACTG ATCCAACTGC TGATTTATTT GCACAGATTT TTCTAGGAGG |
| 5941 | TAAGAACTAA TGAATTTATT TGAATTAAAC GACAACTACA AAACACTTGC TAGTCGGGAT |
| 6001 | GACTTAGATC CAACTATTCT AAAGGATACA TTGGAATCAA TTAAGGATGA TCGGAAGACC |
| 6061 | AAATTGGACA ATCTTGCTTC ATGGGCAGAT CATTTGAAGT CAGAAATTGA TTTTATGACC |
| 6121 | GACAAAAAGA AGTCATGGGA AGAAGAAATT ACTTACCGAA AAAATAAACT TACTTGGATC |
| 6181 | AAGAAATATA TTACTGAAGT TCTTGATGAT GCCGGTATTA AGAAAATAAC TACTGAAAAT |
| 6241 | CACTTACTTA GTGCCCGGAA CTTTAAGGCC TCAACCATTA TTGATAGTGA TAAGAAGCTT |
| 6301 | CCGGATAAGT TCAAAATTAC TGAGACGACT ACTAAGCCAG ATAAGCAGGC CATTTACCAA |
| 6361 | GCACTCAAAG CTGGAGAAGA AGTACCAGGA GCACATTTAA AAGCTAACCG TAACACGGTG |
| 6421 | ATTAAATAAT GTTTGAACTC CGTGATTATC AGCAAGAAAC GATTGATAAC ATCATGAATT |
| 6481 | CTATAAGTGC TGGTCACCGT TCTATCATGG TTCAACAGCC GCCACGAACG GGGAAGACAG |
| 6541 | TTATTATGGC CGAGATTGCT AGACGAGCAA CGGCAAAGGG TAACCGTATC TTGTTCGTGG |
| 6601 | TTCATCGGCA AGAAATTGTC CAGCAGGTTA TCAAGACTTT CAAAGCTAAT GATGTAAATA |
| 6661 | TGGATTTAGC TAAAATCGGC ATGGTTCAAA CGATTACCCG ACACGTTAAT AATTTGGACC |
| 6721 | CACCGGCGAT AATTTTTGTT GATGAGGCCC ATCATGTTCT GGCTAAATCA TATCGAAGGA |
| 6781 | TTCTTGATGC TTTTCCGAAA GCTTATAAGT TACTGTTTAC CGCTACTCCT TATCGGTTAG |
| 6841 | GTGGACAGGG TTTTACTGAT GTGGCTGATG ATTTAATTAT TGGCAAATCA GTCCCCTGGT |
| 6901 | TAATTGACCA TCACTTTTTA GCACCAGTTG ATTATTACGC TCCTTCTTAC ATTGATACTG |
| 6961 | CCAAGTTAAA AGTAAAACGA ACTGGTGAAT ATGACACTGA TTCAATCAAA AAAGCCATGA |
| 7021 | AGCCTAAAAT CTACGGGAAT GCGGTTAAGC ACTATTTGAA ACTTGCTACG GGAATGCAAG |
| 7081 | CCATTGCCTA TACCTATAAC GTTGATAGTG CAATTAAGTT AGCCAATGCA TTTAATGGCT |
| 7141 | ATGGGATAAC TGCAAGCGCC GTTTCCGGAA AAACGCCCAA AGAAGAACGG AATAAAATCA |
| 7201 | TTGAGGACTA TCGCCAAGGA AAAATTCAAA TTGTAACCAA TGCAGAATTA TTTACAGAGG |
| 7261 | GCCTAGATTT ACCAAATGTT GATTGTGTCA TTATGTTACG GCCAACTCAA TCATTATCGT |
| 7321 | TGTACTTACA ATTTGCAATG CGCTCAATGA ATCCACGGGA AGGTAAGACT GCAATAATTA |
| 7381 | TCGATCACGT GGGAAATGTT GAACGATTCG GGCTGCCGAC TGATGAGCGG CAATGGACAT |
| 7441 | TAGAAGGTAG CGGAAAAAAT AAACAACAAC CAGGAACAAC GCTTAAACCT GTATCAGTAT |
| 7501 | GTCCGATATG TTTTGCTTCG TTTTATCGTA CAAGTGATAT TTGCCCTTAT TGCGGGGCGG |
| 7561 | CATTAGGAGA AGAAAAAGAA ATTGAAGTCG TTGATGATGT TCAACTAAAA AAAGTTACTA |
| 7621 | AGTCACGGCT AGCGATTATT AAGAAAATTC AATCGTCAGC AATTATGAAT AATGTTGCTG |
| 7681 | GCAAGCGTCC AAACGAATTG AAGAATCTGA AAGAAATACA AGCCTATGCC AAATTAAAAG |
| 7741 | GTTACAAACC AGGCTGGGCT TACCACTACG CTAAACAGCG TGGATTTATT AAGAAGTGAG |
| 7801 | GTTGATATTA TGAGTATTTT GCCACCAAAT AAGCCACAGA AGGCACGGCG AGTTCCAAGA |
| 7861 | AATTACTTTA TCTACGGAGA TACAATGTCC GGAAAGTCAT ATCTAGCTGA ACGTTTTCCA |
| 7921 | AGTCCGCTAT TTCTTAACAC CGATGGTAAT AGTGAGATGA ACACCGCACC AAGTATTCAA |
| 7981 | TTAAAGAATG TCCGAAAGAG CGATGGAAGC TTGAAAGAGT CAGTGATTGA TCAACTAGAC |
| 8041 | AAGATTATTC TTGCTCTTGG TACTGAAAAT CATGGTTACA AAACAGTAGT TATTGATGTG |
| 8101 | ATTGATGATG TAGTAACACT AATTGAACAG GCCATCTGTT ATGACAATGG AGTAGAAACG |
| 8161 | CTGGGGGATG TTCCTTACGG CAAGGGATAT GCACAATTTA ATACCGTCTT TCAAGCATTT |
| 8221 | GTCACTGAGC TAAAAGCCTT ACCACTGAAT ACGGTTTACA TTAGCCGGTT AATGATGCTA |
| 8281 | ACTGATGAAT CTTCTGGCCA CACCGAAGAC CGACCATCAC TAAAACAGAA ATATTACAAC |
| 8341 | GTGGTTAACG GAAATTGTGA TTTAGTGATT GAAACTAAGC GCTATGGTGA CCGTTATATC |
| 8401 | CGGATGGTTA AAGATCGACG AATTCATTAT GTCAAAGATG ATATTACTGA TCCGGCAATC |
| 8461 | TTACGGGTAC TTGAACATGT AAATGGCGTC TTTGATAAGC CAAAGCAGAC TACTACAAAA |
| 8521 | GAGCAGAATG AAATTGTTAA CAAAATTAAA AAGCAAAATG TAAAGGAAGG TTAATGAATT |
| 8581 | ATGAGTTTAC GAGATGCAAT GAATAAAGCT ACTGAAGGTT TTGATCCAAA GAATGATTCA |
| 8641 | GTTAATAAAT TTAAGGGACT GGAAAGTGGT AAATATACCG TTGTAGTTGC AAAAGTAGAA |
| 8701 | AACCATGAAA CTCCTTGGAA TGCTGAACAG CTTAACTTTG AGTTAGAAGT TGTCGATGGA |
| 8761 | GAATCAGCCG GCCAAAAAGA ATTCTTACAA ATTGGATTAG ATGAATTAAC TTCTAAGGGT |
| 8821 | AATCCCAATC CAATGCTAGA AACTAATTTA CGATTGGTTT CTAAGTTAGC AGCAATTCTA |
| 8881 | GGTGTTGAAA TTCCCGATGA AGTTTGGGAT GACGATACTT TAATCTACGA GAACTTGGCT |
| 8941 | AAAGCATTTG CACCGGCAGT AGGAAAGACC ATGATTATGG ATTTGAAGGT TCGACCAAAC |
| 9001 | AAGAAGAACC CCCAATATCC ATACCGTAAT TATGACTTTG ATGAAGCGGA ACAGCCGGAA |
| 9061 | ACGCCAGAAG TTACAGATGA TGAAATGCCC TTTTAAGTAA ATATTTGAGT CAGTGAACTT |
| 9121 | ATAACACCGT ATGGCTGGGA GGCCATTAAG GAGGAAAAAT GAAAAATCTA GTTAATTACG |
| 9181 | CCTTAGCCTA TCAAGCAAAG GGTTTAAGCG TCCTCCCAAT TGCTGGCAAG CGTCCACTAA |
| 9241 | TTAAGTTTGC TGATCGTGAT CCACTTACCG CCGAAGAAAT AAAGACCATC TGGATAGAAC |
| 9301 | ATCCATATGC TCAAATTGCG TTGCGGACTG ATAAGTTCTT CGTTGTTGAT ATAGACCGCA |
| 9361 | ACCATGCTGA TAACATTGAT GGTTTTGAAT CAATTAAGCA ATTACCAGCG GAATATTTTC |
| 9421 | CGGAAACTTT AACCCAAACC ACCAAGCATG GTGGCCGACA GTTATTTTAT TTGAAACGTT |
| 9481 | CAGATATGCG AGTTAATCAG TTAATTGGTT ATTTACCAGG TGTTGATGTA AAGGCCCACC |
| 9541 | AAAATAATTA TGTTGTGGTT GCGCCATCAG AAGGTTACCA ATGGCTGAAT AAGAAGCCGA |
| 9601 | TTGTTACGGC TCCTAAGTCG TTAGTAGTGA ATATTAACCA AATGCGAGCC AGTAACCGGC |
| 9661 | GAAGTTCTCC AGATGATTTA GTTTTTAAAC CACGTGAACG CAACTCAACT ACTGACTTAC |
| 9721 | TTGAGACCAT CGCAAATGGA TTGGGAGATA AAGGAATAAG AAATAAAAAC TTAGCTGGAA |
| 9781 | TGATTGGTGC TTTATTGTTT CGGGGAGTAG AACCAAAGTC TGCTTATCAA TTAGCGATGA |
| 9841 | TTTGTAATGA GAATACGCCC GATCCACTAC CAGAAGAAGA AGTGAACCGG ACATTTCAAT |
| 9901 | CAATGCTAAG ACGTGATTTG AGAAACGGGG GTGAAATACG TGGCGGATAA TATAATTCGC |
| 9961 | AAACCAATTG AATTTGAATT AAATACTCAA GGCAATCCTA AAACTAATAG TTTGAAGAAT |
| 10021 | ATTGGTTTAA TCCTTGATGG CGATCCACTA CTGCATGGCA CCTTCAAATA TAACGAGTTT |
| 10081 | GCTTATTCAA TTGATGTTGT TAAGGACATT CCACAGCTAT TTATTGAAAA GGGGCAACTT |
| 10141 | GATGATAGCT ATTCAGCAAT TATGCTCCGT TACATTGAGG ATGAGTATGG GGTGATGTTT |
| 10201 | CAAGAAAAAT TGTTAAATAT GGCAATCACT GTTGAAGCAA AAAGCCACCC ATATAATCCG |
| 10261 | GTTAAAGAGT ATATGGAAAA GTGCTATAAG AATTGGGACC ACAAAGAACG AATCAAAGAC |
| 10321 | TTCCTACCAG TCTATTTAGG AGTACCCAGT GGTGAAGTAA CAACGCTGCA GACAAAATTA |
| 10381 | TTCTTAGTCG GAGCGGTGAT GAAAGTCTAT AAGCCGGAAA GTAAATTTGA TTGGGTGTTT |
| 10441 | GATTTAGTTG GTGGCCAAGG CGTTGGTAAA ACTACTTTAC TTAAAAAGTT AGCCCATGGT |
| 10501 | TGGTATACAG ACCAATTTAC GGACTTCAAA GACAAGGATA ATTTTGCCAA TATGCTGCGG |
| 10561 | GCATTGATTG TTAATGACGA TGAAATGACG GCCACAAATA ATTCTGACTT TGAAAATTTG |
| 10621 | AAGAAATTTA TCTCAGCTGA AGAATTAGAG TTCCGGCCAC CATATGGACG ACATACAATC |
| 10681 | CGCCGGCCAA AGAATTTTGT TATGGCCCGG ACTACTAACG AATCAACCTA TTTGAAAGAT |
| 10741 | AAAACCGGTG AGCGGCGTTT CTTACCTAAC ATGGCTGATA AGTCCCAAGC AATGGCTAAT |
| 10801 | CCGGTAACTG ATCTTGATGA TACGATGGTC AATCACATTT GGGGTGAAGC TGTTGGCCTC |
| 10861 | TACAAAGAAG GCTTTTCTTT CATATTGACG AAGGAGCAGC AGAAGCTCAT TGAGGATAAT |
| 10921 | CGAAAGATAT TTATGTACAT TGATGAAACT GAAAATCAAA TTGAACGGGT TCTCAGTACT |
| 10981 | TGGGACGATG ACTGGATTGA AAGCTCAGAA ATTGCTCATC AATTAGGTGA AGATAATCTG |
| 11041 | GTTAAGAATC GTTCATTAGC CAAAAAGATT AAGTATGTAA TGGATAACCG GCATGATTGG |
| 11101 | AAAGCAGGAC AACGGCGAAT TAAAGGAATT AGTCATCGTG GTTATAGAAA AGTGCATACA |
| 11161 | GATAATACAC TATGAATATA GTTAAGTGTA TGCAGAAAAA ACCTATTACA TCAACGTTTA |
| 11221 | TTAAGGTTTG CATACACTAC TACACTATTT TAATAATAAA AAATAAATAT ATATAAATAC |
| 11281 | TATATATGCG TTATAAAAAG TTGAAAGTTA GTGTATGCGT GTATGCAGTA ACTAAATCCG |
| 11341 | TTGAGAGAGT AAGAATTAAG CTGTATACAC TACTGTATGC ATAGTGTATG CAAGGAGGAA |
| 11401 | AATATGAATG AATTAAAAAA CATCAGCACA TCAGAATTAC TTGATGAACT GATTGAACGT |
| 11461 | AATGCATTAT TTCGTGTAGA TTGTGGTCTT TATCGGAATT GGGAGTTGAA GGGAAAATAT |
| 11521 | CAATTTAGCG ATATTAAATT GCCTAGCGCA TATCCTATTT ATGTAGGAAA TTCAATTATT |
| 11581 | GACCGCATGA TTAAGTGGGA GTGTGAGCAT TGACGAGTGA ACATAAAATT CAAAACGATA |
| 11641 | TCCGGGTTGC ACTATCAAAA CATCAGTGTA CAGTGTTCCG GGTAAATGTC GGTTCGGTAA |
| 11701 | AAACACCAGA TGGAAGATTT TTTTCAGCTG GTGTACCTAG TGGTCACCCG GACTTATATG |
| 11761 | GATTTCGTTG GTCGGATCAT CAAGTATTTT ATATTGAAGT GAAAAACGAA AAAGGTAAGC |
| 11821 | CTAGAGCGGA TCAAATTAGA TTTCATGAAA TGCTAACTAA ACGAGAAATT ATTCATGGAA |
| 11881 | TCGCTAGGTC TGCTGGAGAT GCAGTAAAAA TTGTTGAGGA AGGATTGATT GGTTATGGAT |
| 11941 | ATGAAAATAA AAAAGAACCA CCACAACAAG TAAAATTAGT TGAAGTAATT CAGGTTATAA |
| 12001 | CCTCTCGTGG AGCTGGAACA AAGGAGGATC CGATAAGAAA GATTATTCAG TATTGGAGCA |
| 12061 | AAGAAGGCAC ATTATTAGCA GAAAGTTTTG GAAACTAATT TTTCTTTGAG TCACGTTTCT |
| 12121 | CTATGTGCTT TAGTTGTTTT TCAGGAAGAC TATTATAAAA CTTATCAATT TGATTTGGCT |
| 12181 | GTGTTATTAA ATTGTCTGTA ATTATATTTA ACAGATTTAA AAGACTTTTC GCCATTTCTT |
| 12241 | GGTTATCAGT TAAGTCTATC TCACCAGGAT GAACAGCATT ATTTCCAACA ATTCTTACAA |
| 12301 | TATCAAACGC TTGTTGTAAT TTAGCCGGAA GTCCCTTAGA TACTAGATCA CCAATACGAT |
| 12361 | TATCTAATGA ATGTTTATTG GAAAATTCTT TTGTTAGTTT ATCTATTGCC AGACGAGACA |
| 12421 | AAGCAGCAGA TGCTCTTGGT GAATCTCTAA GTACTTTTGC CGCTTCAATA TAAATTTGTT |
| 12481 | TAATATCATC GGGCATATCT GGATTAGGAG CTGTTAATTT TAAAGGAAAA CTAGGAAAGA |
| 12541 | TAAGTATATC TTCATTGGAT TGCCGATTAT GAAACCAAAT AGAATCTAAA TAACATGAAG |
| 12601 | TGCATTTTGC AACTATTAAA TTATATTCGT ATTGTTTTGA TTTTATTTTT TCTGCATATT |
| 12661 | TCCAATCAAA TTGAGCATAT ACATGACAAT AAGGACAAAT AATAGGAGCT TTCCCTGTAA |
| 12721 | AATCATTACT AAAATATTTA GTCATTAAAT TCACCTCAAA TAAGATTATA GCGTAAAGGA |
| 12781 | GTAGATGTAA TGCTACACAA ATATAGAAAG TTACCAATTG TGGAAGCTGA ACAGTTTGAT |
| 12841 | GGGTCAGATG AAATGATTGA AAGGTATTCA GTGCATGTAT TTAATCCTAA TTTAGCTAAA |
| 12901 | AACATCTTCT TTATAGGTAT GAACGTTCTA GCTATTGGTG ATTGGATTGT TAAGGATGAA |
| 12961 | TATGGGAATT ATCAAGTGGT AGCTGATGAT ATATTCCGTA AAAGTTATGA GAGGTGCGAC |
| 13021 | TAATGCACAT TTATGAAGTA ATCGTTGTAG CTGTATTTGG CACAGATATT AGCCACTTTG |
| 13081 | TTGTTGCTAA GAATGCCGAT AATGCTAAGA AAATTATTCT TGATTATTAC AGCACTCGTG |
| 13141 | ATGATGGTAT CAGGCCAACT GTGACAATGT ATGACCTAAC AACAAAATTA ATCAATCTTA |
| 13201 | ATAACTACAT TGATGAGGTG ATGCTTGGAT GAGATTAAGT GACAAAATTA TTATGACTTC |
| 13261 | CTTTTTACTG TTATTAATTG TTTCAGTCGT CTTATCAATA GTTACGGGAA GTAAAGTCTG |
| 13321 | GATATGGATT TTTCTTTTTC TGATGACACT TGAGATGCTT TACAAAATTT GGCGTTAGGA |
| 13381 | GCGTGACTAA TGAAAGCAAG ATGTGGCGAT TGGAGTACTG ATGTTTATCC AGTTGAACCG |
| 13441 | TTTGTTTATG AAGTAGCAGC TGATGGTAAG AAAGAATTTT ATCAATCAGC ATGGGATGCT |
| 13501 | TTTAGTTCTG AATTGCATAT GAAAAATGTG ACGGCGAAGA TTATCAAAAT ACCAGTTGTT |
| 13561 | CCAATGAGTA ATGATGAAAT TAAAGCAGAG AGTGTTGCAT TTGAATTAGG CGAAAAAAGG |
| 13621 | CATTATCCAC ATGTGGAAGA ATTCACTAAT AGTAAACCGA GGAGGAAAGT TACATTTCTT |
| 13681 | CAGGTTGCGG AAGCTTTTGC AATTATAACG TGGATTATTT TAATAATTCT TATAATAAGT |
| 13741 | ACGGCGTTTA TGTAGGAGGT AAGTTACTAA TGACATTTGA AGAGGCGTTA AAACATGAAG |
| 13801 | AAAATAATGT GCCAGTATTC TATAGCGATC GAAAATATTA TGTAATCGGG CACAATGAGT |
| 13861 | TAACCGAACA ATTTACAATT CGTGAGCTAA GTGGTAATCC GTTATTTACG GTGCCGGTTG |
| 13921 | ATGTACAAGC GGAGGAATTG TCATGAGTAT TAAAATTAAT GCGCAAACAG TTGTATTTAA |
| 13981 | GGGACAAAGT TTTATTCCTA GCAAAAGTAA CTGTGAGTAT TGTCAATTTC CATTTAAGAA |
| 14041 | GTTAATGGTT ACTAAAGCAT CTCCAGATCC ATCAGTTAAG ACAGAAGTAC CAATCAAAGT |
| 14101 | TGACGGTGAG GTATTTAATT ATTGTCCTCA TTGTGGACGG AATTTACAAG GGTGAGGCAA |
| 14161 | TGTTACTTTT AATTGTATTA ATGATGATTG TCTTTATTGC TGGGTTCCTA TTGGGTAAGA |
| 14221 | AAAATCCATG ACGAAAAAAG GACCCACCGT AAAAGATGTG TCCTCACTAA AAATATTTAA |
| 14281 | CCATAATTAT TATATCAGAT AGCGAGGGTA CGTCATGCAA ACAAGTTTGA ATTTAGATAT |
| 14341 | TGATTGTCTG AAAACTGCAA GAAAGGTTAC TGACTTTCTT GATAAGAAGC TGGATCGCTA |
| 14401 | TCTGGCTTTA TCGGGGAAGC AACGTTTTGA TTTGAAGTCA CCAGGGATGG ACGGAATGCC |
| 14461 | TAAAGCTCCC AGCCATGGTA ACGGTAGTGA AAATCGAATG CTGAATATCT GGTTGGCAGA |
| 14521 | AGAGGTTGTT GATTGTGTGG GTTGCGCTAT GCGGAATATG ACAAAGGAAT CACAACGGAT |
| 14581 | TTTACTAAAT CGGTATTCGG ATCAGATGTT AACGTATAAC ATTGCTAGGG AGCTAAGTAT |
| 14641 | TAGTTCAGCA ACGTATAGTC GAAAGCAGGA AAAAGCGCTG TGTGAGTTTG CGGACCGGTT |
| 14701 | TGAATTTCAG TTAGTTAAGC ACGGTATTCA TACTGAAATT GATGATCTAC ACGTTTATCT |
| 14761 | TGACGAGGAA GATTGATAAA TTGATGATAG ATTATTGAGC GAGCAATTCA TGATAGAAAT |
| 14821 | GTGATAATAG TATTGTCGAA TGATTCGATA TTCATATAAT AATCTTCCCA AATGAAGTCT |
| 14881 | AGCTATTATG GCTAGGCTTT TGTATTATGT TTAATTGGGT GATTTTTATA TGATTGAATA |
| 14941 | CTTAAAAACA TTTTTGGAGG CTTTGAGTAT GAAGCCAAAA ACTAAATTTG TTGGAGTTAT |
| 15001 | TTTTGGAATA GTACTTTTAT GCTTAAAACC ATTTTTAATT CAATATAATA TGAAATGGTT |
| 15061 | TTACAATAAG TTTTCTTGGA TTATTATTTT AATTACTTTA TTTTTTGTAG CCTCATTAAT |
| 15121 | AATTGAAGTA ATAGTTGAAG TATACGAATG GGGTAGAAAC AAGTATAACA AACATAAAGT |
| 15181 | TGAAAGAGAT TACGAAAAAT ATATCTTAGG CTTGTCTGAT AAAAAGTTGG CAATTGTAAA |
| 15241 | GAAACTTTAC GCTAATGAAC ATCACCAAGG ATATTTAAGA CAAAACGATA CTAATGTTAT |
| 15301 | TGAATTGGTT AATATGTACG TAATTATGCA ACTTAATAAT GAAATTATAG TAAGAGAAAG |
| 15361 | CCAAGTTGAA GATATAAACG ATCCTGAATT TCTTTTTGTA TTACAGCCAC CGGCTTTACA |
| 15421 | CATCATAGAA AAGAATTCAG AAAAATTTAA ATAAATTTAA TTAGTTTAGC TTAACGGCTG |
| 15481 | ACTTTTTACT TTTTAGGAGG TGAGTAGCAT TACTCAAAAA TTAACACAGA AGCAACAACG |
| 15541 | ATTTGTCGAT GAGTACATTA TTTCGGGTAA TGCTACTCAG GCGGCAATTA AAGCTGGATA |
| 15601 | TTCTAAGAAG ACAGCTAAGC AGTCTGGTGC TGAAAACCTA GCAAAACCTT ACTTAAAAGC |
| 15661 | TGCAATCGAA AAACGCAATG CTGAAATTCA ATCCGAGAAA ACAGCTGATA TGACAGAGGT |
| 15721 | GATGGAATAT CTTACTTCAG TTATGCGTGG TGAGCAAACA GAATCGGTTG CTACTGCTAA |
| 15781 | GGGTATTTAT GAAGACGTTG AAGTGTCGGC AAAAGATCGT ATTAAAGCTG CTGAATTAAT |
| 15841 | TGGAAAGCGT CACGGCGCCT GGACTGATAA AAAGGTTATT TCTGGTGATG TTCAGATTGA |
| 15901 | TGTGGGAATG GGGGATTATG ATGATGAAGA GTGAAGAACA ATGGAGAAAA ATCAAAGATC |
| 15961 | ATCCTCATTA CTTGGTTAGC AATAAAGGTA ATGTTTACAG TGAGTATAAA GGCGGCTTGC |
| 16021 | TTAAACAGAT GAAAGATGCT TATGGATATT CTCAGGTTAA TTTAAACCGC CGCTCCAAAA |
| 16081 | AGGTGCATCG TTTAGTAGCG GAAGCTTTTA TCCCAAACCC AGACAAATTG CCTGAAGTTA |
| 16141 | ATCATAAAGA CGAAGATAAA AATAATAACC AGGTGGATAA CTTGGAATGG TGTACTAGCA |
| 16201 | AGTACAACAT GAATTATGGT GACGTGGAGA AAAGGTCAAT TCTTTCACAA CAAAGCCATA |
| 16261 | GTACTTGGAA AATTTATCAA TATGATTTAA ACGGTAATTT GGTAAAAGTA TGGAATTCAG |
| 16321 | CGAGAGAAGC CGACAGACAT GGATTCAACC GTAGAAGTGT GTATCGCTGT TGTGATGGGG |
| 16381 | AAATAAAATC TTTCAAAGGA TACATATGGT CAAGACAAAA GAAGGTGATA CCATGCCAAA |
| 16441 | CATCAAACTA AATTTTCCTA AACCATACAA CGTTTTCAAT AAACAAATTT TTGATAACTT |
| 16501 | GTTTGATTAC AGTCATTTCG TTGAGGTTTG GTACTGACTT ATGGCGGTGC ATCTTCTGGT |
| 16561 | AAATCGCATG GTGTGGTACA GAAAGTTGTA CTTAAATCAC TCCAACACTG GAAACATCCC |
| 16621 | CGCAAAGTGC TATGGCTGCG GAAAGTTGAT CGAACAATTC AAGAATCTAT CTTCGCTGAC |
| 16681 | GTAATTGACT GTTTATCAAA TTGGCAATTG TTATCGTTAT GTAGAGTAAA TAAATCAAAC |
| 16741 | CGTACTGTTC ATTTACCGAA CGGTGCGGTT TTCCTGTTTA AGGGTATGGA TGACCCAGAA |
| 16801 | AAGATTAAGT CAATCAAAGG GTTATCTGAT GTGGTAATGG AAGAAGCTTC CGAATTTACA |
| 16861 | CAGGACGACT TCACGCAGCT TACCCTACGT CTCCGTGAAC CTAAGCATAA GAAACGACAA |
| 16921 | TTGTTTTGTA TGTTTAATCC AGTTAGCAAA TTGAACTGGA CTTATAAGCA ATGGTTTGAT |
| 16981 | CCGAAAGTGA AAGTTAATCC GGAACGAGTA TCAATTCACC AATCAACTTA CAAGGATAAT |
| 17041 | CACTTTTTGG ACGCTGATAA CATTGCAACG ATTGAGAACT TAAAACAAAC CAACCCGGCC |
| 17101 | TACTATAAAA TCTATACGCT GGGCGAGTTT GCTACATTGG ATAAGCTGGT CTTTCCAGAG |
| 17161 | TTTGAAAAAC GTCGGTTAAG TATTCGAACC TTATCACAGC TTCCCTCGTA CTTCGGCTTG |
| 17221 | GACTTTGGGT ACACTAACGA TGAAACAGCC TTTATGCACG TTAAAGTGGA TGAGAGTACC |
| 17281 | CGTAAAATTT ACGTGATGGA AGAGTACGCT AAGCACGGTA TGTTGAACGA TGATATTGCC |
| 17341 | CGAATAATTA AACAAATGGG TTATAGCAAG GAAGTTATTA CTGCTGATGC TGCTGAGCCT |
| 17401 | AAATCGATTG CTGAAATTAA ACGCGATGGT ATCTCGCGGA TTCGTCCAGC TAAAAAGGGG |
| 17461 | AAGGACAGCA TTATACAGGG ACTTTCATTT ATGCAGCAAT ATCACTTAGT CGTTGATGAC |
| 17521 | CGGTGTGTGA AAACGATTGA GGAATTGGAA AATTATACAT ACAAGAAGGA CAAACAAACT |
| 17581 | GGTGAATACA CCAATGAGCC TGTCGATAGT TACAACCACG AAATCGATGC TATCAGGTAT |
| 17641 | GCTTTATCTG AAATCAACGG AATGGCTAGT CCAAAGGCAA CTGTAATGAA AAATATTTAT |
| 17701 | ATTTAGGTGG TGATTGAATG GAAACAGTAA ACGGTAAAGG ACAAATTTTA GATGGCCATA |
| 17761 | TTTTTATCTA TCCAGCTGAT GAAGAAGAAC TTGATCCGCA TGATTTACTG TCGTTCATGA |
| 17821 | GAAGAAATAT TCAGTATGCT AAGGATTACA AGCATAATAT GCAAATGTAT CTAGGTAATC |
| 17881 | ACGATATCTT AGATCAACAG CGGCGGATGT ATGGGCCAGA TAATCGGCTA GTAGCAAATT |
| 17941 | TACCGCATTA TATTGTTGAT ACTTATAATG GATTCTTTAC TGGAATCCCA CCTAAGATTA |
| 18001 | CTTTAGATGA TAAGAATGAG AACGAAGCAT TACAGCAATG GAATGACACT AATTCGTTCC |
| 18061 | AGGACAAATT GAGTGAAATT AGTAAGCAAA CGGATATCTA CGGACGTTCG TTTGCTTTTA |
| 18121 | TTTATCAAGA TGAGAACGCA GACACTTGTA TTGCTTATGC TTCTCCTACA GATGCCTTCA |
| 18181 | TGGTTTACGA TGATACGGTT GCTAGAAAAC CTTTTGCTTT TGTTCGTTAC TGGAAAGATA |
| 18241 | CTGAAAGCGG ATTATGGACC GGAATGGTTT ATTACGCTAA TAAAATTAAA ACCTTTAAAG |
| 18301 | GTAGTGTTGT TGAAGATTCA GATCAAAATA ATATGTATAA TTTAGTGCCA GCAGTTGAAT |
| 18361 | TTTATGGAAA TGAAGAGCGG CAAGGTGTTT TTGATAATGT GAAAACCTTA ATCGACGAAT |
| 18421 | TAGACAGAGT GTTATCACAG AAAGCTAACC AAGTGGAATA TTTTGATAAT GCTTACCTTA |
| 18481 | AAATTCTTGG TCTTGATTTA GATGAGGATG GTGATGGTAG ACCGGATGCT AATTTAATTG |
| 18541 | GTAATCAAAT GATTTATTCG CCTAATGCTG ATGCTGCTAA TGCCGATGTC GAATTCATTT |
| 18601 | CAAAACCAGA TGGTGATAAT ATGCAAGAAC ATATTATTGA CCGGCTTGTT TCAATGATTT |
| 18661 | ACCAGGTAAG TATGGTTGCT AACCTTAATG ATGAAGCGTT TGCTGGTAAT AGTTCTGGGG |
| 18721 | TGGCTTTGCA ATATAAGTTA CTTCCAATGC GAAATATGGC AGCTAATAAA GAGCGTAAAT |
| 18781 | TTACTCAGGC ACTCCGGAAG TTATATCGAA TAGTGTTTAG TGCTGATCAA GTAGTCAAAG |
| 18841 | ATAAGGAAGC CTGGCAAGAC TTGCTCTTTG ATTTCAAACA AAACTTACCG ATTGATGTTT |
| 18901 | CTGAAGAAGC TGATACTTTA CAAAAACTAT CAGGGGTTGT GTCAAAAGAA ACTGCATTCC |
| 18961 | GAAATAGTCG TTTAATTGAT GATCCTAAAA AAGAAGTTGA GCGTATGCAA AAAGAGAAGC |
| 19021 | AGGAAGAAAT AAACCAAGCG CTTCAACATT CTGCTTCTGC TACAGATCAA ATGCTAATGG |
| 19081 | ATGATCAAAA AGAAAATGAT AAAGAGATAG TTGGTTTCCG GAAGAACGGT GAATCCGATG |
| 19141 | ACGAAGAAGA ATAATTATTG GGCTGATCGT ATTGCTCGGG AACGTAAATG GCAAGAAGAG |
| 19201 | CAATTAAGTA AAGATGCTCA ATTTAATCAG CGCCTTCAAC AGTATTATGA TCAAGCAATT |
| 19261 | GTCCAGATTA ATAAAGACAT TGAAGATCAG ATAAATTCTT TAGCTGTCCG GAATAAAGTT |
| 19321 | TCTTATGCTG AAGCTCAAAA AGAAGTGTCC ACTACCGATA TTGCTGATTA TGAAACAGAA |
| 19381 | GCTAAGAAGG TAGTTCAGGA AGCTAATCGT TTAAGAGCAC AAGGGAAGCA TGTTACTTAC |
| 19441 | AATGATTTCT CTGATGAAGT TAATGAACGA TTGAGGAATT ATAATACGGC GATGCGATAT |
| 19501 | AACCGATTGA ATTTATTGAA ATCTAAAATT GGTTTATCGA TGGTTGAAGC CGGAATGAAT |
| 19561 | ATTGATGCTG ATATGCAAGC TAAAATTGGT AAAGATTATA CTGATGAGCT AAAACGTCAG |
| 19621 | TCTGGTATTC TAGATCATTC TACCGAAAAT AGTTCGTTTT GGACTTCTAA AGATGTTGCG |
| 19681 | AAACAAGTAA TGAAGCAAAT TAATGGAGCA ACTTTTAGTC AACGAATTTG GGCTAATCAA |
| 19741 | GATACTTTGA AAGCTCAACT TGATACGGTT ATCACCAACG GAATTTTAAC TGGTAAGAAT |
| 19801 | CCGCGAGTTG TAGCAAGACA ATTAAGAGAT AAAGTAAAAG TCACTGTTAA AAATCACAGT |
| 19861 | TATGTTACTG AACGTATTGC CAGGACAGAA TCAGCACGGG TTCAGTATTC TGCTCAGATT |
| 19921 | GAATTAATCA AAAAGAATGG TTATCAATTT GTCCACTGGA TTGCGGAGCC AAGAGCCTGT |
| 19981 | GATGAGTGTC GAAAGATTGC GACGCAAGAT AATGGCTTTG GTGATGGTAT TTATCGAATT |
| 20041 | AATAAAGTTC CTAAAATACC AGACGATACT CATCCTAATT GTCGCTGTTC AATTAGTGAG |
| 20101 | ACATGGGTCG ATGGTCAACG CAATATAGCA TTATCTGATG ATGAACAGGC GGCATTGAAT |
| 20161 | AATTATATTA GTTCAGATTC ATATAAGATT AATGATGATT TGAGGCGTAA TAAGATTTCT |
| 20221 | AAAAATAAGA AACAATTTAT TGAAAACTTA GATGCCGCAT TGGCTAAAAT GCCAATTTAT |
| 20281 | CATAGTAGCA AGCCACTCCA GCGTGATTAT TTCTTTGATA AACAAGAAGC ATTGGATGAT |
| 20341 | TTTATTAGTA ATTTTGAAAT TGGTGGAGTC TTCACTGATT CATCATACAT TTCAACTTCT |
| 20401 | AAAATTTATT ATGGACAGGG CAAAGAGACA ATTCATGTTA TTATTAAATC AAGTAAGACA |
| 20461 | GGAAGAGATA TCTCTGAGTT TAATTTTAAT GAGCAGGAAG TATTATTCCC CAGAAATAGT |
| 20521 | AAGTTTAGGA TTGATGATGC ATACGTTGAT GATAACGGGA AGATGACAAT GGTTTGGAGT |
| 20581 | GAATTAGATG AGTAACAAGC CTTTTACTGA TAAACGTTGG CGAGATAATT CTTTGGAAGG |
| 20641 | CGTTAAATTT GATAATTCAA AAGTAACGTC AGAACAAAAG AAGAAGACAG AAGAATTCCA |
| 20701 | TGAGTTATTC AAAAAGACGT TTGCTAAACA ATTAAAAGAA AAACACTCAC ATAAAAAGTA |
| 20761 | GGTGATCCAA TGGGAAATAA TGATTTCTTT ACGGTAACGT ACAAAATACT AAGTTATCTT |
| 20821 | AAGTATTGTT ATGAAAATGG AATTAATCCT GATCCTAATA TTCTTAATGC TGATACATTT |
| 20881 | AATATTAGTA AAGTTCAATT TGGAAGAACT CTACAAATGT TAAGCGAGCA TGGTTATATT |
| 20941 | TCAGGAGTGA GATTTACACA AGCCAAAATC GAAGGTACTG TTGTTGGTGG ACTCCACAAT |
| 21001 | ACGTCAATAA CGGTTGAAGG TCTGCAATAT TTAGCTGAAA ACTCAATGAT GAAAAAAGCA |
| 21061 | TATCGAATTT TTAAAGAAGT CAGAGATTGG CTTCCGGGTT TCTAAGCATT CACAAATTAG |
| 21121 | TGAGTGCTTT TTGTTTTGGA CTTTTTACTT GTTGCAGTCG TTAAAGAACA ACCCGGATAT |
| 21181 | TACAGTCCAC CGGACTATAA ACGAGGTGTA TTTATGTTTG AAAAATTACC AATGCGTTTA |
| 21241 | CAATTCTTTG CTGAAGATCC AACGCCAGAT CCAGATAATG ATGGTGCACC TGAAGGAACT |
| 21301 | GATGATGGAG ATAACGGTAA AAGTGAAAAG ACATTTACTC AAGCAGAATT AAACGATATT |
| 21361 | GTCAAAGCCC GAGTCAATCG AGCCTTGAAG AATAAGCAAG AGGAAATTGA CCAGGCTAAG |
| 21421 | AGTGAAGCTA CTAAACTTGC CAAGATGAAT AAGGATCAAA AGCAAGAATA TAAGCTTCAA |
| 21481 | CAAACTGAAA AACGTGCCCA AGATGCTGAA GCAGAATTGG CCCGTTATAA AATGCGTGAT |
| 21541 | ACAGCGAAGC AACAATTAAT TGATGGCGGT TATGACAATC CAACTGATGA AGATATCGAT |
| 21601 | TTAATTGTTA CTGATAAAGC AGAAACAACT AAAGAACGTG GTGAAGCATT TCTTAAAGCT |
| 21661 | TATAACCGAA TTAAAGAAAA TGTTCGTCAA GAACTATTAA AGGGAAAGTC ACCACGAATT |
| 21721 | AATGGTGCTC CTGCTACTGC AATGACTAAA GAACAAATTG CAAAGATCAA GGATCCCGTC |
| 21781 | AAACGGGTCC AAGCCATTCG GGATAACTTA TCCCAATATG AAAAATAAAA GGAGGAATAT |
| 21841 | AAAATGGCTG AAACTAATTT AACGACAAGT ACAGACCTAG TTGCACAATC TATCGACTTT |
| 21901 | GTAGAACAAT TCTCTGGAGG AATCCAAACT TTATTGAATG CATTGGGAGT TATTCGTATG |
| 21961 | CAGCCAATGA CTACTGGTTC ACAGATTAAG ATTTACAAGT CAGAAGTAAC TAAGGTCGAT |
| 22021 | GGTAATGTTG CTGAAGGGGA AGTTATTCCG TTAAGTAAGG TTACTCGCAA GCTAGCTAAT |
| 22081 | ACTTTGACAT TAGGATTTAA AAAGTATCGT AAGGTAACTA CCATTGAAGC TATTCAGTCA |
| 22141 | GCTGGTGGTG CTACACCTGC TATCGTGGAT ACTGATAATA AGCTACTTCG AGAAATTCAA |
| 22201 | AAGGATGTTA AGAAGGACTT ATTTAATTAC ATTACCAAGT CTGATGCAAA CAAGACTACC |
| 22261 | GCCTCTGGTG ACGATTTTCA AAAGGCAATG GCGGCAGCTT TAGGACAACT TTCAGTTAAG |
| 22321 | TGGGAAGACT ACGACACACA AACTGTCGCC TTTGCTAATC CGCTTGATCT ATATGCATGG |
| 22381 | TTAGGTAACC AAACTCTTAC TGTTCAATCT GCCTTTGGTT TGCAATACAT TCAGAATTTC |
| 22441 | CTTGGCTTTG ACACTATTAT TCTAAGTGCT GAAGTACCAC AGGGAACGAT TGCTACAACA |
| 22501 | GTTGCAGATA ATATCAATTA CTTCTACGCT CCAATTTCAT CCGTTGGTCA GTTATTTAAC |
| 22561 | ATGACTTCTG ATGAAACTGG TTTAATTGGT GTAACTCATG ATGCAGTTAA TAACAATTTG |
| 22621 | TCATACGAAA CTGTTGTAAC AATGGCTAAT GTATTGACTA CAGAACGTTT GGACGGCATT |
| 22681 | GTATTGTCAA CAATCAGTGG TGCTAAGTCT GCTGGTAAGT AGGTGATTGA ATGGACCAGA |
| 22741 | ATACGGTTTT GCAAAACTTA AAAGTAATGC TTGAAATTAA AAATGATGAC CGTGATGCTT |
| 22801 | TGCTGAAACT AATCATTGAT AATACAGACC AAGCATTGCG ATTTAAGCTA GAACTAACTG |
| 22861 | AAGACGAAAA TTTACCTGGA GAACTAGGTT ATATTGAATT AGAAGTTTCA GTTCGACGAT |
| 22921 | TCAATCGACT GCAAAACGAA GGGATGAGTC AATATAGTCA AGAAGGGGAA AGTATTACTT |
| 22981 | TTAATTCTTC AGATTTTGAT GATTTCCTTG ATGATATTGA TTTGTGGAAA CGACGACACC |
| 23041 | AGAAAGATGT TAAATCTTTA GGTGCCGTTT CTTTTATTAA TCCTTATGCG GGGATGAGTA |
| 23101 | AAAATGCGAA AAACACAGAT AATTAAGTTT TATTTTCAAG ATGAAAATGG CTATAATCCA |
| 23161 | TATGCAGAAG AAGACACGAT TACTAGTCCT AAGCTAGTAG CACAACGGTA TGCCAATGTT |
| 23221 | ACTGATGTAG GGACAAATCG CCTAGTTGAA TTATTTAGCA GGCTAGATCA GAACGCCAAG |
| 23281 | GTAATTCGAT TGGAGTCTCC AGTAAATGAC TCTTGGTCAT ATCTGACTAT TGATGATTGT |
| 23341 | CCTATCAAGT ATCGTCTTGA AACCTGTCGG AAACCATTAA AAGGCACAAC GCTGATTGTA |
| 23401 | GGTGAAGCCA GTGGCTAATT CTTTTAAGGT TGATGTTAAA GGTACTAAAG AACTAGCGAA |
| 23461 | TTTCTTAAAG AAGAATAAAG ATTTGACTCC AGTTAAACGG ATAGTCGCAA AACATGGAGC |
| 23521 | AGGTCTCAAA AAGCAAACGC AGCAAAATAT GAACAATTTG TATAAGGGCC ACTACGAATG |
| 23581 | GAAGAAGGGG GCTGGGCTAA CAATGGTTAG CCCTACCGGG AATACTAGAC GGTCCGTAAC |
| 23641 | AAATACAATT TCTAATAATG GTTTAACAGC AACGGTTGCT CCACAAACTG AATATTTCCC |
| 23701 | ATATCTTGAA TATGGAACTC GCTTTATGGC AGCACGACCG ACATTACATC CAGCGTTTGC |
| 23761 | AATTGAGTCT ATGAAATTTG CTAATGATTT GAATAAGTTA TTTAAGTAGG TGAGAAAATG |
| 23821 | TCACCAAGTA TTGAAATTTA TGATGCAGTT TTTGCTCAGG TCCAAAAGCA TTATCAGACC |
| 23881 | TACGATCACC CACCGCAGTT AAATGAACCA GTTACCTATC CATTCGTGGT TGTTGATGAT |
| 23941 | AGCCAGTCGA TTTTGACGAA CTATAAAACA GCCACAGGAA TGCGGGTAAC TTTAATAGTC |
| 24001 | CATGTGTGGG GGAAGTCTAA CCAACGTAAG ACTGTTACTA AGATGGTTGA TGAAATTAGT |
| 24061 | CGTCTGGGGA TGCAAGCAGT TCGGACGAAA CATTATGCTT GGCAAGGACG ACCTAATGAG |
| 24121 | CAAGAACAAC AATTATTAAC TGATACGAGT GTTCCGAATA CTGTGTTAAA GCACGGTTAT |
| 24181 | TTAACACTCG TTTTTGATTT GAAATAAAGG AGGATAAATA TGGCAACATA TCCAGTATTG |
| 24241 | GAAGGGAAGA ATGCAGTTCT TTTTGAACGA CTATTAGAAA ATGCAAGGAA AGAGCCGGCA |
| 24301 | CAATTGATCC CGTATCAAAC ATCACTAAGT TATGATCCTA AACGGGATAC TGATTCAACA |
| 24361 | ACTACGAAGA TGGGGAATGT TCCTACTGCT TCTAATATTG AAACAGATTT AGAAGTAGAG |
| 24421 | TTCCTAAATG CAATTTCCAA AGCTGCAGAT GATGTTTATG ATTCTTTGTA CTTTAATAAG |
| 24481 | AAGATTGAAG TATGGAAGGT TCATATTGAT CGAGTCCGGT CAGATGGCAA AGTTTATGCC |
| 24541 | GAATATATGC AAGGAATTGT GTCAGAAGAC TCTAATGATA ACGACCCAGA CGATCATTCA |
| 24601 | ACTCGGGATG TGACCTTTAC GATTGATGGC GTGGCCAAAC GTGGGTGGGT CACTCTACCG |
| 24661 | CCAGAAATCA AGGAAGAAAT TGACTATGTA TTCCGTGGCT TGGCACAGCT TAAAGGCGAT |
| 24721 | GACGACAACG GTGAAGGTGA AGCTTTTGCT GATGGCGATC GTGGTGCTGG TGCAAATGAA |
| 24781 | GCAGTAACAA CTGAATAGGA GGAAGATTAT GAAGTTAAAA ATTAATGGTC AAGACCAATC |
| 24841 | ATTTGTATTC GGAGTTAAGT TTTTGCGAAA ACTTGATGCT TATCGGGGCG CTGAACAAGA |
| 24901 | AATCCAAGGA GTTAAGGTTA AGCTAGGAAT GGGGCTAGCC ATGATGCTTC CCCAATTAAT |
| 24961 | GACTAAGGAT GCGGCCGCTT TGGCAGACGT GTTGTACTGT GCGGCTAAGT CTAGCATTAA |
| 25021 | GTTAGATACA ATTGATGATT ATATTGATAA TTGCAAGGAC TTGGATTCAT TATTTAATCG |
| 25081 | GGTAATGAAT GAAATTAAGG CAAGTAATGC CGCTAAGCCG ATTGCAAAAA ATCTAAAAGC |
| 25141 | CTAGATGGTC CTGAGCTTAG TTCAGAACAA AGCTATCACG AAATTCTTTT GAATTCGTTG |
| 25201 | GCTTATCTAG GCTTTCATAA TATTTCAGAA ATTGAAGAAA TGGGATTGGC TGAATATCAG |
| 25261 | CTCCGGATGG AAGCCTATAA CCTCCAACGG GTTAGCCAGG AACGAGACTT AGCATTGCAA |
| 25321 | GCTTTCCTTA ATCAGTCGGT ACAAGCGACG AAAGGGAGCG AAAAGCACCC AATTCCGAAG |
| 25381 | TATAAGAAGT TTAGCCAATT TTTTGATTAT GATAAATTTG TTGATGATGT TCGTGGGCAC |
| 25441 | TATGAGCCTG ACTATCAGCC AACAAGCAAG GCCAGCCTTG AAAAGAAACG AAATGATCTA |
| 25501 | ATCGTCAAGC GGTGGCGTGA ATTCCGGAAG ATGAAACAAA AACAGAGAGG AGGTAATGGC |
| 25561 | TAGTGTCACA ATCAATGAGA GTTGAAGCGG TATTATCAGC ATACGATGAG AGTTTTAGCG |
| 25621 | CAACCTTAGA TAAGGCGCTT AAATCGATTA ATAATTTAGG CCGTGAAACC CAGTCAACCT |
| 25681 | CTCAAACTGT TAGTGCAGGT GGTTCTAGTA TTTCCAGTAC CTTTAAATCG ATGGCTGGAG |
| 25741 | CAATGGGTGT AGTTGCGATT GCTGGTAAAG CATGGGACGT TGTTAAAGAT TCAATGAGTG |
| 25801 | GCGCCATTAA CCGGTTTGAT ACATTAAACA AGTATCCGGT AGTAATGAAG GCTTTGAATT |
| 25861 | ATTCAACTAA GGATGTTGCA AAGTCAACCG CTATCTTATC TAAGGGAATT GATGGATTAC |
| 25921 | CTACTTCTTT GCAAGACGTT ACAAGTGTTG CCCAACAATT AGCGCCATTA ACTGGTAGTG |
| 25981 | CAACTAAGGC TTCTAAGTCG GCGATTGCCT TGAATAATGC CTTCCTTGCC TCCGGTGCTA |
| 26041 | GTGTTGCCGA TACCTCTCGT GGACTTCAAC AATACACACA AATGCTTTCA ACTGGTAAAG |
| 26101 | TCGATTTAAT GTCTTATCGA ACATTGATGG AAACCATGCC AATTGCATTA CGTAAAGTCG |
| 26161 | CCAATTCATT TGGTTTTACT GGTAAGTCTG CTGAACAAGA CCTTTATAAA GCTTTGCAGT |
| 26221 | CAGGACAAAT TACGGTAGAT CAGTTGAATG ATCGTTTTAT CAAGCTGAAT GGTGGAGTTA |
| 26281 | ATGGTTTTGC TCAATTAGCA AAGAAAAATA GTGAAGGTAT CGGTACATCT TTTGCAAACT |
| 26341 | TAAAAAATGC CGTTGTCAAA AATCTGGCAA ATATGTTATC GGCAATTGAC AATGGTTTTA |
| 26401 | AGCAAGCGGG CTTTGGAAGT ATTGCACAAG TCCTAGACAA CATGAAGGGT AGTATTAATT |
| 26461 | CTGCTTTTCA AGTTATTGGA CCAGTTGTTA CTAATGCTAC TGTTGTAGTT CTTAATTTTG |
| 26521 | CAAAGGTTAT AGGCGGAGCG CTTAAATCTG CTTTCAGTAA TGATATTTTT AAAACAGCAG |
| 26581 | TTGTGGGAAT ATTAGGCTTT GTGGGTGCAG TTATGGCAGC CCATAAGGTT ATTTCAATAT |
| 26641 | TTACAACATT AAGATCTGCA ATAGTTGGTT TAAGTGTGAT TACAAAAGCT GGTAATTTGG |
| 26701 | CAATGGCGTT TAGTGAAGCA ATGTCAACAC TTGCTAAAAC TTCTAAGATT GCTGGTGGAG |
| 26761 | CGATGAAAGC ATTCAGTGCG GTGGCCTCAT TAGGTCCCTG GGGAATTATT GCTGTTGCAA |
| 26821 | TTGCAGCTGT GGTTGCAGCC TTAACTTATT TCTTTACCCA AACGAAAACC GGTAGGGCTT |
| 26881 | TATGGCAAAG TTTTACTACG TGGTTATCTG GAGTATGGCA GAGTTTGGTT GGAGTGGCTA |
| 26941 | CTACTGTTTG GAATGCAATT GGTAATGCTA TTAATGCAGT AGTTAATTTT ATTAAACCTT |
| 27001 | ATTGGCAAGG ATTATTAACA TTCTTTACCG GAATCTGGAC ATCAATTGTG GCGGGTGTTG |
| 27061 | CTCCAATTTG GCAAGGGTTA GTTAATGTCT TTAATAGCAT TATCAGTGCA ATTTTAGCCG |
| 27121 | TTTGGCAGGC TTTAGCTCCA ATTATTGTTC CGATTGTAGC TGGTGTAGTT GCTATCATTG |
| 27181 | GGGCAACCCT AATTACGATT GTTACCGTCT TTCAAACTGT GTGTAATATG CTTGTACCCA |
| 27241 | TTGTTCAAGT TGTATGGCAA TTAATTTCAA CAGTTGTATC TACTGCTATT ACGATGCTAG |
| 27301 | GTACAATAAT CCAAACAGGC TTGGCAATTA TCGTTGCTAT TTGGAATGTG GTCTGGAATA |
| 27361 | CATTCAGTAT TGTTGTAAGT ACGGTATGGA ACGTTATTAC TACTATTATA TCTACCGTGC |
| 27421 | TGAACGTTAT TGCGGGAATA ATTCAAGCTA TCACTGCTGC AATACAAGGC GATTGGTCAG |
| 27481 | GAGCTTGGAA TGCAATTCAG AATGTTGTAT CAACTGTTTG GAATGCAATT GCCAGTATTA |
| 27541 | CTTCTAGTGT ATTGAATGGA ATAAAAGGAA TCTTTGATGG TGTAATGAAT GGTTTAAAGA |
| 27601 | GTATTACTTC TAGTAGTTGG AATGGTATTA AATCGCTATT CAGTGAAGGT GTTAATTTCA |
| 27661 | TTAAATCAGT TGTTCATATA GATTTAGGTG CTGCTGGTAG AGCTATCATG AATTCACTTT |
| 27721 | GGAATGGAAT GAAATCCATT TGGAATAGTA TTAAGAATTG GGTTAGCGGT ATTGCTGATT |
| 27781 | GGATTAAAGA ACATAAAGGA CCAATCAGTT ATGACCGTAA GTTACTTATT CCAGCTGGGC |
| 27841 | AAGCAATTAT GAATGGTCTT AATAACGGAT TGATTAATGG ATTCAGTGAA GTTCAATCAA |
| 27901 | ACGTTAGTGA TATGGCTAAT CAAATTCAGC AAGCTATTAC TAATCCAGGC TTTGATATTG |
| 27961 | GAGCAAGTAT TGGTAACTTG GGTTCAATTA ATTCAAATTA TACTGGTAGC CTGGCAATTC |
| 28021 | AAGATAGTCA GTTACAAATG CAGAATAATG CTTTGCTTCG TCAATTACTT AATAAAGACA |
| 28081 | CGACAATGGT TCTTGACGAT GGCACTCTTG TTGGCTATAC AGCGGATCAA TACGATTATC |
| 28141 | GCTTGGGTCA AAATACAGCA TTGAAGGATA GGTGGAGCCG ATGAAATTCT TAAATAATGA |
| 28201 | CTATTCTTTT CGTGGATTAG GACCCACAAA GGATGATCCA GAATACTTAG AAAATGCAGA |
| 28261 | ATATATCGAC TTTGCCGGTT TTAATTCTTC TGATTATGAT TGGTGGTTGA TTGATCGAAC |
| 28321 | AGCAACTACG CCAGAAGAAC AAGAAATTAC AGAAAGCGTC CCTTACATGC AAGGAGAATA |
| 28381 | TGATTTCTCA ATGTATGATC AGGAACGTTT TTTTAAGACC CGTGAGTTGA CTTATAAGTT |
| 28441 | TGTATATTTT GGTGAAGTTT ATCAGGATCG TAAAGCTTAC GAAGAGGAGC TTAAACGGCA |
| 28501 | ATTACTGCCA CATGGTTTCA CTAAACTAAT TGATTCTCAT GATCCTGTTT ACTACTGGTC |
| 28561 | AGCTAAGTGT ACTAGTGTTG AGGTTGAGGA TGACCAAGAA AAGGGAATGC TCACAGCAAC |
| 28621 | TATTACTTTC AAGGCTTATC CTTTTGCTTA TACTAATCAT AACGAGGGCA CCGATTATTG |
| 28681 | GGATGATGTC GCATTTGATC ATTGGATTTG GCAACCAGTT AAATTCAATG TTAACGGTGA |
| 28741 | TCAGGATGTT AATGTTAAGA ATATCGGCTC ACGACCAGTC GAATGCTCAT TTCAATTGAC |
| 28801 | AGGGTCCATA ACTTTGAAGA ACGATTCAAT TGGTGAAGTA GGTTTAACTC AAGACAATTT |
| 28861 | TAAAACAACC ACGATTGTAT TAGAGATGGG TGACAATAAA ATGCATCTAT CCGGAAACGG |
| 28921 | GACAATTGAA TTTCAATTTA AGCGTGAGGA GATGATTTAG TGTACCGAAT TATTGGTTAT |
| 28981 | AATGAACCAA CAGATAAAGC AGGATTTATT GTACTGGATC CCCGAGTTAA TCGTCATATT |
| 29041 | AGTTCGGGAA AACTCACGCT TAAAGAATCT AATATTGATG ATTTGACTAT TACGGTTAAT |
| 29101 | CAAGCAAGTC CATTATGGGA CAACGTAAGG CCTTATCATA CTCATGTTAA CGTTTATGAT |
| 29161 | GATAATGAAC TTATTTTTCG TGGACGAGCT ATCAAACCTA AAAAGTCGAT GGAAGAAAGC |
| 29221 | GGACAATTCA TTCGTGAATA TGTTTTTGAA GATATTGAAG CATATCTCAT GGATAGCACC |
| 29281 | CAAAGATTTT ATGAAGGTGT TGGTCAAACG CCCAAAGAAT TTTTACAAAC TTTAATCGAT |
| 29341 | GTTCATAATT CACAGGTTCC TGACTATAAA AAGTTTCAAG TCCGGAATGT AAATGTCACT |
| 29401 | AATAATAAGG ATGACCAATA TCGACAAATT GATTATCCCA AAACTAGCGA TGCTATTAAT |
| 29461 | GATAAATTAG TTAAATCTCT TGGTGGTTAT ATTGTGACTA CTTACAACGC TAACGGAATA |
| 29521 | AACTACATTG ACTACTTAAC GGATATTGGG GTTGATCATA AAGATGATAC TCCTATTCAG |
| 29581 | TTAGCTAAAA ATATGAAGTC TGCAAGTATG CAAATTGATC CTACTAAGGT GATTACAAGA |
| 29641 | CTGATTCCAC TGGGAAAGAC ACTAGAACCA TCAAAAGTTG ATGTAAGTGA TGATGATGGA |
| 29701 | GAGGGCGGTT CTGGATCATT AGATAGCCCT GAAGAATTTT GTAAATCAGA AATTAATGCT |
| 29761 | ACTTGGGGTA GTGATATTAA TAATATGAAA CAAGATTTTG CCGCTCGTTC TTCGAGAGTT |
| 29821 | CGGGCTTGGG GAGTGGACGT TAATCGTTTA TATGATGTGG TGAAAAATGC TGGAGTAAGT |
| 29881 | CCTGAATGGT TCTTTGCTTA TGAACTTCAA GAACAAGGAA CTTACTATGG ATGGCTTAAC |
| 29941 | CATACTTATC GACACGGTGA TGCGTATAGT GATGCGCAAT CTGTTTGTGA GTGGATTAAA |
| 30001 | AATTGTTCAA ATAGTAATTC CATTAATCCA GCATGGAGCG CACCGGAAGG ATCAATGGCG |
| 30061 | CCGAATCAAG CATTAGCGGA TAAATGGAAT CAAGAGTTTG GAAAAGGTAC TATTGGCCGC |
| 30121 | GTTTATTTAC AAGGGACTGC CGCTGCTGTT TGGGATTTAG CTGGTCAAAC GCCTAATCCA |
| 30181 | GCTATTGGAA AGCCAATTAG TGGATGCATT TCTTGTATTA AACGTTGGGG TGGTCATTCT |
| 30241 | AATGCAGCTG GTGGTACATG GGGATGGCCT TTTCCTGATG TTGGGGAAGG TCATTTTTCT |
| 30301 | CAAGTTCAGA GTTTCGGAAA TGATGGCGGA TATCGTCAAA ATAGTTATCA CGATGGTGTG |
| 30361 | GATTTTGGAT CAATAGATCA TCCTGGTAGA GAAGTGCATT GTATTCATGG TGGAACGGTA |
| 30421 | ACTATCAAAT CAGCTATGGG TGGCTTAGGT AATTTTGTGG TTATTCATAC GCCGGAAGGA |
| 30481 | TTCAATATCG TTTATCAAGA AGCTTTTAGT TCTCCCTCTA ATATTATTGT TAGTGTTGGG |
| 30541 | CAAAAAGTAA AAACTGGTGA TGTAATTGGA TATCGTGATA CAGACCATGT TCATATTGGC |
| 30601 | GTAACTAAGC AAGATTTTTA TCAAGCAGTT CGAAATTCTT TTTCTCCTGC AGGTGGTTGG |
| 30661 | CTAGATCCAG TAAAACTAAT TAAAGAAGGT GGCGATGGGT CTAAACCACA AGAAGGAAAG |
| 30721 | AAAGATCAAA CTGTTGATAA TAGTAATGCT GCACGTCCTA AATTAACCAT TACTACTGTC |
| 30781 | AATAACGGTA GAGACTATAT TGATATTCCT GATTTACAAA AAGAATTCGG TATTATTGAG |
| 30841 | GGAACTGTTG AATTTGATAA TGTAGATGAT CCGAATGTTT TAATGCAACA AGCTCAAACA |
| 30901 | TGGATAAAGG CTCAAAGAAT ACCTCAAAGT TGGGAAGTTA CAGCTTTAGA ATTACATATG |
| 30961 | ACAAACTTCA AATCTTTTAA GGTTGCTGAT AGGTACATGT TTATTAATCC AAATGTTGCA |
| 31021 | AAACCCCAAT TATTACGAAT TACTCAAAAA GAAATTGATT TACTAAAGCC CCATGCGTCT |
| 31081 | TCATTAACGA TTGGTGATAA GACGATGGGG CTTACTGATT ATCAGTTAGA AAATCAAGTC |
| 31141 | AATTTTCAAC AATTTAAGGA AATTCGAGTG ATGGTTAATC AGGTTGTCCA AACCCAAGAG |
| 31201 | CAATCTGCTA ATAACAATAA TAAGGTTATG CAAAATTTTG CTAGTAGTGC TGATCTTGCA |
| 31261 | CAAATGAGAC AGGATCTAAG AAATCTTCAA GATGATAACG ATCGTGCTCG CAAAGGAATG |
| 31321 | GTTTCCTTAG AAGAATTCAA TAAACTAAAG GAACAAGTAG AAAAACTAAC AACAGGAGGC |
| 31381 | GATGATAATG GCAAGTGAAA CCTATGATTA TGAGTCATTT GATAATACGG ATCATACTAT |
| 31441 | GAAACAAATC GCTGACGCTA TTCGTCACAA GGGTTATGGA AAAGATGTGC GTGAGGCAAT |
| 31501 | TGCACAGGGC TTCGAAAACT TAGATAAACA TTTAAGTAGT ATTGAAGAAG AACTGAAACA |
| 31561 | ACAAGAAAAG AAAAAGTCAT CGTCTATGGA TGATATTTTT AATTCTTTTG GTAAGAAGGA |
| 31621 | GTGATGATAA ATGGCAAAAG AGATTAGTAA TCTGATTACC TTTAACACCT ACAAATTTGA |
| 31681 | ACGAGGCGGC CTTTTGGTTG ATATGTTTAA CCAATTTAAT GCTCGTGTAG GAGATCAAGG |
| 31741 | AACGGAATTA GCCATCCAGT GGGAAACTAG TAAGACTGAA ACTAAAATTA ATTTAAAAGA |
| 31801 | ACGAGGATTA CATTTCTTTG GGACAGGTTC AGTTGGACAG TACCTTGAAA AATTAGAAGA |
| 31861 | TGGAACTGGC TTTAAGATGT CTGCAGATGC ATCTACCGTT GAATGGGAAG ATAAAGATGA |
| 31921 | AGCTGGTAGT TTAGACGATG GAATTACGGT TGTTAAGTTG CCAAAACAAT TTTTCCCTCA |
| 31981 | AAAAGGTATT TTCTTTGGTT ACTTTGGCCT AAAAGATAGA CAAGGTAATA TCTTTACTAG |
| 32041 | TGTTAATGTT TGGTTCCGTG TTCTTGGCGG TGTTCCAACG ATGGGGGCTG CTATTCCTTA |
| 32101 | CTTTGTTACT GAATTTGATG AAGTATTAGA GCGATGCAAT GGTAAGATTA TTGACGCTTT |
| 32161 | AGCAGAATTA CGTGAAAAGT ACCAGGCAGA AGTTAAGAAG AATGAGGATA TGTCTGCGGA |
| 32221 | AACAAGAGCA GCGTTGAGTA AACTTGCTGA TGCTGTTGGT GCAATTCAAG CGCAGATTGA |
| 32281 | TGCAGGTAAT GTAATTACAC GTAAAGAATA TAATAATCTT GCTAATCAGA TTGATAATCG |
| 32341 | CCTTAGTAAA ATGACACAGA ACATTGAGAG TTTTTCATCA CTCGATGACT TAAAAGCTCA |
| 32401 | ATATCCTAAT GGAAAAGATG GACTCTTTGT TACTAATGAC AATAATCATA AGTATCAATA |
| 32461 | CAAGAATGGA TCATGGGTAG ATGAAGGTAT ATGGACAGTT ACTACCTTTG ATCCAGAAAC |
| 32521 | GCGACGACGC CTTACATACC TTGATACATC TAATTCTATT TTGCAAAAAT CACTCAGTGA |
| 32581 | ATTGACTAAA GAAGTCGTTG ATATAAATTG GTTTTTAGGC GAAATTGATG CAAATACTGG |
| 32641 | TAAAATTACA CCTCACGAAA GTTTTAATCG TGCATACTCA TCACTCAAAG TAATTGGTAA |
| 32701 | ATCACATGCT TTTGAATTTT TGCTTAATAC TGATTATTTG CAATATATTA GTATTTTTGA |
| 32761 | ATTCAATTCA AATGGGGAGA TTATTAAGCA CGATAGTGTT GGCGATAAAG CGATATATAC |
| 32821 | ATTTGAAAAG GATACGACTG CGGTAAGGTT TCAGATTACT AGTACTGCTG CTACTATGAA |
| 32881 | GCAAAATGAT TTACGCGATA CTATTCAAAA TAGTGGGCTA AAAATTATCG ATCGAGGACA |
| 32941 | CCGTTCAGTA ATTAATGATA TTGATCATTT ATATATCCGT GAACATTTAA TGCCGATTGA |
| 33001 | AGCAATTGAA GGCTATACAA TTAACACTAA TGTTGATTAT GGTGAAACTG TTGATGTATC |
| 33061 | ACATCCAATA GTAACGTCTG CGTTTCAATA TATTAATCAA GTATGTAAGC CGGGCGATAT |
| 33121 | TTTCGTAATT AATAATCTAT CTGGAGGCTT TAACGCAATG GCTTGGGCCT TTATTGATAG |
| 33181 | CGAGAACCGG TTAATTCAAA AATCTGAAGT AAACTTATCT CAATCTCAGG TTACTTTATA |
| 33241 | TGCGCCAAGT AATGCTGCAA AATTAATTAT CAATAATATG GATAGTAACT GTACAGCATA |
| 33301 | TAGATATACT CCGGATAAAG AACATTTAGC TAAAATTAAT ATTGCTTTAA CTACCAATAT |
| 33361 | TCATCGAAAT ATCAAGTTAG CTTATCAAAC AGGAGAAACA GTCTCATTAA TTCCTGAAAA |
| 33421 | AGTAAGTAAT TACAAATATA TTATATTAGA TTGTAATTTT AATGATGCTT TCAGAATAAA |
| 33481 | GGGTTATGGT GGATTGAATC CACGATTATA TGGATTTATT TCTGAGCAAA ACACATTAAT |
| 33541 | GAATGTTGCG CCTGCAAATG TTAATGATCA ATTAACTGAT GTCTTTATCA AAACGCCAAA |
| 33601 | AGGCGCCAAA AAATTAGTAG TTAATTTCAA TCTTGATCAA CAAGTAAATC CCAAACAAAT |
| 33661 | TGCTGAACTT TATAAGCTTC CAGACATTAA TGTTATTACT GAGCAAAAAA TAAAAAAGAA |
| 33721 | TTTATTGAAT CAACAAACAG TCGATACATT AATTTCTAGT TATATGAATG GTATATTACT |
| 33781 | TGGTAAAGAT TTAGCCAATC ATCTTGCGGA TTTTGCTAAA AGCGGCGATA AAATGGTTCA |
| 33841 | TGTATCAACG TTTTATAAAG TTAACGATAC TCTTTTTATG TCTTATTATG CTAATACAAG |
| 33901 | ATCGGCTTAC GAAGATCCAA CCCAGCATAC AGCACGATTA GTTTATGCAC CGTTTGAGAA |
| 33961 | TCTAAATCAA CAAACGTACA TCGATGTAGC CGACATTGGA CAAGAATATA ATGGTCAAAA |
| 34021 | GATTGAAGCA ATATACGATT CTTTGCTTCT TAAAACAAGT GATGATTCAT ATATGATCTA |
| 34081 | TGCCTTTACT GCTAAGGTAG GTGGTAAGTT CTATATGCTT TACCGTCGTT TTGATCCTAA |
| 34141 | GACTAAGCTG TTGAGCGATA TTCATACAAT GAATTTTAAA GTTGGAGTAA TGACGTCGAC |
| 34201 | ATTTGATACA GTAAGCGTTC ATGATTTGTT AGCTAAGGCA GGAATTGACT ATGATTATGA |
| 34261 | AGATCGTGAT ATTTCCTTTG TCCAAAAATT AAGTCCACGG ATTGAAGATG GCGTAGTGCA |
| 34321 | ATACTATGCA GGTATCGGAA TCTTACATTT TTGCTTTGTA GTAAAATCAA GCGACTTAAT |
| 34381 | CAACTGGACT TTTGTCAGTA CTCCAGACTT TATGTACAAG CCAGAATTTG AACCTTCTGT |
| 34441 | ATATGTTAAG GGAGACAATG TTTATTATTT TTGTCGTCAG CGAGGGACAG AAGGTAATGC |
| 34501 | AGTTTTAGCT AAGTATAATA TCCCTAATGG TCAATGGTCG AATCCGATTC TTGTACCAGA |
| 34561 | TACTCAATCA CGCTATGACT TTTTCGAGAA TAATAGTCAG TTGTACCTTG TTCATTCACC |
| 34621 | GCTAGACCGT AATCATATTA GTTTGATGCA GATTGATCAG AATGTTCTGG AAAAGAGCTA |
| 34681 | TGAAGTAGCA ACAGCAGTAG TACAAGATTG CTTCTACCCA TTTACTCAAA ATATTGATGG |
| 34741 | TCAGATGTAT ATGAGCTTTA CACAAAGTAG GCAACATATT TGGCTAAATA AATTTAATCC |
| 34801 | CCACAGTCTG CTAGATAGTG ATGTAGCAAC TATTTTCAGT AATTTAATTG AGTAGCAAAA |
| 34861 | CATAGTCGCC TTATAAATAC ACAATACATA GAAAGTTGCA TTGCTTAAAT GGAAACTTAA |
| 34921 | TGGGCGGCTT ATAGACTTTT ATTATTTAAA AGAGTATACT GGAATTATTC TTTTTATTGG |
| 34981 | GAGGAAATAT AAAAGTGACA ACTAAAGTGA GAGATAAATC GCTTGATATT ATCCGGGGGA |
| 35041 | GCATCCTTCT AGTTGTCTTG GGTCATATAT CTGGGATACC TTTTGAGTTA AAAAAGTACA |
| 35101 | TATATTCTTT TCATATACCA CTGTTCTTTT TTGTTTCGGG ATATTTGTTC AACTTTGCTA |
| 35161 | AATACAGGTA TTTTTCTTAT AAAGAGTTTA TAAAATATAA AGCTAAAAAA TATATCCTAC |
| 35221 | CTTATTTCAG AATGGGGTTG ATATGTTTGC TTCTATTTGG CATAGTTTAT CCACTATTTG |
| 35281 | CTGAAGGGTT TAGTAAACAG TATATGCTTC AATCTACAAA ATATGTTTTA GGTTTACTAT |
| 35341 | ACTCGCGTGG AGGTCCTAAT TATATGGCCT GGAGTTCGCC ACTTTGGTTT CTAACAGCCT |
| 35401 | TATTTATTGC AGAGATTATT TTTTTTGTGG TTCTAAAATT TAATTTTAAA TATCCATTAA |
| 35461 | TAGTGTTTGG AATCTTAGCT ATATTGAGTT ATATTTACTC GATTACAATT AAAATTCCGT |
| 35521 | TACCGTGGAA TATTGATGTT GCAATGTTTG CAGTGTTATT CATGTACCTA GGCTTTATTA |
| 35581 | CACACAAATA TAATTTAACT AAGCACATTA ACTTACCGGT TTTCTTACTT TTGATTGTTA |
| 35641 | TTTTTGTATT ATCTGTAGCT TACAATAATG AGATTGACAT GAATTTAAGA AACTATGGCA |
| 35701 | ATGGATTTTT AACAATTATT AGTGGAACAA TAGGTACTGT TATATGCTTG CAAATTGCGC |
| 35761 | GATTGTTAAA AGAGAATAAG ATATTAGAGT TTTATGGTAA AAACACATTA TTTATCATGG |
| 35821 | GCTACACGTA TGCTGTGTTT AATTGTATTT TGGCTTTAAG CAGCCATTTT AGTACTGTAA |
| 35881 | AGAATGTTGT AGCTTCGTTT TTGATTCAAA TTATAATTTT AACTTTATTA ATAGTACTGA |
| 35941 | AGAATTTATT CAAACAAATA AAAAAGCCCA TTTATGCATA TACTAGGAAA ATTAACAATT |
| 36001 | AAAAAAACTA ATACGTCCTA CTCAGGGCGT TTTTATTTTA CCTTCAAAGG AGATGATCAA |
| 36061 | CATTGCCCTA TCATTTATTT ATGCTGCACC AAATGCAGAC ATTAGTTGAT GATAAACTAA |
| 36121 | TGTGGGCTTT TACAATCGTG ATGATTGTAG ATTTAATTAC GGGAATGATT AAACCGTATT |
| 36181 | ATGCAAAGAA AACAATTAAG AAAACTAATA GTTCAGTTGG AATCCCTGGG ATAATTAAAC |
| 36241 | ACACAGTAAT TTATTTAGTG GTAGTAATTG CTTATCCATA TCTTTATACG ATTGGAGCAA |
| 36301 | GCACGATGGC TACCACTTTT TTAATTGCTT GGATTTATCA ATATTTAATT TCAATTGTAG |
| 36361 | AAAATTGGAC AGAGATGGGG TGGTGGTTGC CTAAACCAAT CATGGATTTC TTTGAAGCCA |
| 36421 | AATTAGCTAA GGATCAAGAA GATTATGACC CATCTAAGTA CAATTTTCTT GGTAAATATA |
| 36481 | AAGGAGGTAA AAAGTAA. |

### Example 4. Selection of L. reuteri Promoters and Transcription Terminators

Global proteomics analysis of L. reuteri 3632 pellets and culture supernatants is performed to identify potential strong promoters and secretion signals, respectively. The proteins in the pellets and supernatants are ranked by their expression level, and the promoters and secretion signals from highly expressed and secreted proteins are identified by whole-genome sequencing (Example 2). The genetic elements are selected for use in expression cassettes that can be used to generate a genetically-modified bacterium which deliver the desired biomolecules to host animals in need thereof. Expression cassettes would comprise a suitable promoter, a heterologous coding sequence encoding a desired biomolecule, and a transcription terminator. The heterologous coding sequence could comprise a signal sequence for secretion, a cell-wall anchor sequence, and/or a detectable peptide tag.

Six suitable promoters are identified through analysis of the global proteomics data. Those promoter sequences are:

```
Xylulose-5-phosphate phosphoketolase promoter
(SEQ ID NO: 14)
    1 TTAAAGTATT AAAATAGATG TAAAATTTAT TTTTTTCAAA AGAAATTTTA ATTGTACACT
  61 GTTGGTATTG AACGGGGTTA AACAAAGGTA AATTAGCATT TCTGCGGATT AAGATAAATA
121 GAAAAATGTT AAAGAACACC TTAAAAAGAT TAATTTTTTA TAATTGGACC GTATCAATTT
181 GTAAAAAGGT TGACTTTTTG AAAAAAAAGT TTATCATTAA CATTGTAAAT TTAATGATTT
241 ACGTTATGTT GTTATAGAGC ACAGGACGTA TTGATTTATA TAGAAGGAGT GTTTATTAGA;
Elongation factor TU promoter
(SEQ ID NO: 15)
    1 ATGAATGGAC AGATGTTTTA ATCGCTAGAA TAGAAGGAAA GAAAGTCGCA ACAAATACGG
  61 TTTCTAGTAC GTGGCAGGAA CGACTAGGTA AGCAGATTGA CGAATTAATA GAAAAACATT
121 AGTCAAATAC ATTTACAAAT GAACAGATAG TTGATATTAT ATTTAAGAAT TCTTCTTCAG
181 AGCCTAAGAT TAAAGCTTTC AATTGGCGAA AAGAAGTTGT ACAATATGTA TAAAGGTATG
241 TCAGTCACCG AATCAGATGA TCTGGCATTA TACTTGTAAA TTATCAGGAG GTTTTCATTA;
Glyceraldehyde-3-phosphate dehydrogenase promoter
(SEQ ID NO: 16)
    1 ATCTCACGTG CGATCCATTA CACTAAGGGC GCGTCAACAA ATATTATACT ATCTTAAATA
  61 AGAATGAATT GCAAGCATTA TTTGAAAATT TTAATTAAAA TAACGCTTAC ATCAGAAAAA
121 TGTTGTGATT GAATAGACAA TTTTTTTGAA GATGGTATCA TAAGTATCGT AGGAGTTGTA
181 TTATTGCTTA GACCTTACCA CTGCGTCACT TACAATGGTT GAGAGTTGCG ATGCTGATGT
241 AATGTGATAA ACTAAGCAAG TACACTAATT ATGTTTTTTC CTAAAGGAGG AATTTGCAGT;
Glucose-6-phosphate dehydrogenase promoter
(SEQ ID NO: 17)
    1 TTGTTTAAGA TATCTTTCAA AGCTGCGGAA TTTTTCCCAG CTTTTTTAGT TAGTTTTGTT
  61 TTCATAAGCT ATAATTTTAA CCGATTCCAA ATTTCTTTTA AAAGTTTTTT TGATCTAGAC
121 CATTAATTGA TAAACGCTTA CCAAAGACTA ATCAACAAGC CATTTAGCGG TAGTGGTCCA
181 TTTTAACTTT CTAAGACATC TTCTCAGAAA ACGTTTCCTT TGATAGTGCA GATTGTGCTT
241 TAAGAGTATA TAATTGTCAC GGTATAAGAA TTTTCTGAAA TTTCAGAAGG AGTGAACATT;
L-lactate dehydrogenase promoter
(SEQ ID NO: 18)
    1 CTCCTCTATT ATTATTCCTG ATCAATTTTA AATTAATCTC CCTAGATAGG TATATTTTAG
  61 CACAGGTCAC CAACGTTCCA AAGTTTAATC TATGTTTAAA CTTTAATTTT CAAAAAAATG
121 CTATACTATG TTCACGATAC TTTAAGGAAA GGTGATTACA ATAGTGAGTC TCTTAATTGC
181 TATTCTTATC TGCTGGTTGC TATGGAAGAT TGGGGGTTTA ACGGTTAAGT TCATTGGTCT
241 AATCCTTCTT ATTCTATTAA TCGGGACATT AATTCATGTT TTACTTTGGC CAGCGATCCT
301 TTTAGCAGTT ATTATCTTAG GAGCAGGTTT ATTCACTAAC TAATTTATCT ATAAAATCTT
361 ATAGTAATTT TTCTGCGGAA TGTTATAATC ATTACTGTGA GAGAAATCTC AAATAATGTA
421 TACATAAGAT GAAAGGGAGA CTGTTTATT;
and
tuf promoter
(SEQ ID NO: 19)
    1 ACAAATACGG TTTCTAGTAC GTGGCAGGAA CGACTAGGTA AGCAGATTGA CGAATTAATA
  61 GAAAAACATT AGTCAAATAC ATTTACAAAT GAACAGATAG TTGATATTAT ATTTAAGAAT
121 TCTTCTTCAG AGCCTAAGAT TAAAGCTTTC AATTGGCGAA AAGAAGTTGT ACAATATGTA
181 TAAAGGTATG TCAGTCACCG AATCAGATGA TCTGGCATTA TACTTGTAAA TTATCAGGAG
241 GTTTTCATTA.
```

The underlined regions of the above sequences denote the ribosomal binding sites.

Four suitable secretion signal peptides are also identified through analysis of the global proteomics data. Those secretion signal sequences can be derived from the following proteins:

```
C protein alpha-antigen precursor
(SEQ ID NO: 20)
      1 MVSKNNHQFY QQKHAERKQR WGIRKLSVGV ASVLLGTTFM LYGNHAVLAD TVTSPSDDVT
    61 RSTTTQGGNK DKVTEGTTEG TTSTPQTSGD STDKQANGQN VNQQVPTTDT EEATNHQDTP
  121 QGQDTTQNTT NVDKKDTEVT PANDATTPTT QKITAKFTTA KFTTAKFTAA KFKVLAARPV
  181 MKVAGTASLP ISNQDIKLDS QPMLTEIINK PTDNWVYNNL KWYQDTSTEK IKEILQNHTA
  241 NDESGRYYFA GVANYNEHYH AIYLLARSNN LNDNSLYVTI LHTGLGKNIQ EAVVAPGESK
  301 KVEYSGTTHT PIFTNYDGTS ASIDLDGIEK GDNIYGMVVG FAYGHNTGIK GDPASMGNGF
  361 VMTPIPTKMT TTIHYIDQAT GDEIAVPKSF EGVAYQKYTI TGEAPTIDGY TLKKSPETTG
  421 YISPYKVGES YDFRLDKHVV IKQTVIDAQG LVRVTAYYDG EVLNNTTRYL GNKLNVNDRM
  481 SFISHGKWYT YINQITSTND GIVYYYAKDG SEDKSEVRVH YIDVTGSKNS IFVPGDGEEV
  541 ATDKISGKLG ENYNYDVNLP TDYNLATNQA NTVNGTYTID HHDEYVYVVK KTSAELDPTV
  601 PAKTKVDNPT SLTADEKKTI EDKIVEANKD KFPEGTGVTV ANDGKATITY PDKSVDTIEG
  661 NQLVEEKTSA EKLDPTVPAK TKVDNPTSLT ADEKKTIEDK IVEANKDKFP EGTGVTVAND
  721 GKATITYPDK SVDTIEGNQL VEEKTSAEKL DPTVPAKTKV DNPTSLTADE KKTIEDKIVE
  781 ANKDKFPEGT GVTVANDGKA TITYPDKSVD TIEGNQLVEE KTSAEKLDPT VPAKTKVDNP
  841 TSLTADEKKT IEDKIVEANK DKFPEGTGVT VANDGKATIT YPDKSVDTIE GNQLVEEKTS
  901 AEKLDPTVPA KTKVDNPTSL TADEKKTIED KIVEANKDKF PEGTGVTVAN DGKATITYPD
  961 KSVDTIEGNQ LVEEKTSAEK LDPTVPAKTK VDNPTSLTAD EKKTIEDKIV EANKDKFPEG
1021 TGVTVANDGK ATITYPDKSV DTIEGNQLVE EKTSAEKLDP TVPAKTKVDN PTSLTADEKK
1081 TIEDKIVEAN KDKFPEGTGV TVANDGKATI TYPDKSVDTI EGNQLVEEKT SAEKLDPTVP
1141 AKTKVDNPTS LTADEKKTIE DKIVEANKDK FPEGTGVTVA NDGKATITYP DKSVDTIEGN
1201 QLVEEKTSAE KLDPTVPAKT KVDNPTSLTA DEKKTIEDKI VEANKDKFPE GTGVTVANDG
1261 KATITYPDKS VDTIEGNQLV EEKTSAEKLD PTVPAKTKVD NPTSLTADEK KTIEDKIVEA
1321 NKDKFPEGTG VTVANDGKAT ITYPDKSVDT IEGNQLVEEK TSAEKLDPTV PAKTKVDNPT
1381 SLTADEKKTI EDKIVEANKD KFPEGTGVTV ANDGKATITY PDKSVDTIEG NQLVEEKTSA
1441 EKLDPTVPAK TKVDNPTSLT ADEKKTIEDK IVEANKDKFP EGTGVTVAND GKATITYPDK
1501 SVDTIEGNQL VEEKTSAEKL DPTVPAKTKV DNPTSLTADE KKTIEDKIVE ANKDKFPEGT
1561 GVTVANDGKA TITYPDKSVD TIEGNQLVEE KTSAEKLDPT VPAKTKVDNP TSLTADEKKT
1621 IEDKIVEANK DKFPEGTGVT VANDGKATIT YPDKSVDTIE GNQLVEEKTS AEKLDPTVPA
1681 KTKVDNPTSL TADEKKTIED KIVEANKDKF PEGTGVTVAN DGKATITYPD KSVDTIEGNQ
1741 LVEEKTSAEK LDPTVPAKTK VDNPTSLTAD EKKTIEDKIV EANKDKFPEG TGVTVANDGK
1801 ATITYPDKSV DTIEGNQLVE EKTSAEKLDP TVPAKTKVDN PTSLTADEKK TIEDKIVEAN
1861 KDKFPEGTGV TVANDGKATI TYPDKSVDTI EGNQLVEEKT SAEKLDPTVP AKTKVDNPTS
1921 LTADEKKTIE DKIVEANKDK FPEGTGVTVA NDGKATITYP DKSVDTIEGN QLVEEKTSAE
1981 KLDPTVPAKT KVDNPTSLTA DEKKTIEDKI VEANKDKFPE GTGVTVANDG KATITYPDKS
2041 VDTIEGNQLV EEKTSAEKLD PTVPAKTKVD NPTSLTADEK KTIEDKIVEA NKDKFPEGTG
2101 VTVANDGKAT ITYPDKSVDT IEGNQLVEEK TSAEKLDPTV PAKTKVDNPT SLTADEKKTI
2161 EDKIVEANKD KFPEGTGVTV ANDGKATITY PDKSVDTIEG NQLVEEKTSA EKLDPTVPAK
2221 TKVDDPTKLT NDEKKEVEDN IRDHNTGLPE GTKIAVGDNG DTTITYPDKS VDTIEGNQLV
2281 EEKTSAEKLD PTVPAKTKVD DPTKLTNDEK KEVEDNIRDH NTGLPEGTKI AVGDNGDTTI
2341 TYPDNSVDTI PGDKVVEGKS DAAKNEPKVP GDKVKVDDPN KLTEDEKSEV VKAVEDANKD
2401 ENGKSTLPEG SKVTVGDNGD VTVTYPDGSK DTIPGDKVVE GKGTEGQTDA DKNEPKVPGD
2461 KVKVDDPNKL TEDEKSEVVK AVEDANKDEN GKSTLPEGSK VTVGDNGDVT VTYPDGSKDT
2521 IPGDKVVEGK GTEGQTDADK NEPKVPGDKV KVDDPNKLTE DEKSEVVKAV EDANKDENGK
2581 STLPEGSKVT VGDNGDVTVT YPDGSKDTIP GDKVVEGRGT EGQTDADKNE PKVPGDKVKV
2641 DDPTKLTEDE KSDVEQAIKD ANKDENGKST LPEGSKVTVG DNDDVTVTYP DGSKDTIPGD
2701 KVVEGKGTEG QTDADKNEPK VPGDKVKVDD PNKLMEDEKS DVEQAIKDAN KDENGKSTLP
2761 EGSKVTVSDN GDVTITYPDG SKDTIPGDQV IEGKSDADKN TPNVPGGDKV KVDDPTKLTD
2821 NEKNAVKDKV DEANSNLPDG TKVTVGDDGT TTITYPDGST NTISGHDLVT GKTDADKYPL
2881 NPGQAVNVVD PNHLTQAEQD QVKEAIQTTN PTAPIATITV DTAGNVQVTF ADGSTTTLQA
2941 NLHKHVTEAT TGSAIKPGVG TNGGQTKGAT STNQTATKQQ AQQHLPQTGD QPATWAMLSG
3001 LGVAFLGLLG LKKKRED;
Arabinogalactan endo-1, 4-beta-galactosidase precursor
(SEQ ID NO: 21)
      1 MEIKKHFKLY KDGKKWCCAA IATTVLGIGL AIGSPSVLAD ADTITSTSDA NNSLVKNDNT
    61 SDTDSNSEST FTDTNKNSTN EKEINENKNI DSSQQINQEQ TKSNNSEEQT TPVNVKAENT
  121 DIKDSIPEKS TPNSFKEING STYYYGENGD LYRNQFYNNW GRTYYFQANG ARLDNGFYNN
  181 WGRTYYFGSD GARWDNRFYN NWGRTYYFQN DGSRLDNSFY NNWGRTYYFG VDGARWDNRY
  241 MVKWGRAYYF GNDGALLQNQ LKSINGINYW INNEGIIPLK NQFLTANENQ LFYFDGNGSL
  301 VVNKFYHNWG HTYYFGSDGA RYTDQFLNRD GKVYYFDNQG IMYQDQYYKN WGHTYYFGSD
  361 GARYTDQFLN RDGKVYYFDN QGIMYQDQYY KNWGHTYYFG SDGARYTDQF LNRDGKVYYF
  421 DNQGIMYQDQ YYKNWGHTYY FGSDGARYTD QFLNRDGKVY YFDNQGIMVT NQVRVIDGKG
  481 YEFNDNGEAT ETSDMGQTRD TVAKEVAQAL TNQGIKGVKY DWRNTNNDYQ ELALHDIAQE
  541 VAQGDTNPDK NVIEKKLQAN NLLSGKVLVV YSTDFTNDDP QKITNTFMNS YDFTNADNSV
  601 LGVGADLNKN KLVIILFKPG EKAEQPQATS TISASISDIF KKAGVNVDVD NGLTKGSVVN
  661 SADLGNALTN GTAELLKGDK GTIISQEVLK AIFAAFAGNT SAVEGTKNYY NGNDAYHYEF
  721 WLEGQSADDK LNNFLALNKG AKYGDQLKVN YTATLVFGQE TGTNSNESKV PASERTDEQL
  781 DLAYKTGTDT GLRYDSVKVE KIPGMTDDMV RGVDVSSYQA LINAGVKFYD FNGQESNLFK
  841 ILKDSGVNWV RLRVWNDPYN AQGQPYAGGD NNEENLIKMA KEASDNGLKL LIDFQYSDFW
  901 TDPAQQILPK AWRNLSHGEM SQEVYLYTSK ILNDLQKAGA SVKMVQIGNE ITNGAFGLYT
  961 GRNGGGNWAS LWETSDGDQV AKYIQAGSSA VRRIDPTIKV AIQLETPEIN KYRGIMNVLK
1021 KNNVDYDYLG TSYYPFWSTT QGNGWYDNVD LGYGANTPVN LEAIEKMAWN EFGKRTVILE
1081 SGWLNNTNDA DGTHNSVGEN NETTNIDRYS ADPQGQVDEI EDMYNAIIAQ KGLGAFYWEP
1141 AWIPVKAGWN NWQYNKLMSN IYGSGWASQY AKGYAPDSVL YYDGKEAWGG SSWDNISLFD
1201 DHGHPLQSLN VYNGMLNGYE SPKNVKSSLS TQLVKIWNET DVIPNDGLTE GTKLSTDLFG
1261 TTQLSGNDGQ SIGNAELTKL AGRLKDGISS KVYTAANGAR YHYIYWLEGG NNKVNTFVSA
1321 NKDAKYGQPL IANYSATVVV DSEPGTQVAT SPLQIKISQV WNTVNNEEIK IDNPLKQGDL
1381 ITDKSDNAFS GILNSKDIKE ALTGEKGKDV SESTVNDVKS LLPKEVKGSK TYTTADGNQY
1441 YYDFWLASVE TSNVNYGEPI IVNYTASLKW LG;
Chromosome segregation protein
(SEQ ID NO: 22)
      1 MEKTMKKKAL VATTAVAGIT LVGEVTTVHA ADNVQQPVNE QNVNQSSQEE KQAAQNLQNA
    61 QSDVNTATEA NSNAQDNLAS ANNNLSNAKK AVSDQAAKVA DATKAQSDAS TKVDNDNKVV
  121 ADAQQKADQA TPANIENAKQ AIEGQNKVID QDNENIKYSN TDQDKAQNTL NNAQSNEDKA
  181 NATLSNKKSS QASAQNNVKQ AEDALNGTHL VEAQNAFNQA QSNVENAQSK YDQANNQLSD
  241 AQKKVTTNQN DLTAKNKALD NINNQVDTDQ NNVNSNQATA DSASSATQVA QNAVDQTKQS
  301 LDKVIEELNG FSENTIKVPA GAQEAYEAFI DAVDNNADQS QLDSLAKKMY DTLHQGQGTN
  361 GINHFNSSKY DQNQLVDVDH LTTDQLNELT QFAADMINSA RKAWGSDKNA GTLIPTQGVS
  421 EMAQQIAKGY VSDNWHISQG HDVKRVTAAA GLIGLNDAGQ FYEDASEGYV HAWPWEKDSY
  481 TMDNLKEAVY DSILGMLFAD DNSGNGHMTD LLGLHVNRKE DHQYFGLSTN MCPGSYMGQL
  541 HFIIVENDPA YIKDPQTFNA KGGTTKIEYI DPKVQLNQQK DILTTTLSTQ QADLATKQDA
  601 LNKANQNLAN AKKQLSEDQD LQTVAQQNRD SAQKALNDAT AKVSNLQATV NSLSQDLNSA
  661 KATLDQAKKT LESYTADHKA KLDNYNNAKA ALDDANKAVA EAQSAVDTAV NETKIAQNNL
  721 DQKKQAVTDA QNKLANDQEY LATLKQNLAD LQNAPQNLQK AKDQLAKDQI ALDNANKDLQ
  781 NQKDSLDELN KKLEDAQVKV NEAQSAANVT KATLDQAQAK LSDAEATWKE LHNDAHRYGN
  841 VVKVTPITME AGTSLPDPVI ENGFTVNTGT NQLFVSLAAI DSSNNNIPQG TKASWANRSK
  901 ALTDSQNAGS YSEDILITFP DNSTVTVPVD LTVTAKKITE DQKATEGGYH IVNGSVVDKQ
  961 NNLVSGWTVK NGQMVDPEGN VIKTTMSTAQ GVTIEKNNSK SGNTKTNMIQ TSLTIANNKA
1021 TTNKDNQLPQ TGNYNNNTKV LGLAGIALAS ALTMFGYKKR QHN;
D-gamma-glutamyl-meso-diaminopimelic acid endopeptidase Cwls precursor
(SEQ ID NO: 23)
      1 MKSTTKKILA SSLGVAGAMA MGTVTAKADT TVTVNAGDSL NGIAQKYNVS ADDIATANHL
    61 QNKELIFVGQ KLTIPTKDKN ETPANNAEKK DQASKNSQSL QDSVNKAMSY LGTPYVWGGN
  121 KPGGFDCSGL VQYCYGIPQR TTYEQQALGP HIHDNVLNAP YGALVFYGSD DAPYHVAISL
  181 GDGRIIQAPN ENETVKITDQ QYFPGNYYVV MH,
and
Chromosome Partition Protein Smc
(SEQ ID NO: 40)
      1 MNKANQKVAD DTTAVNNKQT DVNNAAEAKK NADEALKNAN DAQTSAQKNK DAKQAIADEA
    61 SVALADANTA VKDAQAKVDA INDKLANFNT ITLPAGYKDD LIAYYNYFGN SNYNQDEANN
  121 LAQDLLKYRD QAMSQNKFKD NLSDDRVVDI DNLNSTDRAE LSQFVASLIN QVRTQMGTNL
  181 VISSPAADDY AEQVSQNYNK DNWNSADNGK HDQSALNNAT DQLNISWNGE NMGLDQSIFT
  241 TDYTVLTDGT KLPTGNKQTI NDLKHLIYDD FISMMFDDAD SAWGHATNFA GIDNFAAEKQ
  301 AVGFSLDKFY NTHYDLVEAN QKVEENSYTL PSINALTQKL ADAKDDLSIK QTDQASKQKA
  361 NDDAQNALSS ANQVLVAAQN DVKDKTATAQ EANDNLTTAQ NDLATLQNQL SADQANQKQA
  421 QTTFDSFDAD LATKQANLQK ATDSLKAEQG RLAIAQADLD NANKALSDAN NNLAQKKQVV
  481 ENDNETLKVD NDKLVQLQNN LSDLQNAPKL LAAAKEQVAT AQKALADAQE AYNVANDKLT
  541 SLKQTAAGTT TNVSKAQQAL AEAKNNEDAA KEVLDQAQQA LTELRQKEAL AKQVAEEQAK
  601 LAAEKEAKDN GYHIENNQVV DAKGNSVNGW TVKGNQIVSP TNATVDPAVS VTTNVNVDSK
  661 GQVQPQTSVT ANSVKTVAAT ESANPVATTT VQTREQYKQQ LKSNNQLPQT GNNDSAVLSL
  721 AGVALAAMLS LFGIKKREY.
```

A person of skill in the art would recognize that, because of the redundancy of the genetic code, multiple nucleic acid sequences could encode the above peptides.

### Example 5. Expression Cassettes

Based on the information provided in the previous examples, expression cassettes that produce high levels of secreted target biomolecules can be designed. For example, one of these expression cassettes (CwIS_C2; SEQ ID NO: 23) has evolved to deliver endopeptidase (cell wall hydrolase) to the cell wall. That endopeptidase plays an important role in cell division and separation. Replacing the endopeptidase with a heterologous coding region would result in the secretion of desired target biomolecules, such as anti-infective biomolecules that target pathogenic bacteria. A specific example is that the novel mersacidins disclosed herein could be expressed at high levels by exchanging the native mersacidin promoter with a strong promoter (Example 4; SEQ ID Nos: 14-19) and optionally a strong secretion signal (Example 4; SEQ ID Nos: 20-23).

Expression vectors are designed and synthesized with different combinations of some or all of the following components: an origin of replication for replication in Z. reuteri (e.g. from the high copy number plasmid; SEQ ID NO: 8), an origin of replication for replication in E. coli, a drug resistance marker for selection, a strong promoter for expression in Lactobacillus, a signal sequence for secretion, a heterologous coding region encoding a desired biomolecule, an expressed peptide tag for detection, a cell-wall anchor for secretion, and terminators for transcription termination.

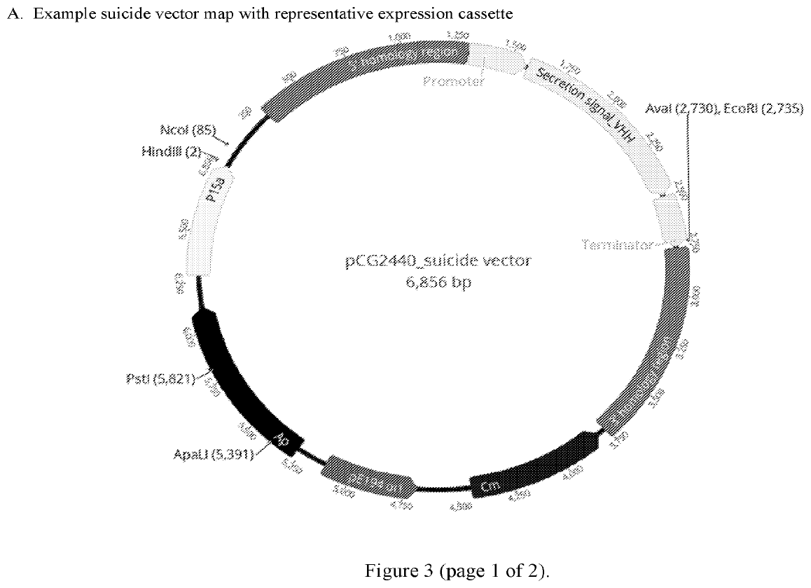

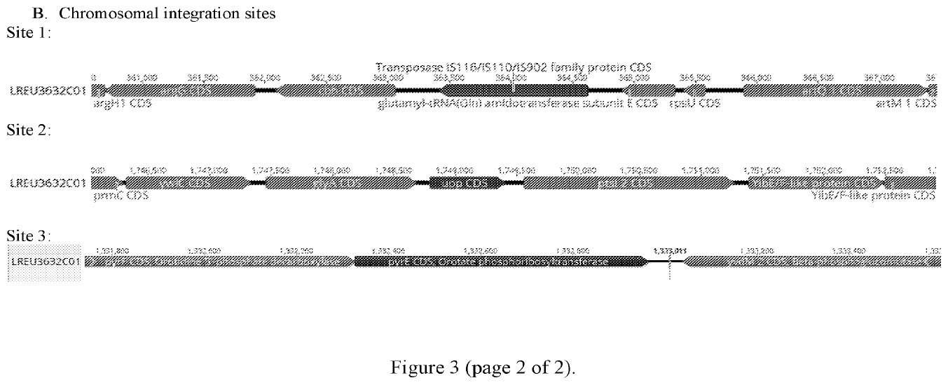

Alternatively, a heterologous coding region encoding a desired biomolecule could be integrated into the chromosome of the genetically-modified microorganism. Chromosomal integration of the expression cassette (a strong promoter for expression in Lactobacillus, a signal sequence for secretion, a heterologous coding region encoding a desired biomolecule, terminators for transcription termination, and optionally an expressed peptide tag for detection and a cell-wall anchor for secretion) is accomplished with a suicide vector. The suicide vector comprises an origin of replication for replication in E. coli, a drug resistance marker for selection, and an expression cassette flanked by nucleic acids homologous to a specific region of the chromosome. An example of such a suicide vector is presented in FIG. 3A.

For chromosomal integration L. reuteri genes may be interrupted by the insertion of the expression cassette. Maps of these chromosomal loci are presented in FIG. 3B. Two genes which may be interrupted are Transposase and Uracil phosphoribosyl (UPP) transferase. Chromosomal integration may also be accomplished within the pyrE genelocus, and pyrE deletion and subsequent re-insertion may serve as a selection marker for integration (Sakaguchi et al., Bioscience of Microbiota, Food and Health, 32:59-68 (2013)). A modified pyrE gene may also be used for biocontainment of recombinant bacteria, as the recombinant bacteria cannot survive in an environment absent metabolic supplements.

### Example 6. Enzymatic Genes for Inclusion in Expression Cassettes

An expression cassette would comprise a heterologous coding region encoding a desired biomolecule. The desired biomolecule may be a biomolecule with anti-infective activity. The anti-infective activity could be lysis of pathogenic bacteria by a lytic enzyme, for example from a bacteriophage, with specificity to a certain genus of pathogenic bacteria.

Lytic enzymes may include PlyCM, a lytic enzyme targeting Clostridium perfringens, encoded by a sequence of:

| (SEQ ID NO: 24) | |
|---|---|
| 1 | ATGGAAAGCC GTAATAACAA TAACCTGAAG GGCATCGATG TGAGCAACTG GAAGGGCAAC |
| 61 | ATCAATTTTC AAAGCGTCAA AAATGACGGT GTTGAAGTTG TTTACATTAA GGCAACCGAA |
| 121 | GGCAACTACT TCAAAGACAA ATATGCTAAG CAAAACTACG AGCGCGCTAA AGAACAGGGT |
| 181 | CTGCGTGTGG GCTTCTACCA CTTTTTCCGC GCAAACAAAG GTGCCAAAGA TCAGGCGAAC |
| 241 | TTCTTCGTGA ATTACCTGAA CGAAATCGGT GCGGTCAATT ATGACTGTAA ACTGGCACTG |
| 301 | GACATCGAGA CTACCGAAGG CGTCGGTGCG CGTGACCTGA CCTCTATGTG CATCGAGTTC |
| 361 | CTGGAAGAGG TGAAGCGTAT TACGGGTAAG GAAGTTGTCG TGTACACCTA TACCAGCTTC |
| 421 | GCGAACAATA ATCTGGATTC CCGTCTGTCT AGCTATCCGG TGTGGATTGC GCACTATGGC |
| 481 | GTCAACACCC CGGGTGCGAA CAATATCTGG AGCGAGTGGG TGGGTTTCCA GTACAGCGAG |
| 541 | AATGGCTCCG TCGCCGGTGT CAGCGGTGGC TGCGATATGA ACGAATTTAC CAATGGTATC |
| 601 | TTTATTGACT CGAACAATTT CACGTTGGAC AATGCAACGA CCAAAAATGT TAGCATTAAG |
| 661 | CTGAACATTC GCGCCAAGGG TACGACCAAC AGCAAAGTTA TTGGTAGCAT TCCGGCGAAC |
| 721 | GAAAAGTTTA AGATCAAATG GGTTGATGAA GATTACCTGG GTTGGTATTA CGTTGAGTAT |
| 781 | AACGGTATCG TGGGTTACGT TAACGCCGAT TACGTCGAGA AACTGCAAAT GGCGACCACG |
| 841 | CATAATGTTA GCACCTTTCT GAATGTACGC GAGGAGGGTT CCTTGAATAG CCGTATTGTG |
| 901 | GACAAGATCA ACACTGGCGA CATCTTTCGT ATTGACTGGG TTGATAGCGA TTTCATTGGT |
| 961 | TGGTATCGTG TGACGACGAA AAACGGCAAG GTCGGCTTTG TTAATGCAGA GTTTGTGAAA |
| 1021 | AAGTTGTAA; |

PlySS2, a lytic enzyme targeting Streptococcus suis and Staphylococcus aureus, encoded by a sequence of:

| (SEQ ID NO: 25) | |
|---|---|
| 1 | ATGACAACAG TAAATGAAGC ATTAAATAAT GTAAGAGCTC AGGTTGGGTC CGGTGCGTCT |
| 61 | GTTGGCAACG GCGAATGCTA CGCTTTGGCT AGTTGGTACG AGCGCATGAT TAGTCCGGAT |
| 121 | GCAACTGTCG GACTTGGCGC TGGTGTGGGC TGGGTCAGCG GTGCAATCGG CGATACAATC |
| 181 | TCTGCCAAAA ACATCGGCTC ATCATACAAC TGGCAAGCTA ACGGCTGGAC AGTTTCCACA |
| 241 | TCTGGTCCAT TTAAAGCAGG TCAGATTGTG ACGCTTGGGG CAACACCAGG AAACCCTTAC |
| 301 | GGACATGTGG TAATCGTCGA AGCAGTGGAC GGCGATAGAT TGACTATTTT GGAGCAAAAC |
| 361 | TACGGCGGGA AACGTTATCC CGTCCGTAAT TATTACAGCG CTGCAAGCTA TCGTCAACAG |
| 421 | GTCGTGCATT ACATCACACC GCCTGGCACG GTCGCACAGT CAGCACCCAA CCTTGCAGGC |
| 481 | TCTCGTTCCT ATCGCGAGAC GGGCACTATG ACTGTCACGG TCGATGCTCT CAATGTTCGC |
| 541 | AGGGCGCCAA ATACTTCAGG CGAGATTGTA GCAGTATACA AGCGTGGTGA ATCATTTGAC |
| 601 | TATGATACTG TCATCATCGA TGTCAATGGC TATGTCTGGG TGTCTTACAT AGGCGGCAGC |
| 661 | GGCAAACGTA ACTACGTTGC GACGGGCGCT ACCAAAGACG GTAAGCGTTT CGGCAATGCT |
| 721 | TGGGGTACAT TTAAATAA; |

and CP025C, a lytic enzyme targeting Clostridium perfringens, encoded by a sequence of:

| (SEQ ID NO: 26) | |
|---|---|
| 1 | ATGTCGAAGA TTTTTGGTTT AGATGCGGGT CATTGTACGA GCGGCGCAGA TACGGGTGCG |
| 61 | CAGGGCAATG GTTACAAAGA ACAAGACTTG ACCCGTCAAGT TGTTACCTA TCTGAGCGAA |
| 121 | TACTTGGAGA AAGAGGGCCA CACTACCAAG TACTGCCATT GCAATAGCGC GAGCACGGTT |
| 181 | AACGAATCCC TGCGCTATCG TGTGAACAAA GCCAACTCCA TCGGTGTCGA CTACTTCGTG |
| 241 | AGCATCCACC TGAACGCCGG TGGCGGCGTT GGTACCGAAA CGTACATCTG CGCGCGTGGC |
| 301 | GGCGAGGCCG AGCGCGTGGC GAAACGCGTC AATTCTAAAC TGGTGCAGTA CGGTTATCGT |
| 361 | GACCGTGGTG TCAAGGTTGG TAATCTGTAT GTGATTAAGA ACACCAATGC ACCGGCTATC |
| 421 | CTGGTTGAGA TCTGTTTCAT TGACAGCAGC AGCGATGTGG CAAAGTTTAA CGCGAAGGCA |
| 481 | ATCGCGAAAG CGATTGCTGA GGGTCTGCTG GATAAAACCA TTGGTGAAGT CGAGAATAAG |
| 541 | TAA. |

Additionally, two of the three productive prophages found in L. reuteri strain 3632 contain putative lytic enzymes. These lytic enzymes may be used to target pathogenic bacteria or to control environmental spreading of genetically-modified Lactobacillus. These two lytic enzymes are: an N-acetylmuramoyl-L-alanine amidase sle1 precursor from prophage locus 1 with a nucleic acid sequence of:

| (SEQ ID NO: 27) | |
|---|---|
| 1 | ATGCGTAATC AATTCATCGA TGTTTCAAGT TATCAACCAG ATACTGTTGC CTTTTTCCAA |
| 61 | GCTGCTAAAG CTCAGGGTGC ATTAGGGGTC GTTGTTAAGT TAACGGAAGG GTCCGAAGAT |
| 121 | GGTTCGGCTT ATGTTAATCC ACGTGCGGCC GCTCAAATTC GTAATGCCTT AGCGGTTGGC |
| 181 | TTGCGCGTTT CCTGTTACCA CTTTGCTCGT TATACATCAG TGACTGATGC ACAAAATGAA |
| 241 | GCTCGATTCT TCGTTAAAAT CGCTAAGCAA TTTGGTATGT ATGACGATAC TTTGATGATT |
| 301 | GATGATGCGG AAGTTCATTC AACTGCAGAT TATCAATCAG TATCCTTAGC CTTTCTTCAA |
| 361 | GAAGTAGAAG CTCTTGGTTA CAAGAATACT GGGATTTACT CCATGAAGTC CTTCTTCACT |
| 421 | GGCGGTATTC TTAATTCACA TGGCTTTGAT TCCCGGAAGA TTTGGATTGC TGGCTATGGT |
| 481 | GTGACTGAAC TGGGGATTGA TAATGCAAGT GCTTGGCAAT ATTCTGATCA TAGCATCATG |
| 541 | GGAATTGATA CTAGTTATGA CTTTGACGGT GCCTTTACGA CTGGTTTAGT ATCAGGCAAT |
| 601 | GTTCCGCAAG CTGTTATTCC AGCACCACAG CCGGTTCAAC ATATTGGTCA CCCAGCTACT |
| 661 | GGAACCTACA TTGTTCAGCC GGGCGATACA TTGAGTGGAA TTGCAGAAAA ATACGGGACT |
| 721 | ACTTATCAGA ACCTAGCAGC AATCAATGGT ATTGGTAATC CAAACCAGAT CAATGTCGGC |
| 781 | CAAGTCCTCA AAGTCACCGG AAAAGTATCA AACGAAAATA CTTACTTTGT TCAATCAGGC |
| 841 | GATACGTTAT CCGGAATTGC CACCAAATTC GGCACCACTG TCTCAGACCT CGTAAGCCGT |
| 901 | AATCACATTA CTAACCCGAA TGTGATCTAC GTTGGGCAAA AACTCTACTT AGCCGGCAAC |
| 961 | GGACAATCCA ATGCTTATAC TGTCCAAGCA GGGGACACAC TAAGCGGAAT TGCGGCTAAG |
| 1021 | TTTGGCAAGA CCTGGCAAGC ATTAGCTCAA AAGAATGGCA TCGCAAATCC TAATATGATT |
| 1081 | TTCATTGGTC AAACAATTCA GATTTAA, |

and a Peptidase family M23 from prophage locus 6 with a nucleic acid sequence of:

| (SEQ ID NO: 28) | |
|---|---|
| 1 | GTGTACCGAA TTATTGGTTA TAATGAACCA ACAGATAAAG CAGGATTTAT TGTACTGGAT |
| 61 | CCCCGAGTTA ATCGTCATAT TAGTTCGGGA AAACTCACGC TTAAAGAATC TAATATTGAT |
| 121 | GATTTGACTA TTACGGTTAA TCAAGCAAGT CCATTATGGG ACAACGTAAG GCCTTATCAT |
| 181 | ACTCATGTTA ACGTTTATGA TGATAATGAA CTTATTTTTC GTGGACGAGC TATCAAACCT |
| 241 | AAAAAGTCGA TGGAAGAAAG CGGACAATTC ATTCGTGAAT ATGTTTTTGA AGATATTGAA |
| 301 | GCATATCTCA TGGATAGCAC CCAAAGATTT TATGAAGGTG TTGGTCAAAC GCCCAAAGAA |
| 361 | TTTTTACAAA CTTTAATCGA TGTTCATAAT TCACAGGTTC CTGACTATAA AAAGTTTCAA |
| 421 | GTCCGGAATG TAAATGTCAC TAATAATAAG GATGACCAAT ATCGACAAAT TGATTATCCC |
| 481 | AAAACTAGCG ATGCTATTAA TGATAAATTA GTTAAATCTC TTGGTGGTTA TATTGTGACT |
| 541 | ACTTACAACG CTAACGGAAT AAACTACATT GACTACTTAA CGGATATTGG GGTTGATCAT |
| 601 | AAAGATGATA CTCCTATTCA GTTAGCTAAA AATATGAAGT CTGCAAGTAT GCAAATTGAT |
| 661 | CCTACTAAGG TGATTACAAG ACTGATTCCA CTGGGAAAGA CACTAGAACC ATCAAAAGTT |
| 721 | GATGTAAGTG ATGATGATGG AGAGGGCGGT TCTGGATCAT TAGATAGCCC TGAAGAATTT |
| 781 | TGTAAATCAG AAATTAATGC TACTTGGGGT AGTGATATTA ATAATATGAA ACAAGATTTT |
| 841 | GCCGCTCGTT CTTCGAGAGT TCGGGCTTGG GGAGTGGACG TTAATCGTTT ATATGATGTG |
| 901 | GTGAAAAATG CTGGAGTAAG TCCTGAATGG TTCTTTGCTT ATGAACTTCA AGAACAAGGA |
| 961 | ACTTACTATG GATGGCTTAA CCATACTTAT CGACACGGTG ATGCGTATAG TGATGCGCAA |
| 1021 | TCTGTTTGTG AGTGGATTAA AAATTGTTCA AATAGTAATT CCATTAATCC AGCATGGAGC |
| 1081 | GCACCGGAAG GATCAATGGC GCCGAATCAA GCATTAGCGG ATAAATGGAA TCAAGAGTTT |
| 1141 | GGAAAAGGTA CTATTGGCCG CGTTTATTTA CAAGGGACTG CCGCTGCTGT TTGGGATTTA |
| 1201 | GCTGGTCAAA CGCCTAATCC AGCTATTGGA AAGCCAATTA GTGGATGCAT TTCTTGTATT |
| 1261 | AAACGTTGGG GTGGTCATTC TAATGCAGCT GGTGGTACAT GGGGATGGCC TTTTCCTGAT |
| 1321 | GTTGGGGAAG GTCATTTTTC TCAAGTTCAG AGTTTCGGAA ATGATGGCGG ATATCGTCAA |
| 1381 | AATAGTTATC ACGATGGTGT GGATTTTGGA TCAATAGATC ATCCTGGTAG AGAAGTGCAT |
| 1441 | TGTATTCATG GTGGAACGGT AACTATCAAA TCAGCTATGG GTGGCTTAGG TAATTTTGTG |
| 1501 | GTTATTCATA CGCCGGAAGG ATTCAATATC GTTTATCAAG AAGCTTTTAG TTCTCCCTCT |
| 1561 | AATATTATTG TTAGTGTTGG GCAAAAAGTA AAAACTGGTG ATGTAATTGG ATATCGTGAT |
| 1621 | ACAGACCATG TTCATATTGG CGTAACTAAG CAAGATTTTT ATCAAGCAGT TCGAAATTCT |
| 1681 | TTTTCTCCTG CAGGTGGTTG GCTAGATCCA GTAAAACTAA TTAAAGAAGG TGGCGATGGG |
| 1741 | TCTAAACCAC AAGAAGGAAA GAAAGATCAA ACTGTTGATA ATAGTAATGC TGCACGTCCT |
| 1801 | AAATTAACCA TTACTACTGT CAATAACGGT AGAGACTATA TTGATATTCC TGATTTACAA |
| 1861 | AAAGAATTCG GTATTATTGA GGGAACTGTT GAATTTGATA ATGTAGATGA TCCGAATGTT |
| 1921 | TTAATGCAAC AAGCTCAAAC ATGGATAAAG GCTCAAAGAA TACCTCAAAG TTGGGAAGTT |
| 1981 | ACAGCTTTAG AATTACATAT GACAAACTTC AAATCTTTTA AGGTTGCTGA TAGGTACATG |
| 2041 | TTTATTAATC CAAATGTTGC AAAACCCCAA TTATTACGAA TTACTCAAAA AGAAATTGAT |
| 2101 | TTACTAAAGC CCCATGCGTC TTCATTAACG ATTGGTGATA AGACGATGGG GCTTACTGAT |
| 2161 | TATCAGTTAG AAAATCAAGT CAATTTTCAA CAATTTAAGG AAATTCGAGT GATGGTTAAT |
| 2221 | CAGGTTGTCC AAACCCAAGA GCAATCTGCT AATAACAATA ATAAGGTTAT GCAAAATTTT |
| 2281 | GCTAGTAGTG CTGATCTTGC ACAAATGAGA CAGGATCTAA GAAATCTTCA AGATGATAAC |
| 2341 | GATCGTGCTC GCAAAGGAAT GGTTTCCTTA GAAGAATTCA ATAAACTAAA GGAACAAGTA |
| 2401 | GAAAAACTAA CAACAGGAGG CGATGATAAT GGCAAGTGA. |

### Example 7. Single Chain Antibodies and their Activity

An expression cassette would comprise a heterologous coding region encoding a desired biomolecule. The desired biomolecule may be a biomolecule with anti-infective activity. The anti-infective activity could be inhibition or neutralization of toxins produced by pathogens. The inhibition or neutralization could be accomplished with single chain antibodies. Lactobacillus has been described as an expression system for single chain antibodies directed against host attachment factors. WO2012/019054. Toxins to be targeted by single chain antibodies include C. perfringens alpha toxin and NetB.

Camelid heavy-chain only (VHH) antibodies against C. perfringens alpha toxin and NetB are prepared by QVQ Holding BV (Utrecht, NL). Briefly, two llama calves each are immunized with either recombinant alpha toxin or NetB variant W262A. Neither of these immunogens are haemolytic. The immunized llamas are boosted twice with toxin peptides. On days 44 and 72 after the primary immunization, blood samples are taken and RNA isolated for phage library construction. Phage libraries are screened for binding activity towards each of the two toxins. The candidate antibodies are sequenced and further screened in bioassays as described below.

Alpha toxin causes membrane damage to a variety of erythrocytes and cultured cells. It is preferentially active towards phosphatidylcholine (PC or lecithin) and sphingomyelin (SM), two major components of the outer leaflet of eukaryotic cell membranes. The N-terminal domain possesses full activity towards phosphatidylcholine but lacks the sphingomyelinase activity and is not haemolytic or cytotoxic. The C-terminal domain is devoid of enzymatic activity, but interaction between the N- and C-terminal domain is essential to confer sphingomyelinase activity, haemolytic activity and cytotoxicity to the toxin. Although alpha toxin is a potent haemolysin, the lysis of erythrocytes is only seen after intravenous administration of toxin in experimental animals or in cases of clostridial septicaemia.

The inhibitory capacity of the VHH antibodies directed towards alpha toxin on the alpha toxin lecithinase activity is determined by measuring its effect on egg yolk lipoproteins. Fresh egg yolk is centrifuged (10,000×g for 20 min at 4° C.) and diluted 1:10 in PBS. The ability of the VHHs to neutralize the alpha toxin activity is assessed by pre-incubating a two-fold dilution series of the VHHs (two wells per dilution, 5 μM starting concentration) with a constant amount of alpha toxin (either 5 μg/ml recombinant alpha toxin or 3.33×10⁻⁴ U/μl alpha toxin from Sigma, P7633) for 30 minutes at 37° C. prior to the addition of 10% egg yolk emulsion. As a positive control, serum from calves immunized with the recombinant alpha toxin is used, starting from a 1:4 dilution. After incubation at 37° C. for 1 hour, the absorbance at 650 nm (A650) was determined. Alpha toxin activity is indicated by the development of turbidity which results in an increase in absorbance.

The control serum is able to neutralize the lecithinase activity of both the commercial and the recombinant alpha toxin. An eight-fold dilution of the antiserum (corresponding to 3.12% serum) is able to completely neutralize the alpha toxin lectihinase activity of the recombinant alpha toxin, whereas only the highest dilution of the antiserum (corresponding to 25% serum) is able to completely neutralize the lecithinase activity of the commercial alpha toxin. Considerable difference in inhibitory capacity is observed between five candidate VHH antibodies. VHH EAT-1F3 had no effect on the lecithinase activity of either of the alpha toxins. The neutralizing capacity of EAT-1A2 and EAT-1C8 is very similar and is the same for both the recombinant and commercial alpha toxin. The maximal inhibitory capacity is preserved until a 32-fold dilution (0.16 μM VHH) of the VHHs. However, both EAT-1A2 and EAT-1C8 are unable to completely neutralize the lecithinase activity, resulting in 40% to 50% residual lecithinase activity. Two other VHHs, EAT-1F2 and EAT-1G4 show a difference in neutralizing capacity towards the recombinant and the commercial alpha toxin. EAT-1F2 has a high neutralizing capacity towards the recombinant alpha toxin but is unable to completely neutralize the commercial alpha toxin, resulting in about 25% residual lecithinase activity. In contrast to EAT-1F2, EAT-1G4 neutralizes 100% of the lecithinase activity of the commercial alpha toxin, but is less capable of neutralizing the recombinant alpha toxin.

Neutralization of the alpha toxin haemolytic activity by the VHH antibodies directed towards alpha toxin is determined by measuring its effect on sheep erythrocytes. Similar to the inhibition of the alpha toxin lecithinase activity, the ability to neutralize the haemolytic activity is assessed by pre-incubating a two-fold dilution series of the VHH antibodies (two wells per dilution, 5 μM starting concentration) with a constant amount of alpha toxin (6.25×10⁻⁵ U/μl alpha toxin from Sigma, P7633) for 30 minutes at 37° C. prior to the addition of 1% sheep erythrocytes. As a positive control, serum from calves immunized with the recombinant alpha toxin is used, starting from a 1:4 dilution. After incubation at 37° C. for 1 hour, the plates are centrifuged to pellet intact red blood cells. The supernatant is transferred to a new 96 well plate and the Asso is determined. Alpha toxin activity is indicated by the increase in absorbance due to release of haemoglobin from the erythrocytes.

The inhibitory capacity of the VHH antibodies towards the alpha toxin haemolytic activity is determined using the commercial alpha toxin only, as the recombinant alpha toxin shows no haemolytic activity. Up to a 16-fold dilution of the control serum (corresponding to 1.56% serum) completely inhibits the alpha toxin haemolysis. To the contrary, none of the candidate VHHs has an effect on the haemolytic activity of alpha toxin. Because the control serum contains polyclonal antibodies, whereas the VHHs are monoclonal, the combined effect of all 5 VHHs towards alpha toxin is determined (1 μM of each VHH in the highest dilution, corresponding to 5 μM VHHs in total). Combining the VHHs has no effect on the alpha toxin haemolysis.

Based on the above results, EAT-1F2 and EAT-1G4 are selected for further characterization. The peptide sequence of EAT-1F2 is: EVOLVESGGGLVQAGGSLRLSCAGSGRTGSLYSMGWFRQAPGKEREFVAAITW RPSSTYYADSVKGRFTISRDDAKNTVYLQMNSLKPEDTAVYFCAARPRGGLSPTP QAYDYWGQGTQVTVSSAAASGSLEQKLISEEDLNGAAHHHHHHGAA (SEQ ID NO: 29), and the peptide sequence of EAT-1G4 is: EVOLVESGGGLVQPGGSLRLSCAASGSIATINDMGWFRQAPGKQRDWVATIVSD GSTAYADSVKGRFTISRDNAKNTVYLQMNSLKPEDTAVYYCSARRHYGQGTQV TVSSAAASGSLEQKLISEEDLNGAAHHHHHHGAA (SEQ ID NO: 30).

An R27H mutant of EAT-IF2 (SEQ ID NO: 49) may also be generated to improve protease resistance of the antibody.

A person of skill in the art would recognize that, because of the redundancy of the genetic code, multiple nucleic acid sequences could encode the above peptides. However, an exemplary sequence encoding EAT-1F2 could be:

| (SEQ ID NO: 31) | |
|---|---|
| 1 | GAGGTGCAGC TCGTGGAAAG TGGCGGAGGT CTTGTTCAGG CTGGGGGATC GCTCCGTCTG |
| 61 | AGCTGTGCGG GGTCTGGCAG AACAGGTAGT CTCTATTCCA TGGGTTGGTT TCGGCAGGCC |
| 121 | CCGGGTAAGG AGCGGGAGTT CGTTGCAGCG ATTACGTGGA GGCCCAGCTC TACCTACTAC |
| 181 | GCGGACAGCG TAAAGGGACG ATTCACCATT AGTAGAGACG ACGCAAAGAA TACTGTATAT |
| 241 | TTGCAGATGA ATTCGTTGAA GCCTGAGGAC ACCGCTGTCT ATTTTTGCGC GGCGCGACCG |
| 301 | AGGGGCGGTC TCTCCCCGAC ACCTCAAGCA TATGATTACT GGGGACAAGG GACCCAAGTC |
| 361 | ACTGTATCCA GTGCGGCCGC GAGCGGCAGC CTTGAACAAA AGCTGATAAG CGAGGAGGAT |
| 421 | CTCAATGGTG CTGCACATCA TCATCACCAT CACGGGGCAG CG, |

and an exemplary sequence encoding EAT-1G4 could be:

| (SEQ ID NO: 32) | |
|---|---|
| 1 | GAAGTTCAGC TTGTAGAGTC CGGTGGGGGT CTTGTACAGC CCGGCGGGAG CTTGCGACTC |
| 61 | TCATGCGCTG CTTCCGGAAG CATTGCGACA ATAAATGATA TGGGTTGGTT TAGACAAGCC |
| 121 | CCCGGGAAGC AGCGTGACTG GGTCGCGACT ATTGTGAGTG ACGGCAGCAC GGCTTATGCG |
| 181 | GACTCAGTGA AAGGGAGATT TACGATTTCG CGAGATAACG CGAAAAACAC TGTATACCTG |
| 241 | CAGATGAATT CACTCAAGCC GGAAGATACA GCTGTGTATT ATTGTTCTGC CCGACGGCAC |
| 301 | TACGGACAGG GGACCCAGGT CACAGTCTCG AGCGCTGCCG CCAGTGGGTC ACTCGAGCAG |
| 361 | AAGCTGATAT CAGAGGAGGA CCTTAACGGT GCGGCGCACC ATCACCACCA TCATGGTGCG |
| 421 | GCG. |

NetB is a heptameric beta-pore-forming toxin that forms single channels in planar phospholipid bilayers. The NetB activity is influenced by membrane fluidity and by cholesterol, which enhances the oligomerization of NetB and plays an important role in pore formation. NetB has high haemolytic activity towards avian red blood cells.

Neutralization of the NetB haemolytic activity by camelid VHH antibodies directed towards NetB is determined by measuring NetB-mediated lysis of chicken erythrocytes. The ability to neutralize the NetB haemolytic activity is assessed by pre-incubating a two-fold dilution series of the VHH antibodies (two wells per dilution, 5 μM starting concentration) with a constant amount of NetB toxin (20 μg recombinant NetB) for 30 minutes at 37° C. prior to the addition of 1% chicken erythrocytes. The non-toxic NetB variant W262A is included as a negative control as this variant displays no haemolytic activity. Positive control serum from rabbits immunized with the recombinant NetB (wild type NetB) is used, starting from a 1:4 dilution. After incubation at 37° C. for 1 hour, the plates are centrifuged to pellet intact red blood cells. The supernatants are transferred to a new 96 well plate and the Asso is determined. NetB activity is indicated by the increase in absorbance due to release of haemoglobin from the erythrocytes.

The control serum is able to neutralize the haemolytic activity of NetB. VHH antibodies ENB-1F4 and ENB-1F10 have no effect on the NetB haemolysis. ENB-1B9 has intermediate inhibitory capacity, while ENB-1D11 and ENB-1A4 are able to neutralize the NetB haemolysis up to a 4- to 8-fold dilution (1.25 μM-0.625 μM VHHs).

Based on the above results, ENB-1A4 and ENB-1D11 are selected for further characterization. The peptide sequence of ENB-1A4 is: EVOLVESGGGLVQAGGSLRLSCAASGSIFSTNVMGWYRQAPGKQREFVAGITIG GTARYPDSVKGRFTISRDNTQNTVYLQMNNLKPEDTAVYYCNAVLPSDQRRWS WGQGTQVTVSSAAASGSLEQKLISEEDLNGAAHHHHHHGAA (SEQ ID NO: 33), and the peptide sequence of ENB-1D11 is: EVQLVESGGGLVQTGGSLRLSCTASGTIDMTYGLIWYRQAPGKERELVASIRRDG RTNYADSVKGRFTISIDNAKNSIHLQMNSLKPDDTARYYCNSPYHAL WGQGTQV TVSSAAASGSLEQKLISEEDLNGAAHHHHHHGAA (SEQ ID NO: 34).

An R56H mutant of ENB-1D1 1 (SEQ ID NO: 50) may also be generated to improve protease resistance of the antibody.

A person of skill in the art would recognize that, because of the redundancy of the genetic code, multiple nucleic acid sequences could encode the above peptides. However, an exemplary sequence encoding ENB-1A4 could be:

| (SEQ ID NO: 35) | |
|---|---|
| 1 | GAGGTACAAC TGGTTGAGAG TGGGGGTGGT TTGGTGCAAG CCGGAGGTTC CTTACGTTTG |
| 61 | TCTTGCGCGG CTAGTGGGAG CATCTTTTCA ACAAACGTAA TGGGGTGGTA CCGCCAAGCC |
| 121 | CCAGGTAAGC AGCGGGAATT TGTGGCCGGG ATAACGATCG GAGGAACTGC GAGGTATCCT |
| 181 | GATAGTGTGA AAGGGCGTTT CACAATTAGT CGAGATAATA CACAGAATAC TGTCTATCTC |
| 241 | CAAATGAATA ATCTCAAGCC CGAAGACACA GCAGTTTATT ATTGTAATGC CGTTCTCCCC |
| 301 | TCTGATCAGC GTCGATGGAG CTGGGGACAA GGCACCCAGG TTACGGTTAG CAGCGCGGCA |
| 361 | GCGTCTGGTT CGCTCGAGCA AAAGCTCATA TCTGAGGAGG ACCTGAACGG GGCAGCCCAC |
| 421 | CATCACCACC ATCACGGAGC AGCT, |

and an exemplary sequence encoding ENB-1G4 could be:

| (SEQ ID NO: 36) | |
|---|---|
| 1 | GAGGTACAGC TGGTGGAGTC CGGCGGTGGT TTGGTGCAAA CCGGGGGTAG TCTGCGGCTT |
| 61 | AGTTGCACGG CGTCTGGGAC AATAGACATG ACTTATGGTC TCATATGGTA CAGGCAAGCG |
| 121 | CCTGGGAAAG AGAGGGAACT CGTTGCGAGT ATCAGAAGGG ACGGCCGCAC AAATTACGCT |
| 181 | GATTCAGTGA AAGGGCGCTT CACTATCTCG ATCGATAATG CGAAAAACAG TATTCACCTT |
| 241 | CAAATGAACT CCCTTAAGCC CGATGATACC GCCAGGTATT ATTGCAACAG CCCATATCAC |
| 301 | GCACTTTGGG GTCAGGGTAC GCAGGTAACA GTGTCTAGTG CGGCAGCCTC TGGTAGTTTG |
| 361 | GAGCAAAAGT TGATAAGTGA GGAGGACTTA AATGGGGCGG CACATCACCA CCACCATCAT |
| 421 | GGGGCGGCT. |

### Example 8 Multifactorial Expression Cassettes

Expression cassettes may be designed and synthesized with different combination of the following components: promoter for expression, signal sequence for secretion, cell-wall anchor for secretion, at least one heterologous coding region encoding a desired biomolecule, and terminators for transcription termination. An expression cassette may contain multiple heterologous coding regions.

One representative expression cassette for the expression of camelid antibodies is:

| (SEQ ID NO: 37) | |
|---|---|
| 1 | AAATTAAAAG GCTGGATTTT TTCGGCCTTT TTTTAGTGCA AATAATTATT TTTTACGTAT |
| 61 | TTATATTATA GGGCTAATCA CTAAACTAAT AATTAGTGGT TGAAGCGCTG AAAATTTTCT |
| 121 | GCTATTTTAT TAATAGTTTG ATAATAAAAT AATGATATTT AATATAAAGA GGGATAAACG |
| 181 | AAATAATGAA ATCAACAACA AAGAAAATTC TTGCATCGTC GTTAGGGGTA GCTGGCGCAA |
| 241 | TGGCAATGGG CACGGTAACT GCAAAGGCTG ATACGACCGT TACGGTCAAT GCTGGCGATA |
| 301 | GTTTGAATGG GATTGCTCAA AAGTATAATG TTAGTGCGGA TGATATTGCA ACCGCTAATC |
| 361 | ACTTGCAAAA TAAAGAGTTG ATTTTTGTGG GACAAAAGTT GACAATTCCA ACCAAAGATA |
| 421 | AAAATGAAAC AGAAGTTCAA TTAGTTGAAA GTGGTGGTGG TTTAGTTCAA CCAGGTGGTA |
| 481 | GTTTACGTTT ATCATGTGCT GCAAGTGGTT CAATTGCAAC TATTAATGAT ATGGGTTGGT |
| 541 | TTCGTCAAGC ACCAGGAAAG CAACGTGATT GGGTTGCTAC TATTGTTAGT GATGGTTCAA |
| 601 | CTGCTTATGC TGATAGTGTT AAAGGTCGTT TTACTATTTC ACGTGATAAT GCTAAGAATA |
| 661 | CTGTTTACCT TCAAATGAAT AGTCTTAAGC CAGAAGATAC TGCAGTTTAC TATTGTTCAG |
| 721 | CTCGTCGTCA TTATGGTCAA GGTACTCAAG TTACTGTTAG TTCAGCTGCA GCTAGTGGTT |
| 781 | CATTAGAACA AAAATTAATT TCAGAAGAAG ATTTAAATGG TGCAGCTCAT CATCATCATC |
| 841 | ATCATGGTGC AGCTTACTTC CCTGGAAATT ACTATGTTGT GATGCATTAA TATTCCCTTT |
| 901 | CACCTCACCT TTAATAATTT AAATTAGTAA TTATCTTGCG CATCACAAAA GAGTGCTATA |
| 961 | TACTATTTCA GATTAGAAAG TTTTATGAGG GAGACAAATT G; |

where nucleotides 1 to 185 represent a promoter sequence from the Cwls endopeptidase (SEQ ID NO: 41), nucleotides 186 to 262 encode the Cwls endopeptidase secretion signal (SEQ ID NO: 23), nucleotides 263 to 407 encode a LysM domain for attachment to extracellular polysaccharides, nucleotides 408 to 431 are a spacer sequence, nucleotides 432 to 854 encode VHH EAT-1G4 (SEQ ID NO: 30) and nucleotides 855 to 890 are another spacer sequence ending in a stop codon for translation termination.

A second representative expression cassette for the expression of camelid antibodies is:

| (SEQ ID NO: 38) | |
|---|---|
| 1 | GAAGTACAAA GTTACTTTAA CTATAATGAA AAACAAGACA ATATAAAGAA AACAACATAT |
| 61 | AAGGTTCAGT TCATAACTGA TTAGATTTAT AATAAATATT GTAAATCGGA CAAAAATAAA |
| 121 | TTAATTTTCA ATTAATTCAA AAAAACCATA TTTTTTTCGT TTTGGCATAT TTGGATTTGC |
| 181 | TACACTAAAG ATGATCAAGA AAGGGGAAAA GATAATCTTC AATCTTGTGT ACTTAGTTTG |
| 241 | TTAATTAATT TATAAATTTA GGGAGGAAAC CTATCATGGA AAAGACTATG AAAAAGAAAG |
| 301 | CTTTAGTTGC AACTACTGCT GTAGCCGGTA TTACTTTAGT AGGAGAGGTT ACTACTGTTC |
| 361 | ATGCCGCTGA CAATGTACAA CAAGAAGTTC AATTAGTTGA AAGTGGTGGT GGTTTAGTTC |
| 421 | AACCAGGTGG TAGTTTACGT TTATCATGTG CTGCAAGTGG TTCAATTGCA ACTATTAATG |
| 481 | ATATGGGTTG GTTTCGTCAA GCACCAGGAA AGCAACGTGA TTGGGTTGCT ACTATTGTTA |
| 541 | GTGATGGTTC AACTGCTTAT GCTGATAGTG TTAAAGGTCG TTTTACTATT TCACGTGATA |
| 601 | ATGCTAAGAA TACTGTTTAC CTTCAAATGA ATAGTCTTAA GCCAGAAGAT ACTGCAGTTT |
| 661 | ACTATTGTTC AGCTCGTCGT CATTATGGTC AAGGTACTCA AGTTACTGTT AGTTCAGCTG |
| 721 | CAGCTAGTGG TTCATTAGAA CAAAAATTAA TTTCAGAAGA AGATTTAAAT GGTGCAGCTC |
| 781 | ATCATCATCA TCATCATGGT GCAGCTAAGA AGATTACAGA AGATCAAAAA GCAACAGAAG |
| 841 | GCGGTTATCA TATTGTTAAT GGATCTGTTG TAGATAAGCA GAATAACTTG GTTAGTGGTT |
| 901 | GGACTGTTAA GAATGGTCAA ATGGTTGATC CTGAAGGTAA CGTTATCAAA ACAACAATGT |
| 961 | CTACAGCCCA AGGTGTTACT ATTGAAAAAA ATAATAGCAA GTCCGGGAAT ACAAAGACAA |
| 1021 | ACATGATTCA AACTTCTTTA ACTATTGCTA ACAACAAGGC AACAACAAAC AAAGACAACC |
| 1081 | AGTTACCACA AACTGGCAAT TACAACAACA ATACAAAGGT ATTAGGATTA GCTGGTATTG |
| 1141 | CACTTGCATC TGCTTTAACT ATGTTTGGAT ACAAGAAGCG CCAACATAAC TAATTTTCTT |
| 1201 | ACTTGATGGG TTTCTAAATA AAAAATGGAC TACTTCAGCT CAAGGTAGCC CATTTTTATT |
| 1261 | ATTATAGTGA AGCAGTTATT CTTACGTATA ACCAGACTAA AATATAATAA AAATCTATTA |
| 1321 | TATATTAATC AACATCTCGG TTTAATCGTT AAAACTCCTC TGAGAGCTAA TTGTTAATAT |
| 1381 | TGAGTTGTAT AGT, |

where nucleotides 1 to 275 represent a promoter sequence from chromosome segregation protein (SEQ ID NO: 42), nucleotides 276 to 383 encode a secretion signal from the chromosome segregation protein (SEQ ID NO: 22), nucleotides 384 to 806 encode VHH EAT-1G4 (SEQ ID NO: 30), nucleotides 807 to 1190 encode the cell wall anchor sequence from the chromosome segregation protein (SEQ ID NO: 22), and nucleotides 1191 to 1193 are a stop codon for translation termination.

A third representative expression cassette for the expression of camelid antibodies is:

| (SEQ ID NO: 39) | |
|---|---|
| 1 | GCAATGCACA AGATGCTGAA ACAAAGGCAC AACAAAATGC AGATCAAGCT TCACCAGCTA |
| 61 | ATATTCAAAA GGCACAAGAT GCTATTGCTA ATCAAGAAAC TCAAATTAGT AAAGACACCG |
| 121 | ATGCTATTAA TGACGCTAAC AAAGCCGTTA GCGATGCACA AAGCACAGTT GATGCAGCGC |
| 181 | AAAAAAAGTT AATGATGCAA CTACTGCTCG TGACAATCAA CAAAAGAATG TTGATACTGC |
| 241 | TAGTGATGCA GTTAAGAATG CTCAAGCTAT TCTTGACAAC AGTGATCAGG CTAAAAAGGA |
| 301 | AGCCCAAGAT GCTTTGAACA AGGCTAACCA AAAAGTTGCT GATGATACTA CTGCCGTTAA |
| 361 | CAACAAACAA ACTGATGTTA ACAATGCAGC AGAAGCTAAG AAGAATGCAG ATGAGGCATT |
| 421 | GAAGAACGCC AATGATGCGC AAACTTCTGC ACAAAAGAAT AAAGATGCTA AGCAAGCAAT |
| 481 | TGCTGATGAG GCAAGTGTAG AAGTTCAATT AGTTGAAAGT GGTGGTGGTT TAGTTCAACC |
| 541 | AGGTGGTAGT TTACGTTTAT CATGTGCTGC AAGTGGTTCA ATTGCAACTA TTAATGATAT |
| 601 | GGGTTGGTTT CGTCAAGCAC CAGGAAAGCA ACGTGATTGG GTTGCTACTA TTGTTAGTGA |
| 661 | TGGTTCAACT GCTTATGCTG ATAGTGTTAA AGGTCGTTTT ACTATTTCAC GTGATAATGC |
| 721 | TAAGAATACT GTTTACCTTC AAATGAATAG TCTTAAGCCA GAAGATACTG CAGTTTACTA |
| 781 | TTGTTCAGCT CGTCGTCATT ATGGTCAAGG TACTCAAGTT ACTGTTAGTT CAGCTGCAGC |
| 841 | TAGTGGTTCA TTAGAACAAA AATTAATTTC AGAAGAAGAT TTAAATGGTG CAGCTCATCA |
| 901 | TCATCATCAT CATGGTGCAG CTGCTGAAAA GGAAGCTAAA GATAATGGCT ACCATATCGA |
| 961 | AAATAACCAA GTTGTTGACG CTAAAGGTAA TAGTGTCAAT GGCTGGACAG TTAAGGGCAA |
| 1021 | CCAAATTGTT AGTCCAACTA ATGCTACTGT AGATCCCGCT GTTTCTGTAA CCACCAATGT |
| 1081 | CAATGTTGAT AGTAAAGGTC AAGTACAACC ACAAACTAGT GTTACTGCTA ATAGTGTTAA |
| 1141 | GACTGTAGCT GCAACTGAAT CAGCAAATCC AGTAGCAACT ACTACTGTGC AAACCCGCGA |
| 1201 | ACAATACAAG CAACAATTGA AGAGCAATAA TCAATTACCA CAAACTGGTA ATAATGATAG |
| 1261 | TGCTGTTCTT TCACTTGCTG GAGTAGCACT TGCAGCAATG TTGAGTTTGT TCGGTATTAA |
| 1321 | GAAACGTGAA TACTAATTTA GAAAATGTAA GTATTATTAT GTAAAAAGGT TCAACCAAAT |
| 1381 | TGGCTGAACC TTTTTGTCTA AAATTTAAGG AGAAGTTTT, |

where nucleotides 1 to 313 represent a promoter sequence from Chromosome Partition Protein Smc (SEQ ID NO: 43), nucleotides 314 to 499 encode a secretion signal from Chromosome Partition Protein Smc (SEQ ID NO: 40), nucleotides 500 to 922 encode VHH EAT-1G4 (SEQ ID NO: 30), nucleotides 923 to 1333 encode the cell wall anchor sequence from Chromosome Partition Protein Smc (SEQ ID NO: 40), and nucleotides 1334 to 1336 are a stop codon for translation termination.

Many other combinations of the disclosed expression cassette elements are also possible.

### Example 9: Anti-Infective Molecules Derived from Bacillus

Certain Bacillus strains may also be used as DFM, for example for poultry. Over one hundred Bacillus isolates are collected from chicken cecum. The chickens are sourced with the state of Indiana, USA Isolated strains are selected to withstand 100° C. for 10 minutes for development as pellet-stable products. The isolated strains are confirmed to be Bacillus using 16S rRNA sequencing. The isolates are tested for antimicrobial activity against various poultry pathogens such as Salmonella Enteritidis, avian pathogenic E. coli, Clostridium perfringens, Enterococcus cecorum and Campylobacter jejuni. Different isolates display different antimicrobial activities against these pathogens. Of all the isolates, five isolates are cytotoxic for all the pathogens tested. All 5 isolates are isolated from samples collected from the same study, suggesting that they may potentially be clonal.—Two isolates, D24 and D72, are selected for further characterization.

The two selected isolates (D24 and D72) are sequenced by ILLUMINA® sequencing and determined to most likely be Bacillus velezensis. The genomic sequences are analyzed for potential bacteriocins, antimicrobial peptides and digestive enzymes. The isolates contain several full loci potentially encoding for antimicrobial peptides and bacteriocins. Considering the feasibility of engineering these antimicrobial peptides and bacteriocins into an L. reuteri expression system, five ribosomal bacteriocins are selected for further study. Based on the sequence homology, the five Bacillus bacteriocins are identified to be
Antimicrobial peptide LCI: AIKPVPSPNGIF AASFELNGTTWIFKYKYYDSSKGYWVGIYESVDK (SEQ ID NO: 44); Circularin A: LASTLGISTAAAKKAIDIIDAASTIASIISLIGIVTGAGAISYAIVATAKTMIKKYGKK YAAAW (SEQ ID NO: 45); Lanthipeptide 3 (Plantaricin C): EFSGGGGAEQRGISQGNDGKLCTLTWECGLCPTHTCWC (SEQ ID NO: 46); Lanthipeptide 5 (Lichenicidin A2): SDATPMTVTPTTITIPISLAGCPTTKCASIVSPCN (SEQ ID NO: 47); and Lanthipeptide 6: SEATPMTVTPTTITIPISLAGCPTTKCASIVSPCND (SEQ ID NO: 47). These bacteriocins have been previously shown to kill various Gram-negative and Gram-positive pathogens.

These Bacillus-derived anti-infective peptides may be incorporated into an expression cassette and expressed recombinantly. Recombinant Bacillus-derived anti-infective peptides may be expressed alone or (without limitation) one or more of the other anti-infective peptides or phage disclosed in Example 2, the lytic enzymes disclosed in Example 6, or antibodies such as those disclosed in Example 7.

### Example 10: In Vivo Effects of L. reuteri

An identified strain can effectively function as a direct feed microbial only if the strain is able colonize the host gastrointestinal tract. The ability of L. reuteri strains to colonize chickens is assessed following different routes of administration.

L. reuteri is administered orally to IO-day old chicks. At various times post inoculation, chicks are sacrificed and the presence of L. reuteri is measured in the crop, small intestine, and cecum. Strain 3632 is able to colonize all three tissues and remain detectable for at least 18 days.

L. reuteri is administered by in ovo inoculation (i.e. injection into the egg) 3 days before hatching. Strain 3632 is found in the tissues of chicks at 3 and 7 days post hatching, while colonization of a control strain declines after day 3.

L. reuteri is administered in ovo by spraying a liquid containing bacteria onto the egg surface. Again, Strains 3630 and 3632 are able to colonize and remain present in chicks 3 and 7 days post hatching. Inoculation by spray is just as efficient as in ovo injection but requires less manipulation of the incubating egg.

Recombinants strains 3630 and 3632 carrying expression cassettes comprising one of the antibodies of Example 7 or the PlyCM lytic enzyme are administered by in ovo inoculation. The recombinant strains are able to efficiently colonize chicken gastrointestinal tracts at comparable levels to unmodified parental strains. Thus, genetic manipulation as contemplated herein does not alter the ability of the modified strains to act as DFM.

Chicks carrying L. reuteri strains or modified strains are assessed for their susceptibility to necrotic enteritis. Chicks are inoculated with L. reuteri live expression systems wherein the expression cassettes contain either a single chain antibody or a lytic enzyme. Inoculation is done orally by providing chicks drinking water containing 10⁸ recombinant L. reuteri.

An experiment is performed as given in Table 2, with 40 one-day-old chicks placed in each of ten groups. Group 1 chicks served as the untreated and unchallenged controls. Group 2 chicks served as untreated challenge controls. Group 3 represents challenged chicks given standard antibiotic therapy. Groups 2-10 each received 2×10⁸ CFU of C. perfringens on each of days 17 and 18. Groups 4-9 each received prophylactic inoculation with L. reuteri continuously from day 1. Group 10 received only therapeutic doses of L. reuteri on days 18-21. On day 21 chicks are sacrificed and small 15 intestinal tissues are examined for the presence of lesions. Survival of chicks within each treatment group is also recorded.

As shown in Table 2, the four antibodies can each ameliorate disease to some degree. A combination of strains may be more effective than any single strain.

TABLE 2
Antibodies produced by recombinant L. reuteri can treat necrotic enteritis.

| | | | | Lesion | Reduction | Mortality | Reduction in |
|---|---|---|---|---|---|---|---|
| Group | Strain | Ab | Challenge | Score (avg) | in lesions | (avg) | mortality |
| 1 | none | none | none | 0 | n/a | 0 | n/a |
| 2 | none | none | Yes | 1.80 | n/a | 27.5 | n/a |
| 3 | none | none | Yes^{a} | 0.375 | 79.17% | 2.5 | 90.91% |
| 4 | 3630 | 1G4 | Yes | 1.35 | 25.00% | 22.5 | 18.18% |
| | | 1F2 | | | | | |
| | | 1D11 | | | | | |
| | | 1A4 | | | | | |
| 5 | 3630 | 1G4 | Yes | 0.5 | 72.22% | 10 | 63.64% |
| 6 | 3630 | 1F2 | Yes | 0.95 | 47.22% | 20 | 27.27% |
| 7 | 3630 | 1D11 | Yes | 1.25 | 30.56% | 25 | 9.09% |
| 8 | 3630 | 1A4 | Yes | 1.45 | 19.44% | 22.5 | 18.18% |
| 9 | 3630 | 1D11 | Yes | 0.675 | 62.50% | 0 | 100.0% |
| | 3632 | 1D11 | | | | | |
| 10 | 3630 | 1G4 | Yes | 1.275 | 29.17% | 17.5 | 36.36% |
| | | 1F2 | | | | | |
| | | 1D11 | | | | | |
| | | 1A4 | | | | | |

^{a}Treated with BMD. Bacitracin methylene disalicylate

1.-42. (canceled)

43. An antibody that binds a toxin produced by Clostridium perfringens, wherein the antibody comprises an amino acid sequence selected from the group consisting of SEQ ID NO: 29, SEQ ID NO: 49, SEQ ID NO: 30, SEQ ID NO: 33, SEQ ID NO: 34, and SEQ ID NO: 50.

44. The antibody of claim 43, wherein the antibody binds Clostridium perfringes alpha toxin, wherein the antibody is selected from SEQ ID NO: 29, SEQ ID NO: 30, and SEQ ID NO: 49.

45. (canceled)

46. (canceled)

47. (canceled)

48. The antibody of claim 44, wherein the antibody neutralizes alpha toxin activity.

49. The antibody of claim 43, wherein the antibody binds Clostridium perfringes NetB, wherein the antibody is selected from SEQ ID NO: 33, SEQ ID NO: 34, and SEQ ID NO: 50.

50. The antibody of claim 49, wherein the antibody neutralizes NetB haemolytic activity.

51. The antibody of claim 43, wherein the antibody is a single chain antibody.

52. A method of reducing colonization of an animal by a C. perfringes bacterium, the method comprising administering to an animal one or more antibody of claim 43.

53. The method of claim 52, wherein the animal is a bird, a human, or a non-human animal.

54. A method of reducing colonization of an animal by a C. perfringes bacterium, the method comprising administering to an animal a genetically-modified microorganism expressing one or more antibody of claim 43, wherein the genetically-modified microorganism is a bacterium selected from the group consisting of Bacillus, Lactobacillus, Lactococcus, and Entercoccus.

55. The method of claim 54, wherein the genetically-modified microorganism is a Lactobacillus reuteri strain.

56. The method of claim 55, wherein the Lactobacillus reuteri strain is selected from strain 3632 ATCC PTA-126788 and strain 3630 ATCC PTA-126787.

57. The method of claim 54, wherein the animal is a bird, a human, or a non-human animal.
